(19) United States
(12) Patent Application Publication
(10) Pub. No.: US 2024/0197861 A1
(43) Pub. Date: Jun. 20, 2024

(54) RECOMBINANT CHIMERIC BOVINE/HUMAN PARAINFLUENZA VIRUS 3 EXPRESSING SARS-COV-2 SPIKE PROTEIN AND ITS USE

(71) Applicant: The U.S.A., as represented by the Secretary, Department of Health and Human Services, Bethesda, MD (US)

(72) Inventors: Ursula J. Buchholz, Silver Spring, MD (US); Shirin Munir, Potomac, MD (US); Cyril Le Nouen, Bethesda, MD (US); Xueqiao Liu, Bethesda, MD (US); Cindy Luongo, Bethesda, MD (US); Peter L. Collins, Silver Spring, MD (US)

(73) Assignee: The U.S.A., as represented by the Secretary, Department of Health and Human Services, Bethesda, MD (US)

(21) Appl. No.: 18/555,137

(22) PCT Filed: Apr. 27, 2022
(86) PCT No.: PCT/US2022/026576

§ 371 Date: Oct. 12, 2023

Related U.S. Application Data
(60) Provisional application No. 63/180,534, filed on Apr. 27, 2021.

(51) Int. Cl. A61K 39/215; A61K 39/00; A61K 39/155; A61P 31/14; A61P 37/04; C07K 14/005; C12N 15/86

(52) U.S. Cl. CPC A61K 39/215; A61K 39/155; A61P 31/14; A61P 37/04; C07K 14/005; C12N 15/86; A61K 2039/5256; A61K 2039/543; A61K 2039/55; C12N 2760/18622; C12N 2760/18634; C12N 2760/18644; C12N 2760/18671; C12N 2770/20022; C12N 2770/20034; C12N 2770/20071

(57) ABSTRACT

Recombinant chimeric bovine/human parainfluenza virus 3 (rB/HPIV3) vectors expressing a recombinant Severe Acute Respiratory Syndrome Coronavirus 2 (SARS-CoV-2) Spike (S) protein as well as methods of their use and manufacture, are provided. The rB/HPIV3 vector comprises a genome comprising a heterologous gene encoding the recombinant SARS-COV-2 S protein. Nucleic acid molecules comprising the sequence of the genome or antigenome of the disclosed rB/HPIV3 vectors are also provided. The disclosed rB/HPIV3 vectors can be used, for example, to induce an immune response to SARS-COV-2 and HPIV3 in a subject.

### CROSS REFERENCE TO RELATED APPLICATIONS

This application claims the benefit of U.S. Provisional Application No. 63/180,534, filed Apr. 27, 2021, which is herein incorporated by reference in its entirety.

### FIELD

This relates to recombinant chimeric bovine/human parainfluenza virus 3 (rB/HPIV3) vectors expressing a recombinant Severe Acute Respiratory Syndrome Coronavirus 2 (SARS-COV-2) Spike (S) protein, and use of the rB/HPIV3 vector, for example, to induce an immune response to SARS-COV-2 S and HPIV3 in a subject.

### BACKGROUND

Coronaviruses are enveloped, positive-sense single-stranded RNA viruses. They have the largest genomes (26-32 kb) among known RNA viruses, and are phylogenetically divided into four genera (α, β, γ, δ), with betacoronaviruses further subdivided into four lineages (A, B, C, D). Coronaviruses infect a wide range of avian and mammalian species, including humans.

In 2019, a novel coronavirus (designated SARS-COV-2 by the World Health Organization) was identified as the causative agent of a coronavirus pandemic that appears to have originated in Wuhan, China. The high case-fatality rate, vaguely defined epidemiology, and absence of prophylactic or therapeutic measures against coronaviruses have created an urgent need for an effective vaccine and related therapeutic agents. As of January 2021, SARS-COV-2 had infected more than 84 million people worldwide, leading to nearly 2 million deaths.

Parainfluenza viruses (PIV) are enveloped non-segmented negative-strand RNA viruses that belong to the family Paramyxoviridae. PIVs include members of the genus Respirovirus [including the species Human respirovirus 1 and 3 (PIV1, PIV3) and Murine respirovirus (Sendai virus)] and the genus Rubulavirus [including the species Human orthorubulavirus 2, 4 and Mammalian orthorubulavirus 5 (PIV2, PIV4, PIV5)]. The human parainfluenza viruses (HPIVs, serotypes 1, 2, and 3) are second only to RSV in causing severe respiratory disease in infants and children worldwide, with HPIV3 being the most relevant of the HPIVs in terms of disease impact. The HPIV3 genome is approximately 15.5 kb, with a gene order of 3′-N-P-M-F-HN-L. Each gene encodes a separate mRNA that encodes a major protein: N, nucleoprotein; P, phosphoprotein; M, matrix protein; F, fusion glycoprotein; HN, hemagglutinin-neuraminidase glycoprotein; L, large polymerase protein, with the P gene containing additional open reading frames encoding the accessory C and V proteins. Development of an effective HPIV vaccine remains elusive.

Major challenges to developing pediatric vaccines against SARS-COV-2 and HPIV3 include the immaturity of the immune system during infancy, immune-suppression by maternal antibodies, and inefficient immune protection at the superficial epithelium of the respiratory tract.

Vaccines for SARS-COV-2 are increasingly available under emergency use authorizations; however, they involve parenteral immunization, which does not directly stimulate local immunity in the respiratory tract, the primary site of SARS-COV-2 infection and shedding. While the major burden of COVID-19 disease is in adults, infants and young children also experience infections and disease, and contribute to viral spread, especially as highly transmissible variants are emerging. Therefore, the development of safe and effective pediatric COVID-19 vaccines is important. Ideally, a vaccine should be effective at a single dose, and should induce mucosal immunity with the ability to restrict SARS-COV-2 infection and respiratory shedding and should easily coordinate with vaccines for other illnesses, such as HPIV3.

### SUMMARY

Recombinant chimeric bovine/human parainfluenza virus 3 (rB/HPIV3) vectors expressing recombinant SARS-COV-2 S protein (“rB/HPIV3-SARS-COV-2/S” vectors) are provided herein. The disclosed rB/HPIV3-SARS-COV-2 S vectors include a genome comprising, in a 3′-to-5′ order, a 3′ leader region, a BPIV3 N gene, a heterologous gene, BPIV3 P and M genes, HPIV3 F and HN genes, a BPIV3 L gene, and a 5′ trailer region. The heterologous gene encodes a recombinant SARS-COV-2 S protein (such as a SARS-COV-2 S protein of a variant of concern) comprising proline substitutions at sites corresponding to K986P and V987P (numbered with reference to SEQ ID NO: 22 and SEQ ID NO: 25) and an amino acid sequence at least 90% identical to SEQ ID NO: 22. In some embodiments, the recombinant SARS-COV-2 S protein further includes F817P, A892P, A899P and A942P substitutions, and/or a RRAR(682-685)GSAS substitution (numbered with reference to SEQ ID NO: 22 and SEQ ID NO: 25, respectively) to remove a S1/S2 furin cleavage site, and an amino acid sequence at least 90% identical to SEQ ID NO: 22. In some embodiments, the HPIV3 HN gene encodes a HPIV3 HN protein comprising threonine and proline residues at positions 263 and 370, respectively. The rB/HPIV3-SARS-COV-2/S vectors disclosed herein are infectious, attenuated, and self-replicating, and can be used to induce an immune response to SARS-COV-2 and HPIV3.

In some embodiments, the heterologous gene encoding the recombinant SARS-COV-2 S protein can be codon-optimized for expression in human cells.

Also provided herein are methods and compositions related to the expression of the disclosed viruses. For example, isolated polynucleotide molecules that include a nucleic acid sequence encoding the genome or antigenome of the described viruses are disclosed.

Immunogenic compositions including the rB/HPIV3-SARS-COV-2/S are also provided. The compositions can further include an adjuvant. Methods of eliciting an immune response in a subject by administering an effective amount of a disclosed rB/HPIV3-SARS-COV-2/S to the subject are also disclosed. In some embodiments, the subject is a human subject, for example, a human subject between 1 and 6 months of age, or between 1 and 12 months of age, or between 1 and 18 months of age, or older.

The foregoing and other objects and features of the disclosure will become more apparent from the following detailed description, which proceeds with reference to the accompanying figures.

### BRIEF DESCRIPTION OF THE DRAWINGS

FIGS. 1A-1C: B/HPIV3 vectors expressing wild-type and prefusion-stabilized versions of the SARS-COV-2 S spike protein with S1/S2 cleavage site ablated. (FIG. 1A) Map of the B/HPIV3 genome with the added SARS-COV-2 S gene: BPIV3 genes (N, P, M and L), HPIV3 genes (F and HN), and the SARS-COV-2 S gene are indicated. Each gene, including the SARS-COV-2 S gene, begins and ends with PIV3 gene start (GS) and gene end (GE) transcription signals (light and dark grey bars, respectively). The S gene encodes either the wild-type (S) or a prefusion-stabilized (S-2P or S-6P) versions of the S protein with the S1/S2 cleavage site ablated, and was inserted into an AscI restriction site to place it between the B/HPIV3 N and P genes. The stabilizing proline substitutions [“2P”; aa K986P and V987P, and “6P”; aa K986P and V987P, plus F817P, A892P, A899P and A942P of SEQ ID NO: 22] and four amino acid substitutions that ablate the furin cleavage site (RRAR to GSAS, aa 682-685 of SEQ ID NO: 22) in the prefusion-stabilized versions of the S protein (S-2P and S-6P) are indicated. (FIG. 1B) Stability of SARS-CoV-2 expression, analyzed by dual-staining immunoplaque assay. Virus stocks were titrated by serial dilutions on Vero cells, and analyzed by double-staining immunoplaque assay essentially as described previously (Liang et al., J Virol 89:9499-510), using a goat hyperimmune antiserum against a recombinantly-expressed secreted version of S-2P protein, and rabbit hyperimmune antiserum against HPIV3 virions. HPIV3-specific and SARS-COV-2 S-specific staining is shown. The percentage of plaques staining positive for both HIPIV3 and SARS-COV-2 S protein is indicated at the bottom. (FIG. 1C) Multicycle replication of B/HPIV3 vectors on Vero cells. Vero cells in 6-well plates were infected in triplicate with indicated viruses at a multiplicity of infection (MOI) of 0.01 PFU per cell and incubated at 32° C. for a total of 7 days. At 24 h intervals, aliquots of culture medium were collected and flash-frozen for subsequent immunoplaque titration on Vero cells; virus titers (Log₁₀ PFU/ml are shown (FIG. 1C).

FIGS. 2A-2F: Viral proteins in lysates of cells infected with B/HPIV3, B/HPIV3/S, and B/HPIV3/S-2P, and purified virions. (FIG. 2A) A549 or Vero cells in 6-well plates were infected with B/HPIV3, B/HPIV3/S, or B/HPIV3/S-2P at a MOI of 1 PFU per cell and incubated at 32° C. for 48 h. Cell lysates were prepared, denatured, reduced, and analyzed by Western blotting. SARS-COV-2 S protein was detected by a goat hyperimmune serum to the S protein, and the BPIV3 proteins were detected by a hyperimmune serum raised against sucrose-purified HPIV3, followed by immunostaining with infrared fluorophore labelled secondary antibodies and infrared imaging. Images were acquired and analyzed using Image Studio software (Licor). Immunostaining for glyceraldehyde-3-phosphate dehydrogenase (GAPDH) was included as a loading control. (FIG. 2B) Relative expression of N, P, HN, and F proteins in Vero cells by B/HPIV3/S and B/HPIV3/S-2P, normalized to B/HPIV3, and relative expression of SARS-COV S protein, normalized to B/HPIV3/S. To obtain these data, three additional, replicate infections and Western blot analyses were preformed and analyzed in Vero cells as described in Part A. (FIG. 2C) Representative Western blot image as described for FIG. 1A, used for quantitative analysis shown in FIG. 2B. A549 (FIG. 2C) and Vero cells (FIG. 2D) were infected with indicated viruses at a MOI of 1 PFU/cell, and cell lysates were prepared 48 h post-infection, separated by gel electrophoresis under denaturing and reducing conditions, and subjected to Western blot analysis. GAPDH was included as control. For the experiments in Vero cells, the expression of each protein was normalized to that of B/HPIV3 from the same experiment or, in case of the SARS-COV-2 S protein, to B/HPIV3/S from the same experiment, and the relative levels of expression, determined in 3 independent experiments, are shown in FIG. 2B. (FIGS. 2E, 2F) Silver staining (FIG. 2E) and Western blot analysis (FIG. 2F) of sucrose-purified B/HPIV3, B/HPIV3/S, and B/HPIV3/S-2P. Virus was purified from medium supernatants of Vero cells infected with the indicated viruses by centrifugation through 30%/60% discontinuous sucrose gradients, and gently pelleted by centrifugation to remove sucrose as described previously (Munir et al., 2008. J Virol 82:8780-96). 1 μg of protein per lane was used for SDS-PAGE, and gels were subjected to silver staining (FIG. 2E) and Western blot analysis performed as in FIG. 2A (FIG. 2F).

FIGS. 3A-3J: Replication and immunogenicity in the hamster model. Six-week-old golden Syrian hamsters in groups of 30 were inoculated intranasally with 5 log 10 PFU of the indicated viruses. On days 3 and 5, six animals per group per day were sacrificed and virus titers in nasal turbinates (FIG. 3A) and lungs (FIG. 3B) were determined by dual-staining immunoplaque assay. Individual animal titers are shown by symbols and group means are shown immediately below the dotted line; the maximum mean peak titer irrespective of day for each group is in bold and underlined. The limit of detection (LOD), indicated by a dotted line, was 50 PFU/g of tissue. In FIG. 3B, the average percentage of dual-stained plaques is indicated immediately above the x axis, indicating stability of S expression of the B/HPIV3 vectors during in-vivo replication. (FIG. 3C) On days 3 and 5, lung tissues were obtained (n=2 animals per group) and processed for immunohistochemistry analysis. Serial sections were immunostained for HPIV3 and SARS-COV-2 antigen using hyperimmune antisera against HPIV3 virions and secreted S-2P protein, respectively. Representative images from day 5 are shown. Areas with bronchial epithelial cells positive for HPIV3 and SARS-COV-S are marked by arrowheads (20× magnification; a size bar 50 μM in length is shown in the bottom right corners). (FIGS. 3D, 3E, 3F, 3G, 3H, 3I). Sera were collected on day 28 and serum antibody titers were evaluated (n=14 animals per group) to determine the 50% SARS-COV-2 neutralizing titers (ND₅₀) on Vero cells against isolates WA1/2020 (lineage A), USA/CA_CDC_5574/2020 (lineage B.1.1.7/Alpha), and USA/MD-HP01542/2021(lineage B.1.351/Beta) (FIGS. 3D, 3E, 3F), or IgG ELISA titers to a secreted form of the S-2P protein (FIG. 3G) or to a fragment of the S protein (aa 328-531) containing SARS-COV-2 receptor binding domain [RBD; (FIG. 3H)]. (FIG. 3I) The sera were also analyzed to determine 60% plaque reduction neutralization titers (PRNT₆₀) to B/HPIV3. (FIG. 3J) IgA titers to a secreted form of the S-2P protein, determined by dissociation-enhanced lanthanide time-resolved fluorescent immunoassay (DELFIA- TRF). Mean log₁₀ antibody titers are indicated immediately above the x-axes; for C, natural numbers for the reciprocal neutralizing titers also are provided (brackets). Asterisks indicate the significance of differences between the groups (****=P<0.0001).

FIGS. 4A-4D: Immunogenicity of B/HPIV3 vectors (Experiment 2). Six-week-old golden Syrian hamsters in groups of 10 were inoculated intranasally with 5 log 10 PFU of the indicated viruses, following the same procedure as in Experiment 1. (FIGS. 4A, 4B, 4C) On day 27, serum antibody titers were evaluated (n=10 animals per group) to determine the 50% SARS-COV-2 neutralizing titers (ND₅₀) on Vero cells (FIG. 4A), the IgG titers to the S protein (B) or the SARS-COV-2 receptor binding domain [RBD; (FIG. 4C)], determined by ELISA. (FIG. 4D) The level of serum BHPIV3-neutralizing antibodies induced by each virus was also evaluated. The 60% plaque reduction neutralization titers (PRNT₆₀) were determined. Mean log₁₀ antibody titers are indicated below the dotted line; natural numbers for the reciprocal neutralizing titers are also provided (FIG. 4A, in brackets). LOD, level of detection. Asterisks indicate the significance of differences between the groups

FIGS. 5A-5E: Protection of vector-immunized hamsters against SARS-COV-2 challenge. Hamsters in groups of 10 were immunized intranasally as described in FIG. 3 and on day 30 were challenged intranasally with 4.5 log₁₀ TCID₅₀ per animal of SARS-COV-2, strain WA1/2020. Animals were monitored for weight loss (FIG. 5A). On days 3 and 5 post-challenge, five animals per group were euthanized, and tissues were collected. (FIGS. 5B, 5C). Tissue homogenates were prepared, and total RNA was extracted from lung homogenates. cDNA was synthesized from 350 ng of RNA, and analyzed by qPCR using a custom-made 16-gene hamster-specific Taqman array, including beta-actin which was used as housekeeping gene. qPCR results were analyzed using the comparative threshold cycle (ΔΔCT) method, normalized to beta-actin, and expressed for each gene as fold-increase over the average expression of 3 non-immunized, non-infected hamsters. (FIG. 5B) Relative gene expression of C-X-C motif chemokine ligand 10 (CXCL10) and of myxovirus resistance protein 2 (Mx2), a type 1 interferon stimulated gene, in hamster lung tissue on days 3 and 5 after SARS-COV-2 challenge. (FIG. 5C). Heat maps showing expression of 12 immune response genes in lung tissue on day 3 after SARS-COV-2 challenge, presented as fold-increase or decrease of gene expression over the mean of 3 unimmunized, unchallenged controls. (FIGS. 5D, 5E) on days 3 and 5 post-challenge, challenge virus titers were determined in nasal turbinates (FIG. 5D) and lungs (FIG. 5E) of 5 animals per group. Individual titers, means, and standard deviations are shown for each group. Asterisks indicate the significance of differences of B/HPIV3/S and B/HPIV3/S-2P compared to the B/HPIV3 control immunized group, or (FIGS. 5D, 5E) the differences between each group. ns, not significant.

FIGS. 6A-6E: Viral proteins in lysates of cells infected with B/HPIV3, B/HPIV3/S-2P, and B/HPIV3/S-6P, and purified virions. Vero cells (FIG. 6A) or A549 cells (FIG. 6B) in 6-well plates were infected with B/HPIV3, B/HPIV3/S-2P, and B/HPIV3/S-6P at an MOI of 1 PFU per cell and incubated at 32° C. for 48 h. Cell lysates were prepared, denatured, and analyzed by Western blotting. SARS-COV-2 S protein was detected by a goat hyperimmune serum to the S protein, and the BPIV3 proteins were detected by a hyperimmune serum raised against sucrose-purified HPIV3, followed by immunostaining with infrared fluorophore labelled secondary antibodies and infrared imaging. Images were acquired and analyzed using Image Studio software (Licor). Immunostaining for GAPDH was included as a loading control. (FIG. 6C) Western blot analysis of sucrose-purified B/HPIV3, B/HPIV3/S-2P, and B/HPIV3/S-6P. Virus was purified from medium supernatants of Vero cells infected with the indicated viruses by centrifugation through 30%/60% discontinuous sucrose gradients, and gently pelleted by centrifugation to remove sucrose as described previously (Munir et al., 2008. J Virol 82:8780-96). One μg of protein per lane was used for SDS-PAGE, and gels were subjected to Western blot analysis performed as in Part A. Multicycle replication of B/HPIV3 vectors on Vero cells (FIG. 6D) and A549 cells (FIG. 6E). Vero or A549 cells in 6-well plates were infected in triplicate with indicated viruses at a MOI of 0.01 PFU per cell and incubated at 32° C. for a total of 7 days. At 24 h intervals, aliquots of culture medium were collected and flash-frozen for subsequent immunoplaque titration on Vero cells.

FIGS. 7A-7N: Replication and immunogenicity of B/HPIV3, B/HPIV3/S-2P, and B/HPIV3/S-6P in the hamster model. In experiment 1, six-week-old golden Syrian hamsters in groups of 27 were inoculated intranasally with 5 log₁₀ PFU of the indicated viruses. On days 3, 5, and 7, five animals per group per day were sacrificed and virus titers in nasal turbinates (FIG. 7A) and lungs (FIG. 7B) were determined by dual-staining immunoplaque assay. Individual animal titers are shown by symbols and group means are shown immediately below the dotted line; the maximum mean peak titer irrespective of day for each group is in bold and underlined. The limit of detection (LOD), indicated by a dotted line, was 50 PFU/g of tissue. (FIGS. 7C, 7D, 3E) Sera were collected on day 28 and serum antibody titers were evaluated (n=12 animals per group) to determine the 50% SARS-COV-2 neutralizing titers (ND₅₀) on Vero cells against isolate WA1/2020 (lineage A) (FIG. 7C), or IgG ELISA titers to a secreted form of the S-2P protein (FIG. 7D) or to a fragment of the S protein (aa 328-531) containing SARS-COV-2 receptor binding domain [RBD; (FIG. 7E)]. (FIG. 7F) The sera also were analyzed to determine 60% plaque reduction neutralization titers (PRNT₆₀) to B/HPIV3. Mean log₁₀ antibody titers are indicated immediately above the x-axes; asterisks indicate the significance of differences between the groups. (FIG. 7G) In experiment 2, six-week-old golden Syrian hamsters in groups of 45 were inoculated intranasally with 5 log₁₀ PFU of the indicated viruses. On days 26 or 27, sera were obtained [n=45 per group] to determine IgG ELISA titers to a secreted form of the S-2P protein or to a fragment of the S protein (aa 328-531) containing SARS-COV-2 receptor-binding domain (RBD) (FIG. 7H), to determine IgA titers to S-2P or the RBD by dissociation-enhanced lanthanide time-resolved fluorescent immunoassay (DELFIA-TRF) (FIG. 7I), to determine the 50% SARS-COV-2 neutralizing titers (ND₅₀) on Vero E6 cells against the vaccine-matched strain WA1/2020, USA/CA_CDC_5574/2020 (B.1.1.7/Alpha variant), and USA/MD-HP01542/2021 (B.1.351/Beta variant) in live-virus SARS-COV-2 neutralization assays performed at BSL3 (FIG. 7J). (FIG. 7K) Ten sera from each group were randomly selected for BSL2 neutralization assays using pseudoviruses bearing spike proteins from SARS-COV-2 B.1.617.2/Delta and B.1.1.529/Omicron. The 50% inhibitory concentration (IC50) titers of sera were determined. (FIG. 7L) Sera were also analyzed to determine the 60% plaque reduction neutralization titers (PRNT₆₀) to HPIV3. Each hamster is represented by a symbol and mean log₁₀ antibody titers and standard deviations are indicated. The limit of detection is shown. For FIGS. 7H-7L, *=P<0.05; ***=P<0.001; ****=P<0.0001. (FIGS. 7M, 7N) On day 5, NT (FIG. 7M) and lung tissues (FIG. 7N) were obtained (n=2 additional animals per group, and n=1 uninfected control animal) and processed for immunohistochemistry analysis. Serial sections were immunostained for HPIV3 and SARS-COV-2 antigen using hyperimmune antisera raised against HPIV3 virions and a secreted form of the S-2P protein, respectively. Areas with bronchial epithelial cells positive for HPIV3 and SARS-COV-S are marked by arrowheads (20 um or 100 um size bars are shown in the bottom right corners).

FIGS. 8A-8G: Protection of B/HPIV3, B/HPIV3/S-2P, and B/HPIV3/S-6P immunized hamsters against intranasal challenge with SARS-COV-2 of three major lineages. Hamsters in groups of 45 were immunized intranasally as described in FIG. 7. On day 33, 15 hamsters per group were challenged intranasally with 4.5 log₁₀ TCID₅₀ per animal of SARS-COV-2, strain WA1/2020 (lineage A), isolate USA/CA_CDC_5574/2020 (lineage B.1.1.7/Alpha), or USA/MD-HP01542/2021 (lineage B.1.351/Beta). Animals were monitored for weight loss for 14 days after challenge (FIG. 8A). (FIG. 8B) Expression of inflammatory cytokines in lung tissues on days 3 and 5 post-challenge. Five animals per group were euthanized and tissues were collected. Total RNA was extracted from lung homogenates. cDNA was synthesized from 350 ng of RNA and analyzed by hamster-specific Taqman assays. Relative gene expression of C-X-C motif chemokine ligand 10 (CXCL10) and of myxovirus resistance protein 2 (Mx2), a type 1 IFN-inducible antiviral response gene, and interferon lambda (IFN-L) compared to the mean level of expression of unimmunized, unchallenged controls (dashed line). qPCR results were analyzed using the comparative threshold cycle (ΔΔC_{T}) method, normalized to beta-actin. Each hamster is represented by a symbol. The means and SD are shown. *=P<0.05; **=P<0.01; ***=P<0.001; ****=P<0.0001. (FIGS. 8C-8E) On days 3 and 5 post-challenge, challenge virus titers were determined in nasal turbinates (left panels) and NTs (right panels) of 5 animals per group. Individual titers, means, and standard deviations are shown for each group. GMTs are indicated above the x axes. Asterisks indicate the significance of differences between each group. ns, not significant. (FIG. 8F) SARS-COV-2 lung viral loads after challenge, expressed in log₁₀ genome copies per g. To detect viral genomic N (gN), E (gE), and subgenomic E mRNA (sgE) of the SARS-COV-2 challenge viruses, cDNA was synthesized from total RNA from lung homogenates as described above, and Taqman qPCRs were performed (n=5 animals per time point). (FIG. 8G) Sera from 5 animals per group were collected on day 21 after challenge, and serum neutralizing titers to the vaccine-matched virus WA1/2020 (left panel), B.1.1.7/Alpha (middle panel) or B.1.351/Beta (left panel) were determined for animals immunized with B/HPIV3 (circles), B/HPIV3/S-2P (squares), and B/HPIV3/S-6P (triangles) and challenged with the indicated SARS-COV-2 virus. Each hamster is represented by a symbol. The limit of detection is indicated by a dashed line. *=P<0.05; **=P<0.01; ***=P<0.001; ****=P<0.0001.

FIGS. 9A-9D: Replication and immunogenicity of B/HPIV3 and B/HPIV3/S-6P in rhesus macaques. Rhesus macaques (n=4 per group), seronegative for HPIV3 as determined by a 60% plaque reduction neutralization assay, were immunized intranasally and intratracheally with 6 log₁₀ PFU of B/HPIV3 or B/HPIV3/S-6P under light sedation. Serum was collected on days −3, 14, 21 and 28 post-inoculation for serology. (FIGS. 9A, 9B). Nasopharyngeal (NP) swabs were collected daily on days 0 through 10 and day 12, and tracheal lavage (TL) samples were collected on days 2, 4, 6, 8 10, and 12 to analyze vaccine virus shedding. B/HPIV3 and B/HPIV3/S-6P vaccine virus shedding was analyzed by dual-staining immunoplaque assay. (FIGS. 9C, 9D) Serum IgG titers to a secreted form of the S-2P protein (FIG. 9C) or to a fragment of the S protein (aa 328-531) containing SARS-COV-2 RBD (FIG. 9D)] were determined by ELISA. Human COVID-19 convalescent plasma sera (de-identified samples) were included for comparison and as benchmarks (diamonds, FIGS. 9C, D).

FIGS. 10A-10C: Genome organization of B/HPIV3/S-6P and vaccine replication following intranasal/intratracheal immunization of rhesus macaques. (FIG. 10A) Diagram of the genome of B/HPIV3/S-6P. BPIV3 genes (N, P, M and L) and HPIV3 genes (F and HN) are indicated. The full-length SARS-COV-2 S ORF (aa 1-1,273) from the WA1/2020 isolate was inserted between the N and P ORFs. The S sequence includes RRAR-to-GSAS substitutions that ablate the S1/S2 cleavage site and contains 6 stabilizing proline substitutions (S-6P). Each gene begins and ends with PIV3 gene start and gene end transcription signals (light and dark bars, respectively). (FIGS. 10B-10C) Replication of B/HPIV3/S-6P and B/HPIV3 control in the upper and lower airways of rhesus macaques (RMs). Two groups of 4 RMs were immunized intranasally and intratracheally with 6.3 log₁₀ PFU of B/HPIV3/S-6P or B/HPIV3. Nasopharyngeal swabs (FIG. 10B) and tracheal lavages (FIG. 10C) were performed daily and every other day, respectively, from day 0 to day 12 post-immunization (pi). Vaccine virus titers of each sample were determined by immunoplaque assay. Titers are expressed as log₁₀ PFU/ml. The limit of detection was 0.7 log₁₀ PFU/mL (dotted line). Each RM is indicated by a symbol. * p≤0.05, ** p<0.01, **** p<0.0001.

FIGS. 11A-11B: Intranasal/intratracheal immunization with B/HPIV3/S-6P induces mucosal antibody responses to SARS-COV-2 S in RMs. Rhesus macaques (n=4 per group) were immunized with B/HPIV3/S-6P or B/HPIV3 (control) by the intranasal/intratracheal route (FIG. 16). (FIGS. 11A-11B) Nasal washes (NW) were collected before immunization and on days 14, 21, and 28 post-immunization (pi), and bronchoalveolar lavages (BAL) were collected before immunization and on days 9, 21, and 28 pi. Endpoint titers expressed in log₁₀ for mucosal IgA and IgG to a secreted prefusion-stabilized form (aa 1-1,208; S-2P) of the S protein (left panels) or to a fragment of the S protein (aa 328-531) containing SARS-COV-2 receptor-binding domain (RBD) (right panels). S- and RBD-specific IgA and IgG responses were analyzed by time-resolved dissociation-enhance lanthanide fluorescence (DELFIA-TRF) immunoassay. The limit of detection is 1.6 log₁₀ (dotted line). Each RM is represented by a symbol. * p≤0.05.

FIGS. 12A-12D: B/HPIV3/S-6P induces serum binding antibody responses to SARS-COV-2 S and neutralizing antibody responses to VoCs in R.Ms. (FIGS. 12A-12C) Sera collected from RMs before immunization and on days 14, 21, and 28 pi. (FIG. 12A) Endpoint ELISA titers, expressed in log₁₀ for serum IgM, IgA and IgG to S-2P (left panels) or to the RBD (right panels). Twenty-three plasma samples from COVID-19 convalescent individuals were evaluated in parallel for serum IgG to S-2P or the RBD. The limits of detection are 3 log₁₀ for IgM and 2 log₁₀ for IgA and IgG, respectively. (FIG. 12B) Neutralization assays using pseudoviruses bearing spike proteins from SARS-COV-2 WA/12020, B.1.1.7/Alpha, B.1.351/Beta, B.1.617.2/Delta and B.1.1.529/Omicron. The 50% inhibitory concentration (IC₅₀) titers of sera were determined. (FIG. 12C) The 50% SARS-COV-2 serum neutralizing titers (ND₅₀) were determined on Vero E6 cells against vaccine-matched WA1/2020, or viruses from lineages B.1.17/Alpha or B.1.351/Beta. The limit of detection is 0.75 log₁₀. (FIG. 12D) Sera were analyzed by a 60% plaque reduction neutralization test (PRNT₆₀) to evaluate the levels of HPIV3 neutralizing antibodies. The limit of detection is 1 log₁₀. Each RM is represented by a symbol. * p≤0.05, ** p≤0.01, *** p≤0.001, **** p≤0.0001.

FIGS. 13A-13J: Intranasal/intratracheal immunization with B/HPIV3/S-6P induces S-specific CD4+ and CD8+ T-cell responses in blood and lower airways. PBMCs (FIGS. 13A, 13C, 13D, 13G, 13H) or BAL mononuclear cells (FIGS. 13B, 13E, 13F, 13I, 13J), collected on indicated days post-immunization (pi), were stimulated with overlapping S or (BAL only) N peptides or left unstimulated, and processed for flow cytometry. Phenotypic analyses were performed on non-naïve non-regulatory (CD95+/Foxp3−) CD4+ or CD8+ T cells (see FIG. 20 for gating); frequencies are relative to that population. (FIGS. 13A-13B) IFNγ and TNFα expression by CD4+ or CD8+ T cells from blood (FIG. 13A) or BAL (FIG. 13B) of representative B/HPIV3 (top) or B/HPIV3/S-6P-immunized (bottom) RMs. (FIGS. 13C, 13D, 13E, 13F) Background-corrected frequencies of S-specific IFNγ+/TNFα+ CD4+ (FIGS. 13C, 13E) or CD8+ (FIGS. 13D, 13F) T cells from blood (FIGS. 13C, 13D) or BAL (FIGS. 13E, 13F). (FIGS. 13G, 13H, 13I, 13J) Expression of proliferation marker Ki-67 by IFNγ+/TNFα+ CD4+ or CD8+ T cells from blood (FIGS. 13G-13H) or BAL (FIGS. 131-13J) of 4 B/HPIV3/S-6P-immunized RM. (FIGS. 13G, 13I) Gating and histograms showing Ki-67 expression and (FIGS. 13H, 13J) % and median fluorescence intensity (MFI) in IFNγ+/TNFα+ T cells from blood (FIG. 13H) or BAL (FIG. 13J) of 4 B/HPIV3/S-6P-immunized RMs, represented by different symbols. BAL, bronchoalveolar lavage.

FIGS. 14A-14H: Phenotype of SARS-COV-2 S-specific CD4+ and CD8+ T cells in lower airways of B/HPIV3/S-6P-immunized R.M. (FIGS. 14A, 14B) Representative dot plots showing gating on S-specific IFNγ⁺/TNFα⁺ and IFNγ⁻/TNFα⁻ cells obtained by bronchoalveolar lavage (BAL) following stimulation with overlapping S peptides (gated from non-naïve non-regulatory CD95⁺/Foxp3⁻ T cells; FIG. 20); histograms show expression of IL-2 (for CD4⁺ T cells only), CD107ab and granzyme B by the IFNγ⁺/TNFα⁺ T cells on the indicated days. (FIGS. 14C, 14D) Frequencies of IL-2⁺, CD107ab⁺ and granzyme B⁺ by IFNγ⁺/TNFα⁺ S-specific CD4 (FIG. 14C) or CD8 (FIG. 14D) T cells in 4 BHPIV3/S-6P-immunized RMs on indicated days. Each macaque is represented by a different symbol. (FIGS. 14E, 14G) Representative dot plots showing gating on S-specific IFNγ⁺/TNFα⁺ and IFNγ⁻/TNFα⁻ CD95⁺/Foxp3⁻ T cells (left panels). CD69 and CD103 were used to differentiate circulating (CD69⁻CD103⁻) and tissue resident memory (Trm; CD69⁺ CD103⁻, CD69⁺ CD103⁺ and CD69⁻CD103⁺; % indicated) S-specific IFNγ⁺/TNFα⁺ T cells isolated from BAL (right panels). (FIGS. 14F, 14H) The median % of the circulating and each of the 3 Trm S-specific IFNγ⁺/TNFα⁺ CD4 (FIG. 14F) or CD8 (FIG. 14H) T-cell subsets present in BAL of 4 B/HPIV3/S-6P-immunized RMs are stacked on indicated days.

FIGS. 15A-15C: No detection of SARS-COV-2 challenge virus replication in the upper and lower airways and lung tissues of B/HPIV3/S-6P-immunized R.Ms. Rhesus macaques that had been immunized by a single intranasal/intratracheal dose of B/HPIV3/S-6P or B/HPIV3 (n=4 per group) were challenged intranasally/intratracheally on day 30 post-immunization with 5.8 TCID₅₀ of SARS-COV-2. Nasal swabs (NS) and bronchoalveolar lavage fluid were collected on days 2, 4, and 6 post-challenge (pc), and viral RNA was extracted. Animals were euthanized on day 6 after challenge, and RNA was extracted from indicated areas of lung tissue. SARS-COV-2 genomic N RNA and subgenomic E mRNA were quantified by RT-qPCR using RNA extracted from NS (FIG. 15A), BAL samples (FIG. 15B), or from the indicated area of the lungs on day 6 pc (FIG. 15C). The number of B/HPIV3/S-6P-immunized- or B/HPIV3-immunized RMs with detectable genomic N RNA or subgenomic E mRNA in each set of samples is indicated. The limit of detection was 2.57 log₁₀ copies per ml of NS or BAL fluid and 3.32 log₁₀ copies per g of lung tissue. Each RM is indicated by a symbol. * p≤0.05.

FIG. 16: Timeline of the rhesus macaque experiment and sampling. Experimental timeline for the immodication of groups of 4 RMs with the B/HPIV3/S-6P vaccine candidate or the empty B/HPIV3 vector used as a control. Challenge with the SARS-COV-2 A WA/2020 isolate was performed on day 30 post-immunization. Pre- and post-challenge sampling schedules are summarized.

FIGS. 17A-17B: S expression by B/HPIV3/S-6P is stable in rhesus macaques. The stability of S expression by B/HPIV3/S-6P in RMs was evaluated by dual-staining immunoplaque assay on Vero cells from NS (FIG. 17A) and TL (FIG. 17B) samples collected at the peak of vaccine shedding (days 5 through 7). Plaques were immunostained with an HPIV3-specific rabbit hyperimmune serum to detect B/HPIV3 antigens, and a goat hyperimmune serum to the secreted SARS-COV-2 S to detect co-expression of the S protein, followed by infrared-dye secondary antibodies. Fluorescent staining for PIV3 proteins and SARS-CoV-2 S was performed and the percentage of plaques expressing both HPIV3 and S proteins was determined.

FIG. 18: Vital signs of rhesus macaques after immunization with the B/HPIV3 vector or B/HPIV3/S-2P and challenge with SARS-COV-2. Groups of 4 macaques were immunized with B/HPIV3/S-6P or with the B/HPIV3 empty vector used as a control. On day 30 post-immunization (pi), animals were challenged in BSL3 facility with the SARS-COV-2 WA1/2020 isolate. Animals were euthanized on day 36 pi (day 6 post-challenge). The body weight, rectal temperature, respiration rate, heart rate, and oxygen saturation rate were monitored at the indicated day pi. Timing of immunization and SARS-CoV-2 challenge are indicated by a dashed line and arrows. Each animal is represented by a symbol.

FIGS. 19A-19H: Phenotype of SARS-COV-2 S specific CD4+ and CD8+ T cells in the blood of the B/HPIV3/S-6P immunized rhesus macaques. (FIG. 19A) Dot blot of the CD4+ T cells in the blood of a representative B/HPIV3/S-6P-immunized RM describing the gating of the S-specific IFNγ+ TNFα+ cells. The level of expression of IL-2, CD107ab and granzyme B by the IFNγ+ TNFα+ CD4+ T cells from the same RM are shown as histograms at the indicated day pi with the IFNγ− TNFα− CD4+ T cells used for reference. (FIG. 19B) % of IFNγ+ TNFα+ CD4+ T cells in the blood of the 4 B/HPIV3/S-6P-immunized RMs that expressed IL-2, CD107ab or granzyme B at the indicated day pi. (FIG. 19C) Dot blot of the CD8+ T cells in the blood of a representative B/HPIV3/S-6P-immunized RM describing the gating of the S-specific IFNγ+ TNFα+ cells. The level of expression of CD107ab and granzyme B by the IFNγ+ TNFα+ CD4+ T cells from the same RM are shown as histograms at the indicated day pi with the IFNγ− TNFα− CD4+ T cells used for reference. (FIG. 19D) % CD107ab+ or granzyme B+ of IFNγ+ TNFα+ CD8+ T cells at the indicated day pi in the blood of the 4 B/HPIV3/S-6P-immunized RMs. Each macaque is represented by a different symbol. (FIGS. 19E, 19G) Representative dot plots showing gating on S-specific IFNγ⁺/TNFα⁺ CD95⁺/Foxp3⁻ T-cells (left panels). CD69 and CD103 were used to differentiate circulating (CD69⁻ CD103⁻) and tissue-resident memory (Trm; CD69⁺ CD103⁻, CD69⁺ CD103⁺ and CD69⁻ CD103⁺; % indicated) S-specific IFNY*/TNFα+ T-cells isolated from blood (right panels). (FIGS. 19F, 19H) The median % of the circulating and each of the 3 Trm S-specific IFNγ⁺/TNFα⁺ CD4+ (FIG. 19F) or CD8+ (FIG. 19H) T-cell subsets present in blood of 4 B/HPIV3/S-6P-immunized RMs are stacked on indicated days.

FIG. 20: Gating strategy of the CD4+ and CD8+ T cells isolated from BAL of rhesus macaques. Representative flow cytometry dot plots of the lung cells isolated from a BAL sample, visualizing the typical gating strategy used to identify the CD4+ and CD8+ T cell populations described in FIGS. 12 and 13. The same gating strategy was applied to identify and analyze the CD4+ and CD8+ T cells from PBMC isolated from the blood (FIGS. 3 and 19). Live cells were first gated based on a live dead staining and forward scatter area. Live lymphocytes were identified based on forward and side scatter areas. Then, singlets were selected using a first gate based on forward scatter height and forward scatter area followed by a second gate based on side scatter height and side scatter area. An additional live/dead gating was performed to discard any remaining dead cells. The live single CD3+ IFNγ+ T cells were next gated using CD3 and IFNγ. As CD3 expression can be downregulated on activated T cells, a wide CD3 gate has been applied. IFNγ+ CD4+ or CD8+ T cells were next identified using a CD4 or CD8 antibody. Non-naïve, non-regulatory CD4+ or CD8+ T cells were finally gated using CD95 and Foxp3, respectively. The phenotypic analysis described in FIGS. 12, 13 and 19 was performed on live single CD3+ CD4+ CD95+ Foxp3⁻ or live single CD3+ CD8+ CD95+ Foxp3⁻ T cells.

FIGS. 21A-21D: Comparable phenotype of circulating (CD69⁻ CD103⁻) and tissue-resident memory (CD69⁺ CD103⁻ and CD69⁺ CD103⁺) S-specific IFNY⁺/TNFα⁺ S-specific CD4 and CD8 T cells. (FIGS. 21A, 21C) Histograms representing IL-2 expression (FIG. 21A only), CD107ab, and granzyme B expression by S-specific circulating and tissue-resident memory (Trm) IFNγ⁺/TNFα CD4 (FIG. 21A) or CD8 (FIG. 21C) T cells on indicated days pi. (FIGS. 21B, 21D) % and level of expression (MFI) of IL-2 (FIG. 21B only), CD107ab and granzyme B by the S-specific circulating and Trm IFNγ⁺/TNFα⁺ CD4 (FIG. 21B) and the CD8 (FIG. 21D) T cells in the 4 B/HPIV3/S-6P-immunized RMs. Due to the low frequency of CD69⁺ CD103⁺ T cells on day 9 pi, the % positive and MFI of IL-2, CD107ab and granzyme B by this subset are only indicated on days 14 and 28 pi. In FIG. 21B and FIG. 21D, each RM is indicated by a symbol.

FIG. 22: Quantification of SARS-Cov-2 from rectal swabs. SARS-COV-2 genomic N RNA and subgenomic E mRNA were quantified by RT-qPCR using RNA extracted from rectal swabs at the indicated day post challenge (pc). The number of B/HPIV3/S-6P-immunized- or B/HPIV3-immunized RMs with detectable genomic N RNA or subgenomic E mRNA in each set of samples is indicated. The limit of detection was 2.57 log₁₀ copies per ml of rectal swab fluid. Each RM indicated by a symbol.

FIGS. 23A-23D: Expression of proliferation marker Ki-67 by IFNY⁺/TNFα⁺ S-specific CD4 and CD8 T cells at 4 days after SARS-COV-2 challenge. (FIGS. 23A, 23B) Background-corrected frequencies of S-specific IFNγ⁺/TNFα⁺ CD4 or CD8 T cells from blood (FIG. 23A) or airways (FIG. 23B) on day 28 and 34 pi (equivalent to day 4 post challenge as challenge was performed at day 30 pi). These frequencies are similar to the frequencies shown in FIGS. 13C, 1D and FIGS. 13E, 13F for the blood and airways, respectively. (FIG. 23C, 23D) % and MFI of proliferation marker Ki-67 by IFNγ⁺/TNFα⁺ CD4 or CD8 T cells from blood (FIG. 23C) or airways (FIG. 23D) of the 4 B/HPIV3/S-6P-immunized RM with RMs represented by different symbols.

FIG. 24: Dual-staining assays of Vero cell plaques of B/HPIV3 expressing S proteins of SARS CoV-2 Delta or Omicron Variants of Concern. B/HPIV3 vectors expressing prefusion-stabilized versions of the SARS-COV-2 S spike protein with S1/S2 cleavage site ablated of B.1.617.2/Delta (B/HPIV3/S-6P/B.1.617.2) and B.1.529/Omicron variants (B/HPIV3/S-6P/B.1.1.529), with S open reading frames codon-optimized for human cells. Virus stocks were titrated by serial dilutions on Vero cells, and analyzed by double-staining immunoplaque assay essentially as described previously (Liang et al., J Virol 89:9499-510), using a goat hyperimmune antiserum against a recombinantly-expressed secreted version of the S-2P protein, and rabbit hyperimmune antiserum against HPIV3 virions. SARS-COV-2 S-specific and HPIV3-specific staining is shown.

### SEQUENCE LISTING

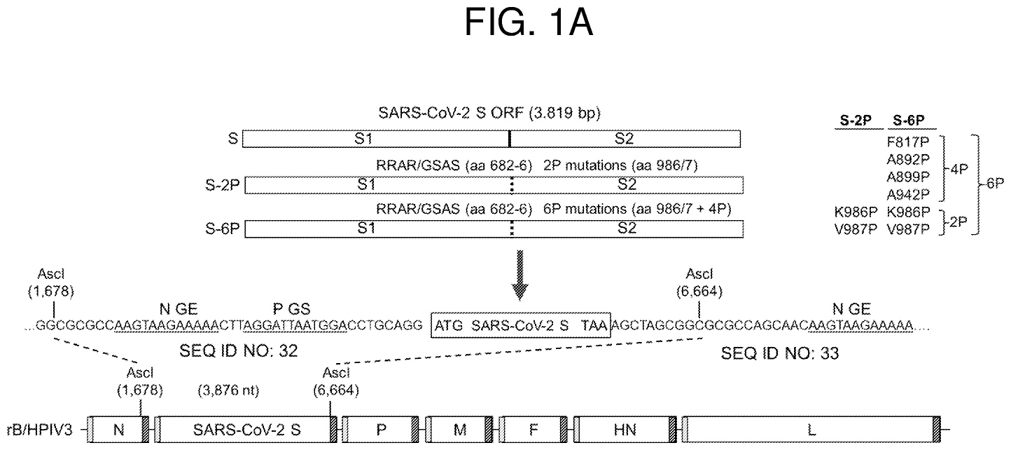

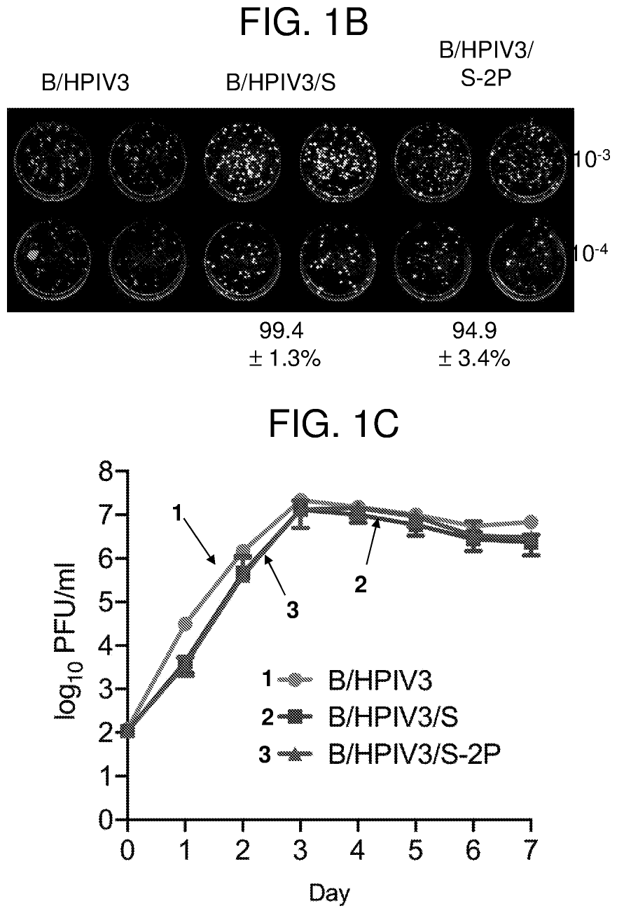

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on Apr. 27, 2022, 295 KB, which is incorporated by reference herein. In the accompanying sequence listing:
SEQ ID NO: 1 is an exemplary amino acid sequence of the BPIV3 N protein.
SEQ ID NO: 2 is an exemplary amino acid sequence of the BPIV3 P protein.
SEQ ID NO: 3 is an exemplary amino acid sequence of the BPIV3 C protein.
SEQ ID NO: 4 is an exemplary amino acid sequence of the BPIV3 V protein.
SEQ ID NO: 5 is an exemplary amino acid sequence of the BPIV3 M protein.
SEQ ID NO: 6 is an exemplary amino acid sequence of the HPIV3 F protein.
SEQ ID NO: 7 is an exemplary amino acid sequence of the HPIV3 HN protein.
SEQ ID NO: 8 is an exemplary amino acid sequence of the HPIV3 HN protein.
SEQ ID NO: 9 is a nucleic acid sequence encoding an exemplary HPIV3 HN protein.
SEQ ID NO: 10 is an exemplary amino acid sequence of the BPIV3 L protein.
SEQ ID NO: 11 is a BPIV3 gene junction sequence.
SEQ ID NOs: 12-21 are gene start and gene end sequences for BPIV3 N, P, M, F, HN and L genes.
SEQ ID NO: 22 is an exemplary amino acid sequence for a wild-type SARS-COV-2 S protein.
SEQ ID NOs: 23-26 are exemplary recombinant SARS-COV-2 S protein sequences.
SEQ ID NO: 27 is a codon-optimized nucleic acid sequence encoding a wild-type SARS-COV-2 S protein.
SEQ ID NOs: 28-29 are codon-optimized nucleic acid sequences encoding recombinant SARS-CoV-2 S protein sequences.
SEQ ID NOs: 30-31 are exemplary rB/HPIV3-SARS-COV-2/S antigenomic cDNA sequences.
SEQ ID NOs: 32-33 are nucleic acid sequence fragments shown in FIG. 1A.
SEQ ID NOs: 34-35 are BPIV3 gene junction sequences.
SEQ ID NO: 36 is an exemplary BPIV3 genome sequence (Kansas stain) deposited under GENBANK™ Accession No. AF178654.1.
SEQ ID NO: 37 is exemplary HPIV3 genome sequence (JS strain) deposited under GENBANK™ Accession No. Z11575.1.
SEQ ID NOs: 38-39 are exemplary recombinant SARS-COV-2 S protein sequences.
SEQ ID NOs: 40-41 are codon-optimized nucleic acid sequences encoding recombinant SARS-CoV-2 S protein sequences.
SEQ ID NOs: 42-43 are exemplary rB/HPIV3-SARS-COV-2/S antigenomic cDNA sequences.

### DETAILED DESCRIPTION

Described herein is a pediatric vector vaccine for intranasal immunization, targeting the primary respiratory mucosal site of SARS-COV-2 infection. The vaccine is based on a parainfluenza virus type 3 (PIV3) vector named B/HPIV3. In response to the SARS-COV-2 pandemic, the B/HPIV3 platform was used to express a wildtype version or the 2P or 6P prefusion-stabilized versions of the SARS-COV-2 spike protein. As discussed in the examples, these recombinant viruses were evaluated in vitro and in a hamster model. The insertion of the S gene did not significantly reduce B/HPIV3 vector replication in vitro or in animal models, and a single intranasal immunization with each of these viruses induced potent serum neutralizing antibodies. While the B/HPIV3 vector encoding the wild-type S (B/HPIV3/S) was not fully protective in the upper respiratory tract of hamsters, a single dose of the B/HPIV3 vector encoding either version of the prefusion-stabilized S protein (B/HPIV3/S-2P or B/HPIV3/S-6P) induced protection in the upper and lower respiratory tract against intranasal SARS-COV-2 challenge virus replication in hamsters. The replication and immunogenicity of the B/HPIV3/S-6P stabilized version were also evaluated in a nonhuman primate model. Following administration by the intranasal/intratracheal route, B/HPIV3/S-6P replicated over several days in the respiratory tract of rhesus macaques, and induced serum immunoglobulin G (IgG) titers to the SARS-COV-2 S protein at levels comparable to those of human COVID-19 convalescent plasma specimens. Based on the efficacy against respiratory mucosal replication in the highly susceptible hamster model, B/HPIV3/S-2P and B/HPIV3/S-6P are suitable for clinical development as bivalent intranasal vaccines against COVID-19 and HPIV3, particularly for young infants and children. Alternative versions of B/HPIV3/S-6P using stabilized S proteins from Delta (SEQ ID NO: 38) or Omicron (SEQ ID NO: 39) variants are also contemplated.

Furthermore, in a rhesus macaque model, a single intranasal/intratracheal immunization with B/HPIV3/S-6P efficiently induced mucosal IgA and IgG in the upper airway and lower airway, as well as strong serum IgM, IgG and IgG responses to SARS-COV-2 S protein. Serum antibodies from immunized animals efficiently neutralized the vaccine-matched SARS-COV-2 WA1/2020 strain and variants of concern (VoCs) of the B.1.1.7/Alpha and B.1.617.2/Delta lineages. Furthermore, B/HPIV3/S-6P induced robust systemic and pulmonary S-specific CD4⁺ and CD8⁺ T-cell responses in rhesus macaques, including tissue-resident memory cells in lungs. Moreover, immunized animals were fully protected from SARS-COV-2 challenge 1 month after immunization and no SARS-COV-2 challenge virus replication was detectable in the upper or lower airways or in lung tissues of immunized animals. Together these data demonstrated that a single topical immunization with B/HPIV3/S-6P was highly immunogenic and protective against SARS-CoV-2 in rhesus macaques. The data disclosed herein support the use of B/HPIV3/S-6P as a stand-alone vaccine and/or as part of prime/boost combinations with an injectable mRNA-based vaccine for infants and young children.

### I. Abbreviations

BAL bronchoalveolar lavage
B/HPIV3 chimeric bovine/human parainfluenza virus type 3
BPIV3 bovine parainfluenza virus type 3
COVID-19 coronavirus disease 2019
DELFIA dissociation-enhanced lanthanide fluorescent immunoassay
eGFP enhanced green fluorescent protein
ELISA enzyme-linked immunosorbent assay
EM electron micrograph
GAPDH glyceraldehyde-3-phosphate dehydrogenase
HPIV3 human parainfluenza virus type 3
IC50 inhibitory concentration 50
IN intranasal
LA lower airway
LRT lower respiratory tract
MOI multiplicity of infection
ND₅₀ neutralizing dose 50
NS nasal swab
NW nasal wash
ORF open reading frame
pc post challenge
PFU plaque forming unit
pi post infection
PIV parainfluenza virus
PRNT₆₀ plaque reduction neutralization tier 60
RBD receptor binding domain
RLU relative light unit
RM rhesus macaque
S spike protein
SARS-COV-2 severe acute respiratory syndrome coronavirus 2
TCID₅₀ tissue culture infectious dose 50
TL tracheal lavage
TRF time resolved fluorescence
UA upper airway
URT upper respiratory tract
VoC variant of concern

### II. Terms

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the term “comprises” means “includes.” Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

To facilitate review of the various embodiments, the following explanations of terms are provided:

Adjuvant: A vehicle used to enhance antigenicity. Adjuvants include a suspension of minerals (alum, aluminum hydroxide, or phosphate) on which antigen is adsorbed; or water-in-oil emulsion, for example, in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). Immunostimulatory oligonucleotides (such as those including a CpG motif) can also be used as adjuvants. Adjuvants include biological molecules (a “biological adjuvant”), such as costimulatory molecules. Exemplary adjuvants include IL-2, RANTES, GM-CSF, TNF-α, IFN-γ, G-CSF, LFA-3, CD72, B7-1, B7-2, OX-40L, 4-1BBL, immune stimulating complex (ISCOM) matrix, and toll-like receptor (TLR) agonists, such as TLR-9 agonists, Poly I:C, or PolyICLC. Adjuvants are described, for example, in Singh (ed.) Vaccine Adjuvants and Delivery Systems. Wiley-Interscience, 2007.

Administration: The introduction of a composition into a subject by a chosen route. Administration can be local or systemic. For example, if the chosen route is intranasal, the composition (such as a composition including a disclosed rB/HPIV3-SARS-COV-2/S vector) is administered by introducing the composition into the nasal passages of the subject. Exemplary routes of administration include, but are not limited to, intranasal, intratracheal, oral, injection (such as subcutaneous, intramuscular, intradermal, intraperitoneal, and intravenous), sublingual, rectal, transdermal (for example, topical), intranasal, vaginal, and inhalation routes.

Amino acid substitution: The replacement of one amino acid in a polypeptide with a different amino acid.

Attenuated: A virus that is “attenuated” or that has an “attenuated phenotype” refers to a virus that has decreased virulence compared to a reference virus under similar conditions of infection. Attenuation usually is associated with decreased virus replication as compared to replication of a reference wild-type virus under similar conditions of infection, and thus “attenuation” and “restricted replication” often are used synonymously. In some hosts (typically non-natural hosts, including experimental animals), disease is not evident during infection with a reference virus in question, and restriction of virus replication can be used as a surrogate marker for attenuation. In some embodiments, a disclosed rB/HPIV3-SARS-COV-2/S vector that is attenuated exhibits at least about 10-fold or greater decrease, such as at least about 100-fold or greater decrease in virus titer in the upper or lower respiratory tract of a mammal compared to non-attenuated, wild type virus titer in the upper or lower respiratory tract, respectively, of a mammal of the same species under the same conditions of infection. Examples of mammals include, but are not limited to, humans, mice, rabbits, rats, hamsters, such as for example Mesocricetus auratus, and non-human primates, such as for example Macaca mulatta or Chlorocebus aethiops. An attenuated rB/HPIV3-SARS-COV-2/S vector may display different phenotypes including without limitation altered growth, temperature sensitive growth, host range restricted growth, or plaque size alteration.

Control: A reference standard. In some embodiments, the control is a negative control sample obtained from a healthy patient. In other embodiments, the control is a positive control sample obtained from a patient diagnosed with a disease or condition, such as SARS-COV-2 infection. In still other embodiments, the control is a historical control or standard reference value or range of values (such as a previously tested control sample, such as a group of patients infected with a SARS-COV-2 with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Coronavirus: A large family of positive-sense, single-stranded RNA viruses that can infect humans and non-human animals. Coronaviruses get their name from the crown-like spikes on their surface. The viral envelope is comprised of a lipid bilayer containing the viral membrane (M), envelope (E) and spike (S) proteins. Most coronaviruses cause mild to moderate upper respiratory tract illness, such as the common cold. However, three coronaviruses have emerged that can cause more serious illness and death: severe acute respiratory syndrome coronavirus (SARS-COV), SARS-COV-2, and Middle East respiratory syndrome coronavirus (MERS-COV). Other coronaviruses that infect humans include human coronavirus HKU1 (HKUI-CoV), human coronavirus OC43 (OC43-CoV), human coronavirus 229E (229E-CoV), and human coronavirus NL63 (NL63-CoV).

COVID-19: The disease caused by the coronavirus SARS-COV-2.

Degenerate variant: In the context of the present disclosure, a “degenerate variant” refers to a polynucleotide encoding a polypeptide that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

Effective amount: An amount of agent, such as an rB/HPIV2-SARS-COV-2 S vector as described herein, that is sufficient to elicit a desired response, such as an immune response in a subject. It is understood that to obtain a protective immune response against an antigen of interest can require multiple administrations of a disclosed immunogen, and/or administration of a disclosed immunogen as the “prime” in a prime boost protocol wherein the boost immunogen can be different from the prime immunogen. Accordingly, an effective amount of a disclosed immunogen can be the amount of the immunogen sufficient to elicit a priming immune response in a subject that can be subsequently boosted with the same or a different immunogen to elicit a protective immune response.

In one example, a desired response is to inhibit or reduce or prevent SARS-COV-2 infection or associated disease. The SARS-COV-2 infection does not need to be completely eliminated or reduced or prevented for the method to be effective. For example, administration of an effective amount of the agent can induce an immune response that decreases the SARS-COV-2 infection (for example, as measured by infection of cells, or by number or percentage of subjects infected by the SARS-COV-2) by a desired amount, for example by at least 50%, at least 60%, at least 70%, at least 80%, at least 90%, at least 95%, at least 98%, or even at least 100% (elimination or prevention of detectable SARS-COV-2 infection), as compared to a suitable control.

Gene: A nucleic acid sequence that comprises control and coding sequences necessary for the transcription of an RNA, whether an mRNA or otherwise. For instance, a gene may comprise a promoter, one or more enhancers or silencers, a nucleic acid sequence that encodes a RNA and/or a polypeptide, downstream regulatory sequences and, possibly, other nucleic acid sequences involved in regulation of the expression of an mRNA.

A “gene” of a rB/HPIV3 vector as described herein refers to a portion of the rB/HPIV3 genome encoding an mRNA and typically begins at the upstream (3′) end with a gene-start (GS) signal and ends at the downstream (5′) end with the gene-end (GE) signal. In this context, the term gene also embraces what is referred to as a “translational open reading frame”, or ORF, particularly in the case where a protein, such as C, is expressed from an additional ORF rather than from a unique mRNA. To construct a disclosed rB/HPIV3 vector, one or more genes or genome segments may be deleted, inserted or substituted in whole or in part.

Heterologous: Originating from a different genetic source. A heterologous gene included in a recombinant genome is a gene that does not originate from that genome. In one specific, non-limiting example, a heterologous gene encoding a recombinant SARS-COV-2 S protein is included in the genome of a rB/HPIV3 vector as described herein.

Host cells: Cells in which a vector can be propagated and its nucleic acid expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Infectious and self-replicating virus: A virus that is capable of entering and replicating in a cultured cell or cell of an animal or human host to produce progeny virus capable of the same activity. Immune response: A response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response, and results in the production of specific antibodies.

Immunogenic composition: A preparation of immunogenic material capable of stimulating an immune response, which in some examples can be administered for the prevention, amelioration, or treatment of infectious or other types of disease. The immunogenic material may include attenuated or killed microorganisms (such as bacteria or viruses), or antigenic proteins, peptides or DNA derived from them. Immunogenic compositions comprise an antigen (such as a virus) that induces a measurable T cell response against the antigen, or induces a measurable B cell response (such as production of antibodies) against the antigen. In one example, an immunogenic composition comprises a disclosed rB/HPIV3-SARS-CoV-2/S that induces a measurable CTL response against SARS-COV-2 and HPIV3, or induces a measurable B cell response (such as production of antibodies) against SARS-COV-2 and HPIV3, when administered to a subject. For in vivo use, the immunogenic composition will typically include a recombinant virus in a pharmaceutically acceptable carrier and may also include other agents, such as an adjuvant.

Isolated: An “isolated” biological component has been substantially separated or purified away from other biological components, such as other biological components in which the component occurs, such as other chromosomal and extrachromosomal DNA, RNA, and proteins. Proteins, peptides, nucleic acids, and viruses that have been “isolated” include those purified by standard purification methods. Isolated does not require absolute purity, and can include protein, peptide, nucleic acid, or virus molecules that are at least 50% pure, such as at least 75%, 80%, 90%, 95%, 98%, 99%, or even 99.9% pure.

Nucleic acid molecule: A polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “polynucleotide.” A nucleic acid molecule is usually at least 10 bases in length, unless otherwise specified. The term includes single- and double-stranded forms of DNA. A nucleic acid molecule may include either or both naturally occurring and modified nucleotides linked together by naturally occurring and/or non-naturally occurring nucleotide linkages.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and, where necessary to join two protein-coding regions, in the same reading frame.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop, such as a reduction in viral load. “Ameliorating” refers to the reduction in the number or severity of signs or symptoms of a disease, such as a coronavirus infection.

Parainfluenza virus (PIV): A number of enveloped non-segmented negative-sense single-stranded RNA viruses from family Paramyxoviridae that are descriptively grouped together. This includes all of the members of genus Respirovirus (e.g., HPIV1, HPIV3) and a number of members of genus Rubulavirus (e.g. HPIV2, HPIV4, PIV5). PIVs are made up of two structural modules: (1) an internal ribonucleoprotein core, or nucleocapsid, containing the viral genome, and (2) an outer, roughly spherical lipoprotein envelope. The PIV genome is approximately 15,000 nucleotides in length and encodes at least eight polypeptides. These proteins include the nucleocapsid structural protein (NP, NC, or N depending on the genera), the phosphoprotein (P), the matrix protein (M), the fusion glycoprotein (F), the hemagglutinin-neuraminidase glycoprotein (HN), the large polymerase protein (L), and the C and D proteins. The gene order is 3′-N-P-M-F-HN-L-5′, and each gene encodes a separate protein encoding mRNA, with the P gene containing one or more additional open reading frames (ORFs) encoding accessory proteins.

Pharmaceutically acceptable carriers: The pharmaceutically acceptable carriers of use are conventional. Remington's Pharmaceutical Sciences, by E. W. Martin, Mack Publishing Co., Easton, PA, 19th Edition, 1995, describes compositions and formulations suitable for pharmaceutical delivery of the disclosed immunogens.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. In particular embodiments, suitable for administration to a subject the carrier may be sterile, and/or suspended or otherwise contained in a unit dosage form containing one or more measured doses of the composition suitable to induce the desired immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

Polypeptide: Any chain of amino acids, regardless of length or post-translational modification (e.g., glycosylation or phosphorylation). “Polypeptide” applies to amino acid polymers including naturally occurring amino acid polymers and non-naturally occurring amino acid polymer as well as in which one or more amino acid residue is a non-natural amino acid, for example an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

Recombinant: A recombinant nucleic acid, vector or virus is one that has a sequence that is not naturally occurring or has a sequence that is made by an artificial combination of two otherwise separated segments of sequence. This artificial combination can be accomplished, for example, by the artificial manipulation of isolated segments of nucleic acids, for example, using genetic engineering techniques.

Recombinant chimeric bovine/human parainfluenza virus 3 (rB/HPIV3): A chimeric PIV3 comprising a genome comprising a combination of BPIV3 and HPIV3 genes that together make up the full complement of PIV3 genes in the PIV3 genome (N, P, M, F, HN, and L genes). The disclosed rB/HPIV3 vectors are based on a BPIV3 genome having F and HN genes replaced with the corresponding genes from HPIV3 (one example of which is discussed in Schmidt AC et al., J. Virol. 74:8922-8929, 2000). The structural and functional genetic elements that control gene expression, such as gene start and gene end sequences and genome and anti-genome promoters, are BPIV3 structural and functional genetic elements. The rB/HPIV3 vectors described herein are infectious, self-replicating, and attenuated.

In some embodiments, a heterologous gene encoding a recombinant SARS-COV-2 S protein is inserted between the N and P genes of the rB/HPIV3 genome to generate a rB/HPIV3-SARS-COV-2/S vector. The disclosed rB/HPIV3-SARS-COV-2/S vectors are infectious, self-replicating, and attenuated, and can be used to induce a bivalent immune response to SARS-COV-2 and HPIV3 in a subject.

SARS-COV-2: A positive-sense, single stranded RNA virus of the genus betacoronavirus that has emerged as a highly fatal cause of severe acute respiratory infection. SARS-COV-2 is also known as 2019-nCoV, or 2019 novel coronavirus. The viral genome is capped, polyadenylated, and covered with nucleocapsid proteins. The SARS-COV-2 virion includes a viral envelope with large spike glycoproteins. The SARS-COV-2 genome, like most coronaviruses, has a common genome organization with the replicase gene included in the 5′-two thirds of the genome, and structural genes included in the 3′-third of the genome. The SARS-COV-2 genome encodes the canonical set of structural protein genes in the order 5′-spike (S)-envelope (E)-membrane (M) and nucleocapsid (N)-3′. Symptoms of SARS-COV-2 infection include fever and respiratory illness, such as dry cough and shortness of breath. Cases of severe infection can progress to severe pneumonia, multi-organ failure, and death. The time from exposure to onset of symptoms is approximately 2 to 14 days.

Standard methods for detecting viral infection may be used to detect SARS-COV-2 infection, including but not limited to, assessment of patient symptoms and background and genetic tests such as reverse transcription-polymerase chain reaction (rRT-PCR). The test can be done on patient samples such as respiratory or blood samples.

SARS-COV-2 Spike (S): A class I fusion glycoprotein initially synthesized as a precursor protein of approximately 1270 amino acids in size. Individual precursor S polypeptides form a homotrimer and undergo glycosylation within the Golgi apparatus as well as processing to remove the signal peptide. The S polypeptide includes S1 and S2 proteins separated by a protease cleavage site between approximately position 685/686. Cleavage at this site generates separate S1 and S2 polypeptide chains, which remain associated as S1/S2 protomers within the homotrimer. It is believed that the beta coronaviruses are generally not cleaved prior to the low pH cleavage that occurs in the late endosome-early lysosome by the transmembrane protease serine 2 (TMPRSS2), at an additional proteolytic cleavage site S2/S2′ at the start of the fusion peptide. Cleavage between S1/S2 is not required for function and is not observed in all viral spikes. The SI subunit is distal to the virus membrane and contains the receptor-binding domain (RBD) that is believed to mediate virus attachment to its host receptor. The S2 subunit is believed to contain the fusion protein machinery, such as the fusion peptide, two heptad-repeat sequences (HR1 and HR2) and a central helix typical of fusion glycoproteins, a transmembrane domain, and the cytosolic tail domain.

The numbering used in the disclosed SARS-COV-2 S proteins and fragments thereof is relative to the S protein of SARS-COV-2, the sequence of which is provided as SEQ ID NO: 22, and deposited as NCBI Ref. No. YP_009724390.1, which is incorporated by reference herein in its entirety.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs, orthologs, or variants of a polypeptide will possess a relatively high degree of sequence identity when aligned using standard methods.

When determining sequence identity between two sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Sambrook et al. (Molecular Cloning: A Laboratory Manual, 4^{th} ed, Cold Spring Harbor, New York, 2012) and Ausubel et al. (In Current Protocols in Molecular Biology, John Wiley & Sons, New York, through supplement 104, 2013).

Another example of algorithms that are suitable for determining percent sequence identity and sequence similarity are the BLAST and the BLAST 2.0 algorithm, which are described in Altschul et al., J. Mol. Biol. 215:403-410, 1990 and Altschul et al., Nucleic Acids Res. 25:3389-3402, 1977. Software for performing BLAST analyses is publicly available through the National Center for Biotechnology Information (ncbi.nlm.nih.gov). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. USA 89:10915, 1989).

In one example, once aligned, the number of matches is determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a peptide sequence that has 1166 matches when aligned with a test sequence having 1554 amino acids is 75.0 percent identical to the test sequence (1166'1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer.

Homologs and variants of a polypeptide (such as a SARS-COV-2 S protein) are typically characterized by possession of at least about 75%, for example at least about 80%, 85%, 90%, 91%, 92%, 93%, 94%, 95%, 96%, 97%, 98% or 99% sequence identity counted over the full-length alignment with the amino acid sequence of interest. As used herein, reference to “at least 90% identity” or similar language refers to “at least 90%, at least 91%, at least 92%, at least 93%, at least 94%, at least 95%, at least 96%, at least 97%, at least 98%, at least 99%, or even 100% identity” to a specified reference sequence.

Subject: Living multi-cellular vertebrate organisms, a category that includes human and non-human mammals. In an example, a subject is a human. In a particular example, the subject is a newborn infant. In an additional example, the selected subject is in need of inhibiting a SARS-COV-2 infection and/or a HPIV3 infection. For example, the subject is either uninfected and at risk of SARS-COV-2 infection and/or HPIV3 infection or is infected and in need of treatment.

Vaccine: A preparation of immunogenic material capable of stimulating an immune response, administered for the prevention, amelioration, or treatment of infectious or other types of disease. The immunogenic material may include attenuated or killed microorganisms (such as bacteria or viruses), or antigenic proteins, peptides or DNA derived from them. An attenuated vaccine is a virulent organism that has been modified to produce a less virulent form, but nevertheless retains the ability to elicit antibodies and cell-mediated immunity against the virulent form. An inactivated (killed) vaccine is a previously virulent organism that has been inactivated with chemicals, heat, or other treatment, but elicits antibodies against the organism. Vaccines may elicit both prophylactic (preventative or protective) and therapeutic responses. Methods of administration vary according to the vaccine, but may include inoculation, ingestion, inhalation or other forms of administration. Vaccines may be administered with an adjuvant to boost the immune response.

Vector: An entity containing a DNA or RNA molecule bearing a promoter(s) that is operationally linked to the coding sequence of an antigen(s) of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant nucleic acid vectors having at least some nucleic acid sequences derived from one or more viruses.

### III. rB/HPIV3-SARS-COV-2/S Vectors

Recombinant chimeric viral vectors comprising a BPIV3 genome with the encoding sequences of the BPIV3 HN and F genes replaced by encoding sequences of the corresponding HPIV3 HN and F gene, and further comprising a heterologous gene encoding a recombinant SARS-COV-2 S protein are provided herein. These recombinant chimeric viral vectors are referred as “rB/HPIV3-SARS-COV-2/S” vectors.

The rB/HPIV3-SARS-COV-2/S genome contains a full complement of PIV3 genes. Therefore, the rB/HPIV3-SARS-COV-2/S vectors are infectious and replication-competent, but are attenuated in rhesus monkeys and humans due to the BPIV3 backbone, and the presence of the heterologous gene.

The genome of the rB/HPIV3-SARS-COV-2/S vectors includes the heterologous gene encoding recombinant SARS-COV2 S protein, HPIV3 F and HN genes, BPIV3 N, P, M, and L genes, and BPIV3 genomic promoter (3′ leader region) and 5′ trailer region, with the order of 3′-leader region—BPIV3 N, heterologous gene, BPIV3 P, BPIV3 M, HPIV3 F, HPIV3 HN, BPIV3 L - 5′-trailer. Exemplary nucleic acid sequences of these genes and proteins encoded thereby are provided herein, as are structural and functional genetic elements that control gene expression, such as gene start and gene end sequences and genome and anti-genome promoters.

An exemplary BPIV3 genome sequence (Kansas stain) is provided as SEQ ID NO: 36 (deposited under GENBANK™ Accession No. AF178654.1, which is incorporated by reference herein in its entirety). An exemplary HPIV3 genome sequence (JS strain) is provided as SEQ ID NO: 37 (deposited under GENBANK™ Accession No. Z11575.1, which is incorporated by reference herein in its entirety). In some embodiments, sequences from these strains can be used to construct the rB/HPIV3 aspect of the rB/HPIV3-SARS-COV-2/S vector, for example, as described in Schmidt et al., (J. Virol. 74:8922-8929, 2000). In some such embodiments, the HN protein encoded by the HPIV3 HN gene can be modified to have threonine and proline residues at positions 263 and 370, respectively.

In some embodiments, the rB/HPIV3-SARS-COV-2/S vector comprises a genome comprising HPIV3 F and HN genes and BPIV3 N, P, M, and L genes encoding HPIV3 F and HN proteins and BPIV3 N, P, C, V, M, and L proteins as set forth below, or encoding HPIV3 F and HN proteins and BPIV3 N, P, C, V, M, and L proteins individually having at least 90% (such as at least 95% or at least 98%) sequence identity to the corresponding HPIV3 F and HN protein or BPIV3 N, P, C, V, M, and L protein set forth below:

BPIV3 N (GENBANK ™ Accession No.: AAF28254.1, encoded by nucleotides 111-1658 of
GENBANK ™ Accession No. AF178654.1)
(SEQ ID NO: 1)
MLSLFDTFSARRQENITKSAGGAVIPGQKNTVSIFALGPSITDDNDKMTLALLFLSHSLDNEKQHAQRAGFLVSLLSMAY
ANPELYLTSNGSNADVKYVIYMIEKDPGRQKYGGFVVKTREMVYEKTTDWMFGSDLEYDQDNMLQNGRSTSTIEDLVHTF
GYPSCLGALIIQVWIILVKAITSISGLRKGFFTRLEAFRQDGTVKSSLVLSGDAVEQIGSIMRSQQSLVTLMVETLITMN
TGRNDLTTIEKNIQIVGNYIRDAGLASFENTIRYGIETRMAALTLSTLRPDINRLKALIELYLSKGPRAPFICILRDPVH
GEFAPGNYPALWSYAMGVAVVQNKAMQQYVTGRSYLDIEMFQLGQAVARDAESQMSSILEDELGVTQEAKQSLKKHMKNI
SSSDTTFHKPTGGSAIEMAIDEEAGQPESRGDQDQGDEPRSSIVPYAWADETGNDNQTESTTEIDSIKTEQRNIRDRINK
RLNEKRKQSDPRSTDITNNTNQTEIDDLESAFGSN
BPIV3 P (GENBANK ™ Accession No.: AAF28255, encoded by nucleotides 1784-3574 of
GENBANK ™ Accession No. AF178654)
(SEQ ID NO: 2)
MEDNVQNNQIMDSWEEGSGDKSSDISSALDIIEFILSTDSQENTADSNEINTGTTRLSTTIYQPESKTTETSKENSGPAN
KNRQFGASHERATETKDRNVNQETVQGGYRRGSSPDSRTETMVTRRISRSSPDPNNGTQIQEDIDYNEVGEMDKDSTKRE
MRQFKDVPVKVSGSDAIPPTKQDGDGDDGRGLESISTFDSGYTSIVTAATLDDEEELLMKNNRPRKYQSTPQNSDKGIKK
GVGRPKDTDKQSSILDYELNFKGSKKSQKILKASTNTGEPTRPQNGSQGKRITSWNILNSESGNRTESTNQTHQTSTSGQ
NHTMGPSRTTSEPRIKTQKTDGKEREDTEESTRFTERAITLLQNLGVIQSAAKLDLYQDKRVVCVANVLNNADTASKIDE
LAGLMIGVSMDHDTKLNQIQNEILSLKTDLKKMDESHRRLIENQKEQLSLITSLISNLKIMTERGGKKDQPEPSGRISMI
KTKAKEEKIKKVREDPLMETQGIEKNIPDLYRSIEKTPENDTQIKSEINRLNDESNATRLVPRRISSTMRSLIIIINNSN
LSSKAKQSYINELKLCKSDEEVSELMDMENEDVSSQ
BPIV3 C (encoded by nucleotide 1794-2399 of GENBANK ™ Accession No. AF178654)
(SEQ ID NO: 3)
MFKTIKSWILGKRDQEINHLTSHRPSTSLNSYSAPTPKRTRQTAMKSTQEPQDLARQSTNLNPKQQKQARKIVDQLTKID
SLGHHTNVPQRQKIEMLIRRLYREDIGEEAAQIVELRLWSLEESPEAAQILTMEPKSRKILITMKLERWIRTLLRGKCDN
LKMFQSRYQEVMPFLQQNKMETVMMEEAWNLSVHLIQDIPV
BPIV3 V (encoded by nucleotide 1784-3018 of GENBANK ™ Accession No. AF178654 with an
inserted nucleotide g between nucleotide 2505-2506 at a gene editing site located
at nucleotide 2500-2507)
(SEQ ID NO: 4)
MEDNVQNNQIMDSWEEGSGDKSSDISSALDIIEFILSTDSQENTADSNEINTGTTRLSTTIYQPESKTTETSKENSGPAN
KNRQFGASHERATETKDRNVNQETVQGGYRRGSSPDSRTETMVTRRISRSSPDPNNGTQIQEDIDYNEVGEMDKDSTKRE
MRQFKDVPVKVSGSDAIPPTKQDGDGDDGRGLESISTFDSGYTSIVTAATLDDEEELLMKNNRPRKYQSTPQNSDKGIKK
GGWKAKRHRQTIINIGLRTQLQRIEEEPENPQSQHEYRRINKTTEWIPGEENHILEHPQQRERQSNRINKPNPSDINLGT
EPHNGTKQNNLRTKDQDTKDGWKGKRGHRREHSIYRKGDYIITESWCNPICSKIRPIPRQESCVCGECPKQCRYCIKDRL
PSREDDRSVNGS
BPIV3 M (GENBANK ™ Accession No .: AAF28256, encoded by nucleotides 3735-4790 of
GENBANK ™ Accession No. AF178654)
(SEQ ID NO: 5)
MSITNSTIYTFPESSFSENGNIEPLPLKVNEQRKAIPHIRVVKIGDPPKHGSRYLDVFLLGFFEMERSKDRYGSISDLDD
DPSYKVCGSGSLPLGLARYTGNDQELLQAATKLDIEVRRTVKATEMIVYTVQNIKPELYPWSSRLRKGMLFDANKVALAP
QCLPLDRGIKFRVIFVNCTAIGSITLFKIPKSMALLSLPNTISINLQVHIKTGVQTDSKGVVQILDEKGEKSLNFMVHLG
LIKRKMGRMYSVEYCKQKIEKMRLLFSLGLVGGISFHVNATGSISKTLASQLAFKREICYPLMDLNPHLNSVIWASSVEI
TRVDAVLQPSLPGEFRYYPNIIAKGVGKIRQ
HPIV3 F (encoded by nucleotides 5072-6691 of GENBANK ™ Accession No. Z11575)
(SEQ ID NO: 6)
MPTSILLIITTMIMASFCQIDITKLQHVGVLVNSPKGMKISQNFETRYLILSLIPKIEDSNSCGDQQIKQYKKLLDRLII
PLYDGLRLQKDVIVTNQESNENTDPRTKRFFGGVIGTIALGVATSAQITAAVALVEAKQARSDIEKLKEAIRDTNKAVQS
VQSSIGNLIVAIKSVQDYVNKEIVPSIARLGCEAAGLQLGIALTQHYSELTNIFGDNIGSLQEKGIKLQGIASLYRTNIT
EIFTTSTVDKYDIYDLLFTESIKVRVIDVDLNDYSITLQVRLPLLTRLLNTQIYKVDSISYNIQNREWYIPLPSHIMTKG
AFLGGADVKECIEAFSSYICPSDPGFVLNHEIESCLSGNISQCPRITVTSDIVPRYAFVNGGVVANCITTTCTCNGIGNR
INQPPDQGVKIITHKECSTIGINGMLFNINKEGTLAFYTPNDITLNNSVALDPIDISIELNKAKSDLEESKEWIRRSNQK
LDSIGNWHQSSTTIIIILIMIIILFIINITIITIAIKYYRIQKRNRVDQNDKPYVLINK
HPIV3 wt HN (encoded by nucleotides 6806-8524 of GENBANK ™ Accession No. Z11575)
(SEQ ID NO: 7)
MEYWKHTNHGKDAGNELETSMATHGNKLINKIIYILWTIILVLLSIVFIIVLINSIKSEKAHESLLQDINNEFMEITEKI
QMASDNTNDLIQSGVNTRLLTIQSHVQNYIPISLTQQMSDLRKFISEITIRNDNQEVLPQRITHDVGIKPLNPDDFWRCT
SGLPSLMKTPKIRLMPGPGLLAMPTTVDGCVRTPSLVINDLIYAYTSNLITRGCQDIGKSYQVLQIGIITVNSDLVPDLN
PRISHTFNINDNRKSCSLALLNTDVYQLCSTPKVDERSDYASSGIEDIVLDIVNYDGSISTTREKNNNISFDQPYAALYP
SVGPGIYYKGKIIFLGYGGLEHPINENVICNTTGCPGKTQRDCNQASHSPWFSDRRMVNSIIVVDKGLNSIPKLKVWTIS
MRQNYWGSEGRLLLLGNKIYIYTRSTSWHSKLQLGIIDITDYSDIRIKWTWHNVLSRPGNNECPWGHSCPDGCITGVYTD
AYPLNPTGSIVSSVILDSQKSRVNPVITYSTATERVNELAILNRTLSAGYTTTSCITHYNKGYCFHIVEINHKSLNTFQP
MLFKTEIPKSCS

In some embodiment, the HPIV3 HN gene in rB/HPIV3 vector encodes a HPIV3 HN protein comprising the amino acid sequence set forth as:

(SEQ ID NO: 8)
MEYWKHTNHGKDAGNELETSMATHGNKLINKIIYILWTIILVLLSIVFIIVLINSIKSEKAHESLLQDINNEFMEITEKI
QMASDNTNDLIQSGVNTRLLTIQSHVQNYIPISLTQQMSDLRKFISEITIRNDNQEVLPQRITHDVGIKPLNPDDEWRCT
SGLPSLMKTPKIRLMPGPGLLAMPTTVDGCVRTPSLVINDLIYAYTSNLITRGCQDIGKSYQVLQIGIITVNSDLVPDLN
PRISHTFNINDNRKSCSLALLNIDVYQLCSTPKVDERSDYASSGIEDIVLDIVNYDGSISTTREKNNNISFDQPYAALYP
SVGPGIYYKGKIIFLGYGGLEHPINENVICNTTGCPGKTQRDCNQASHSTWFSDRRMVNSIIVVDKGLNSIPKLKVWTIS
MRQNYWGSEGRLLLLGNKIYIYTRSTSWHSKLQLGIIDITDYSDIRIKWTWHNVLSRPGNNECPWGHSCPDGCITGVYTD
AYPLNPTGSIVSSVILDSQKSRVNPVITYSTATERVNELAILNRTLSAGYTTTSCITHYNKGYCFHIVEINHKSLNTFQP
MLFKTEIPKSCS

The HIN protein shown as SEQ ID NO: 7 comprises 263T and 370P amino acid assignments. As discussed in the examples, rB/HPIV3-SARS-COV-2/S including an HN protein with 263T and 370P amino acid assignments can be recovered and passaged with substantially reduced occurrence of adventitious mutations, which increases the efficiency of virus production, analysis, and manufacture. Any of the rB/HPIV3-SARS-COV-2/S vectors provided herein can comprise a HPIV3 HN gene encoding HN protein with 263T and 370P amino acid assignments (for example, introduced into the HN protein by I263T and T370P amino acid substitutions). An exemplary DNA sequence encoding SEQ ID NO: 7 is provided as follows:

(SEQ ID NO: 9)
atggaatactggaagcataccaatcacggaaaggatgctggtaatgagctggagacgtctatggctactcatggcaacaa
gctcactaataagataatatacatattatggacaataatcctggtgttattatcaatagtcttcatcatagtgctaatta
attccatcaaaagtgaaaaggcccacgaatcattgctgcaagacataaataatgagtttatggaaattacagaaaagatc
caaatggcatcggataataccaatgatctaatacagtcaggagtgaatacaaggcttcttacaattcagagtcatgtcca
gaattacataccaatatcattgacacaacagatgtcagatcttaggaaattcattagtgaaattacaattagaaatgata
atcaagaagtgctgccacaaagaataacacatgatgtaggtataaaacctttaaatccagatgatttttggagatgcacg
tctggtcttccatctttaatgaaaactccaaaaataaggttaatgccagggccgggattattagctatgccaacgactgt
tgatggctgtgttagaactccgtctttagttataaatgatctgatttatgcttatacctcaaatctaattactcgaggtt
gtcaggatataggaaaatcatatcaagtcttacagatagggataataactgtaaactcagacttggtacctgacttaaat
cctaggatctctcatacctttaacataaatgacaataggaagtcatgttctctagcactcctaaatatagatgtatatca
actgtgttcaactcccaaagttgatgaaagatcagattatgcatcatcaggcatagaagatattgtacttgatattgtca
attatgatggttcaatctcaacaacaagatttaagaataataacataagctttgatcaaccatatgctgcactataccca
tctgttggaccagggatatactacaaaggcaaaataatatttctcgggtatggaggtcttgaacatccaataaatgagaa
tgtaatctgcaacacaactgggtgccccgggaaaacacagagagactgtaatcaagcatctcatagtacttggttttcag
ataggaggatggtcaactccatcattgttgttgacaaaggcttaaactcaattccaaaattgaaagtatggacgatatct
atgcgacaaaattactgggggtcagaaggaaggttacttctactaggtaacaagatctatatatatacaagatctacaag
ttggcatagcaagttacaattaggaataattgatattactgattacagtgatataaggataaaatggacatggcataatg
tgctatcaagaccaggaaacaatgaatgtccatggggacattcatgtccagatggatgtataacaggagtatatactgat
gcatatccactcaatcccacagggagcattgtgtcatctgtcatattagactcacaaaaatcgagagtgaacccagtcat
aacttactcaacagcaaccgaaagagtaaacgagctggccatcctaaacagaacactctcagctggatatacaacaacaa
gctgcattacacactataacaaaggatattgttttcatatagtagaaataaatcataaaagcttaaacacatttcaaccc
atgttgttcaaaacagagattccaaaaagctgcagttaa
BPIV3 L (GENBANK ™ Accession No.: AAF28259, encoded by nucleotides 8640-15341 of
GENBANK ™ Accession No. AF178654)
(SEQ ID NO: 10)
MDTESHSGTTSDILYPECHLNSPIVKGKIAQLHTIMSLPQPYDMDDDSILIITRQKIKLNKLDKRQRSIRKLRSVLMERV
SDLGKYTFIRYPEMSSEMFQLCIPGINNKINELLSKASKTYNQMTDGLRDLWVTILSKLASKNDGSNYDINEDISNISNV
HMTYQSDKWYNPFKTWFTIKYDMRRLQKAKNEITFNRHKDYNLLEDQKNILLIHPELVLILDKQNYNGYIMTPELVLMYC
DVVEGRWNISSCAKLDPKLQSMYYKGNNLWEIIDGLFSTLGERTFDIISLLEPLALSLIQTYDPVKQLRGAFLNHVLSEM
ELIFAAECTTEEIPNVDYIDKILDVFKESTIDEIAEIFSFFRTFGHPPLEASIAAEKVRKYMYTEKCLKFDTINKCHAIF
CTIIINGYRERHGGQWPPVTLPVHAHEFIINAYGSNSAISYENAVDYYKSFIGIKFDKFIEPQLDEDLTIYMKDKALSPK
KSNWDTVYPASNLLYRTNVSHDSRRLVEVFIADSKFDPHQVLDYVESGYWLDDPEFNISYSLKEKEIKQEGRLFAKMTYK
MRATQVLSETLLANNIGKFFQENGMVKGEIELLKRLTTISMSGVPRYNEVYNNSKSHTEELQAYNAISSSNLSSNQKSKK
FEFKSTDIYNDGYETVSCFLTTDLKKYCLNWRYESTALFGDTCNQIFGLKELFNWLHPRLEKSTIYVGDPYCPPSDIEHL
PLDDHPDSGFYVHNPKGGIEGFCQKLWTLISISAIHLAAVKIGVRVTAMVQGDNQAIAVTTRVPNNYDYKVKKEIVYKDV
VRFFDSLREVMDDLGHELKLNETIISSKMFIYSKRIYYDGRILPQALKALSRCVFWSETIIDETRSASSNLATSFAKAIE
NGYSPVLGYVCSIFKNIQQLYIALGMNINPTITQNIKDQYFRNIHWMQYASLIPASVGGFNYMAMSRCFVRNIGDPTVAA
LADIKRFIKANLLDRGVLYRIMNQEPGESSFLDWASDPYSCNLPQSQNITTMIKNITARNVLQDSPNPLLSGLFTSTMIE
EDEELAEFLMDRRIILPRVAHDILDNSLTGIRNAIAGMLDTTKSLIRVGISRGGLTYNLLRKISNYDLVQYETLSKTLRL
IVSDKIKYEDMCSVDLAISLRQKMWMHLSGGRMINGLETPDPLELLSGVIITGSEHCRICYSTEGESPYTWMYLPGNLNI
GSAETGIASLRVPYFGSVTDERSEAQLGYIKNLSKPAKAAIRIAMIYTWAFGNDEISWMEASQIAQTRANFTLDSLKILT
PVTTSTNLSHRLKDTATQMKFSSTSLIRVSRFITISNDNMSIKEANETKDTNLIYQQVMLTGLSVFEYLFRLEESTGHNP
MVMHLHIEDGCCIKESYNDEHINPESTLELIKYPESNEFIYDKDPLKDIDLSKLMVIRDHSYTIDMNYWDDTDIVHAISI
CTAVTIADTMSQLDRDNLKELVVIANDDDINSLITEFLTLDILVFLKTFGGLLVNQFAYTLYGLKIEGRDPIWDYIMRTL
KDTSHSVLKVLSNALSHPKVFKRFWDCGVLNPIYGPNTASQDQVKLALSICEYSLDLFMREWLNGASLEIYICDSDMEIA
NDRRQAFLSRHLAFVCCLAEIASFGPNLLNLTYLERLDELKQYLDLNIKEDPTLKYVQVSGLLIKSFPSTVTYVRKTAIK
YLRIRGINPPETIEDWDPIEDENILDNIVKTVNDNCSDNQKRNKSSYFWGLALKNYQVVKIRSITSDSEVNEASNVTTHG
MTLPQGGSYLSHQLRLFGVNSTSCLKALELSQILMREVKKDKDRLFLGEGAGAMLACYDATLGPAINYYNSGLNITDVIG
QRELKIFPSEVSLVGKKLGNVTQILNRVRVLENGNPNSTWIGNMECESLIWSELNDKSIGLVHCDMEGAIGKSEETVLHE
HYSIIRITYLIGDDDVVLVSKIIPTITPNWSKILYLYKLYWKDVSVVSLKTSNPASTELYLISKDAYCTVMEPSNLVLSK
LKRISSIEENNLLKWIILSKRKNNEWLQHEIKEGERDYGIMRPYHTALQIFGFQINLNHLAREFLSTPDLTNINNIIQSF
TRTIKDVMFEWVNITHDNKRHKLGGRYNLFPLKNKGKLRLLSRRLVLSWISLSLSTRLLTGRFPDEKFENRAQTGYVSLA
DIDLESLKLLSRNIVKNYKEHIGLISYWELTKEVKILMKLIGGVKLLGIPKQYKELEDRSSQGYEYDNEFDID

The encoding sequences of the HPIV3 F and HN genes and the BPIV3 N, P, M, and L genes in the rB/HPIV3-SARS-COV-2/S vector are flanked by appropriate gene start and gene-end sequences to facilitate expression from the viral genome. For example, in some embodiments, the encoding sequences of the HPIV3 F and HN genes and the BPIV3 N, P, M, and L genes can be flanked by BPIV3 gene-start and gene end sequences as follows:

| Gene | Gene start | SEQ ID NO: | Gene end | SEQ ID NO: |
|---|---|---|---|---|
| N | aggattaaag | 12 | aaataagaaaaa | 16 |
| P | aggattaaag | 12 | aaataagaaaaa | 17 |
| M | aggattaaag | 12 | aaataaaggataatcaaaaa | 18 |
| F | aggacaaaag | 13 | aattataaaaaa | 19 |
| HN | aggagtaaag | 14 | aaatataaaaaa | 20 |
| L | aggagcaaag | 15 | aaagtaagaaaaa | 21 |

Further, the rB/HPIV3-SARS-COV-2/S vector includes appropriate genome and anti-genome promoters, such as those of the BPIV3 Kansas strain as set forth in GENBANK™ Accession No. AF178654 (SEQ ID NO: 36), which provides genomic promoter as nucleotides 1-96 and the antigenomic promoter as nucleotides 15361-15456.

The genome of the rB/HPIV3-SARS-COV-2/S comprises a heterologous gene encoding a recombinant SARS-COV-2 S protein with one or modifications, including to stabilize the SARS-COV2 S protein in its prefusion conformation. An exemplary sequence of native SARS-COV-2 S is provided as SEQ ID NO: 22 (NCBI Ref. No. YP_009724390.1, incorporated by reference herein):

MFVFLVLLPLVSSQCVNLTTRTQLPPAYTNSFTRGVYYPDKVFRSSVLHSTQDLFLPFFSNVTWFHAIHVSGINGTKRED
NPVLPFNDGVYFASTEKSNIIRGWIFGTTLDSKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETKCTLKSFTVEKGIYQTSNERV
QPTESIVRFPNITNLCPFGEVENATRFASVYAWNRKRISNCVADYSVLYNSASFSTFKCYGVSPTKLNDLCFTNVYADSE
VIRGDEVRQIAPGQTGKIADYNYKLPDDFIGCVIAWNSNNLDSKVGGNYNYLYRLERKSNLKPFERDISTEIYQAGSTPC
NGVEGENCYFPLQSYGFQPTNGVGYQPYRVVVLSFELLHAPATVCGPKKSTNLVKNKCVNFNFNGLTGTGVLTESNKKEL
PFQQFGRDIADTTDAVRDPQTLEILDITPCSFGGVSVITPGTNTSNQVAVLYQDVNCTEVPVAIHADQLTPTWRVYSTGS
NVFQTRAGCLIGAEHVNNSYECDIPIGAGICASYQTQTNSPRRARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLICAQKENGLTVLPPLLTDEMIAQYTSALLAG
TITSGWTFGAGAALQIPFAMQMAYRENGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRASANLAATKMSECVLGQSKRV
DFCGKGYHLMSFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDDSEPVLKGVKLHYT

The SARS-COV-2 S protein encoded by the heterologous gene of the rB/HPIV3 vector provide herein is stabilized in a prefusion conformation by one or more amino acid substitutions. In some embodiments, the recombinant SARS-COV-2 S protein is stabilized in the prefusion conformation by K986P and V987P substitutions (“2P”). In some embodiments, the recombinant SARS-COV-2 S protein is stabilized in the prefusion conformation by the one or more proline substitutions (such as K986P and V987P substitutions) and comprises one or more additional modifications for stabilization in the prefusion conformation. For example, the recombinant SARS-COV-2 S protein is stabilized in the prefusion conformation by K986P, V987P, F817P, A892P, A899P, and A942P substitutions (“6P”).

In some embodiments, the recombinant SARS-COV-2 S protein comprises a mutation of the S1/S2 protease cleavage site to prevent cleavage and formation of distinct S1 and S2 polypeptide chains. In some embodiments, the S1 and S2 polypeptides of SARS-COV-2 S are joined by a linker, such as a peptide linker. Examples of peptide linkers that can be used include glycine, serine, and glycine-serine linkers. In some embodiments, the S1/S2 protease cleavage site is mutated by a RRAR(682-685)GSAS substitution. Any of the prefusion stabilizing mutations (or combinations thereof) disclosed herein can be included in the SARS-CoV-2 S protein with the mutated S1/S2 cleavage site as long as the SARS-COV-2 S protein retains the desired properties (e.g., the prefusion conformation).

An exemplary sequence of recombinant SARS-COV-2 S protein including K986P and V987P substitutions for nrefusion stabilization is provided as:

SEQ ID NO: 23
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

In some embodiments, the heterologous gene in the rB/HPIV3 vector encodes a recombinant SARS-CoV-2 protein comprising SEQ ID NO: 23. In some embodiments, the heterologous gene in the rB/HPIV3 vector encodes a recombinant SARS-COV-2 protein comprising K986P and V987P substitutions and an amino acid sequence at least 90% (such as at least 95%, at least 98%, or at least 99%) identical to SEQ ID NO: 23.

An exemplary sequence of recombinant SARS-COV-2 S protein including K986P, V987P, F817P, A892P, A899P, and A942P substitutions for prefusion stabilization is provided as:

SEQ ID NO: 24
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADITDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSPIEDLLFNKVTLADAGFIKQYGDCLGDIAARDLI
CAQKFNGLTVLPPLLTDEMIAQYTSALLAGTITSGWTFGAGPALQIPFPM
QMAYRFNGIGVTQNVLYENQKLIANQFNSAIGKIQDSLSSTPSALGKLQD
VVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

In some embodiments, the heterologous gene in the rB/HPIV3 vector encodes a recombinant SARS-CoV-2 protein comprising SEQ ID NO: 24. In some embodiments, the heterologous gene in the rB/HPIV3 vector encodes a recombinant SARS-COV-2 protein comprising K986P, V987P, F817P, A892P, A899P, and A942P substitutions and an amino acid sequence at least 90% (such as at least 95%, at least 98%, or at least 99%) identical to SEQ ID NO: 24.

An exemplary sequence of recombinant SARS-COV-2 S protein including K986P and V987P substitutions for prefusion stabilization and a RRAR(682-685)GSAS substitution to remove the S1/S2 protease cleavage site is provided as:

SEQ ID NO: 25
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPGSASSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

In some embodiments, the heterologous gene in the rB/HPIV3 vector encodes a recombinant SARS-CoV-2 protein comprising SEQ ID NO: 25. In some embodiments, the heterologous gene in the rB/HPIV3 vector encodes a recombinant SARS-COV-2 protein comprising K986P and V987P substitutions and a RRAR(682-685)GSAS substitution and an amino acid sequence at least 90% (such as at least 95%, at least 98%, or at least 99%) identical to SEQ ID NO: 25.

An exemplary sequence of recombinant SARS-COV-2 S protein including K986P, V987P, F817P, A892P, A899P, and A942P substitutions for prefusion stabilization and a RRAR(682-685)GSAS substitution to remove the S1/S2 protease cleavage site is provided as:

SEQ ID NO: 26
MFVFLVLLPLVSSQCVNLTTRTQLPPAYTNSFTRGVYYPDKVFRSSVLHS
TQDLFLPFFSNVTWFHAIHVSGINGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPGSASSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSPIEDLLFNKVTLADAGFIKQYGDCLGDIAARDLI
CAQKFNGLTVLPPLLTDEMIAQYTSALLAGTITSGWTFGAGPALQIPFPM
QMAYRFNGIGVTQNVLYENQKLIANQFNSAIGKIQDSLSSTPSALGKLQD
VVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

In some embodiments, the heterologous gene in the rB/HPIV3 vector encodes a recombinant SARS-CoV-2 protein comprising SEQ ID NO: 26. In some embodiments, the heterologous gene in the rB/HPIV3 vector encodes a recombinant SARS-COV-2 protein comprising K986P, V987P, F817P, A892P, A899P, and A942P substitutions and a RRAR(682-685)GSAS substitution to remove the S1/S2 protease cleavage site and an amino acid sequence at least 90% (such as at least 95%, at least 98%, or at least 99%) identical to SEQ ID NO: 26.

Also provided is an exemplary amino acid sequence of recombinant SARS-COV-2 S protein with amino acid modifications characteristic of a B.1.617.2/Delta representative, designed to include proline substitutions K986P, V987P, F817P, A892P, A899P, and A942P for prefusion stabilization and a RRAR(682-685)GSAS substitution to remove the S1/S2 protease cleavage site (in boldface below). The sequence is provided as:

S-6P/B.1.617.2/Delta,
SEQ ID NO: 38
MFVFLVLLPLVSSQCVNLRTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESGVYSSANNCTFEYVSQPFLMDLEGKQGNFKNLREFVFKNIDGYFK
IYSKHTPINLVRDLPQGFSALEPLVDLPIGINITRFQTLLALHRSYLTPG
DSSSGWTAGAAAYYVGYLQPRTFLLKYNENGTITDAVDCALDPLSETKCT
LKSFTVEKGIYQTSNFRVQPTESIVRFPNITNLCPFGEVFNATRFASVYA
WNRKRISNCVADYSVLYNSASFSTFKCYGVSPTKLNDLCFTNVYADSFVI
RGDEVRQIAPGQTGKIADYNYKLPDDFTGCVIAWNSNNLDSKVGGNYNYR
YRLFRKSNLKPFERDISTEIYQAGSKPCNGVEGFNCYFPLQSYGFQPTNG
VGYQPYRVVVLSFELLHAPATVCGPKKSTNLVKNKCVNFNFNGLTGTGVL
TESNKKFLPFQQFGRDIADTTDAVRDPQTLEILDITPCSFGGVSVITPGT
NTSNQVAVLYQGVNCTEVPVAIHADQLTPTWRVYSTGSNVFQTRAGCLIG
AEHVNNSYECDIPIGAGICASYQTQTNSRGSASSVASQSIIAYTMSLGAE
NSVAYSNNSIAIPTNFTISVTTEILPVSMTKTSVDCTMYICGDSTECSNL
LLQYGSFCTQLNRALTGIAVEQDKNTQEVFAQVKQIYKTPPIKDFGGFNF
SQILPDPSKPSKRSPIEDLLFNKVTLADAGFIKQYGDCLGDIAARDLICA
QKFNGLTVLPPLLTDEMIAQYTSALLAGTITSGWTFGAGPALQIPFPMQM
AYRFNGIGVTQNVLYENQKLIANQFNSAIGKIQDSLSSTPSALGKLQNVV
NQNAQALNTLVKQLSSNFGAISSVLNDILSRLDPPEAEVQIDRLITGRLQ
SLQTYVTQQLIRAAEIRASANLAATKMSECVLGQSKRVDFCGKGYHLMSF
PQSAPHGVVFLHVTYVPAQEKNFTTAPAICHDGKAHFPREGVFVSNGTHW
FVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQE
LGKYEQYIKWPWYIWLGFIAGLIAIVMVTIMLCCMTSCCSCLKGCCSCGS
CCKFDEDDSEPVLKGVKLHYT

In some embodiments, the heterologous gene in the rB/HPIV3 vector encodes a recombinant SARS-CoV-2 protein comprising SEQ ID NO: 38. In some embodiments, the heterologous gene in the rB/HPIV3 vector encodes a recombinant SARS-COV-2 protein comprising K986P, V987P, F817P, A892P, A899P, and A942P substitutions and an amino acid sequence at least 90% (such as at least 95%, at least 98%, or at least 99%) identical to SEQ ID NO: 38.

Further provided is an exemplary amino acid sequence of recombinant SARS-COV-2 S protein with amino acid modifications characteristic of a B.1.529.1/Omicron representative, designed to include proline substitutions K986P, V987P, F817P, A892P, A899P, and A942P for prefusion stabilization and a RRAR(682-685)GSAS substitution to remove the S1/S2 protease cleavage site (in boldface below). The sequence is provided as:

S-6P/B.1.529/Omicron,
SEQ ID NO: 39
MFVFLVLLPLVSSQCVNLTTRTQLPPAYTNSFTRGVYYPDKVFRSSVLHS
TQDLFLPFFSNVTWFHVISGINGTKRFDNPVLPFNDGVYFASIEKSNIIR
GWIFGTTLDSKTQSLLIVNNATNVVIKVCEFQFCNDPFLDHKNNKSWMES
EFRVYSSANNCTFEYVSQPFLMDLEGKQGNFKNLREFVFKNIDGYFKIYS
KHTPIIVREPEDLPQGFSALEPLVDLPIGINITRFQTLLALHRSYLTPGD
SSSGWTAGAAAYYVGYLQPRTFLLKYNENGTITDAVDCALDPLSETKCTL
KSFTVEKGIYQTSNFRVQPTESIVRFPNITNLCPFDEVFNATRFASVYAW
NRKRISNCVADYSVLYNLAPFFTFKCYGVSPTKLNDLCFTNVYADSFVIR
GDEVRQIAPGQTGNIADYNYKLPDDFTGCVIAWNSNKLDSKVSGNYNYLY
RLFRKSNLKPFERDISTEIYQAGNKPCNGVAGFNCYFPLRSYSFRPTYGV
GHQPYRVVVLSFELLHAPATVCGPKKSTNLVKNKCVNFNFNGLKGTGVLT
ESNKKFLPFQQFGRDIADTTDAVRDPQTLEILDITPCSFGGVSVITPGTN
TSNQVAVLYQGVNCTEVPVAIHADQLTPTWRVYSTGSNVFQTRAGCLIGA
EYVNNSYECDIPIGAGICASYQTQTKSHGSASSVASQSIIAYTMSLGAEN
SVAYSNNSIAIPTNFTISVTTEILPVSMTKTSVDCTMYICGDSTECSNLL
LQYGSFCTQLKRALTGIAVEQDKNTQEVFAQVKQIYKTPPIKYFGGFNFS
QILPDPSKPSKRSPIEDLLFNKVTLADAGFIKQYGDCLGDIAARDLICAQ
KFKGLTVLPPLLTDEMIAQYTSALLAGTITSGWTFGAGPALQIPFPMQMA
YRFNGIGVTQNVLYENQKLIANQFNSAIGKIQDSLSSTPSALGKLQDVVN
HNAQALNTLVKQLSSKFGAISSVLNDIFSRLDPPEAEVQIDRLITGRLQS
LQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWF
VTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCGSC
CKFDEDDSEPVLKGVKLHYT

In some embodiments, the heterologous gene in the rB/HPIV3 vector encodes a recombinant SARS-CoV-2 protein comprising SEQ ID NO: 39. In some embodiments, the heterologous gene in the rB/HPIV3 vector encodes a recombinant SARS-COV-2 protein comprising K986P, V987P, F817P, A892P, A899P, and A942P substitutions and an amino acid sequence at least 90% (such as at least 95%, at least 98%, or at least 99%) identical to SEQ ID NO: 39.

In some embodiments, the SARS-COV-2 S protein further comprises one or more of A67V, a H69 deletion, V70 deletion, T95I, a N211 deletion, L212I, an insertion of 3 codons 214EPE, G142D, a 3-codon deletion V143, Y144, Y145, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F substitutions (numbered with reference to SEQ ID NO: 22).

In some embodiments, the SARS-COV-2 S protein further comprises one or more mutations associated with increased virulence, transmissibility or antigenic differences, such as one or more of L18F, T19R, T20N, P26S, A67V, codon deletions 69-70, D80A, T95I, D138Y, G142D, codon deletions 142-144 or 143-145, Y145D, codon deletions 156-157, R158G, R190S, N211I, L212V, L212I, codon deletions 1213-214, codon insertions 213-214RE, D215G, R216E, G339D, S373P, S375F, K417N, N439K, N440K, G446S, L452R, S477G, S477N, T478K, E484K, E484A, E484Q, Q493R, S494P, G496S, Q498R, N501Y, Y505H, T547K, A570D, D614G, H655Y, N679K, P681H, P681R, A701V, T716K, N764K, D796Y, N856K, D950N, Q954H, N969K, L981F, S982A, T1027I, and D1118H substitutions (numbered with reference to SEQ ID NO: 22).

In some embodiments, the SARS-COV-2 S protein further comprises one or more of K417N, D614G, E484K, N501Y, S477G, S477N, and P681H substitutions. In some embodiments, the SARS-COV-2 S protein further comprises K417N, E484K, N501Y, D614G, and A701V substitutions. In some embodiments, the SARS-COV-2 S protein further comprises K417N, E484K, and N501Y substitutions. In some embodiments, the SARS-COV-2 S protein further comprises one or more deletions of amino acids H69, V70, Y144, L242, A243, and L244 (numbered with reference to SEQ ID NO: 22).

In additional embodiments, the heterologous gene of the rB/HPIV3-SARS-COV-2/S comprises a SARS-COV-2 S protein-coding sequence that has been codon-optimized for expression in a human cell. For example, the encoding sequence of the heterologous gene can be codon-optimized for human expression using a GeneArt (GA-opt), DNA2.0 (D2), or GenScript (GS-opt) optimization algorithm. Non-limiting examples of nucleic acid sequences encoding the recombinant SARS-COV-2 S protein that have been codon-optimized for expression in a human cell are provided as follows:

SARS-COV-2 S-WT GS-opt
(SEQ ID NO: 27)
ATGTTCGTGTTTCTGGTGCTGCTGCCTCTGGTGAGCTCCCAGTGCGTGAACCTGACCACAAGGACCCAGCTGCCCCCTGC
CTATACCAATTCCTTCACACGGGGCGTGTACTATCCCGACAAGGTGTTTAGATCTAGCGTGCTGCACTCCACACAGGATC
TGTTTCTGCCTTTCTTTTCTAACGTGACCTGGTTCCACGCCATCCACGTGAGCGGCACCAATGGCACAAAGCGGTTCGAC
AATCCAGTGCTGCCCTTTAACGATGGCGTGTACTTCGCCTCCACCGAGAAGTCTAACATCATCAGAGGCTGGATCTTTGG
CACCACACTGGACAGCAAGACACAGTCCCTGCTGATCGTGAACAATGCCACCAACGTGGTCATCAAGGTGTGCGAGTTCC
AGTTTTGTAATGATCCATTCCTGGGCGTGTACTATCACAAGAACAATAAGTCTTGGATGGAGAGCGAGTTTCGCGTGTAT
TCCTCTGCCAACAATTGCACATTTGAGTACGTGTCCCAGCCCTTCCTGATGGACCTGGAGGGCAAGCAGGGCAATTTCAA
GAACCTGAGGGAGTTCGTGTTTAAGAATATCGATGGCTACTTCAAGATCTACTCCAAGCACACCCCAATCAACCTGGTGC
GCGACCTGCCACAGGGCTTCTCTGCCCTGGAGCCACTGGTGGATCTGCCCATCGGCATCAACATCACCCGGTTTCAGACA
CTGCTGGCCCTGCACAGAAGCTACCTGACACCAGGCGACAGCTCCTCTGGATGGACCGCAGGAGCTGCCGCCTACTATGT
GGGCTATCTGCAGCCCAGGACCTTCCTGCTGAAGTACAACGAGAATGGCACCATCACAGACGCCGTGGATTGCGCCCTGG
ATCCCCTGTCTGAGACCAAGTGTACACTGAAGAGCTTTACCGTGGAGAAGGGCATCTATCAGACAAGCAATTTCAGGGTG
CAGCCTACCGAGTCCATCGTGCGCTTTCCCAATATCACAAACCTGTGCCCTTTTGGCGAGGTGTTCAACGCAACCCGCTT
CGCCAGCGTGTACGCCTGGAATAGGAAGCGCATCTCCAACTGCGTGGCCGACTATTCTGTGCTGTACAACAGCGCCTCCT
TCTCTACCTTTAAGTGCTATGGCGTGAGCCCCACAAAGCTGAATGACCTGTGCTTTACCAACGTGTACGCCGATTCCTTC
GTGATCAGGGGCGACGAGGTGCGCCAGATCGCCCCTGGCCAGACAGGCAAGATCGCCGACTACAATTATAAGCTGCCTGA
CGATTTCACCGGCTGCGTGATCGCCTGGAACTCTAACAATCTGGATAGCAAAGTGGGCGGCAACTACAATTATCTGTACC
GGCTGTTTAGAAAGTCTAATCTGAAGCCATTCGAGAGGGACATCTCCACAGAGATCTACCAGGCCGGCTCTACCCCCTGC
AATGGCGTGGAGGGCTTTAACTGTTATTTCCCTCTGCAGAGCTACGGCTTCCAGCCAACAAACGGCGTGGGCTATCAGCC
CTACCGCGTGGTGGTGCTGTCTTTTGAGCTGCTGCACGCACCTGCAACAGTGTGCGGACCAAAGAAGAGCACCAATCTGG
TGAAGAACAAGTGCGTGAACTTCAACTTCAACGGACTGACCGGCACAGGCGTGCTGACCGAGTCCAACAAGAAGTTCCTG
CCTTTTCAGCAGTTCGGCAGGGACATCGCAGATACCACAGACGCCGTGCGCGACCCTCAGACCCTGGAGATCCTGGATAT
CACACCATGCTCCTTCGGCGGCGTGTCTGTGATCACACCAGGCACCAATACAAGCAACCAGGTGGCCGTGCTGTATCAGG
ACGTGAATTGTACCGAGGTGCCCGTGGCAATCCACGCAGATCAGCTGACCCCTACATGGCGGGTGTACTCTACCGGCAGC
AACGTGTTCCAGACAAGAGCCGGATGCCTGATCGGAGCCGAGCACGTGAACAATAGCTATGAGTGCGACATCCCTATCGG
CGCCGGCATCTGTGCCTCCTACCAGACCCAGACAAACTCCCCACGGAGAGCCCGGTCTGTGGCCAGCCAGTCCATCATCG
CCTATACCATGAGCCTGGGCGCCGAGAATTCCGTGGCCTACTCCAACAATTCTATCGCCATCCCTACCAACTTCACAATC
TCCGTGACCACAGAGATCCTGCCAGTGAGCATGACCAAGACATCCGTGGACTGCACAATGTATATCTGTGGCGATTCCAC
CGAGTGCTCTAACCTGCTGCTGCAGTACGGCTCTTTTTGTACCCAGCTGAATAGAGCCCTGACAGGCATCGCCGTGGAGC
AGGACAAGAACACACAGGAGGTGTTCGCCCAGGTGAAGCAGATCTACAAGACCCCACCCATCAAGGACTTTGGCGGCTTC
AACTTCAGCCAGATCCTGCCCGATCCTAGCAAGCCATCCAAGCGGTCTTTTATCGAGGACCTGCTGTTCAACAAGGTGAC
CCTGGCCGATGCCGGCTTCATCAAGCAGTATGGCGATTGCCTGGGCGACATCGCCGCCAGAGACCTGATCTGTGCCCAGA
AGTTTAATGGCCTGACCGTGCTGCCTCCACTGCTGACAGATGAGATGATCGCCCAGTACACATCTGCCCTGCTGGCCGGC
ACCATCACAAGCGGATGGACCTTCGGCGCAGGAGCCGCCCTGCAGATCCCCTTTGCCATGCAGATGGCCTATCGGTTCAA
CGGCATCGGCGTGACCCAGAATGTGCTGTACGAGAACCAGAAGCTGATCGCCAATCAGTTTAACTCCGCCATCGGCAAGA
TCCAGGACTCTCTGAGCTCCACAGCCAGCGCCCTGGGCAAGCTGCAGGATGTGGTGAATCAGAACGCCCAGGCCCTGAAT
ACCCTGGTGAAGCAGCTGTCTAGCAACTTCGGCGCCATCTCCTCTGTGCTGAATGATATCCTGAGCAGGCTGGACAAGGT
GGAGGCAGAGGTGCAGATCGACCGGCTGATCACAGGCAGACTGCAGTCCCTGCAGACCTACGTGACACAGCAGCTGATCA
GGGCAGCAGAGATCAGGGCCTCTGCCAATCTGGCCGCCACCAAGATGAGCGAGTGCGTGCTGGGCCAGTCCAAGAGAGTG
GACTTTTGTGGCAAGGGCTATCACCTGATGAGCTTCCCACAGTCCGCCCCTCACGGAGTGGTGTTTCTGCACGTGACCTA
CGTGCCAGCCCAGGAGAAGAACTTCACCACAGCACCAGCAATCTGCCACGATGGCAAGGCACACTTTCCTAGGGAGGGCG
TGTTCGTGAGCAACGGCACCCACTGGTTTGTGACACAGCGCAATTTCTACGAGCCACAGATCATCACCACAGACAATACA
TTCGTGTCCGGCAACTGTGACGTGGTCATCGGCATCGTGAACAATACCGTGTATGATCCTCTGCAGCCAGAGCTGGACTC
TTTTAAGGAGGAGCTGGATAAGTACTTCAAGAATCACACCAGCCCCGACGTGGATCTGGGCGACATCTCTGGCATCAATG
CCAGCGTGGTGAACATCCAGAAGGAGATCGACAGGCTGAACGAGGTGGCCAAGAATCTGAACGAGTCCCTGATCGATCTG
CAGGAGCTGGGCAAGTATGAGCAGTACATCAAGTGGCCCTGGTATATCTGGCTGGGCTTCATCGCCGGCCTGATCGCCAT
CGTGATGGTGACCATCATGCTGTGCTGTATGACAAGCTGCTGTTCCTGCCTGAAGGGCTGCTGTTCTTGTGGCAGCTGCT
GTAAGTTTGATGAGGACGATAGCGAGCCTGTGCTGAAGGGCGTGAAGCTGCACTACACCTGA
SARS-COV-2 S-2P RRAR(682-685)GSAS GS-opt
(SEQ ID NO: 28)
ATGTTCGTGTTTCTGGTGCTGCTGCCTCTGGTGAGCTCCCAGTGCGTGAACCTGACCACAAGGACCCAGCTGCCCCCTGC
CTATACCAATTCCTTCACACGGGGCGTGTACTATCCCGACAAGGTGTTTAGATCTAGCGTGCTGCACTCCACACAGGATC
TGTTTCTGCCTTTCTTTTCTAACGTGACCTGGTTCCACGCCATCCACGTGAGCGGCACCAATGGCACAAAGCGGTTCGAC
AATCCAGTGCTGCCCTTTAACGATGGCGTGTACTTCGCCTCCACCGAGAAGTCTAACATCATCAGAGGCTGGATCTTTGG
CACCACACTGGACAGCAAGACACAGTCCCTGCTGATCGTGAACAATGCCACCAACGTGGTCATCAAGGTGTGCGAGTTCC
AGTTTTGTAATGATCCATTCCTGGGCGTGTACTATCACAAGAACAATAAGTCTTGGATGGAGAGCGAGTTTCGCGTGTAT
TCCTCTGCCAACAATTGCACATTTGAGTACGTGTCCCAGCCCTTCCTGATGGACCTGGAGGGCAAGCAGGGCAATTTCAA
GAACCTGAGGGAGTTCGTGTTTAAGAATATCGATGGCTACTTCAAGATCTACTCCAAGCACACCCCAATCAACCTGGTGC
GCGACCTGCCACAGGGCTTCTCTGCCCTGGAGCCACTGGTGGATCTGCCCATCGGCATCAACATCACCCGGTTTCAGACA
CTGCTGGCCCTGCACAGAAGCTACCTGACACCAGGCGACAGCTCCTCTGGATGGACCGCAGGAGCTGCCGCCTACTATGT
GGGCTATCTGCAGCCCAGGACCTTCCTGCTGAAGTACAACGAGAATGGCACCATCACAGACGCCGTGGATTGCGCCCTGG
ATCCCCTGTCTGAGACCAAGTGTACACTGAAGAGCTTTACCGTGGAGAAGGGCATCTATCAGACAAGCAATTTCAGGGTG
CAGCCTACCGAGTCCATCGTGCGCTTTCCCAATATCACAAACCTGTGCCCTTTTGGCGAGGTGTTCAACGCAACCCGCTT
CGCCAGCGTGTACGCCTGGAATAGGAAGCGCATCTCCAACTGCGTGGCCGACTATTCTGTGCTGTACAACAGCGCCTCCT
TCTCTACCTTTAAGTGCTATGGCGTGAGCCCCACAAAGCTGAATGACCTGTGCTTTACCAACGTGTACGCCGATTCCTTC
GTGATCAGGGGCGACGAGGTGCGCCAGATCGCCCCTGGCCAGACAGGCAAGATCGCCGACTACAATTATAAGCTGCCTGA
CGATTTCACCGGCTGCGTGATCGCCTGGAACTCTAACAATCTGGATAGCAAAGTGGGCGGCAACTACAATTATCTGTACC
GGCTGTTTAGAAAGTCTAATCTGAAGCCATTCGAGAGGGACATCTCCACAGAGATCTACCAGGCCGGCTCTACCCCCTGC
AATGGCGTGGAGGGCTTTAACTGTTATTTCCCTCTGCAGAGCTACGGCTTCCAGCCAACAAACGGCGTGGGCTATCAGCC
CTACCGCGTGGTGGTGCTGTCTTTTGAGCTGCTGCACGCACCTGCAACAGTGTGCGGACCAAAGAAGAGCACCAATCTGG
TGAAGAACAAGTGCGTGAACTTCAACTTCAACGGACTGACCGGCACAGGCGTGCTGACCGAGTCCAACAAGAAGTTCCTG
CCTTTTCAGCAGTTCGGCAGGGACATCGCAGATACCACAGACGCCGTGCGCGACCCTCAGACCCTGGAGATCCTGGATAT
CACACCATGCTCCTTCGGCGGCGTGTCTGTGATCACACCAGGCACCAATACAAGCAACCAGGTGGCCGTGCTGTATCAGG
ACGTGAATTGTACCGAGGTGCCCGTGGCAATCCACGCAGATCAGCTGACCCCTACATGGCGGGTGTACTCTACCGGCAGC
AACGTGTTCCAGACAAGAGCCGGATGCCTGATCGGAGCCGAGCACGTGAACAATAGCTATGAGTGCGACATCCCTATCGG
CGCCGGCATCTGTGCCTCCTACCAGACCCAGACAAACTCCCCAgGGtctGCCtccTCTGTGGCCAGCCAGTCCATCATCG
CCTATACCATGAGCCTGGGCGCCGAGAATTCCGTGGCCTACTCCAACAATTCTATCGCCATCCCTACCAACTTCACAATC
TCCGTGACCACAGAGATCCTGCCAGTGAGCATGACCAAGACATCCGTGGACTGCACAATGTATATCTGTGGCGATTCCAC
CGAGTGCTCTAACCTGCTGCTGCAGTACGGCTCTTTTTGTACCCAGCTGAATAGAGCCCTGACAGGCATCGCCGTGGAGC
AGGACAAGAACACACAGGAGGTGTTCGCCCAGGTGAAGCAGATCTACAAGACCCCACCCATCAAGGACTTTGGCGGCTTC
AACTTCAGCCAGATCCTGCCCGATCCTAGCAAGCCATCCAAGCGGTCTTTTATCGAGGACCTGCTGTTCAACAAGGTGAC
CCTGGCCGATGCCGGCTTCATCAAGCAGTATGGCGATTGCCTGGGCGACATCGCCGCCAGAGACCTGATCTGTGCCCAGA
AGTTTAATGGCCTGACCGTGCTGCCTCCACTGCTGACAGATGAGATGATCGCCCAGTACACATCTGCCCTGCTGGCCGGC
ACCATCACAAGCGGATGGACCTTCGGCGCAGGAGCCGCCCTGCAGATCCCCTTTGCCATGCAGATGGCCTATCGGTTCAA
CGGCATCGGCGTGACCCAGAATGTGCTGTACGAGAACCAGAAGCTGATCGCCAATCAGTTTAACTCCGCCATCGGCAAGA
TCCAGGACTCTCTGAGCTCCACAGCCAGCGCCCTGGGCAAGCTGCAGGATGTGGTGAATCAGAACGCCCAGGCCCTGAAT
ACCCTGGTGAAGCAGCTGTCTAGCAACTTCGGCGCCATCTCCTCTGTGCTGAATGATATCCTGAGCAGGCTGGACcctcc
aGAGGCAGAGGTGCAGATCGACCGGCTGATCACAGGCAGACTGCAGTCCCTGCAGACCTACGTGACACAGCAGCTGATCA
GGGCAGCAGAGATCAGGGCCTCTGCCAATCTGGCCGCCACCAAGATGAGCGAGTGCGTGCTGGGCCAGTCCAAGAGAGTG
GACTTTTGTGGCAAGGGCTATCACCTGATGAGCTTCCCACAGTCCGCCCCTCACGGAGTGGTGTTTCTGCACGTGACCTA
CGTGCCAGCCCAGGAGAAGAACTTCACCACAGCACCAGCAATCTGCCACGATGGCAAGGCACACTTTCCTAGGGAGGGCG
TGTTCGTGAGCAACGGCACCCACTGGTTTGTGACACAGCGCAATTTCTACGAGCCACAGATCATCACCACAGACAATACA
TTCGTGTCCGGCAACTGTGACGTGGTCATCGGCATCGTGAACAATACCGTGTATGATCCTCTGCAGCCAGAGCTGGACTC
TTTTAAGGAGGAGCTGGATAAGTACTTCAAGAATCACACCAGCCCCGACGTGGATCTGGGCGACATCTCTGGCATCAATG
CCAGCGTGGTGAACATCCAGAAGGAGATCGACAGGCTGAACGAGGTGGCCAAGAATCTGAACGAGTCCCTGATCGATCTG
CAGGAGCTGGGCAAGTATGAGCAGTACATCAAGTGGCCCTGGTATATCTGGCTGGGCTTCATCGCCGGCCTGATCGCCAT
CGTGATGGTGACCATCATGCTGTGCTGTATGACAAGCTGCTGTTCCTGCCTGAAGGGCTGCTGTTCTTGTGGCAGCTGCT
GTAAGTTTGATGAGGACGATAGCGAGCCTGTGCTGAAGGGCGTGAAGCTGCACTACACCTGA
SARS-COV-2 S-6P RRAR(682-685)GSAS GS-opt
(SEQ ID NO: 29)
ATGTTCGTGTTTCTGGTGCTGCTGCCTCTGGTGAGCTCCCAGTGCGTGAACCTGACCACAAGGACCCAGCTGCCCCCTGC
CTATACCAATTCCTTCACACGGGGCGTGTACTATCCCGACAAGGTGTTTAGATCTAGCGTGCTGCACTCCACACAGGATC
TGTTTCTGCCTTTCTTTTCTAACGTGACCTGGTTCCACGCCATCCACGTGAGCGGCACCAATGGCACAAAGCGGTTCGAC
AATCCAGTGCTGCCCTTTAACGATGGCGTGTACTTCGCCTCCACCGAGAAGTCTAACATCATCAGAGGCTGGATCTTTGG
CACCACACTGGACAGCAAGACACAGTCCCTGCTGATCGTGAACAATGCCACCAACGTGGTCATCAAGGTGTGCGAGTTCC
AGTTTTGTAATGATCCATTCCTGGGCGTGTACTATCACAAGAACAATAAGTCTTGGATGGAGAGCGAGTTTCGCGTGTAT
TCCTCTGCCAACAATTGCACATTTGAGTACGTGTCCCAGCCCTTCCTGATGGACCTGGAGGGCAAGCAGGGCAATTTCAA
GAACCTGAGGGAGTTCGTGTTTAAGAATATCGATGGCTACTTCAAGATCTACTCCAAGCACACCCCAATCAACCTGGTGC
GCGACCTGCCACAGGGCTTCTCTGCCCTGGAGCCACTGGTGGATCTGCCCATCGGCATCAACATCACCCGGTTTCAGACA
CTGCTGGCCCTGCACAGAAGCTACCTGACACCAGGCGACAGCTCCTCTGGATGGACCGCAGGAGCTGCCGCCTACTATGT
GGGCTATCTGCAGCCCAGGACCTTCCTGCTGAAGTACAACGAGAATGGCACCATCACAGACGCCGTGGATTGCGCCCTGG
ATCCCCTGTCTGAGACCAAGTGTACACTGAAGAGCTTTACCGTGGAGAAGGGCATCTATCAGACAAGCAATTTCAGGGTG
CAGCCTACCGAGTCCATCGTGCGCTTTCCCAATATCACAAACCTGTGCCCTTTTGGCGAGGTGTTCAACGCAACCCGCTT
CGCCAGCGTGTACGCCTGGAATAGGAAGCGCATCTCCAACTGCGTGGCCGACTATTCTGTGCTGTACAACAGCGCCTCCT
TCTCTACCTTTAAGTGCTATGGCGTGAGCCCCACAAAGCTGAATGACCTGTGCTTTACCAACGTGTACGCCGATTCCTTC
GTGATCAGGGGCGACGAGGTGCGCCAGATCGCCCCTGGCCAGACAGGCAAGATCGCCGACTACAATTATAAGCTGCCTGA
CGATTTCACCGGCTGCGTGATCGCCTGGAACTCTAACAATCTGGATAGCAAAGTGGGCGGCAACTACAATTATCTGTACC
GGCTGTTTAGAAAGTCTAATCTGAAGCCATTCGAGAGGGACATCTCCACAGAGATCTACCAGGCCGGCTCTACCCCCTGC
AATGGCGTGGAGGGCTTTAACTGTTATTTCCCTCTGCAGAGCTACGGCTTCCAGCCAACAAACGGCGTGGGCTATCAGCC
CTACCGCGTGGTGGTGCTGTCTTTTGAGCTGCTGCACGCACCTGCAACAGTGTGCGGACCAAAGAAGAGCACCAATCTGG
TGAAGAACAAGTGCGTGAACTTCAACTTCAACGGACTGACCGGCACAGGCGTGCTGACCGAGTCCAACAAGAAGTTCCTG
CCTTTTCAGCAGTTCGGCAGGGACATCGCAGATACCACAGACGCCGTGCGCGACCCTCAGACCCTGGAGATCCTGGATAT
CACACCATGCTCCTTCGGCGGCGTGTCTGTGATCACACCAGGCACCAATACAAGCAACCAGGTGGCCGTGCTGTATCAGG
ACGTGAATTGTACCGAGGTGCCCGTGGCAATCCACGCAGATCAGCTGACCCCTACATGGCGGGTGTACTCTACCGGCAGC
AACGTGTTCCAGACAAGAGCCGGATGCCTGATCGGAGCCGAGCACGTGAACAATAGCTATGAGTGCGACATCCCTATCGG
CGCCGGCATCTGTGCCTCCTACCAGACCCAGACAAACTCCCCAgGGtctGCCtccTCTGTGGCCAGCCAGTCCATCATCG
CCTATACCATGAGCCTGGGCGCCGAGAATTCCGTGGCCTACTCCAACAATTCTATCGCCATCCCTACCAACTTCACAATC
TCCGTGACCACAGAGATCCTGCCAGTGAGCATGACCAAGACATCCGTGGACTGCACAATGTATATCTGTGGCGATTCCAC
CGAGTGCTCTAACCTGCTGCTGCAGTACGGCTCTTTTTGTACCCAGCTGAATAGAGCCCTGACAGGCATCGCCGTGGAGC
AGGACAAGAACACACAGGAGGTGTTCGCCCAGGTGAAGCAGATCTACAAGACCCCACCCATCAAGGACTTTGGCGGCTTC
AACTTCAGCCAGATCCTGCCCGATCCTAGCAAGCCATCCAAGCGGTCTCCTATCGAGGACCTGCTGTTCAACAAGGTGAC
CCTGGCCGATGCCGGCTTCATCAAGCAGTATGGCGATTGCCTGGGCGACATCGCCGCCAGAGACCTGATCTGTGCCCAGA
AGTTTAATGGCCTGACCGTGCTGCCTCCACTGCTGACAGATGAGATGATCGCCCAGTACACATCTGCCCTGCTGGCCGGC
ACCATCACAAGCGGATGGACCTTCGGCGCAGGACCCGCCCTGCAGATCCCCTTTCCCATGCAGATGGCCTATCGGTTCAA
CGGCATCGGCGTGACCCAGAATGTGCTGTACGAGAACCAGAAGCTGATCGCCAATCAGTTTAACTCCGCCATCGGCAAGA
TCCAGGACTCTCTGAGCTCCACACCCAGCGCCCTGGGCAAGCTGCAGGATGTGGTGAATCAGAACGCCCAGGCCCTGAAT
ACCCTGGTGAAGCAGCTGTCTAGCAACTTCGGCGCCATCTCCTCTGTGCTGAATGATATCCTGAGCAGGCTGGACcctcc
aGAGGCAGAGGTGCAGATCGACCGGCTGATCACAGGCAGACTGCAGTCCCTGCAGACCTACGTGACACAGCAGCTGATCA
GGGCAGCAGAGATCAGGGCCTCTGCCAATCTGGCCGCCACCAAGATGAGCGAGTGCGTGCTGGGCCAGTCCAAGAGAGTG
GACTTTTGTGGCAAGGGCTATCACCTGATGAGCTTCCCACAGTCCGCCCCTCACGGAGTGGTGTTTCTGCACGTGACCTA
CGTGCCAGCCCAGGAGAAGAACTTCACCACAGCACCAGCAATCTGCCACGATGGCAAGGCACACTTTCCTAGGGAGGGCG
TGTTCGTGAGCAACGGCACCCACTGGTTTGTGACACAGCGCAATTTCTACGAGCCACAGATCATCACCACAGACAATACA
TTCGTGTCCGGCAACTGTGACGTGGTCATCGGCATCGTGAACAATACCGTGTATGATCCTCTGCAGCCAGAGCTGGACTC
TTTTAAGGAGGAGCTGGATAAGTACTTCAAGAATCACACCAGCCCCGACGTGGATCTGGGCGACATCTCTGGCATCAATG
CCAGCGTGGTGAACATCCAGAAGGAGATCGACAGGCTGAACGAGGTGGCCAAGAATCTGAACGAGTCCCTGATCGATCTG
CAGGAGCTGGGCAAGTATGAGCAGTACATCAAGTGGCCCTGGTATATCTGGCTGGGCTTCATCGCCGGCCTGATCGCCAT
CGTGATGGTGACCATCATGCTGTGCTGTATGACAAGCTGCTGTTCCTGCCTGAAGGGCTGCTGTTCTTGTGGCAGCTGCT
GTAAGTTTGATGAGGACGATAGCGAGCCTGTGCTGAAGGGCGTGAAGCTGCACTACACCTGA

In some embodiments, the genome of the rB/HPIV3-SARS-COV-2/S vector comprises an antigenomic eDNA sequence set forth as SEQ ID NO: 30.

rB/HPIV3-SARS-COV-2/S-2P RRAR(682-685)GSAS
(SEQ ID NO: 30)
ACCAAACAAGAGAAGAGACTGGTTTGGGAATATTAATTCAAATAAAAATTAACTTAGGATTAAAGAACTTTACCGAAAGG
TAAGGGGAAAGAAATCCTAAGAGCTTAGCCATGTTGAGTCTATTCGACACATTCAGTGCGCGTAGGCAGGAGAACATAAC
GAAATCAGCTGGTGGGGCTGTTATTCCCGGGCAAAAAAACACTGTGTCTATATTTGCTCTTGGACCATCAATAACAGATG
ACAATGATAAAATGACATTGGCTCTTCTCTTTTTGTCTCATTCTTTAGACAATGAAAAGCAGCATGCGCAAAGAGCTGGA
TTTTTAGTTTCTCTGTTATCAATGGCTTATGCCAACCCAGAATTATATTTAACATCAAATGGTAGTAATGCAGATGTTAA
ATATGTTATCTACATGATAGAGAAAGACCCAGGAAGACAGAAATATGGTGGGTTTGTCGTCAAGACTAGAGAGATGGTTT
ATGAAAAGACAACTGATTGGATGTTCGGGAGTGATCTTGAGTATGATCAAGACAATATGTTGCAAAATGGTAGAAGCACT
TCTACAATCGAGGATCTTGTTCATACTTTTGGATATCCATCGTGTCTTGGAGCCCTTATAATCCAAGTTTGGATAATACT
TGTTAAGGCTATAACCAGTATATCAGGATTGAGGAAAGGATTCTTTACTCGGTTAGAAGCATTTCGACAAGATGGAACAG
TTAAATCCAGTCTAGTGTTGAGCGGTGATGCAGTAGAACAAATTGGATCAATTATGAGGTCCCAACAGAGCTTGGTAACA
CTCATGGTTGAAACACTGATAACAATGAACACAGGCAGGAATGATCTGACAACAATAGAAAAGAATATACAGATTGTAGG
AAACTACATCAGAGATGCAGGTCTTGCTTCATTTTTCAACACAATCAGATATGGCATTGAGACTAGAATGGCAGCTCTAA
CTCTGTCTACCCTTAGACCGGATATCAACAGACTCAAGGCACTGATCGAGTTATATCTATCAAAGGGGCCACGTGCTCCT
TTTATATGCATTTTGAGAGATCCCGTGCATGGTGAGTTTGCACCAGGCAACTATCCTGCCCTCTGGAGTTATGCGATGGG
TGTAGCAGTTGTACAAAACAAGGCCATGCAACAGTATGTAACAGGAAGGTCTTATCTGGATATTGAAATGTTCCAACTTG
GTCAAGCAGTGGCACGTGATGCCGAGTCGCAGATGAGTTCAATATTAGAGGATGAACTGGGGGTCACACAAGAAGCCAAG
CAAAGCTTGAAGAAACACATGAAGAACATCAGCAGTTCAGATACAACCTTTCATAAGCCTACAGGGGGATCAGCCATAGA
AATGGCGATAGATGAAGAAGCAGGGCAGCCTGAATCCAGAGGAGATCAGGATCAAGGAGATGAGCCTCGGTCATCCATAG
TTCCTTATGCATGGGCAGACGAAACCGGGAATGACAATCAAACTGAATCAACTACAGAAATTGACAGCATCAAAACTGAA
CAAAGAAACATCAGAGACAGGCTGAACAAAAGACTCAACGAGAAAAGGAAACAGAGTGACCCGAGATCAACTGACATCAC
AAACAACACAAATCAAACTGAAATAGATGATTTGTTCAGTGCATTCGGAAGCAACTAGTCACAAAGAGATGACCAGGCGC
GCCAAGTAAGAAAAACTTAGGATTAATGGACCTGCAGGATGTTCGTGTTTCTGGTGCTGCTGCCTCTGGTGAGCTCCCAG
TGCGTGAACCTGACCACAAGGACCCAGCTGCCCCCTGCCTATACCAATTCCTTCACACGGGGCGTGTACTATCCCGACAA
GGTGTTTAGATCTAGCGTGCTGCACTCCACACAGGATCTGTTTCTGCCTTTCTTTTCTAACGTGACCTGGTTCCACGCCA
TCCACGTGAGCGGCACCAATGGCACAAAGCGGTTCGACAATCCAGTGCTGCCCTTTAACGATGGCGTGTACTTCGCCTCC
ACCGAGAAGTCTAACATCATCAGAGGCTGGATCTTTGGCACCACACTGGACAGCAAGACACAGTCCCTGCTGATCGTGAA
CAATGCCACCAACGTGGTCATCAAGGTGTGCGAGTTCCAGTTTTGTAATGATCCATTCCTGGGCGTGTACTATCACAAGA
ACAATAAGTCTTGGATGGAGAGCGAGTTTCGCGTGTATTCCTCTGCCAACAATTGCACATTTGAGTACGTGTCCCAGCCC
TTCCTGATGGACCTGGAGGGCAAGCAGGGCAATTTCAAGAACCTGAGGGAGTTCGTGTTTAAGAATATCGATGGCTACTT
CAAGATCTACTCCAAGCACACCCCAATCAACCTGGTGCGCGACCTGCCACAGGGCTTCTCTGCCCTGGAGCCACTGGTGG
ATCTGCCCATCGGCATCAACATCACCCGGTTTCAGACACTGCTGGCCCTGCACAGAAGCTACCTGACACCAGGCGACAGC
TCCTCTGGATGGACCGCAGGAGCTGCCGCCTACTATGTGGGCTATCTGCAGCCCAGGACCTTCCTGCTGAAGTACAACGA
GAATGGCACCATCACAGACGCCGTGGATTGCGCCCTGGATCCCCTGTCTGAGACCAAGTGTACACTGAAGAGCTTTACCG
TGGAGAAGGGCATCTATCAGACAAGCAATTTCAGGGTGCAGCCTACCGAGTCCATCGTGCGCTTTCCCAATATCACAAAC
CTGTGCCCTTTTGGCGAGGTGTTCAACGCAACCCGCTTCGCCAGCGTGTACGCCTGGAATAGGAAGCGCATCTCCAACTG
CGTGGCCGACTATTCTGTGCTGTACAACAGCGCCTCCTTCTCTACCTTTAAGTGCTATGGCGTGAGCCCCACAAAGCTGA
ATGACCTGTGCTTTACCAACGTGTACGCCGATTCCTTCGTGATCAGGGGCGACGAGGTGCGCCAGATCGCCCCTGGCCAG
ACAGGCAAGATCGCCGACTACAATTATAAGCTGCCTGACGATTTCACCGGCTGCGTGATCGCCTGGAACTCTAACAATCT
GGATAGCAAAGTGGGCGGCAACTACAATTATCTGTACCGGCTGTTTAGAAAGTCTAATCTGAAGCCATTCGAGAGGGACA
TCTCCACAGAGATCTACCAGGCCGGCTCTACCCCCTGCAATGGCGTGGAGGGCTTTAACTGTTATTTCCCTCTGCAGAGC
TACGGCTTCCAGCCAACAAACGGCGTGGGCTATCAGCCCTACCGCGTGGTGGTGCTGTCTTTTGAGCTGCTGCACGCACC
TGCAACAGTGTGCGGACCAAAGAAGAGCACCAATCTGGTGAAGAACAAGTGCGTGAACTTCAACTTCAACGGACTGACCG
GCACAGGCGTGCTGACCGAGTCCAACAAGAAGTTCCTGCCTTTTCAGCAGTTCGGCAGGGACATCGCAGATACCACAGAC
GCCGTGCGCGACCCTCAGACCCTGGAGATCCTGGATATCACACCATGCTCCTTCGGCGGCGTGTCTGTGATCACACCAGG
CACCAATACAAGCAACCAGGTGGCCGTGCTGTATCAGGACGTGAATTGTACCGAGGTGCCCGTGGCAATCCACGCAGATC
AGCTGACCCCTACATGGCGGGTGTACTCTACCGGCAGCAACGTGTTCCAGACAAGAGCCGGATGCCTGATCGGAGCCGAG
CACGTGAACAATAGCTATGAGTGCGACATCCCTATCGGCGCCGGCATCTGTGCCTCCTACCAGACCCAGACAAACTCCCC
AgGGtctGCCtccTCTGTGGCCAGCCAGTCCATCATCGCCTATACCATGAGCCTGGGCGCCGAGAATTCCGTGGCCTACT
CCAACAATTCTATCGCCATCCCTACCAACTTCACAATCTCCGTGACCACAGAGATCCTGCCAGTGAGCATGACCAAGACA
TCCGTGGACTGCACAATGTATATCTGTGGCGATTCCACCGAGTGCTCTAACCTGCTGCTGCAGTACGGCTCTTTTTGTAC
CCAGCTGAATAGAGCCCTGACAGGCATCGCCGTGGAGCAGGACAAGAACACACAGGAGGTGTTCGCCCAGGTGAAGCAGA
TCTACAAGACCCCACCCATCAAGGACTTTGGCGGCTTCAACTTCAGCCAGATCCTGCCCGATCCTAGCAAGCCATCCAAG
CGGTCTTTTATCGAGGACCTGCTGTTCAACAAGGTGACCCTGGCCGATGCCGGCTTCATCAAGCAGTATGGCGATTGCCT
GGGCGACATCGCCGCCAGAGACCTGATCTGTGCCCAGAAGTTTAATGGCCTGACCGTGCTGCCTCCACTGCTGACAGATG
AGATGATCGCCCAGTACACATCTGCCCTGCTGGCCGGCACCATCACAAGCGGATGGACCTTCGGCGCAGGAGCCGCCCTG
CAGATCCCCTTTGCCATGCAGATGGCCTATCGGTTCAACGGCATCGGCGTGACCCAGAATGTGCTGTACGAGAACCAGAA
GCTGATCGCCAATCAGTTTAACTCCGCCATCGGCAAGATCCAGGACTCTCTGAGCTCCACAGCCAGCGCCCTGGGCAAGC
TGCAGGATGTGGTGAATCAGAACGCCCAGGCCCTGAATACCCTGGTGAAGCAGCTGTCTAGCAACTTCGGCGCCATCTCC
TCTGTGCTGAATGATATCCTGAGCAGGCTGGACcctccaGAGGCAGAGGTGCAGATCGACCGGCTGATCACAGGCAGACT
GCAGTCCCTGCAGACCTACGTGACACAGCAGCTGATCAGGGCAGCAGAGATCAGGGCCTCTGCCAATCTGGCCGCCACCA
AGATGAGCGAGTGCGTGCTGGGCCAGTCCAAGAGAGTGGACTTTTGTGGCAAGGGCTATCACCTGATGAGCTTCCCACAG
TCCGCCCCTCACGGAGTGGTGTTTCTGCACGTGACCTACGTGCCAGCCCAGGAGAAGAACTTCACCACAGCACCAGCAAT
CTGCCACGATGGCAAGGCACACTTTCCTAGGGAGGGCGTGTTCGTGAGCAACGGCACCCACTGGTTTGTGACACAGCGCA
ATTTCTACGAGCCACAGATCATCACCACAGACAATACATTCGTGTCCGGCAACTGTGACGTGGTCATCGGCATCGTGAAC
AATACCGTGTATGATCCTCTGCAGCCAGAGCTGGACTCTTTTAAGGAGGAGCTGGATAAGTACTTCAAGAATCACACCAG
CCCCGACGTGGATCTGGGCGACATCTCTGGCATCAATGCCAGCGTGGTGAACATCCAGAAGGAGATCGACAGGCTGAACG
AGGTGGCCAAGAATCTGAACGAGTCCCTGATCGATCTGCAGGAGCTGGGCAAGTATGAGCAGTACATCAAGTGGCCCTGG
TATATCTGGCTGGGCTTCATCGCCGGCCTGATCGCCATCGTGATGGTGACCATCATGCTGTGCTGTATGACAAGCTGCTG
TTCCTGCCTGAAGGGCTGCTGTTCTTGTGGCAGCTGCTGTAAGTTTGATGAGGACGATAGCGAGCCTGTGCTGAAGGGCG
TGAAGCTGCACTACACCTGATAGTAACTAGCGGCGCGCCAGCAACAAGTAAGAAAAACTTAGGATTAATGGAAATTATCC
AATCCAGAGACGGAAGGACAAATCCAGAATCCAACCACAACTCAATCAACCAAAGATTCATGGAAGACAATGTTCAAAAC
AATCAAATCATGGATTCTTGGGAAGAGGGATCAGGAGATAAATCATCTGACATCTCATCGGCCCTCGACATCATTGAATT
CATACTCAGCACCGACTCCCAAGAGAACACGGCAGACAGCAATGAAATCAACACAGGAACCACAAGACTTAGCACGACAA
TCTACCAACCTGAATCCAAAACAACAGAAACAAGCAAGGAAAATAGTGGACCAGCTAACAAAAATCGACAGTTTGGGGCA
TCACACGAACGTGCCACAGAGACAAAAGATAGAAATGTTAATCAGGAGACTGTACAGGGAGGATATAGGAGAGGAAGCAG
CCCAGATAGTAGAACTGAGACTATGGTCACTCGAAGAATCTCCAGAAGCAGCCCAGATCCTAACAATGGAACCCAAATCC
AGGAAGATATTGATTACAATGAAGTTGGAGAGATGGATAAGGACTCTACTAAGAGGGAAATGCGACAATTTAAAGATGTT
CCAGTCAAGGTATCAGGAAGTGATGCCATTCCTCCAACAAAACAAGATGGAGACGGTGATGATGGAAGAGGCCTGGAATC
TATCAGTACATTTGATTCAGGATATACCAGTATAGTGACTGCCGCAACACTAGATGACGAAGAAGAACTCCTTATGAAGA
ACAACAGGCCAAGAAAGTATCAATCAACACCCCAGAACAGTGACAAGGGAATTAAAAAAGGGGTTGGAAGGCCAAAAGAC
ACAGACAAACAATCATCAATATTGGACTACGAACTCAACTTCAAAGGATCGAAGAAGAGCCAGAAAATCCTCAAAGCCAG
CACGAATACAGGAGAACCAACAAGACCACAGAATGGATCCCAGGGGAAGAGAATCACATCCTGGAACATCCTCAACAGCG
AGAGCGGCAATCGAACAGAATCAACAAACCAAACCCATCAGACATCAACCTCGGGACAGAACCACACAATGGGACCAAGC
AGAACAACCTCCGAACCAAGGATCAAGACACAAAAGACGGATGGAAAGGAAAGAGAGGACACAGAAGAGAGCACTCGATT
TACAGAAAGGGCGATTACATTATTACAGAATCTTGGTGTAATCCAATCTGCAGCAAAATTAGACCTATACCAAGACAAGA
GAGTTGTGTGTGTGGCGAATGTCCTAAACAATGCAGATACTGCATCAAAGATAGACTTCCTAGCAGGTTTGATGATAGGA
GTGTCAATGGATCATGATACCAAATTAAATCAGATTCAGAACGAGATATTAAGTTTGAAAACTGATCTTAAAAAGATGGA
TGAATCACATAGAAGACTAATTGAGAATCAAAAAGAACAATTATCACTGATCACATCATTAATCTCAAATCTTAAAATTA
TGACAGAGAGAGGAGGGAAGAAGGACCAACCAGAACCTAGCGGGAGGACATCCATGATCAAGACAAAAGCAAAAGAAGAG
AAAATAAAGAAAGTCAGGTTTGACCCTCTTATGGAAACACAGGGCATCGAGAAAAACATCCCTGACCTCTATAGATCAAT
AGAGAAAACACCAGAAAACGACACACAGATCAAATCAGAAATAAACAGATTGAATGATGAATCCAATGCCACTAGATTAG
TACCTAGAAGAATAAGCAGTACAATGAGATCATTAATAATAATCATTAACAACAGCAATTTATCATCAAAAGCAAAGCAA
TCATACATCAACGAACTCAAGCTCTGCAAGAGTGACGAGGAAGTGTCTGAGTTGATGGACATGTTCAATGAGGATGTCAG
CTCCCAGTAAACCGCCAACCAAGGGTCAACACCAAGAAAACCAATAGCACAAAACAGCCAATCAGAGACCACCCCAATAC
ACCAAACCAATCAACACATAACAAAGATCGCGGCCGCATAGATGATTAAGAAAAACTTAGGATGAAAGGACTAATCAATC
CTCCGAAACAATGAGCATCACCAACTCCACAATCTACACATTCCCAGAATCCTCTTTCTCCGAGAATGGCAACATAGAGC
CGTTACCACTCAAGGTCAATGAACAGAGAAAGGCCATACCTCATATTAGGGTTGTCAAGATAGGAGATCCGCCCAAACAT
GGATCCAGATATCTGGATGTCTTTTTACTGGGCTTCTTTGAGATGGAAAGGTCAAAAGACAGGTATGGGAGCATAAGTGA
TCTAGATGATGATCCAAGTTACAAGGTTTGTGGCTCTGGATCATTGCCACTTGGGTTGGCTAGATACACCGGAAATGATC
AGGAACTCCTACAGGCTGCAACCAAGCTCGATATAGAAGTAAGAAGAACTGTAAAGGCTACGGAGATGATAGTTTACACT
GTACAAAACATCAAACCTGAACTATATCCATGGTCCAGTAGATTAAGAAAAGGGATGTTATTTGACGCTAATAAGGTTGC
ACTTGCTCCTCAATGTCTTCCACTAGATAGAGGGATAAAATTCAGGGTGATATTTGTGAACTGCACAGCAATTGGATCAA
TAACTCTATTCAAAATCCCTAAGTCCATGGCATTGTTATCATTGCCTAATACAATATCAATAAATCTACAAGTACATATC
AAAACAGGAGTTCAGACAGATTCCAAAGGAGTAGTTCAGATTCTAGATGAAAAAGGTGAAAAATCACTAAATTTCATGGT
TCATCTCGGGTTGATCAAAAGGAAGATGGGCAGAATGTACTCAGTTGAATATTGTAAGCAGAAGATCGAGAAGATGAGAT
TATTATTCTCATTGGGATTAGTTGGAGGGATCAGCTTCCACGTCAACGCAACTGGCTCTATATCAAAGACATTAGCAAGT
CAATTAGCATTCAAAAGAGAAATCTGCTATCCCCTAATGGATCTGAATCCACACTTAAATTCAGTTATATGGGCATCATC
AGTTGAAATTACAAGGGTAGATGCAGTTCTCCAGCCTTCATTACCTGGCGAATTCAGATACTACCCAAACATCATAGCAA
AAGGGGTCGGGAAAATCAGACAGTAAAATCAACAACCCTGATATCCACCGGTGTATTAAGCCGAAGCAAATAAAGGATAA
TCAAAAACTTAGGACAAAAGAGGTCAATACCAACAACTATTAGCAGTCACACTCGCAAGAATAAGAGAGAAGGGACCAAA
AAAGTCAAATAGGAGAAATCAAAACAAAAGGTACAGAACACCAGAACAACAAAATCAAAACATCCAACTCACTCAAAACA
AAAATTCCAAAAGAGACCGGCAACACAACAAGCACTGAACACAATGCCAACTTCAATACTGCTAATTATTACAACCATGA
TCATGGCATCTTTCTGCCAAATAGATATCACAAAACTACAGCACGTAGGTGTATTGGTCAACAGTCCCAAAGGGATGAAG
ATATCACAAAACTTTGAAACAAGATATCTAATTTTGAGCCTCATACCAAAAATAGAAGACTCTAACTCTTGTGGTGACCA
ACAGATCAAGCAATACAAGAAGTTATTGGATAGACTGATCATCCCTTTATATGATGGATTAAGATTACAGAAAGATGTGA
TAGTAACCAATCAAGAATCCAATGAAAACACTGATCCCAGAACAAAACGATTCTTTGGAGGGGTAATTGGAACCATTGCT
CTGGGAGTAGCAACCTCAGCACAAATTACAGCGGCAGTTGCTCTGGTTGAAGCCAAGCAGGCAAGATCAGACATCGAAAA
ACTCAAAGAAGCAATTAGGGACACAAACAAAGCAGTGCAGTCAGTTCAGAGCTCCATAGGAAATTTAATAGTAGCAATTA
AATCAGTCCAGGATTATGTTAACAAAGAAATCGTGCCATCGATTGCGAGGCTAGGTTGTGAAGCAGCAGGACTTCAATTA
GGAATTGCATTAACACAGCATTACTCAGAATTAACAAACATATTTGGTGATAACATAGGATCGTTACAAGAAAAAGGAAT
AAAATTACAAGGTATAGCATCATTATACCGCACAAATATCACAGAAATATTCACAACATCAACAGTTGATAAATATGATA
TCTATGATCTGTTATTTACAGAATCAATAAAGGTGAGAGTTATAGATGTTGACTTGAATGATTACTCAATCACCCTCCAA
GTCAGACTCCCTTTATTAACTAGGCTGCTGAACACTCAGATCTACAAAGTAGATTCCATATCATATAACATCCAAAACAG
AGAATGGTATATCCCTCTTCCCAGCCATATCATGACGAAAGGGGCATTTCTAGGTGGAGCAGACGTCAAAGAATGTATAG
AAGCATTCAGCAGCTATATATGCCCTTCTGATCCAGGATTTGTATTAAACCATGAAATAGAGAGCTGCTTATCAGGAAAC
ATATCCCAATGTCCAAGAACAACGGTCACATCAGACATTGTTCCAAGATATGCATTTGTCAATGGAGGAGTGGTTGCAAA
CTGTATAACAACCACCTGTACATGCAACGGAATTGGTAATAGAATCAATCAACCACCTGATCAAGGAGTAAAAATTATAA
CACATAAAGAATGTAGTACAATAGGTATCAACGGAATGCTGTTCAATACAAATAAAGAAGGAACTCTTGCATTCTATACA
CCAAATGATATAACACTAAACAATTCTGTTGCACTTGATCCAATTGACATATCAATCGAGCTCAACAAGGCCAAATCAGA
TCTAGAAGAATCAAAAGAATGGATAAGAAGGTCAAATCAAAAACTAGATTCTATTGGAAATTGGCATCAATCTAGCACTA
CAATCATAATTATTTTGATAATGATCATTATATTGTTTATAATTAATATAACGATAATTACAATTGCAATTAAGTATTAC
AGAATTCAAAAGAGAAATCGAGTGGATCAAAATGACAAGCCATATGTACTAACAAACAAATAACATATCTACAGATCATT
AGATATTAAAATTATAAAAAACTTAGGAGTAAAGTTACGCAATCCAACTCTACTCATATAATTGAGGAAGGACCCAATAG
ACAAATCCAAATTCGAGATGGAATACTGGAAGCATACCAATCACGGAAAGGATGCTGGTAATGAGCTGGAGACGTCTATG
GCTACTCATGGCAACAAGCTCACTAATAAGATAATATACATATTATGGACAATAATCCTGGTGTTATTATCAATAGTCTT
CATCATAGTGCTAATTAATTCCATCAAAAGTGAAAAGGCCCACGAATCATTGCTGCAAGACATAAATAATGAGTTTATGG
AAATTACAGAAAAGATCCAAATGGCATCGGATAATACCAATGATCTAATACAGTCAGGAGTGAATACAAGGCTTCTTACA
ATTCAGAGTCATGTCCAGAATTACATACCAATATCATTGACACAACAGATGTCAGATCTTAGGAAATTCATTAGTGAAAT
TACAATTAGAAATGATAATCAAGAAGTGCTGCCACAAAGAATAACACATGATGTAGGTATAAAACCTTTAAATCCAGATG
ATTTTTGGAGATGCACGTCTGGTCTTCCATCTTTAATGAAAACTCCAAAAATAAGGTTAATGCCAGGGCCGGGATTATTA
GCTATGCCAACGACTGTTGATGGCTGTGTTAGAACTCCGTCTTTAGTTATAAATGATCTGATTTATGCTTATACCTCAAA
TCTAATTACTCGAGGTTGTCAGGATATAGGAAAATCATATCAAGTCTTACAGATAGGGATAATAACTGTAAACTCAGACT
TGGTACCTGACTTAAATCCTAGGATCTCTCATACCTTTAACATAAATGACAATAGGAAGTCATGTTCTCTAGCACTCCTA
AATACAGATGTATATCAACTGTGTTCAACTCCCAAAGTTGATGAAAGATCAGATTATGCATCATCAGGCATAGAAGATAT
TGTACTTGATATTGTCAATTATGATGGTTCAATCTCAACAACAAGATTTAAGAATAATAACATAAGCTTTGATCAACCAT
ATGCTGCACTATACCCATCTGTTGGACCAGGGATATACTACAAAGGCAAAATAATATTTCTCGGGTATGGAGGTCTTGAA
CATCCAATAAATGAGAATGTAATCTGCAACACAACTGGGTGCCCCGGGAAAACACAGAGAGACTGTAATCAAGCATCTCA
TAGTcCaTGGTTTTCAGATAGGAGGATGGTCAACTCCATCATTGTTGTTGACAAAGGCTTAAACTCAATTCCAAAATTGA
AAGTATGGACGATATCTATGCGACAAAATTACTGGGGGTCAGAAGGAAGGTTACTTCTACTAGGTAACAAGATCTATATA
TATACAAGATCTACAAGTTGGCATAGCAAGTTACAATTAGGAATAATTGATATTACTGATTACAGTGATATAAGGATAAA
ATGGACATGGCATAATGTGCTATCAAGACCAGGAAACAATGAATGTCCATGGGGACATTCATGTCCAGATGGATGTATAA
CAGGAGTATATACTGATGCATATCCACTCAATCCCACAGGGAGCATTGTGTCATCTGTCATATTAGACTCACAAAAATCG
AGAGTGAACCCAGTCATAACTTACTCAACAGCAACCGAAAGAGTAAACGAGCTGGCCATCCTAAACAGAACACTCTCAGC
TGGATATACAACAACAAGCTGCATTACACACTATAACAAAGGATATTGTTTTCATATAGTAGAAATAAATCATAAAAGCT
TAAACACATTTCAACCCATGTTGTTCAAAACAGAGATTCCAAAAAGCTGCAGTTAATCATAATTAACCATAATATGCATC
AATCTATCTATAATACAAGTATATGATAAGTAATCAGCAATCAGACAATAGACGTACGGAAATAATAAAAAACTTAGGAG
AAAAGTGTGCAAGAAAAATGGACACCGAGTCCCACAGCGGCACAACATCTGACATTCTGTACCCTGAATGTCACCTCAAT
TCTCCTATAGTTAAAGGAAAGATAGCACAACTGCATACAATAATGAGTTTGCCTCAGCCCTACGATATGGATGATGATTC
AATACTGATTATTACTAGACAAAAAATTAAACTCAATAAATTAGATAAAAGACAACGGTCAATTAGGAAATTAAGATCAG
TCTTAATGGAAAGAGTAAGTGATCTAGGTAAATATACCTTTATCAGATATCCAGAGATGTCTAGTGAAATGTTCCAATTA
TGTATACCCGGAATTAATAATAAAATAAATGAATTGCTAAGTAAAGCAAGTAAAACATATAATCAAATGACTGATGGATT
AAGAGATCTATGGGTTACTATACTATCGAAGTTAGCATCGAAAAATGATGGAAGTAATTATGATATCAATGAAGATATTA
GCAATATATCAAATGTTCACATGACTTATCAATCAGACAAATGGTATAATCCATTCAAGACATGGTTTACTATTAAGTAT
GACATGAGAAGATTACAAAAAGCCAAAAATGAGATTACATTCAATAGGCATAAAGATTATAATCTATTAGAAGACCAAAA
GAATATATTGCTGATACATCCAGAACTCGTCTTAATATTAGATAAACAAAATTACAATGGGTATATAATGACTCCTGAAT
TGGTACTAATGTATTGTGATGTAGTTGAAGGGAGGTGGAATATAAGTTCATGTGCAAAATTGGATCCTAAGTTACAATCA
ATGTATTATAAGGGTAACAATTTATGGGAAATAATAGATGGACTATTCTCGACCTTAGGAGAAAGAACATTTGACATAAT
ATCACTATTAGAACCACTTGCATTATCGCTCATTCAAACTTATGACCCGGTTAAACAGCTCAGGGGGGCTTTTTTAAATC
ACGTGTTATCAGAAATGGAATTAATATTTGCAGCTGAGTGTACAACAGAGGAAATACCTAATGTGGATTATATAGATAAA
ATTTTAGATGTGTTCAAAGAATCAACAATAGATGAAATAGCAGAAATTTTCTCTTTCTTCCGAACTTTTGGACACCCTCC
ATTAGAGGCGAGTATAGCAGCAGAGAAAGTTAGAAAGTATATGTATACTGAGAAATGCTTGAAATTTGATACTATCAATA
AATGTCATGCTATTTTTTGTACAATAATTATAAATGGATATAGAGAAAGACATGGTGGTCAATGGCCTCCAGTTACATTA
CCTGTCCATGCACATGAATTTATCATAAATGCATACGGATCAAATTCTGCCATATCATATGAGAATGCTGTAGATTATTA
TAAGAGCTTCATAGGAATAAAATTTGACAAGTTTATAGAGCCTCAATTGGATGAAGACTTAACTATTTATATGAAAGATA
AAGCATTATCCCCAAAGAAATCAAACTGGGACACAGTCTATCCAGCTTCAAACCTGTTATACCGCACTAATGTGTCTCAT
GATTCACGAAGATTGGTTGAAGTATTTATAGCAGATAGTAAATTTGATCCCCACCAAGTATTAGATTACGTAGAATCAGG
ATATTGGCTGGATGATCCTGAATTTAATATCTCATATAGTTTAAAAGAGAAAGAAATAAAACAAGAAGGTAGACTTTTTG
CAAAAATGACATACAAGATGAGGGCTACACAAGTATTATCAGAAACATTATTGGCGAATAATATAGGGAAATTCTTCCAA
GAGAATGGGATGGTTAAAGGAGAAATTGAATTACTCAAGAGACTAACAACAATATCTATGTCTGGAGTTCCGCGGTATAA
TGAGGTATACAATAATTCAAAAAGTCACACAGAAGAACTTCAAGCTTATAATGCAATTAGCAGTTCCAATTTATCTTCTA
ATCAGAAGTCAAAGAAGTTTGAATTTAAATCTACAGATATATACAATGATGGATACGAAACCGTAAGCTGCTTCTTAACG
ACAGATCTTAAAAAATATTGTTTAAATTGGAGGTATGAATCAACAGCTTTATTCGGTGATACTTGTAATCAGATATTTGG
GTTAAAGGAATTATTTAATTGGCTGCACCCTCGCCTTGAAAAGAGTACAATATATGTTGGAGATCCTTATTGCCCGCCAT
CAGATATTGAACATTTACCACTTGATGACCATCCTGATTCAGGATTTTATGTTCATAATCCTAAAGGAGGAATAGAAGGG
TTTTGCCAAAAGTTATGGACACTCATATCTATCAGTGCAATACATTTAGCAGCTGTCAAAATCGGTGTAAGAGTTACTGC
AATGGTTCAAGGGGATAATCAAGCCATAGCTGTTACCACAAGAGTACCTAATAATTATGATTATAAAGTTAAGAAAGAGA
TTGTTTATAAAGATGTGGTAAGATTTTTTGATTCCTTGAGAGAGGTGATGGATGATCTGGGTCATGAGCTCAAACTAAAT
GAAACTATAATAAGTAGTAAAATGTTTATATATAGCAAAAGGATATACTATGACGGAAGAATCCTTCCTCAGGCATTAAA
AGCATTGTCTAGATGTGTTTTTTGGTCTGAAACAATCATAGATGAGACAAGATCAGCATCCTCAAATCTGGCTACATCGT
TTGCAAAGGCCATTGAGAATGGCTACTCACCTGTATTGGGATATGTATGCTCAATCTTCAAAAATATCCAACAGTTGTAT
ATAGCGCTTGGAATGAATATAAACCCAACTATAACCCAAAATATTAAAGATCAATATTTCAGGAATATTCATTGGATGCA
ATATGCCTCCTTAATCCCTGCTAGTGTCGGAGGATTTAATTATATGGCCATGTCAAGGTGTTTTGTCAGAAACATTGGAG
ATCCTACAGTCGCTGCGTTAGCCGATATTAAAAGATTTATAAAAGCAAATTTGTTAGATCGAGGTGTCCTTTACAGAATT
ATGAATCAAGAACCAGGCGAGTCTTCTTTTTTAGACTGGGCCTCAGATCCCTATTCATGTAACTTACCACAATCTCAAAA
TATAACCACCATGATAAAGAATATAACTGCAAGAAATGTACTACAGGACTCACCAAACCCATTACTATCTGGATTATTTA
CAAGTACAATGATAGAAGAGGATGAGGAATTAGCTGAGTTCCTAATGGACAGGAGAATAATCCTCCCAAGAGTTGCACAT
GACATTTTAGATAATTCTCTTACTGGAATTAGGAATGCTATAGCTGGTATGTTGGATACAACAAAATCACTAATTCGAGT
AGGGATAAGCAGAGGAGGATTAACCTATAACTTATTAAGAAAGATAAGCAACTATGATCTTGTACAATATGAGACACTTA
GTAAAACTTTAAGACTAATAGTCAGTGACAAGATTAAGTATGAAGATATGTGCTCAGTAGACCTAGCCATATCATTAAGA
CAAAAAATGTGGATGCATTTATCAGGAGGAAGAATGATAAATGGACTTGAAACTCCAGATCCTTTAGAGTTACTGTCTGG
AGTAATAATAACAGGATCTGAACATTGTAGGATATGTTATTCAACTGAAGGTGAAAGCCCATATACATGGATGTATTTAC
CAGGCAATCTTAATATAGGATCAGCTGAGACAGGAATAGCATCATTAAGGGTCCCTTACTTTGGATCAGTTACAGATGAG
AGATCTGAAGCACAATTAGGGTATATCAAAAATCTAAGCAAACCAGCTAAGGCTGCTATAAGAATAGCAATGATATATAC
TTGGGCATTTGGGAATGACGAAATATCTTGGATGGAAGCATCACAGATTGCACAAACACGTGCAAACTTTACATTGGATA
GCTTAAAGATTTTGACACCAGTGACAACATCAACAAATCTATCACACAGGTTAAAAGATACTGCTACTCAGATGAAATTT
TCTAGTACATCACTTATTAGAGTAAGCAGGTTCATCACAATATCTAATGATAATATGTCTATTAAAGAAGCAAATGAAAC
TAAAGATACAAATCTTATTTATCAACAGGTAATGTTAACAGGATTAAGTGTATTTGAATATCTATTTAGGTTAGAGGAGA
GTACAGGACATAACCCTATGGTCATGCATCTACATATAGAGGATGGATGTTGTATAAAAGAGAGTTACAATGATGAGCAT
ATCAATCCGGAGTCTACATTAGAGTTAATCAAATACCCTGAGAGTAATGAATTTATATATGATAAGGACCCTTTAAAGGA
TATAGATCTATCAAAATTAATGGTTATAAGAGATCATTCTTATACAATTGACATGAATTACTGGGATGACACAGATATTG
TACATGCAATATCAATATGTACTGCAGTTACAATAGCAGATACAATGTCGCAGCTAGATCGGGATAATCTTAAGGAGCTG
GTTGTGATTGCAAATGATGATGATATTAACAGTCTGATAACTGAATTTCTGACCCTAGATATACTAGTGTTTCTCAAAAC
ATTTGGAGGGTTACTCGTGAATCAATTTGCATATACCCTTTATGGATTGAAAATAGAAGGAAGGGATCCCATTTGGGATT
ATATAATGAGAACATTAAAAGACACCTCACATTCAGTACTTAAAGTATTATCTAATGCACTATCTCATCCAAAAGTGTTT
AAGAGATTTTGGGATTGTGGAGTTTTGAATCCTATTTATGGTCCTAATACTGCTAGTCAAGATCAAGTTAAGCTTGCTCT
CTCGATTTGCGAGTACTCCTTGGATCTATTTATGAGAGAATGGTTGAATGGAGCATCACTTGAGATCTATATCTGTGATA
GTGACATGGAAATAGCAAATGACAGAAGACAAGCATTTCTCTCAAGACATCTTGCCTTTGTGTGTTGTTTAGCAGAGATA
GCATCTTTTGGACCAAATTTATTAAATCTAACATATCTAGAGAGACTTGATGAATTAAAACAATACTTAGATCTGAACAT
CAAAGAAGATCCTACTCTTAAATATGTGCAAGTATCAGGACTGTTAATTAAATCATTCCCCTCAACTGTTACGTATGTAA
GGAAAACTGCGATTAAGTATCTGAGGATTCGTGGTATTAATCCGCCTGAAACGATTGAAGATTGGGATCCCATAGAAGAT
GAGAATATCTTAGACAATATTGTTAAAACTGTAAATGACAATTGCAGTGATAATCAAAAGAGAAATAAAAGTAGTTATTT
CTGGGGATTAGCTCTAAAGAATTATCAAGTCGTGAAAATAAGATCCATAACGAGTGATTCTGAAGTTAATGAAGCTTCGA
ATGTTACTACACATGGAATGACACTTCCTCAGGGAGGAAGTTATCTATCACATCAGCTGAGGTTATTTGGAGTAAACAGT
ACAAGTTGTCTTAAAGCTCTTGAATTATCACAAATCTTAATGAGGGAAGTTAAAAAAGATAAAGATAGACTCTTTTTAGG
AGAAGGAGCAGGAGCTATGTTAGCATGTTATGATGCTACACTCGGTCCTGCAATAAATTATTATAATTCTGGTTTAAATA
TTACAGATGTAATTGGTCAACGGGAATTAAAAATCTTCCCATCAGAAGTATCATTAGTAGGTAAAAAACTAGGAAATGTA
ACACAGATTCTTAATCGGGTGAGGGTGTTATTTAATGGGAATCCCAATTCAACATGGATAGGAAATATGGAATGTGAGAG
TTTAATATGGAGTGAATTAAATGATAAGTCAATTGGTTTAGTACATTGTGACATGGAGGGAGCGATAGGCAAATCAGAAG
AAACTGTTCTACATGAACATTATAGTATTATTAGGATTACATATTTAATCGGGGATGATGATGTTGTCCTAGTATCAAAA
ATTATACCAACTATTACTCCGAATTGGTCTAAAATACTCTATCTATACAAGTTGTATTGGAAGGATGTAAGTGTAGTGTC
CCTTAAAACATCCAATCCTGCCTCAACAGAGCTTTATTTAATTTCAAAAGATGCTTACTGTACTGTAATGGAACCCAGTA
ATCTTGTTTTATCAAAACTTAAAAGGATATCATCAATAGAAGAAAATAATCTATTAAAGTGGATAATCTTATCAAAAAGG
AAGAATAACGAGTGGTTACAGCATGAAATCAAAGAAGGAGAAAGGGATTATGGGATAATGAGGCCATATCATACAGCACT
GCAAATTTTTGGATTCCAAATTAACTTAAATCACTTAGCTAGAGAATTTTTATCAACTCCTGATTTAACCAACATTAATA
ATATAATTCAAAGTTTTACAAGAACAATTAAAGATGTTATGTTCGAATGGGTCAATATCACTCATGACAATAAAAGACAT
AAATTAGGAGGAAGATATAATCTATTCCCGCTTAAAAATAAGGGGAAATTAAGATTATTATCACGAAGATTAGTACTAAG
CTGGATATCATTATCCTTATCAACCAGATTACTGACGGGCCGTTTTCCAGATGAAAAATTTGAAAATAGGGCACAGACCG
GATATGTATCATTGGCTGATATTGATTTAGAATCCTTAAAGTTATTATCAAGAAATATTGTCAAAAATTACAAAGAACAC
ATAGGATTAATATCATACTGGTTTTTGACCAAAGAGGTCAAAATACTAATGAAGCTTATAGGAGGAGTCAAACTACTAGG
AATTCCTAAACAGTACAAAGAGTTAGAGGATCGATCATCTCAGGGTTATGAATATGATAATGAATTTGATATTGATTAAT
ACATAAAAACAaAAAATAAAACACCTATTCCTCACCCATTCACTTCCAACAAAATGAAAAGTAAGAAAAACATGTAATAT
ATATATACCAAACAGAGTTTTTCTCTTGTTTGGT

In some embodiments, the genome of the rB/HPIV3-SARS-COV-2/S vector comprises an antigenomic cDNA sequence set forth as SEQ ID NO: 31.

rB/HPIV3-SARS-COV-2/S-6P RRAR(682-685)GSAS
(SEQ ID NO: 31)
ACCAAACAAGAGAAGAGACTGGTTTGGGAATATTAATTCAAATAAAAATTAACTTAGGATTAAAGAACTTTACCGAAAGG
TAAGGGGAAAGAAATCCTAAGAGCTTAGCCATGTTGAGTCTATTCGACACATTCAGTGCGCGTAGGCAGGAGAACATAAC
GAAATCAGCTGGTGGGGCTGTTATTCCCGGGCAAAAAAACACTGTGTCTATATTTGCTCTTGGACCATCAATAACAGATG
ACAATGATAAAATGACATTGGCTCTTCTCTTTTTGTCTCATTCTTTAGACAATGAAAAGCAGCATGCGCAAAGAGCTGGA
TTTTTAGTTTCTCTGTTATCAATGGCTTATGCCAACCCAGAATTATATTTAACATCAAATGGTAGTAATGCAGATGTTAA
ATATGTTATCTACATGATAGAGAAAGACCCAGGAAGACAGAAATATGGTGGGTTTGTCGTCAAGACTAGAGAGATGGTTT
ATGAAAAGACAACTGATTGGATGTTCGGGAGTGATCTTGAGTATGATCAAGACAATATGTTGCAAAATGGTAGAAGCACT
TCTACAATCGAGGATCTTGTTCATACTTTTGGATATCCATCGTGTCTTGGAGCCCTTATAATCCAAGTTTGGATAATACT
TGTTAAGGCTATAACCAGTATATCAGGATTGAGGAAAGGATTCTTTACTCGGTTAGAAGCATTTCGACAAGATGGAACAG
TTAAATCCAGTCTAGTGTTGAGCGGTGATGCAGTAGAACAAATTGGATCAATTATGAGGTCCCAACAGAGCTTGGTAACA
CTCATGGTTGAAACACTGATAACAATGAACACAGGCAGGAATGATCTGACAACAATAGAAAAGAATATACAGATTGTAGG
AAACTACATCAGAGATGCAGGTCTTGCTTCATTTTTCAACACAATCAGATATGGCATTGAGACTAGAATGGCAGCTCTAA
CTCTGTCTACCCTTAGACCGGATATCAACAGACTCAAGGCACTGATCGAGTTATATCTATCAAAGGGGCCACGTGCTCCT
TTTATATGCATTTTGAGAGATCCCGTGCATGGTGAGTTTGCACCAGGCAACTATCCTGCCCTCTGGAGTTATGCGATGGG
TGTAGCAGTTGTACAAAACAAGGCCATGCAACAGTATGTAACAGGAAGGTCTTATCTGGATATTGAAATGTTCCAACTTG
GTCAAGCAGTGGCACGTGATGCCGAGTCGCAGATGAGTTCAATATTAGAGGATGAACTGGGGGTCACACAAGAAGCCAAG
CAAAGCTTGAAGAAACACATGAAGAACATCAGCAGTTCAGATACAACCTTTCATAAGCCTACAGGGGGATCAGCCATAGA
AATGGCGATAGATGAAGAAGCAGGGCAGCCTGAATCCAGAGGAGATCAGGATCAAGGAGATGAGCCTCGGTCATCCATAG
TTCCTTATGCATGGGCAGACGAAACCGGGAATGACAATCAAACTGAATCAACTACAGAAATTGACAGCATCAAAACTGAA
CAAAGAAACATCAGAGACAGGCTGAACAAAAGACTCAACGAGAAAAGGAAACAGAGTGACCCGAGATCAACTGACATCAC
AAACAACACAAATCAAACTGAAATAGATGATTTGTTCAGTGCATTCGGAAGCAACTAGTCACAAAGAGATGACCAGGCGC
GCCAAGTAAGAAAAACTTAGGATTAATGGACCTGCAGGATGTTCGTGTTTCTGGTGCTGCTGCCTCTGGTGAGCTCCCAG
TGCGTGAACCTGACCACAAGGACCCAGCTGCCCCCTGCCTATACCAATTCCTTCACACGGGGCGTGTACTATCCCGACAA
GGTGTTTAGATCTAGCGTGCTGCACTCCACACAGGATCTGTTTCTGCCTTTCTTTTCTAACGTGACCTGGTTCCACGCCA
TCCACGTGAGCGGCACCAATGGCACAAAGCGGTTCGACAATCCAGTGCTGCCCTTTAACGATGGCGTGTACTTCGCCTCC
ACCGAGAAGTCTAACATCATCAGAGGCTGGATCTTTGGCACCACACTGGACAGCAAGACACAGTCCCTGCTGATCGTGAA
CAATGCCACCAACGTGGTCATCAAGGTGTGCGAGTTCCAGTTTTGTAATGATCCATTCCTGGGCGTGTACTATCACAAGA
ACAATAAGTCTTGGATGGAGAGCGAGTTTCGCGTGTATTCCTCTGCCAACAATTGCACATTTGAGTACGTGTCCCAGCCC
TTCCTGATGGACCTGGAGGGCAAGCAGGGCAATTTCAAGAACCTGAGGGAGTTCGTGTTTAAGAATATCGATGGCTACTT
CAAGATCTACTCCAAGCACACCCCAATCAACCTGGTGCGCGACCTGCCACAGGGCTTCTCTGCCCTGGAGCCACTGGTGG
ATCTGCCCATCGGCATCAACATCACCCGGTTTCAGACACTGCTGGCCCTGCACAGAAGCTACCTGACACCAGGCGACAGC
TCCTCTGGATGGACCGCAGGAGCTGCCGCCTACTATGTGGGCTATCTGCAGCCCAGGACCTTCCTGCTGAAGTACAACGA
GAATGGCACCATCACAGACGCCGTGGATTGCGCCCTGGATCCCCTGTCTGAGACCAAGTGTACACTGAAGAGCTTTACCG
TGGAGAAGGGCATCTATCAGACAAGCAATTTCAGGGTGCAGCCTACCGAGTCCATCGTGCGCTTTCCCAATATCACAAAC
CTGTGCCCTTTTGGCGAGGTGTTCAACGCAACCCGCTTCGCCAGCGTGTACGCCTGGAATAGGAAGCGCATCTCCAACTG
CGTGGCCGACTATTCTGTGCTGTACAACAGCGCCTCCTTCTCTACCTTTAAGTGCTATGGCGTGAGCCCCACAAAGCTGA
ATGACCTGTGCTTTACCAACGTGTACGCCGATTCCTTCGTGATCAGGGGCGACGAGGTGCGCCAGATCGCCCCTGGCCAG
ACAGGCAAGATCGCCGACTACAATTATAAGCTGCCTGACGATTTCACCGGCTGCGTGATCGCCTGGAACTCTAACAATCT
GGATAGCAAAGTGGGCGGCAACTACAATTATCTGTACCGGCTGTTTAGAAAGTCTAATCTGAAGCCATTCGAGAGGGACA
TCTCCACAGAGATCTACCAGGCCGGCTCTACCCCCTGCAATGGCGTGGAGGGCTTTAACTGTTATTTCCCTCTGCAGAGC
TACGGCTTCCAGCCAACAAACGGCGTGGGCTATCAGCCCTACCGCGTGGTGGTGCTGTCTTTTGAGCTGCTGCACGCACC
TGCAACAGTGTGCGGACCAAAGAAGAGCACCAATCTGGTGAAGAACAAGTGCGTGAACTTCAACTTCAACGGACTGACCG
GCACAGGCGTGCTGACCGAGTCCAACAAGAAGTTCCTGCCTTTTCAGCAGTTCGGCAGGGACATCGCAGATACCACAGAC
GCCGTGCGCGACCCTCAGACCCTGGAGATCCTGGATATCACACCATGCTCCTTCGGCGGCGTGTCTGTGATCACACCAGG
CACCAATACAAGCAACCAGGTGGCCGTGCTGTATCAGGACGTGAATTGTACCGAGGTGCCCGTGGCAATCCACGCAGATC
AGCTGACCCCTACATGGCGGGTGTACTCTACCGGCAGCAACGTGTTCCAGACAAGAGCCGGATGCCTGATCGGAGCCGAG
CACGTGAACAATAGCTATGAGTGCGACATCCCTATCGGCGCCGGCATCTGTGCCTCCTACCAGACCCAGACAAACTCCCC
AgGGtctGCCtccTCTGTGGCCAGCCAGTCCATCATCGCCTATACCATGAGCCTGGGCGCCGAGAATTCCGTGGCCTACT
CCAACAATTCTATCGCCATCCCTACCAACTTCACAATCTCCGTGACCACAGAGATCCTGCCAGTGAGCATGACCAAGACA
TCCGTGGACTGCACAATGTATATCTGTGGCGATTCCACCGAGTGCTCTAACCTGCTGCTGCAGTACGGCTCTTTTTGTAC
CCAGCTGAATAGAGCCCTGACAGGCATCGCCGTGGAGCAGGACAAGAACACACAGGAGGTGTTCGCCCAGGTGAAGCAGA
TCTACAAGACCCCACCCATCAAGGACTTTGGCGGCTTCAACTTCAGCCAGATCCTGCCCGATCCTAGCAAGCCATCCAAG
CGGTCTCCTATCGAGGACCTGCTGTTCAACAAGGTGACCCTGGCCGATGCCGGCTTCATCAAGCAGTATGGCGATTGCCT
GGGCGACATCGCCGCCAGAGACCTGATCTGTGCCCAGAAGTTTAATGGCCTGACCGTGCTGCCTCCACTGCTGACAGATG
AGATGATCGCCCAGTACACATCTGCCCTGCTGGCCGGCACCATCACAAGCGGATGGACCTTCGGCGCAGGACCCGCCCTG
CAGATCCCCTTTCCCATGCAGATGGCCTATCGGTTCAACGGCATCGGCGTGACCCAGAATGTGCTGTACGAGAACCAGAA
GCTGATCGCCAATCAGTTTAACTCCGCCATCGGCAAGATCCAGGACTCTCTGAGCTCCACACCCAGCGCCCTGGGCAAGC
TGCAGGATGTGGTGAATCAGAACGCCCAGGCCCTGAATACCCTGGTGAAGCAGCTGTCTAGCAACTTCGGCGCCATCTCC
TCTGTGCTGAATGATATCCTGAGCAGGCTGGACcctccaGAGGCAGAGGTGCAGATCGACCGGCTGATCACAGGCAGACT
GCAGTCCCTGCAGACCTACGTGACACAGCAGCTGATCAGGGCAGCAGAGATCAGGGCCTCTGCCAATCTGGCCGCCACCA
AGATGAGCGAGTGCGTGCTGGGCCAGTCCAAGAGAGTGGACTTTTGTGGCAAGGGCTATCACCTGATGAGCTTCCCACAG
TCCGCCCCTCACGGAGTGGTGTTTCTGCACGTGACCTACGTGCCAGCCCAGGAGAAGAACTTCACCACAGCACCAGCAAT
CTGCCACGATGGCAAGGCACACTTTCCTAGGGAGGGCGTGTTCGTGAGCAACGGCACCCACTGGTTTGTGACACAGCGCA
ATTTCTACGAGCCACAGATCATCACCACAGACAATACATTCGTGTCCGGCAACTGTGACGTGGTCATCGGCATCGTGAAC
AATACCGTGTATGATCCTCTGCAGCCAGAGCTGGACTCTTTTAAGGAGGAGCTGGATAAGTACTTCAAGAATCACACCAG
CCCCGACGTGGATCTGGGCGACATCTCTGGCATCAATGCCAGCGTGGTGAACATCCAGAAGGAGATCGACAGGCTGAACG
AGGTGGCCAAGAATCTGAACGAGTCCCTGATCGATCTGCAGGAGCTGGGCAAGTATGAGCAGTACATCAAGTGGCCCTGG
TATATCTGGCTGGGCTTCATCGCCGGCCTGATCGCCATCGTGATGGTGACCATCATGCTGTGCTGTATGACAAGCTGCTG
TTCCTGCCTGAAGGGCTGCTGTTCTTGTGGCAGCTGCTGTAAGTTTGATGAGGACGATAGCGAGCCTGTGCTGAAGGGCG
TGAAGCTGCACTACACCTGATAGTAACTAGCGGCGCGCCAGCAACAAGTAAGAAAAACTTAGGATTAATGGAAATTATCC
AATCCAGAGACGGAAGGACAAATCCAGAATCCAACCACAACTCAATCAACCAAAGATTCATGGAAGACAATGTTCAAAAC
AATCAAATCATGGATTCTTGGGAAGAGGGATCAGGAGATAAATCATCTGACATCTCATCGGCCCTCGACATCATTGAATT
CATACTCAGCACCGACTCCCAAGAGAACACGGCAGACAGCAATGAAATCAACACAGGAACCACAAGACTTAGCACGACAA
TCTACCAACCTGAATCCAAAACAACAGAAACAAGCAAGGAAAATAGTGGACCAGCTAACAAAAATCGACAGTTTGGGGCA
TCACACGAACGTGCCACAGAGACAAAAGATAGAAATGTTAATCAGGAGACTGTACAGGGAGGATATAGGAGAGGAAGCAG
CCCAGATAGTAGAACTGAGACTATGGTCACTCGAAGAATCTCCAGAAGCAGCCCAGATCCTAACAATGGAACCCAAATCC
AGGAAGATATTGATTACAATGAAGTTGGAGAGATGGATAAGGACTCTACTAAGAGGGAAATGCGACAATTTAAAGATGTT
CCAGTCAAGGTATCAGGAAGTGATGCCATTCCTCCAACAAAACAAGATGGAGACGGTGATGATGGAAGAGGCCTGGAATC
TATCAGTACATTTGATTCAGGATATACCAGTATAGTGACTGCCGCAACACTAGATGACGAAGAAGAACTCCTTATGAAGA
ACAACAGGCCAAGAAAGTATCAATCAACACCCCAGAACAGTGACAAGGGAATTAAAAAAGGGGTTGGAAGGCCAAAAGAC
ACAGACAAACAATCATCAATATTGGACTACGAACTCAACTTCAAAGGATCGAAGAAGAGCCAGAAAATCCTCAAAGCCAG
CACGAATACAGGAGAACCAACAAGACCACAGAATGGATCCCAGGGGAAGAGAATCACATCCTGGAACATCCTCAACAGCG
AGAGCGGCAATCGAACAGAATCAACAAACCAAACCCATCAGACATCAACCTCGGGACAGAACCACACAATGGGACCAAGC
AGAACAACCTCCGAACCAAGGATCAAGACACAAAAGACGGATGGAAAGGAAAGAGAGGACACAGAAGAGAGCACTCGATT
TACAGAAAGGGCGATTACATTATTACAGAATCTTGGTGTAATCCAATCTGCAGCAAAATTAGACCTATACCAAGACAAGA
GAGTTGTGTGTGTGGCGAATGTCCTAAACAATGCAGATACTGCATCAAAGATAGACTTCCTAGCAGGTTTGATGATAGGA
GTGTCAATGGATCATGATACCAAATTAAATCAGATTCAGAACGAGATATTAAGTTTGAAAACTGATCTTAAAAAGATGGA
TGAATCACATAGAAGACTAATTGAGAATCAAAAAGAACAATTATCACTGATCACATCATTAATCTCAAATCTTAAAATTA
TGACAGAGAGAGGAGGGAAGAAGGACCAACCAGAACCTAGCGGGAGGACATCCATGATCAAGACAAAAGCAAAAGAAGAG
AAAATAAAGAAAGTCAGGTTTGACCCTCTTATGGAAACACAGGGCATCGAGAAAAACATCCCTGACCTCTATAGATCAAT
AGAGAAAACACCAGAAAACGACACACAGATCAAATCAGAAATAAACAGATTGAATGATGAATCCAATGCCACTAGATTAG
TACCTAGAAGAATAAGCAGTACAATGAGATCATTAATAATAATCATTAACAACAGCAATTTATCATCAAAAGCAAAGCAA
TCATACATCAACGAACTCAAGCTCTGCAAGAGTGACGAGGAAGTGTCTGAGTTGATGGACATGTTCAATGAGGATGTCAG
CTCCCAGTAAACCGCCAACCAAGGGTCAACACCAAGAAAACCAATAGCACAAAACAGCCAATCAGAGACCACCCCAATAC
ACCAAACCAATCAACACATAACAAAGATCGCGGCCGCATAGATGATTAAGAAAAACTTAGGATGAAAGGACTAATCAATC
CTCCGAAACAATGAGCATCACCAACTCCACAATCTACACATTCCCAGAATCCTCTTTCTCCGAGAATGGCAACATAGAGC
CGTTACCACTCAAGGTCAATGAACAGAGAAAGGCCATACCTCATATTAGGGTTGTCAAGATAGGAGATCCGCCCAAACAT
GGATCCAGATATCTGGATGTCTTTTTACTGGGCTTCTTTGAGATGGAAAGGTCAAAAGACAGGTATGGGAGCATAAGTGA
TCTAGATGATGATCCAAGTTACAAGGTTTGTGGCTCTGGATCATTGCCACTTGGGTTGGCTAGATACACCGGAAATGATC
AGGAACTCCTACAGGCTGCAACCAAGCTCGATATAGAAGTAAGAAGAACTGTAAAGGCTACGGAGATGATAGTTTACACT
GTACAAAACATCAAACCTGAACTATATCCATGGTCCAGTAGATTAAGAAAAGGGATGTTATTTGACGCTAATAAGGTTGC
ACTTGCTCCTCAATGTCTTCCACTAGATAGAGGGATAAAATTCAGGGTGATATTTGTGAACTGCACAGCAATTGGATCAA
TAACTCTATTCAAAATCCCTAAGTCCATGGCATTGTTATCATTGCCTAATACAATATCAATAAATCTACAAGTACATATC
AAAACAGGAGTTCAGACAGATTCCAAAGGAGTAGTTCAGATTCTAGATGAAAAAGGTGAAAAATCACTAAATTTCATGGT
TCATCTCGGGTTGATCAAAAGGAAGATGGGCAGAATGTACTCAGTTGAATATTGTAAGCAGAAGATCGAGAAGATGAGAT
TATTATTCTCATTGGGATTAGTTGGAGGGATCAGCTTCCACGTCAACGCAACTGGCTCTATATCAAAGACATTAGCAAGT
CAATTAGCATTCAAAAGAGAAATCTGCTATCCCCTAATGGATCTGAATCCACACTTAAATTCAGTTATATGGGCATCATC
AGTTGAAATTACAAGGGTAGATGCAGTTCTCCAGCCTTCATTACCTGGCGAATTCAGATACTACCCAAACATCATAGCAA
AAGGGGTCGGGAAAATCAGACAGTAAAATCAACAACCCTGATATCCACCGGTGTATTAAGCCGAAGCAAATAAAGGATAA
TCAAAAACTTAGGACAAAAGAGGTCAATACCAACAACTATTAGCAGTCACACTCGCAAGAATAAGAGAGAAGGGACCAAA
AAAGTCAAATAGGAGAAATCAAAACAAAAGGTACAGAACACCAGAACAACAAAATCAAAACATCCAACTCACTCAAAACA
AAAATTCCAAAAGAGACCGGCAACACAACAAGCACTGAACACAATGCCAACTTCAATACTGCTAATTATTACAACCATGA
TCATGGCATCTTTCTGCCAAATAGATATCACAAAACTACAGCACGTAGGTGTATTGGTCAACAGTCCCAAAGGGATGAAG
ATATCACAAAACTTTGAAACAAGATATCTAATTTTGAGCCTCATACCAAAAATAGAAGACTCTAACTCTTGTGGTGACCA
ACAGATCAAGCAATACAAGAAGTTATTGGATAGACTGATCATCCCTTTATATGATGGATTAAGATTACAGAAAGATGTGA
TAGTAACCAATCAAGAATCCAATGAAAACACTGATCCCAGAACAAAACGATTCTTTGGAGGGGTAATTGGAACCATTGCT
CTGGGAGTAGCAACCTCAGCACAAATTACAGCGGCAGTTGCTCTGGTTGAAGCCAAGCAGGCAAGATCAGACATCGAAAA
ACTCAAAGAAGCAATTAGGGACACAAACAAAGCAGTGCAGTCAGTTCAGAGCTCCATAGGAAATTTAATAGTAGCAATTA
AATCAGTCCAGGATTATGTTAACAAAGAAATCGTGCCATCGATTGCGAGGCTAGGTTGTGAAGCAGCAGGACTTCAATTA
GGAATTGCATTAACACAGCATTACTCAGAATTAACAAACATATTTGGTGATAACATAGGATCGTTACAAGAAAAAGGAAT
AAAATTACAAGGTATAGCATCATTATACCGCACAAATATCACAGAAATATTCACAACATCAACAGTTGATAAATATGATA
TCTATGATCTGTTATTTACAGAATCAATAAAGGTGAGAGTTATAGATGTTGACTTGAATGATTACTCAATCACCCTCCAA
GTCAGACTCCCTTTATTAACTAGGCTGCTGAACACTCAGATCTACAAAGTAGATTCCATATCATATAACATCCAAAACAG
AGAATGGTATATCCCTCTTCCCAGCCATATCATGACGAAAGGGGCATTTCTAGGTGGAGCAGACGTCAAAGAATGTATAG
AAGCATTCAGCAGCTATATATGCCCTTCTGATCCAGGATTTGTATTAAACCATGAAATAGAGAGCTGCTTATCAGGAAAC
ATATCCCAATGTCCAAGAACAACGGTCACATCAGACATTGTTCCAAGATATGCATTTGTCAATGGAGGAGTGGTTGCAAA
CTGTATAACAACCACCTGTACATGCAACGGAATTGGTAATAGAATCAATCAACCACCTGATCAAGGAGTAAAAATTATAA
CACATAAAGAATGTAGTACAATAGGTATCAACGGAATGCTGTTCAATACAAATAAAGAAGGAACTCTTGCATTCTATACA
CCAAATGATATAACACTAAACAATTCTGTTGCACTTGATCCAATTGACATATCAATCGAGCTCAACAAGGCCAAATCAGA
TCTAGAAGAATCAAAAGAATGGATAAGAAGGTCAAATCAAAAACTAGATTCTATTGGAAATTGGCATCAATCTAGCACTA
CAATCATAATTATTTTGATAATGATCATTATATTGTTTATAATTAATATAACGATAATTACAATTGCAATTAAGTATTAC
AGAATTCAAAAGAGAAATCGAGTGGATCAAAATGACAAGCCATATGTACTAACAAACAAATAACATATCTACAGATCATT
AGATATTAAAATTATAAAAAACTTAGGAGTAAAGTTACGCAATCCAACTCTACTCATATAATTGAGGAAGGACCCAATAG
ACAAATCCAAATTCGAGATGGAATACTGGAAGCATACCAATCACGGAAAGGATGCTGGTAATGAGCTGGAGACGTCTATG
GCTACTCATGGCAACAAGCTCACTAATAAGATAATATACATATTATGGACAATAATCCTGGTGTTATTATCAATAGTCTT
CATCATAGTGCTAATTAATTCCATCAAAAGTGAAAAGGCCCACGAATCATTGCTGCAAGACATAAATAATGAGTTTATGG
AAATTACAGAAAAGATCCAAATGGCATCGGATAATACCAATGATCTAATACAGTCAGGAGTGAATACAAGGCTTCTTACA
ATTCAGAGTCATGTCCAGAATTACATACCAATATCATTGACACAACAGATGTCAGATCTTAGGAAATTCATTAGTGAAAT
TACAATTAGAAATGATAATCAAGAAGTGCTGCCACAAAGAATAACACATGATGTAGGTATAAAACCTTTAAATCCAGATG
ATTTTTGGAGATGCACGTCTGGTCTTCCATCTTTAATGAAAACTCCAAAAATAAGGTTAATGCCAGGGCCGGGATTATTA
GCTATGCCAACGACTGTTGATGGCTGTGTTAGAACTCCGTCTTTAGTTATAAATGATCTGATTTATGCTTATACCTCAAA
TCTAATTACTCGAGGTTGTCAGGATATAGGAAAATCATATCAAGTCTTACAGATAGGGATAATAACTGTAAACTCAGACT
TGGTACCTGACTTAAATCCTAGGATCTCTCATACCTTTAACATAAATGACAATAGGAAGTCATGTTCTCTAGCACTCCTA
AATAcAGATGTATATCAACTGTGTTCAACTCCCAAAGTTGATGAAAGATCAGATTATGCATCATCAGGCATAGAAGATAT
TGTACTTGATATTGTCAATTATGATGGTTCAATCTCAACAACAAGATTTAAGAATAATAACATAAGCTTTGATCAACCAT
ATGCTGCACTATACCCATCTGTTGGACCAGGGATATACTACAAAGGCAAAATAATATTTCTCGGGTATGGAGGTCTTGAA
CATCCAATAAATGAGAATGTAATCTGCAACACAACTGGGTGCCCCGGGAAAACACAGAGAGACTGTAATCAAGCATCTCA
TAGTcCaTGGTTTTCAGATAGGAGGATGGTCAACTCCATCATTGTTGTTGACAAAGGCTTAAACTCAATTCCAAAATTGA
AAGTATGGACGATATCTATGCGACAAAATTACTGGGGGTCAGAAGGAAGGTTACTTCTACTAGGTAACAAGATCTATATA
TATACAAGATCTACAAGTTGGCATAGCAAGTTACAATTAGGAATAATTGATATTACTGATTACAGTGATATAAGGATAAA
ATGGACATGGCATAATGTGCTATCAAGACCAGGAAACAATGAATGTCCATGGGGACATTCATGTCCAGATGGATGTATAA
CAGGAGTATATACTGATGCATATCCACTCAATCCCACAGGGAGCATTGTGTCATCTGTCATATTAGACTCACAAAAATCG
AGAGTGAACCCAGTCATAACTTACTCAACAGCAACCGAAAGAGTAAACGAGCTGGCCATCCTAAACAGAACACTCTCAGC
TGGATATACAACAACAAGCTGCATTACACACTATAACAAAGGATATTGTTTTCATATAGTAGAAATAAATCATAAAAGCT
TAAACACATTTCAACCCATGTTGTTCAAAACAGAGATTCCAAAAAGCTGCAGTTAATCATAATTAACCATAATATGCATC
AATCTATCTATAATACAAGTATATGATAAGTAATCAGCAATCAGACAATAGACGTACGGAAATAATAAAAAACTTAGGAG
AAAAGTGTGCAAGAAAAATGGACACCGAGTCCCACAGCGGCACAACATCTGACATTCTGTACCCTGAATGTCACCTCAAT
TCTCCTATAGTTAAAGGAAAGATAGCACAACTGCATACAATAATGAGTTTGCCTCAGCCCTACGATATGGATGATGATTC
AATACTGATTATTACTAGACAAAAAATTAAACTCAATAAATTAGATAAAAGACAACGGTCAATTAGGAAATTAAGATCAG
TCTTAATGGAAAGAGTAAGTGATCTAGGTAAATATACCTTTATCAGATATCCAGAGATGTCTAGTGAAATGTTCCAATTA
TGTATACCCGGAATTAATAATAAAATAAATGAATTGCTAAGTAAAGCAAGTAAAACATATAATCAAATGACTGATGGATT
AAGAGATCTATGGGTTACTATACTATCGAAGTTAGCATCGAAAAATGATGGAAGTAATTATGATATCAATGAAGATATTA
GCAATATATCAAATGTTCACATGACTTATCAATCAGACAAATGGTATAATCCATTCAAGACATGGTTTACTATTAAGTAT
GACATGAGAAGATTACAAAAAGCCAAAAATGAGATTACATTCAATAGGCATAAAGATTATAATCTATTAGAAGACCAAAA
GAATATATTGCTGATACATCCAGAACTCGTCTTAATATTAGATAAACAAAATTACAATGGGTATATAATGACTCCTGAAT
TGGTACTAATGTATTGTGATGTAGTTGAAGGGAGGTGGAATATAAGTTCATGTGCAAAATTGGATCCTAAGTTACAATCA
ATGTATTATAAGGGTAACAATTTATGGGAAATAATAGATGGACTATTCTCGACCTTAGGAGAAAGAACATTTGACATAAT
ATCACTATTAGAACCACTTGCATTATCGCTCATTCAAACTTATGACCCGGTTAAACAGCTCAGGGGGGCTTTTTTAAATC
ACGTGTTATCAGAAATGGAATTAATATTTGCAGCTGAGTGTACAACAGAGGAAATACCTAATGTGGATTATATAGATAAA
ATTTTAGATGTGTTCAAAGAATCAACAATAGATGAAATAGCAGAAATTTTCTCTTTCTTCCGAACTTTTGGACACCCTCC
ATTAGAGGCGAGTATAGCAGCAGAGAAAGTTAGAAAGTATATGTATACTGAGAAATGCTTGAAATTTGATACTATCAATA
AATGTCATGCTATTTTTTGTACAATAATTATAAATGGATATAGAGAAAGACATGGTGGTCAATGGCCTCCAGTTACATTA
CCTGTCCATGCACATGAATTTATCATAAATGCATACGGATCAAATTCTGCCATATCATATGAGAATGCTGTAGATTATTA
TAAGAGCTTCATAGGAATAAAATTTGACAAGTTTATAGAGCCTCAATTGGATGAAGACTTAACTATTTATATGAAAGATA
AAGCATTATCCCCAAAGAAATCAAACTGGGACACAGTCTATCCAGCTTCAAACCTGTTATACCGCACTAATGTGTCTCAT
GATTCACGAAGATTGGTTGAAGTATTTATAGCAGATAGTAAATTTGATCCCCACCAAGTATTAGATTACGTAGAATCAGG
ATATTGGCTGGATGATCCTGAATTTAATATCTCATATAGTTTAAAAGAGAAAGAAATAAAACAAGAAGGTAGACTTTTTG
CAAAAATGACATACAAGATGAGGGCTACACAAGTATTATCAGAAACATTATTGGCGAATAATATAGGGAAATTCTTCCAA
GAGAATGGGATGGTTAAAGGAGAAATTGAATTACTCAAGAGACTAACAACAATATCTATGTCTGGAGTTCCGCGGTATAA
TGAGGTATACAATAATTCAAAAAGTCACACAGAAGAACTTCAAGCTTATAATGCAATTAGCAGTTCCAATTTATCTTCTA
ATCAGAAGTCAAAGAAGTTTGAATTTAAATCTACAGATATATACAATGATGGATACGAAACCGTAAGCTGCTTCTTAACG
ACAGATCTTAAAAAATATTGTTTAAATTGGAGGTATGAATCAACAGCTTTATTCGGTGATACTTGTAATCAGATATTTGG
GTTAAAGGAATTATTTAATTGGCTGCACCCTCGCCTTGAAAAGAGTACAATATATGTTGGAGATCCTTATTGCCCGCCAT
CAGATATTGAACATTTACCACTTGATGACCATCCTGATTCAGGATTTTATGTTCATAATCCTAAAGGAGGAATAGAAGGG
TTTTGCCAAAAGTTATGGACACTCATATCTATCAGTGCAATACATTTAGCAGCTGTCAAAATCGGTGTAAGAGTTACTGC
AATGGTTCAAGGGGATAATCAAGCCATAGCTGTTACCACAAGAGTACCTAATAATTATGATTATAAAGTTAAGAAAGAGA
TTGTTTATAAAGATGTGGTAAGATTTTTTGATTCCTTGAGAGAGGTGATGGATGATCTGGGTCATGAGCTCAAACTAAAT
GAAACTATAATAAGTAGTAAAATGTTTATATATAGCAAAAGGATATACTATGACGGAAGAATCCTTCCTCAGGCATTAAA
AGCATTGTCTAGATGTGTTTTTTGGTCTGAAACAATCATAGATGAGACAAGATCAGCATCCTCAAATCTGGCTACATCGT
TTGCAAAGGCCATTGAGAATGGCTACTCACCTGTATTGGGATATGTATGCTCAATCTTCAAAAATATCCAACAGTTGTAT
ATAGCGCTTGGAATGAATATAAACCCAACTATAACCCAAAATATTAAAGATCAATATTTCAGGAATATTCATTGGATGCA
ATATGCCTCCTTAATCCCTGCTAGTGTCGGAGGATTTAATTATATGGCCATGTCAAGGTGTTTTGTCAGAAACATTGGAG
ATCCTACAGTCGCTGCGTTAGCCGATATTAAAAGATTTATAAAAGCAAATTTGTTAGATCGAGGTGTCCTTTACAGAATT
ATGAATCAAGAACCAGGCGAGTCTTCTTTTTTAGACTGGGCCTCAGATCCCTATTCATGTAACTTACCACAATCTCAAAA
TATAACCACCATGATAAAGAATATAACTGCAAGAAATGTACTACAGGACTCACCAAACCCATTACTATCTGGATTATTTA
CAAGTACAATGATAGAAGAGGATGAGGAATTAGCTGAGTTCCTAATGGACAGGAGAATAATCCTCCCAAGAGTTGCACAT
GACATTTTAGATAATTCTCTTACTGGAATTAGGAATGCTATAGCTGGTATGTTGGATACAACAAAATCACTAATTCGAGT
AGGGATAAGCAGAGGAGGATTAACCTATAACTTATTAAGAAAGATAAGCAACTATGATCTTGTACAATATGAGACACTTA
GTAAAACTTTAAGACTAATAGTCAGTGACAAGATTAAGTATGAAGATATGTGCTCAGTAGACCTAGCCATATCATTAAGA
CAAAAAATGTGGATGCATTTATCAGGAGGAAGAATGATAAATGGACTTGAAACTCCAGATCCTTTAGAGTTACTGTCTGG
AGTAATAATAACAGGATCTGAACATTGTAGGATATGTTATTCAACTGAAGGTGAAAGCCCATATACATGGATGTATTTAC
CAGGCAATCTTAATATAGGATCAGCTGAGACAGGAATAGCATCATTAAGGGTCCCTTACTTTGGATCAGTTACAGATGAG
AGATCTGAAGCACAATTAGGGTATATCAAAAATCTAAGCAAACCAGCTAAGGCTGCTATAAGAATAGCAATGATATATAC
TTGGGCATTTGGGAATGACGAAATATCTTGGATGGAAGCATCACAGATTGCACAAACACGTGCAAACTTTACATTGGATA
GCTTAAAGATTTTGACACCAGTGACAACATCAACAAATCTATCACACAGGTTAAAAGATACTGCTACTCAGATGAAATTT
TCTAGTACATCACTTATTAGAGTAAGCAGGTTCATCACAATATCTAATGATAATATGTCTATTAAAGAAGCAAATGAAAC
TAAAGATACAAATCTTATTTATCAACAGGTAATGTTAACAGGATTAAGTGTATTTGAATATCTATTTAGGTTAGAGGAGA
GTACAGGACATAACCCTATGGTCATGCATCTACATATAGAGGATGGATGTTGTATAAAAGAGAGTTACAATGATGAGCAT
ATCAATCCGGAGTCTACATTAGAGTTAATCAAATACCCTGAGAGTAATGAATTTATATATGATAAGGACCCTTTAAAGGA
TATAGATCTATCAAAATTAATGGTTATAAGAGATCATTCTTATACAATTGACATGAATTACTGGGATGACACAGATATTG
TACATGCAATATCAATATGTACTGCAGTTACAATAGCAGATACAATGTCGCAGCTAGATCGGGATAATCTTAAGGAGCTG
GTTGTGATTGCAAATGATGATGATATTAACAGTCTGATAACTGAATTTCTGACCCTAGATATACTAGTGTTTCTCAAAAC
ATTTGGAGGGTTACTCGTGAATCAATTTGCATATACCCTTTATGGATTGAAAATAGAAGGAAGGGATCCCATTTGGGATT
ATATAATGAGAACATTAAAAGACACCTCACATTCAGTACTTAAAGTATTATCTAATGCACTATCTCATCCAAAAGTGTTT
AAGAGATTTTGGGATTGTGGAGTTTTGAATCCTATTTATGGTCCTAATACTGCTAGTCAAGATCAAGTTAAGCTTGCTCT
CTCGATTTGCGAGTACTCCTTGGATCTATTTATGAGAGAATGGTTGAATGGAGCATCACTTGAGATCTATATCTGTGATA
GTGACATGGAAATAGCAAATGACAGAAGACAAGCATTTCTCTCAAGACATCTTGCCTTTGTGTGTTGTTTAGCAGAGATA
GCATCTTTTGGACCAAATTTATTAAATCTAACATATCTAGAGAGACTTGATGAATTAAAACAATACTTAGATCTGAACAT
CAAAGAAGATCCTACTCTTAAATATGTGCAAGTATCAGGACTGTTAATTAAATCATTCCCCTCAACTGTTACGTATGTAA
GGAAAACTGCGATTAAGTATCTGAGGATTCGTGGTATTAATCCGCCTGAAACGATTGAAGATTGGGATCCCATAGAAGAT
GAGAATATCTTAGACAATATTGTTAAAACTGTAAATGACAATTGCAGTGATAATCAAAAGAGAAATAAAAGTAGTTATTT
CTGGGGATTAGCTCTAAAGAATTATCAAGTCGTGAAAATAAGATCCATAACGAGTGATTCTGAAGTTAATGAAGCTTCGA
ATGTTACTACACATGGAATGACACTTCCTCAGGGAGGAAGTTATCTATCACATCAGCTGAGGTTATTTGGAGTAAACAGT
ACAAGTTGTCTTAAAGCTCTTGAATTATCACAAATCTTAATGAGGGAAGTTAAAAAAGATAAAGATAGACTCTTTTTAGG
AGAAGGAGCAGGAGCTATGTTAGCATGTTATGATGCTACACTCGGTCCTGCAATAAATTATTATAATTCTGGTTTAAATA
TTACAGATGTAATTGGTCAACGGGAATTAAAAATCTTCCCATCAGAAGTATCATTAGTAGGTAAAAAACTAGGAAATGTA
ACACAGATTCTTAATCGGGTGAGGGTGTTATTTAATGGGAATCCCAATTCAACATGGATAGGAAATATGGAATGTGAGAG
TTTAATATGGAGTGAATTAAATGATAAGTCAATTGGTTTAGTACATTGTGACATGGAGGGAGCGATAGGCAAATCAGAAG
AAACTGTTCTACATGAACATTATAGTATTATTAGGATTACATATTTAATCGGGGATGATGATGTTGTCCTAGTATCAAAA
ATTATACCAACTATTACTCCGAATTGGTCTAAAATACTCTATCTATACAAGTTGTATTGGAAGGATGTAAGTGTAGTGTC
CCTTAAAACATCCAATCCTGCCTCAACAGAGCTTTATTTAATTTCAAAAGATGCTTACTGTACTGTAATGGAACCCAGTA
ATCTTGTTTTATCAAAACTTAAAAGGATATCATCAATAGAAGAAAATAATCTATTAAAGTGGATAATCTTATCAAAAAGG
AAGAATAACGAGTGGTTACAGCATGAAATCAAAGAAGGAGAAAGGGATTATGGGATAATGAGGCCATATCATACAGCACT
GCAAATTTTTGGATTCCAAATTAACTTAAATCACTTAGCTAGAGAATTTTTATCAACTCCTGATTTAACCAACATTAATA
ATATAATTCAAAGTTTTACAAGAACAATTAAAGATGTTATGTTCGAATGGGTCAATATCACTCATGACAATAAAAGACAT
AAATTAGGAGGAAGATATAATCTATTCCCGCTTAAAAATAAGGGGAAATTAAGATTATTATCACGAAGATTAGTACTAAG
CTGGATATCATTATCCTTATCAACCAGATTACTGACGGGCCGTTTTCCAGATGAAAAATTTGAAAATAGGGCACAGACCG
GATATGTATCATTGGCTGATATTGATTTAGAATCCTTAAAGTTATTATCAAGAAATATTGTCAAAAATTACAAAGAACAC
ATAGGATTAATATCATACTGGTTTTTGACCAAAGAGGTCAAAATACTAATGAAGCTTATAGGAGGAGTCAAACTACTAGG
AATTCCTAAACAGTACAAAGAGTTAGAGGATCGATCATCTCAGGGTTATGAATATGATAATGAATTTGATATTGATTAAT
ACATAAAAACAaAAAATAAAACACCTATTCCTCACCCATTCACTTCCAACAAAATGAAAAGTAAGAAAAACATGTAATAT
ATATATACCAAACAGAGTTTTTCTCTTGTTTGGT

In additional embodiments, the heterologous gene of the rB/HPIV3-SARS-COV-2/S comprises a SARS-COV-2 S protein-coding sequence that has been codon-optimized for expression in a human cell. For example, the encoding sequence of the heterologous gene can be codon-optimized for human expression using a GenScript (GS-opt) optimization algorithm. Non-limiting examples of nucleic acid sequences encoding the recombinant SARS-COV-2 S protein with amino acid modifications characteristic of B.1.617.2/Delta (SEQ ID NO: 38) that have been codon-optimized for expression in a human cell are provided as follows:

S-6P/B.1.617.2/Delta nucleotide sequence,
SEQ ID NO: 40
ATGTTCGTGTTTCTGGTGCTGCTGCCTCTGGTGAGCTCCCAGTGCGTGAACCTGAggACAAGGACCCAGCTGCCCCCTGC
CTATACCAATTCCTTCACACGGGGCGTGTACTATCCCGACAAGGTGTTTAGATCTAGCGTGCTGCACTCCACACAGGATC
TGTTTCTGCCTTTCTTTTCTAACGTGACCTGGTTCCACGCCATCCACGTGAGCGGCACCAATGGCACAAAGCGGTTCGAC
AATCCAGTGCTGCCCTTTAACGATGGCGTGTACTTCGCCTCCACCGAGAAGTCTAACATCATCAGAGGCTGGATCTTTGG
CACCACACTGGACAGCAAGACACAGTCCCTGCTGATCGTGAACAATGCCACCAACGTGGTCATCAAGGTGTGCGAGTTCC
AGTTTTGTAATGATCCATTCCTGGGCGTGTACTATCACAAGAACAATAAGTCTTGGATGGAGAGCgGCGTGTATTCCTCT
GCCAACAATTGCACATTTGAGTACGTGTCCCAGCCCTTCCTGATGGACCTGGAGGGCAAGCAGGGCAATTTCAAGAACCT
GAGGGAGTTCGTGTTTAAGAATATCGATGGCTACTTCAAGATCTACTCCAAGCACACCCCAATCAACCTGGTGCGCGACC
TGCCACAGGGCTTCTCTGCCCTGGAGCCACTGGTGGATCTGCCCATCGGCATCAACATCACCCGGTTTCAGACACTGCTG
GCCCTGCACAGAAGCTACCTGACACCAGGCGACAGCTCCTCTGGATGGACCGCAGGAGCTGCCGCCTACTATGTGGGCTA
TCTGCAGCCCAGGACCTTCCTGCTGAAGTACAACGAGAATGGCACCATCACAGACGCCGTGGATTGCGCCCTGGATCCCC
TGTCTGAGACCAAGTGTACACTGAAGAGCTTTACCGTGGAGAAGGGCATCTATCAGACAAGCAATTTCAGGGTGCAGCCT
ACCGAGTCCATCGTGCGCTTTCCCAATATCACAAACCTGTGCCCTTTTGGCGAGGTGTTCAACGCAACCCGCTTCGCCAG
CGTGTACGCCTGGAATAGGAAGCGCATCTCCAACTGCGTGGCCGACTATTCTGTGCTGTACAACAGCGCCTCCTTCTCTA
CCTTTAAGTGCTATGGCGTGAGCCCCACAAAGCTGAATGACCTGTGCTTTACCAACGTGTACGCCGATTCCTTCGTGATC
AGGGGCGACGAGGTGCGCCAGATCGCCCCTGGCCAGACAGGCAAGATCGCCGACTACAATTATAAGCTGCCTGACGATTT
CACCGGCTGCGTGATCGCCTGGAACTCTAACAATCTGGATAGCAAAGTGGGCGGCAACTACAATTATCgGTACCGGCTGT
TTAGAAAGTCTAATCTGAAGCCATTCGAGAGGGACATCTCCACAGAGATCTACCAGGCCGGCTCTAagCCCTGCAATGGC
GTGGAGGGCTTTAACTGTTATTTCCCTCTGCAGAGCTACGGCTTCCAGCCAACAAACGGCGTGGGCTATCAGCCCTACCG
CGTGGTGGTGCTGTCTTTTGAGCTGCTGCACGCACCTGCAACAGTGTGCGGACCAAAGAAGAGCACCAATCTGGTGAAGA
ACAAGTGCGTGAACTTCAACTTCAACGGACTGACCGGCACAGGCGTGCTGACCGAGTCCAACAAGAAGTTCCTGCCTTTT
CAGCAGTTCGGCAGGGACATCGCAGATACCACAGACGCCGTGCGCGACCCTCAGACCCTGGAGATCCTGGATATCACACC
ATGCTCCTTCGGCGGCGTGTCTGTGATCACACCAGGCACCAATACAAGCAACCAGGTGGCCGTGCTGTATCAGGgCGTGA
ATTGTACCGAGGTGCCCGTGGCAATCCACGCAGATCAGCTGACCCCTACATGGCGGGTGTACTCTACCGGCAGCAACGTG
TTCCAGACAAGAGCCGGATGCCTGATCGGAGCCGAGCACGTGAACAATAGCTATGAGTGCGACATCCCTATCGGCGCCGG
CATCTGTGCCTCCTACCAGACCCAGACAAACTCCagAgGGtctGCCtccTCTGTGGCCAGCCAGTCCATCATCGCCTATA
CCATGAGCCTGGGCGCCGAGAATTCCGTGGCCTACTCCAACAATTCTATCGCCATCCCTACCAACTTCACAATCTCCGTG
ACCACAGAGATCCTGCCAGTGAGCATGACCAAGACATCCGTGGACTGCACAATGTATATCTGTGGCGATTCCACCGAGTG
CTCTAACCTGCTGCTGCAGTACGGCTCTTTTTGTACCCAGCTGAATAGAGCCCTGACAGGCATCGCCGTGGAGCAGGACA
AGAACACACAGGAGGTGTTCGCCCAGGTGAAGCAGATCTACAAGACCCCACCCATCAAGGACTTTGGCGGCTTCAACTTC
AGCCAGATCCTGCCCGATCCTAGCAAGCCATCCAAGCGGTCTCCTATCGAGGACCTGCTGTTCAACAAGGTGACCCTGGC
CGATGCCGGCTTCATCAAGCAGTATGGCGATTGCCTGGGCGACATCGCCGCCAGAGACCTGATCTGTGCCCAGAAGTTTA
ATGGCCTGACCGTGCTGCCTCCACTGCTGACAGATGAGATGATCGCCCAGTACACATCTGCCCTGCTGGCCGGCACCATC
ACAAGCGGATGGACCTTCGGCGCAGGACCCGCCCTGCAGATCCCCTTTCCCATGCAGATGGCCTATCGGTTCAACGGCAT
CGGCGTGACCCAGAATGTGCTGTACGAGAACCAGAAGCTGATCGCCAATCAGTTTAACTCCGCCATCGGCAAGATCCAGG
ACTCTCTGAGCTCCACACCCAGCGCCCTGGGCAAGCTGCAGaacGTGGTGAATCAGAACGCCCAGGCCCTGAATACCCTG
GTGAAGCAGCTGTCTAGCAACTTCGGCGCCATCTCCTCTGTGCTGAATGATATCCTGAGCAGGCTGGACcctccaGAGGC
AGAGGTGCAGATCGACCGGCTGATCACAGGCAGACTGCAGTCCCTGCAGACCTACGTGACACAGCAGCTGATCAGGGCAG
CAGAGATCAGGGCCTCTGCCAATCTGGCCGCCACCAAGATGAGCGAGTGCGTGCTGGGCCAGTCCAAGAGAGTGGACTTT
TGTGGCAAGGGCTATCACCTGATGAGCTTCCCACAGTCCGCCCCTCACGGAGTGGTGTTTCTGCACGTGACCTACGTGCC
AGCCCAGGAGAAGAACTTCACCACAGCACCAGCAATCTGCCACGATGGCAAGGCACACTTTCCTAGGGAGGGCGTGTTCG
TGAGCAACGGCACCCACTGGTTTGTGACACAGCGCAATTTCTACGAGCCACAGATCATCACCACAGACAATACATTCGTG
TCCGGCAACTGTGACGTGGTCATCGGCATCGTGAACAATACCGTGTATGATCCTCTGCAGCCAGAGCTGGACTCTTTTAA
GGAGGAGCTGGATAAGTACTTCAAGAATCACACCAGCCCCGACGTGGATCTGGGCGACATCTCTGGCATCAATGCCAGCG
TGGTGAACATCCAGAAGGAGATCGACAGGCTGAACGAGGTGGCCAAGAATCTGAACGAGTCCCTGATCGATCTGCAGGAG
CTGGGCAAGTATGAGCAGTACATCAAGTGGCCCTGGTATATCTGGCTGGGCTTCATCGCCGGCCTGATCGCCATCGTGAT
GGTGACCATCATGCTGTGCTGTATGACAAGCTGCTGTTCCTGCCTGAAGGGCTGCTGTTCTTGTGGCAGCTGCTGTAAGT
TTGATGAGGACGATAGCGAGCCTGTGCTGAAGGGCGTGAAGCTGCACTACACCTGA

Non-limiting examples of nucleic acid sequences encoding the recombinant SARS-COV-2 S protein with amino acid modifications characteristic of B.1.529/Omicron that have been codon-optimized for expression in a human cell include the following:

S-6P/B.1.529/Omicron nucleotide sequence,
SEQ ID NO: 41
ATGTTCGTGTTTCTGGTGCTGCTGCCTCTGGTGAGCTCCCAGTGCGTGAACCTGACCACAAGGACCCAGCTGCCCCCTGC
CTATACCAATTCCTTCACACGGGGCGTGTACTATCCCGACAAGGTGTTTAGATCTAGCGTGCTGCACTCCACACAGGATC
TGTTTCTGCCTTTCTTTTCTAACGTGACCTGGTTCCACGtgATCAGCGGCACCAATGGCACAAAGCGGTTCGACAATCCA
GTGCTGCCCTTTAACGATGGCGTGTACTTCGCCTCCAtCGAGAAGTCTAACATCATCAGAGGCTGGATCTTTGGCACCAC
ACTGGACAGCAAGACACAGTCCCTGCTGATCGTGAACAATGCCACCAACGTGGTCATCAAGGTGTGCGAGTTCCAGTTTT
GTAATGATCCATTCCTGGaCCACAAGAACAATAAGTCTTGGATGGAGAGCGAGTTTCGCGTGTATTCCTCTGCCAACAAT
TGCACATTTGAGTACGTGTCCCAGCCCTTCCTGATGGACCTGGAGGGCAAGCAGGGCAATTTCAAGAACCTGAGGGAGTT
CGTGTTTAAGAATATCGATGGCTACTTCAAGATCTACTCCAAGCACACCCCAATCaTcGTGCGCGAGCCAGAAGACCTGC
CACAGGGCTTCTCTGCCCTGGAGCCACTGGTGGATCTGCCCATCGGCATCAACATCACCCGGTTTCAGACACTGCTGGCC
CTGCACAGAAGCTACCTGACACCAGGCGACAGCTCCTCTGGATGGACCGCAGGAGCTGCCGCCTACTATGTGGGCTATCT
GCAGCCCAGGACCTTCCTGCTGAAGTACAACGAGAATGGCACCATCACAGACGCCGTGGATTGCGCCCTGGATCCCCTGT
CTGAGACCAAGTGTACACTGAAGAGCTTTACCGTGGAGAAGGGCATCTATCAGACAAGCAATTTCAGGGTGCAGCCTACC
GAGTCCATCGTGCGCTTTCCCAATATCACAAACCTGTGCCCTTTTGaCGAGGTGTTCAACGCAACCCGCTTCGCCAGCGT
GTACGCCTGGAATAGGAAGCGCATCTCCAACTGCGTGGCCGACTATTCTGTGCTGTACAACcttGCtccaTTCTtcACCT
TTAAGTGCTATGGCGTGAGCCCCACAAAGCTGAATGACCTGTGCTTTACCAACGTGTACGCCGATTCCTTCGTGATCAGG
GGCGACGAGGTGCGCCAGATCGCCCCTGGCCAGACAGGCAACATCGCCGACTACAATTATAAGCTGCCTGACGATTTCAC
CGGCTGCGTGATCGCCTGGAACTCTAACAAgCTGGATAGCAAAGTGaGCGGCAACTACAATTATCTGTACCGGCTGTTTA
GAAAGTCTAATCTGAAGCCATTCGAGAGGGACATCTCCACAGAGATCTACCAGGCCGGCaacAagCCCTGCAATGGCGTG
GccGGCTTTAACTGTTATTTCCCTCTGagGAGCTACaGCTTCagGCCAACAtACGGCGTGGGacATCAGCCCTACCGCGT
GGTGGTGCTGTCTTTTGAGCTGCTGCACGCACCTGCAACAGTGTGCGGACCAAAGAAGAGCACCAATCTGGTGAAGAACA
AGTGCGTGAACTTCAACTTCAACGGACTGAagGGCACAGGCGTGCTGACCGAGTCCAACAAGAAGTTCCTGCCTTTTCAG
CAGTTCGGCAGGGACATCGCAGATACCACAGACGCCGTGCGCGACCCTCAGACCCTGGAGATCCTGGATATCACACCATG
CTCCTTCGGCGGCGTGTCTGTGATCACACCAGGCACCAATACAAGCAACCAGGTGGCCGTGCTGTATCAGGgCGTGAATT
GTACCGAGGTGCCCGTGGCAATCCACGCAGATCAGCTGACCCCTACATGGCGGGTGTACTCTACCGGCAGCAACGTGTTC
CAGACAAGAGCCGGATGCCTGATCGGAGCCGAGtACGTGAACAATAGCTATGAGTGCGACATCCCTATCGGCGCCGGCAT
CTGTGCCTCCTACCAGACCCAGACAAAgTCCCacgGGtctGCCtccTCTGTGGCCAGCCAGTCCATCATCGCCTATACCA
TGAGCCTGGGCGCCGAGAATTCCGTGGCCTACTCCAACAATTCTATCGCCATCCCTACCAACTTCACAATCTCCGTGACC
ACAGAGATCCTGCCAGTGAGCATGACCAAGACATCCGTGGACTGCACAATGTATATCTGTGGCGATTCCACCGAGTGCTC
TAACCTGCTGCTGCAGTACGGCTCTTTTTGTACCCAGCTGAAgAGAGCCCTGACAGGCATCGCCGTGGAGCAGGACAAGA
ACACACAGGAGGTGTTCGCCCAGGTGAAGCAGATCTACAAGACCCCACCCATCAAGtACTTTGGCGGCTTCAACTTCAGC
CAGATCCTGCCCGATCCTAGCAAGCCATCCAAGCGGTCTCCTATCGAGGACCTGCTGTTCAACAAGGTGACCCTGGCCGA
TGCCGGCTTCATCAAGCAGTATGGCGATTGCCTGGGCGACATCGCCGCCAGAGACCTGATCTGTGCCCAGAAGTTTAAgG
GCCTGACCGTGCTGCCTCCACTGCTGACAGATGAGATGATCGCCCAGTACACATCTGCCCTGCTGGCCGGCACCATCACA
AGCGGATGGACCTTCGGCGCAGGACCCGCCCTGCAGATCCCCTTTCCCATGCAGATGGCCTATCGGTTCAACGGCATCGG
CGTGACCCAGAATGTGCTGTACGAGAACCAGAAGCTGATCGCCAATCAGTTTAACTCCGCCATCGGCAAGATCCAGGACT
CTCTGAGCTCCACACCCAGCGCCCTGGGCAAGCTGCAGGATGTGGTGAATCACAACGCCCAGGCCCTGAATACCCTGGTG
AAGCAGCTGTCTAGCAAgTTCGGCGCCATCTCCTCTGTGCTGAATGATATCtTcAGCAGGCTGGACcctccaGAGGCAGA
GGTGCAGATCGACCGGCTGATCACAGGCAGACTGCAGTCCCTGCAGACCTACGTGACACAGCAGCTGATCAGGGCAGCAG
AGATCAGGGCCTCTGCCAATCTGGCCGCCACCAAGATGAGCGAGTGCGTGCTGGGCCAGTCCAAGAGAGTGGACTTTTGT
GGCAAGGGCTATCACCTGATGAGCTTCCCACAGTCCGCCCCTCACGGAGTGGTGTTTCTGCACGTGACCTACGTGCCAGC
CCAGGAGAAGAACTTCACCACAGCACCAGCAATCTGCCACGATGGCAAGGCACACTTTCCTAGGGAGGGCGTGTTCGTGA
GCAACGGCACCCACTGGTTTGTGACACAGCGCAATTTCTACGAGCCACAGATCATCACCACAGACAATACATTCGTGTCC
GGCAACTGTGACGTGGTCATCGGCATCGTGAACAATACCGTGTATGATCCTCTGCAGCCAGAGCTGGACTCTTTTAAGGA
GGAGCTGGATAAGTACTTCAAGAATCACACCAGCCCCGACGTGGATCTGGGCGACATCTCTGGCATCAATGCCAGCGTGG
TGAACATCCAGAAGGAGATCGACAGGCTGAACGAGGTGGCCAAGAATCTGAACGAGTCCCTGATCGATCTGCAGGAGCTG
GGCAAGTATGAGCAGTACATCAAGTGGCCCTGGTATATCTGGCTGGGCTTCATCGCCGGCCTGATCGCCATCGTGATGGT
GACCATCATGCTGTGCTGTATGACAAGCTGCTGTTCCTGCCTGAAGGGCTGCTGTTCTTGTGGCAGCTGCTGTAAGTTTG
ATGAGGACGATAGCGAGCCTGTGCTGAAGGGCGTGAAGCTGCACTACACCTGA

In some embodiments, the genome of the rB/HPIV3-SARS-COV-2/S vector comprises an antigenomic cDNA sequence set forth as SEQ ID NO: 42.

rB/HPIV3/S-6P/B.1.617.2
(SEQ ID NO: 42)
ACCAAACAAGAGAAGAGACTGGTTTGGGAATATTAATTCAAATAAAAATTAACTTAGGATTAAAGAACTTTACCGAAAGG
TAAGGGGAAAGAAATCCTAAGAGCTTAGCCATGTTGAGTCTATTCGACACATTCAGTGCGCGTAGGCAGGAGAACATAAC
GAAATCAGCTGGTGGGGCTGTTATTCCCGGGCAAAAAAACACTGTGTCTATATTTGCTCTTGGACCATCAATAACAGATG
ACAATGATAAAATGACATTGGCTCTTCTCTTTTTGTCTCATTCTTTAGACAATGAAAAGCAGCATGCGCAAAGAGCTGGA
TTTTTAGTTTCTCTGTTATCAATGGCTTATGCCAACCCAGAATTATATTTAACATCAAATGGTAGTAATGCAGATGTTAA
ATATGTTATCTACATGATAGAGAAAGACCCAGGAAGACAGAAATATGGTGGGTTTGTCGTCAAGACTAGAGAGATGGTTT
ATGAAAAGACAACTGATTGGATGTTCGGGAGTGATCTTGAGTATGATCAAGACAATATGTTGCAAAATGGTAGAAGCACT
TCTACAATCGAGGATCTTGTTCATACTTTTGGATATCCATCGTGTCTTGGAGCCCTTATAATCCAAGTTTGGATAATACT
TGTTAAGGCTATAACCAGTATATCAGGATTGAGGAAAGGATTCTTTACTCGGTTAGAAGCATTTCGACAAGATGGAACAG
TTAAATCCAGTCTAGTGTTGAGCGGTGATGCAGTAGAACAAATTGGATCAATTATGAGGTCCCAACAGAGCTTGGTAACA
CTCATGGTTGAAACACTGATAACAATGAACACAGGCAGGAATGATCTGACAACAATAGAAAAGAATATACAGATTGTAGG
AAACTACATCAGAGATGCAGGTCTTGCTTCATTTTTCAACACAATCAGATATGGCATTGAGACTAGAATGGCAGCTCTAA
CTCTGTCTACCCTTAGACCGGATATCAACAGACTCAAGGCACTGATCGAGTTATATCTATCAAAGGGGCCACGTGCTCCT
TTTATATGCATTTTGAGAGATCCCGTGCATGGTGAGTTTGCACCAGGCAACTATCCTGCCCTCTGGAGTTATGCGATGGG
TGTAGCAGTTGTACAAAACAAGGCCATGCAACAGTATGTAACAGGAAGGTCTTATCTGGATATTGAAATGTTCCAACTTG
GTCAAGCAGTGGCACGTGATGCCGAGTCGCAGATGAGTTCAATATTAGAGGATGAACTGGGGGTCACACAAGAAGCCAAG
CAAAGCTTGAAGAAACACATGAAGAACATCAGCAGTTCAGATACAACCTTTCATAAGCCTACAGGGGGATCAGCCATAGA
AATGGCGATAGATGAAGAAGCAGGGCAGCCTGAATCCAGAGGAGATCAGGATCAAGGAGATGAGCCTCGGTCATCCATAG
TTCCTTATGCATGGGCAGACGAAACCGGGAATGACAATCAAACTGAATCAACTACAGAAATTGACAGCATCAAAACTGAA
CAAAGAAACATCAGAGACAGGCTGAACAAAAGACTCAACGAGAAAAGGAAACAGAGTGACCCGAGATCAACTGACATCAC
AAACAACACAAATCAAACTGAAATAGATGATTTGTTCAGTGCATTCGGAAGCAACTAGTCACAAAGAGATGACCAGGCGC
GCCAAGTAAGAAAAACTTAGGATTAATGGACCTGCAGGATGTTCGTGTTTCTGGTGCTGCTGCCTCTGGTGAGCTCCCAG
TGCGTGAACCTGAggACAAGGACCCAGCTGCCCCCTGCCTATACCAATTCCTTCACACGGGGCGTGTACTATCCCGACAA
GGTGTTTAGATCTAGCGTGCTGCACTCCACACAGGATCTGTTTCTGCCTTTCTTTTCTAACGTGACCTGGTTCCACGCCA
TCCACGTGAGCGGCACCAATGGCACAAAGCGGTTCGACAATCCAGTGCTGCCCTTTAACGATGGCGTGTACTTCGCCTCC
ACCGAGAAGTCTAACATCATCAGAGGCTGGATCTTTGGCACCACACTGGACAGCAAGACACAGTCCCTGCTGATCGTGAA
CAATGCCACCAACGTGGTCATCAAGGTGTGCGAGTTCCAGTTTTGTAATGATCCATTCCTGGGCGTGTACTATCACAAGA
ACAATAAGTCTTGGATGGAGAGCqGCGTGTATTCCTCTGCCAACAATTGCACATTTGAGTACGTGTCCCAGCCCTTCCTG
ATGGACCTGGAGGGCAAGCAGGGCAATTTCAAGAACCTGAGGGAGTTCGTGTTTAAGAATATCGATGGCTACTTCAAGAT
CTACTCCAAGCACACCCCAATCAACCTGGTGCGCGACCTGCCACAGGGCTTCTCTGCCCTGGAGCCACTGGTGGATCTGC
CCATCGGCATCAACATCACCCGGTTTCAGACACTGCTGGCCCTGCACAGAAGCTACCTGACACCAGGCGACAGCTCCTCT
GGATGGACCGCAGGAGCTGCCGCCTACTATGTGGGCTATCTGCAGCCCAGGACCTTCCTGCTGAAGTACAACGAGAATGG
CACCATCACAGACGCCGTGGATTGCGCCCTGGATCCCCTGTCTGAGACCAAGTGTACACTGAAGAGCTTTACCGTGGAGA
AGGGCATCTATCAGACAAGCAATTTCAGGGTGCAGCCTACCGAGTCCATCGTGCGCTTTCCCAATATCACAAACCTGTGC
CCTTTTGGCGAGGTGTTCAACGCAACCCGCTTCGCCAGCGTGTACGCCTGGAATAGGAAGCGCATCTCCAACTGCGTGGC
CGACTATTCTGTGCTGTACAACAGCGCCTCCTTCTCTACCTTTAAGTGCTATGGCGTGAGCCCCACAAAGCTGAATGACC
TGTGCTTTACCAACGTGTACGCCGATTCCTTCGTGATCAGGGGCGACGAGGTGCGCCAGATCGCCCCTGGCCAGACAGGC
AAGATCGCCGACTACAATTATAAGCTGCCTGACGATTTCACCGGCTGCGTGATCGCCTGGAACTCTAACAATCTGGATAG
CAAAGTGGGCGGCAACTACAATTATCgGTACCGGCTGTTTAGAAAGTCTAATCTGAAGCCATTCGAGAGGGACATCTCCA
CAGAGATCTACCAGGCCGGCTCTAagCCCTGCAATGGCGTGGAGGGCTTTAACTGTTATTTCCCTCTGCAGAGCTACGGC
TTCCAGCCAACAAACGGCGTGGGCTATCAGCCCTACCGCGTGGTGGTGCTGTCTTTTGAGCTGCTGCACGCACCTGCAAC
AGTGTGCGGACCAAAGAAGAGCACCAATCTGGTGAAGAACAAGTGCGTGAACTTCAACTTCAACGGACTGACCGGCACAG
GCGTGCTGACCGAGTCCAACAAGAAGTTCCTGCCTTTTCAGCAGTTCGGCAGGGACATCGCAGATACCACAGACGCCGTG
CGCGACCCTCAGACCCTGGAGATCCTGGATATCACACCATGCTCCTTCGGCGGCGTGTCTGTGATCACACCAGGCACCAA
TACAAGCAACCAGGTGGCCGTGCTGTATCAGGgCGTGAATTGTACCGAGGTGCCCGTGGCAATCCACGCAGATCAGCTGA
CCCCTACATGGCGGGTGTACTCTACCGGCAGCAACGTGTTCCAGACAAGAGCCGGATGCCTGATCGGAGCCGAGCACGTG
AACAATAGCTATGAGTGCGACATCCCTATCGGCGCCGGCATCTGTGCCTCCTACCAGACCCAGACAAACTCCagAgGGtc
tGCCtccTCTGTGGCCAGCCAGTCCATCATCGCCTATACCATGAGCCTGGGCGCCGAGAATTCCGTGGCCTACTCCAACA
ATTCTATCGCCATCCCTACCAACTTCACAATCTCCGTGACCACAGAGATCCTGCCAGTGAGCATGACCAAGACATCCGTG
GACTGCACAATGTATATCTGTGGCGATTCCACCGAGTGCTCTAACCTGCTGCTGCAGTACGGCTCTTTTTGTACCCAGCT
GAATAGAGCCCTGACAGGCATCGCCGTGGAGCAGGACAAGAACACACAGGAGGTGTTCGCCCAGGTGAAGCAGATCTACA
AGACCCCACCCATCAAGGACTTTGGCGGCTTCAACTTCAGCCAGATCCTGCCCGATCCTAGCAAGCCATCCAAGCGGTCT
CCTATCGAGGACCTGCTGTTCAACAAGGTGACCCTGGCCGATGCCGGCTTCATCAAGCAGTATGGCGATTGCCTGGGCGA
CATCGCCGCCAGAGACCTGATCTGTGCCCAGAAGTTTAATGGCCTGACCGTGCTGCCTCCACTGCTGACAGATGAGATGA
TCGCCCAGTACACATCTGCCCTGCTGGCCGGCACCATCACAAGCGGATGGACCTTCGGCGCAGGACCCGCCCTGCAGATC
CCCTTTCCCATGCAGATGGCCTATCGGTTCAACGGCATCGGCGTGACCCAGAATGTGCTGTACGAGAACCAGAAGCTGAT
CGCCAATCAGTTTAACTCCGCCATCGGCAAGATCCAGGACTCTCTGAGCTCCACACCCAGCGCCCTGGGCAAGCTGCAGa
acGTGGTGAATCAGAACGCCCAGGCCCTGAATACCCTGGTGAAGCAGCTGTCTAGCAACTTCGGCGCCATCTCCTCTGTG
CTGAATGATATCCTGAGCAGGCTGGACcctccaGAGGCAGAGGTGCAGATCGACCGGCTGATCACAGGCAGACTGCAGTC
CCTGCAGACCTACGTGACACAGCAGCTGATCAGGGCAGCAGAGATCAGGGCCTCTGCCAATCTGGCCGCCACCAAGATGA
GCGAGTGCGTGCTGGGCCAGTCCAAGAGAGTGGACTTTTGTGGCAAGGGCTATCACCTGATGAGCTTCCCACAGTCCGCC
CCTCACGGAGTGGTGTTTCTGCACGTGACCTACGTGCCAGCCCAGGAGAAGAACTTCACCACAGCACCAGCAATCTGCCA
CGATGGCAAGGCACACTTTCCTAGGGAGGGCGTGTTCGTGAGCAACGGCACCCACTGGTTTGTGACACAGCGCAATTTCT
ACGAGCCACAGATCATCACCACAGACAATACATTCGTGTCCGGCAACTGTGACGTGGTCATCGGCATCGTGAACAATACC
GTGTATGATCCTCTGCAGCCAGAGCTGGACTCTTTTAAGGAGGAGCTGGATAAGTACTTCAAGAATCACACCAGCCCCGA
CGTGGATCTGGGCGACATCTCTGGCATCAATGCCAGCGTGGTGAACATCCAGAAGGAGATCGACAGGCTGAACGAGGTGG
CCAAGAATCTGAACGAGTCCCTGATCGATCTGCAGGAGCTGGGCAAGTATGAGCAGTACATCAAGTGGCCCTGGTATATC
TGGCTGGGCTTCATCGCCGGCCTGATCGCCATCGTGATGGTGACCATCATGCTGTGCTGTATGACAAGCTGCTGTTCCTG
CCTGAAGGGCTGCTGTTCTTGTGGCAGCTGCTGTAAGTTTGATGAGGACGATAGCGAGCCTGTGCTGAAGGGCGTGAAGC
TGCACTACACCTGATAGTAACTAGCGGCGCGCCAGCAACAAGTAAGAAAAACTTAGGATTAATGGAAATTATCCAATCCA
GAGACGGAAGGACAAATCCAGAATCCAACCACAACTCAATCAACCAAAGATTCATGGAAGACAATGTTCAAAACAATCAA
ATCATGGATTCTTGGGAAGAGGGATCAGGAGATAAATCATCTGACATCTCATCGGCCCTCGACATCATTGAATTCATACT
CAGCACCGACTCCCAAGAGAACACGGCAGACAGCAATGAAATCAACACAGGAACCACAAGACTTAGCACGACAATCTACC
AACCTGAATCCAAAACAACAGAAACAAGCAAGGAAAATAGTGGACCAGCTAACAAAAATCGACAGTTTGGGGCATCACAC
GAACGTGCCACAGAGACAAAAGATAGAAATGTTAATCAGGAGACTGTACAGGGAGGATATAGGAGAGGAAGCAGCCCAGA
TAGTAGAACTGAGACTATGGTCACTCGAAGAATCTCCAGAAGCAGCCCAGATCCTAACAATGGAACCCAAATCCAGGAAG
ATATTGATTACAATGAAGTTGGAGAGATGGATAAGGACTCTACTAAGAGGGAAATGCGACAATTTAAAGATGTTCCAGTC
AAGGTATCAGGAAGTGATGCCATTCCTCCAACAAAACAAGATGGAGACGGTGATGATGGAAGAGGCCTGGAATCTATCAG
TACATTTGATTCAGGATATACCAGTATAGTGACTGCCGCAACACTAGATGACGAAGAAGAACTCCTTATGAAGAACAACA
GGCCAAGAAAGTATCAATCAACACCCCAGAACAGTGACAAGGGAATTAAAAAAGGGGTTGGAAGGCCAAAAGACACAGAC
AAACAATCATCAATATTGGACTACGAACTCAACTTCAAAGGATCGAAGAAGAGCCAGAAAATCCTCAAAGCCAGCACGAA
TACAGGAGAACCAACAAGACCACAGAATGGATCCCAGGGGAAGAGAATCACATCCTGGAACATCCTCAACAGCGAGAGCG
GCAATCGAACAGAATCAACAAACCAAACCCATCAGACATCAACCTCGGGACAGAACCACACAATGGGACCAAGCAGAACA
ACCTCCGAACCAAGGATCAAGACACAAAAGACGGATGGAAAGGAAAGAGAGGACACAGAAGAGAGCACTCGATTTACAGA
AAGGGCGATTACATTATTACAGAATCTTGGTGTAATCCAATCTGCAGCAAAATTAGACCTATACCAAGACAAGAGAGTTG
TGTGTGTGGCGAATGTCCTAAACAATGCAGATACTGCATCAAAGATAGACTTCCTAGCAGGTTTGATGATAGGAGTGTCA
ATGGATCATGATACCAAATTAAATCAGATTCAGAACGAGATATTAAGTTTGAAAACTGATCTTAAAAAGATGGATGAATC
ACATAGAAGACTAATTGAGAATCAAAAAGAACAATTATCACTGATCACATCATTAATCTCAAATCTTAAAATTATGACAG
AGAGAGGAGGGAAGAAGGACCAACCAGAACCTAGCGGGAGGACATCCATGATCAAGACAAAAGCAAAAGAAGAGAAAATA
AAGAAAGTCAGGTTTGACCCTCTTATGGAAACACAGGGCATCGAGAAAAACATCCCTGACCTCTATAGATCAATAGAGAA
AACACCAGAAAACGACACACAGATCAAATCAGAAATAAACAGATTGAATGATGAATCCAATGCCACTAGATTAGTACCTA
GAAGAATAAGCAGTACAATGAGATCATTAATAATAATCATTAACAACAGCAATTTATCATCAAAAGCAAAGCAATCATAC
ATCAACGAACTCAAGCTCTGCAAGAGTGACGAGGAAGTGTCTGAGTTGATGGACATGTTCAATGAGGATGTCAGCTCCCA
GTAAACCGCCAACCAAGGGTCAACACCAAGAAAACCAATAGCACAAAACAGCCAATCAGAGACCACCCCAATACACCAAA
CCAATCAACACATAACAAAGATCGCGGCCGCATAGATGATTAAGAAAAACTTAGGATGAAAGGACTAATCAATCCTCCGA
AACAATGAGCATCACCAACTCCACAATCTACACATTCCCAGAATCCTCTTTCTCCGAGAATGGCAACATAGAGCCGTTAC
CACTCAAGGTCAATGAACAGAGAAAGGCCATACCTCATATTAGGGTTGTCAAGATAGGAGATCCGCCCAAACATGGATCC
AGATATCTGGATGTCTTTTTACTGGGCTTCTTTGAGATGGAAAGGTCAAAAGACAGGTATGGGAGCATAAGTGATCTAGA
TGATGATCCAAGTTACAAGGTTTGTGGCTCTGGATCATTGCCACTTGGGTTGGCTAGATACACCGGAAATGATCAGGAAC
TCCTACAGGCTGCAACCAAGCTCGATATAGAAGTAAGAAGAACTGTAAAGGCTACGGAGATGATAGTTTACACTGTACAA
AACATCAAACCTGAACTATATCCATGGTCCAGTAGATTAAGAAAAGGGATGTTATTTGACGCTAATAAGGTTGCACTTGC
TCCTCAATGTCTTCCACTAGATAGAGGGATAAAATTCAGGGTGATATTTGTGAACTGCACAGCAATTGGATCAATAACTC
TATTCAAAATCCCTAAGTCCATGGCATTGTTATCATTGCCTAATACAATATCAATAAATCTACAAGTACATATCAAAACA
GGAGTTCAGACAGATTCCAAAGGAGTAGTTCAGATTCTAGATGAAAAAGGTGAAAAATCACTAAATTTCATGGTTCATCT
CGGGTTGATCAAAAGGAAGATGGGCAGAATGTACTCAGTTGAATATTGTAAGCAGAAGATCGAGAAGATGAGATTATTAT
TCTCATTGGGATTAGTTGGAGGGATCAGCTTCCACGTCAACGCAACTGGCTCTATATCAAAGACATTAGCAAGTCAATTA
GCATTCAAAAGAGAAATCTGCTATCCCCTAATGGATCTGAATCCACACTTAAATTCAGTTATATGGGCATCATCAGTTGA
AATTACAAGGGTAGATGCAGTTCTCCAGCCTTCATTACCTGGCGAATTCAGATACTACCCAAACATCATAGCAAAAGGGG
TCGGGAAAATCAGACAGTAAAATCAACAACCCTGATATCCACCGGTGTATTAAGCCGAAGCAAATAAAGGATAATCAAAA
ACTTAGGACAAAAGAGGTCAATACCAACAACTATTAGCAGTCACACTCGCAAGAATAAGAGAGAAGGGACCAAAAAAGTC
AAATAGGAGAAATCAAAACAAAAGGTACAGAACACCAGAACAACAAAATCAAAACATCCAACTCACTCAAAACAAAAATT
CCAAAAGAGACCGGCAACACAACAAGCACTGAACACAATGCCAACTTCAATACTGCTAATTATTACAACCATGATCATGG
CATCTTTCTGCCAAATAGATATCACAAAACTACAGCACGTAGGTGTATTGGTCAACAGTCCCAAAGGGATGAAGATATCA
CAAAACTTTGAAACAAGATATCTAATTTTGAGCCTCATACCAAAAATAGAAGACTCTAACTCTTGTGGTGACCAACAGAT
CAAGCAATACAAGAAGTTATTGGATAGACTGATCATCCCTTTATATGATGGATTAAGATTACAGAAAGATGTGATAGTAA
CCAATCAAGAATCCAATGAAAACACTGATCCCAGAACAAAACGATTCTTTGGAGGGGTAATTGGAACCATTGCTCTGGGA
GTAGCAACCTCAGCACAAATTACAGCGGCAGTTGCTCTGGTTGAAGCCAAGCAGGCAAGATCAGACATCGAAAAACTCAA
AGAAGCAATTAGGGACACAAACAAAGCAGTGCAGTCAGTTCAGAGCTCCATAGGAAATTTAATAGTAGCAATTAAATCAG
TCCAGGATTATGTTAACAAAGAAATCGTGCCATCGATTGCGAGGCTAGGTTGTGAAGCAGCAGGACTTCAATTAGGAATT
GCATTAACACAGCATTACTCAGAATTAACAAACATATTTGGTGATAACATAGGATCGTTACAAGAAAAAGGAATAAAATT
ACAAGGTATAGCATCATTATACCGCACAAATATCACAGAAATATTCACAACATCAACAGTTGATAAATATGATATCTATG
ATCTGTTATTTACAGAATCAATAAAGGTGAGAGTTATAGATGTTGACTTGAATGATTACTCAATCACCCTCCAAGTCAGA
CTCCCTTTATTAACTAGGCTGCTGAACACTCAGATCTACAAAGTAGATTCCATATCATATAACATCCAAAACAGAGAATG
GTATATCCCTCTTCCCAGCCATATCATGACGAAAGGGGCATTTCTAGGTGGAGCAGACGTCAAAGAATGTATAGAAGCAT
TCAGCAGCTATATATGCCCTTCTGATCCAGGATTTGTATTAAACCATGAAATAGAGAGCTGCTTATCAGGAAACATATCC
CAATGTCCAAGAACAACGGTCACATCAGACATTGTTCCAAGATATGCATTTGTCAATGGAGGAGTGGTTGCAAACTGTAT
AACAACCACCTGTACATGCAACGGAATTGGTAATAGAATCAATCAACCACCTGATCAAGGAGTAAAAATTATAACACATA
AAGAATGTAGTACAATAGGTATCAACGGAATGCTGTTCAATACAAATAAAGAAGGAACTCTTGCATTCTATACACCAAAT
GATATAACACTAAACAATTCTGTTGCACTTGATCCAATTGACATATCAATCGAGCTCAACAAGGCCAAATCAGATCTAGA
AGAATCAAAAGAATGGATAAGAAGGTCAAATCAAAAACTAGATTCTATTGGAAATTGGCATCAATCTAGCACTACAATCA
TAATTATTTTGATAATGATCATTATATTGTTTATAATTAATATAACGATAATTACAATTGCAATTAAGTATTACAGAATT
CAAAAGAGAAATCGAGTGGATCAAAATGACAAGCCATATGTACTAACAAACAAATAACATATCTACAGATCATTAGATAT
TAAAATTATAAAAAACTTAGGAGTAAAGTTACGCAATCCAACTCTACTCATATAATTGAGGAAGGACCCAATAGACAAAT
CCAAATTCGAGATGGAATACTGGAAGCATACCAATCACGGAAAGGATGCTGGTAATGAGCTGGAGACGTCTATGGCTACT
CATGGCAACAAGCTCACTAATAAGATAATATACATATTATGGACAATAATCCTGGTGTTATTATCAATAGTCTTCATCAT
AGTGCTAATTAATTCCATCAAAAGTGAAAAGGCCCACGAATCATTGCTGCAAGACATAAATAATGAGTTTATGGAAATTA
CAGAAAAGATCCAAATGGCATCGGATAATACCAATGATCTAATACAGTCAGGAGTGAATACAAGGCTTCTTACAATTCAG
AGTCATGTCCAGAATTACATACCAATATCATTGACACAACAGATGTCAGATCTTAGGAAATTCATTAGTGAAATTACAAT
TAGAAATGATAATCAAGAAGTGCTGCCACAAAGAATAACACATGATGTAGGTATAAAACCTTTAAATCCAGATGATTTTT
GGAGATGCACGTCTGGTCTTCCATCTTTAATGAAAACTCCAAAAATAAGGTTAATGCCAGGGCCGGGATTATTAGCTATG
CCAACGACTGTTGATGGCTGTGTTAGAACTCCGTCTTTAGTTATAAATGATCTGATTTATGCTTATACCTCAAATCTAAT
TACTCGAGGTTGTCAGGATATAGGAAAATCATATCAAGTCTTACAGATAGGGATAATAACTGTAAACTCAGACTTGGTAC
CTGACTTAAATCCTAGGATCTCTCATACCTTTAACATAAATGACAATAGGAAGTCATGTTCTCTAGCACTCCTAAATAcA
GATGTATATCAACTGTGTTCAACTCCCAAAGTTGATGAAAGATCAGATTATGCATCATCAGGCATAGAAGATATTGTACT
TGATATTGTCAATTATGATGGTTCAATCTCAACAACAAGATTTAAGAATAATAACATAAGCTTTGATCAACCATATGCTG
CACTATACCCATCTGTTGGACCAGGGATATACTACAAAGGCAAAATAATATTTCTCGGGTATGGAGGTCTTGAACATCCA
ATAAATGAGAATGTAATCTGCAACACAACTGGGTGCCCCGGGAAAACACAGAGAGACTGTAATCAAGCATCTCATAGTcC
aTGGTTTTCAGATAGGAGGATGGTCAACTCCATCATTGTTGTTGACAAAGGCTTAAACTCAATTCCAAAATTGAAAGTAT
GGACGATATCTATGCGACAAAATTACTGGGGGTCAGAAGGAAGGTTACTTCTACTAGGTAACAAGATCTATATATATACA
AGATCTACAAGTTGGCATAGCAAGTTACAATTAGGAATAATTGATATTACTGATTACAGTGATATAAGGATAAAATGGAC
ATGGCATAATGTGCTATCAAGACCAGGAAACAATGAATGTCCATGGGGACATTCATGTCCAGATGGATGTATAACAGGAG
TATATACTGATGCATATCCACTCAATCCCACAGGGAGCATTGTGTCATCTGTCATATTAGACTCACAAAAATCGAGAGTG
AACCCAGTCATAACTTACTCAACAGCAACCGAAAGAGTAAACGAGCTGGCCATCCTAAACAGAACACTCTCAGCTGGATA
TACAACAACAAGCTGCATTACACACTATAACAAAGGATATTGTTTTCATATAGTAGAAATAAATCATAAAAGCTTAAACA
CATTTCAACCCATGTTGTTCAAAACAGAGATTCCAAAAAGCTGCAGTTAATCATAATTAACCATAATATGCATCAATCTA
TCTATAATACAAGTATATGATAAGTAATCAGCAATCAGACAATAGACGTACGGAAATAATAAAAAACTTAGGAGAAAAGT
GTGCAAGAAAAATGGACACCGAGTCCCACAGCGGCACAACATCTGACATTCTGTACCCTGAATGTCACCTCAATTCTCCT
ATAGTTAAAGGAAAGATAGCACAACTGCATACAATAATGAGTTTGCCTCAGCCCTACGATATGGATGATGATTCAATACT
GATTATTACTAGACAAAAAATTAAACTCAATAAATTAGATAAAAGACAACGGTCAATTAGGAAATTAAGATCAGTCTTAA
TGGAAAGAGTAAGTGATCTAGGTAAATATACCTTTATCAGATATCCAGAGATGTCTAGTGAAATGTTCCAATTATGTATA
CCCGGAATTAATAATAAAATAAATGAATTGCTAAGTAAAGCAAGTAAAACATATAATCAAATGACTGATGGATTAAGAGA
TCTATGGGTTACTATACTATCGAAGTTAGCATCGAAAAATGATGGAAGTAATTATGATATCAATGAAGATATTAGCAATA
TATCAAATGTTCACATGACTTATCAATCAGACAAATGGTATAATCCATTCAAGACATGGTTTACTATTAAGTATGACATG
AGAAGATTACAAAAAGCCAAAAATGAGATTACATTCAATAGGCATAAAGATTATAATCTATTAGAAGACCAAAAGAATAT
ATTGCTGATACATCCAGAACTCGTCTTAATATTAGATAAACAAAATTACAATGGGTATATAATGACTCCTGAATTGGTAC
TAATGTATTGTGATGTAGTTGAAGGGAGGTGGAATATAAGTTCATGTGCAAAATTGGATCCTAAGTTACAATCAATGTAT
TATAAGGGTAACAATTTATGGGAAATAATAGATGGACTATTCTCGACCTTAGGAGAAAGAACATTTGACATAATATCACT
ATTAGAACCACTTGCATTATCGCTCATTCAAACTTATGACCCGGTTAAACAGCTCAGGGGGGCTTTTTTAAATCACGTGT
TATCAGAAATGGAATTAATATTTGCAGCTGAGTGTACAACAGAGGAAATACCTAATGTGGATTATATAGATAAAATTTTA
GATGTGTTCAAAGAATCAACAATAGATGAAATAGCAGAAATTTTCTCTTTCTTCCGAACTTTTGGACACCCTCCATTAGA
GGCGAGTATAGCAGCAGAGAAAGTTAGAAAGTATATGTATACTGAGAAATGCTTGAAATTTGATACTATCAATAAATGTC
ATGCTATTTTTTGTACAATAATTATAAATGGATATAGAGAAAGACATGGTGGTCAATGGCCTCCAGTTACATTACCTGTC
CATGCACATGAATTTATCATAAATGCATACGGATCAAATTCTGCCATATCATATGAGAATGCTGTAGATTATTATAAGAG
CTTCATAGGAATAAAATTTGACAAGTTTATAGAGCCTCAATTGGATGAAGACTTAACTATTTATATGAAAGATAAAGCAT
TATCCCCAAAGAAATCAAACTGGGACACAGTCTATCCAGCTTCAAACCTGTTATACCGCACTAATGTGTCTCATGATTCA
CGAAGATTGGTTGAAGTATTTATAGCAGATAGTAAATTTGATCCCCACCAAGTATTAGATTACGTAGAATCAGGATATTG
GCTGGATGATCCTGAATTTAATATCTCATATAGTTTAAAAGAGAAAGAAATAAAACAAGAAGGTAGACTTTTTGCAAAAA
TGACATACAAGATGAGGGCTACACAAGTATTATCAGAAACATTATTGGCGAATAATATAGGGAAATTCTTCCAAGAGAAT
GGGATGGTTAAAGGAGAAATTGAATTACTCAAGAGACTAACAACAATATCTATGTCTGGAGTTCCGCGGTATAATGAGGT
ATACAATAATTCAAAAAGTCACACAGAAGAACTTCAAGCTTATAATGCAATTAGCAGTTCCAATTTATCTTCTAATCAGA
AGTCAAAGAAGTTTGAATTTAAATCTACAGATATATACAATGATGGATACGAAACCGTAAGCTGCTTCTTAACGACAGAT
CTTAAAAAATATTGTTTAAATTGGAGGTATGAATCAACAGCTTTATTCGGTGATACTTGTAATCAGATATTTGGGTTAAA
GGAATTATTTAATTGGCTGCACCCTCGCCTTGAAAAGAGTACAATATATGTTGGAGATCCTTATTGCCCGCCATCAGATA
TTGAACATTTACCACTTGATGACCATCCTGATTCAGGATTTTATGTTCATAATCCTAAAGGAGGAATAGAAGGGTTTTGC
CAAAAGTTATGGACACTCATATCTATCAGTGCAATACATTTAGCAGCTGTCAAAATCGGTGTAAGAGTTACTGCAATGGT
TCAAGGGGATAATCAAGCCATAGCTGTTACCACAAGAGTACCTAATAATTATGATTATAAAGTTAAGAAAGAGATTGTTT
ATAAAGATGTGGTAAGATTTTTTGATTCCTTGAGAGAGGTGATGGATGATCTGGGTCATGAGCTCAAACTAAATGAAACT
ATAATAAGTAGTAAAATGTTTATATATAGCAAAAGGATATACTATGACGGAAGAATCCTTCCTCAGGCATTAAAAGCATT
GTCTAGATGTGTTTTTTGGTCTGAAACAATCATAGATGAGACAAGATCAGCATCCTCAAATCTGGCTACATCGTTTGCAA
AGGCCATTGAGAATGGCTACTCACCTGTATTGGGATATGTATGCTCAATCTTCAAAAATATCCAACAGTTGTATATAGCG
CTTGGAATGAATATAAACCCAACTATAACCCAAAATATTAAAGATCAATATTTCAGGAATATTCATTGGATGCAATATGC
CTCCTTAATCCCTGCTAGTGTCGGAGGATTTAATTATATGGCCATGTCAAGGTGTTTTGTCAGAAACATTGGAGATCCTA
CAGTCGCTGCGTTAGCCGATATTAAAAGATTTATAAAAGCAAATTTGTTAGATCGAGGTGTCCTTTACAGAATTATGAAT
CAAGAACCAGGCGAGTCTTCTTTTTTAGACTGGGCCTCAGATCCCTATTCATGTAACTTACCACAATCTCAAAATATAAC
CACCATGATAAAGAATATAACTGCAAGAAATGTACTACAGGACTCACCAAACCCATTACTATCTGGATTATTTACAAGTA
CAATGATAGAAGAGGATGAGGAATTAGCTGAGTTCCTAATGGACAGGAGAATAATCCTCCCAAGAGTTGCACATGACATT
TTAGATAATTCTCTTACTGGAATTAGGAATGCTATAGCTGGTATGTTGGATACAACAAAATCACTAATTCGAGTAGGGAT
AAGCAGAGGAGGATTAACCTATAACTTATTAAGAAAGATAAGCAACTATGATCTTGTACAATATGAGACACTTAGTAAAA
CTTTAAGACTAATAGTCAGTGACAAGATTAAGTATGAAGATATGTGCTCAGTAGACCTAGCCATATCATTAAGACAAAAA
ATGTGGATGCATTTATCAGGAGGAAGAATGATAAATGGACTTGAAACTCCAGATCCTTTAGAGTTACTGTCTGGAGTAAT
AATAACAGGATCTGAACATTGTAGGATATGTTATTCAACTGAAGGTGAAAGCCCATATACATGGATGTATTTACCAGGCA
ATCTTAATATAGGATCAGCTGAGACAGGAATAGCATCATTAAGGGTCCCTTACTTTGGATCAGTTACAGATGAGAGATCT
GAAGCACAATTAGGGTATATCAAAAATCTAAGCAAACCAGCTAAGGCTGCTATAAGAATAGCAATGATATATACTTGGGC
ATTTGGGAATGACGAAATATCTTGGATGGAAGCATCACAGATTGCACAAACACGTGCAAACTTTACATTGGATAGCTTAA
AGATTTTGACACCAGTGACAACATCAACAAATCTATCACACAGGTTAAAAGATACTGCTACTCAGATGAAATTTTCTAGT
ACATCACTTATTAGAGTAAGCAGGTTCATCACAATATCTAATGATAATATGTCTATTAAAGAAGCAAATGAAACTAAAGA
TACAAATCTTATTTATCAACAGGTAATGTTAACAGGATTAAGTGTATTTGAATATCTATTTAGGTTAGAGGAGAGTACAG
GACATAACCCTATGGTCATGCATCTACATATAGAGGATGGATGTTGTATAAAAGAGAGTTACAATGATGAGCATATCAAT
CCGGAGTCTACATTAGAGTTAATCAAATACCCTGAGAGTAATGAATTTATATATGATAAGGACCCTTTAAAGGATATAGA
TCTATCAAAATTAATGGTTATAAGAGATCATTCTTATACAATTGACATGAATTACTGGGATGACACAGATATTGTACATG
CAATATCAATATGTACTGCAGTTACAATAGCAGATACAATGTCGCAGCTAGATCGGGATAATCTTAAGGAGCTGGTTGTG
ATTGCAAATGATGATGATATTAACAGTCTGATAACTGAATTTCTGACCCTAGATATACTAGTGTTTCTCAAAACATTTGG
AGGGTTACTCGTGAATCAATTTGCATATACCCTTTATGGATTGAAAATAGAAGGAAGGGATCCCATTTGGGATTATATAA
TGAGAACATTAAAAGACACCTCACATTCAGTACTTAAAGTATTATCTAATGCACTATCTCATCCAAAAGTGTTTAAGAGA
TTTTGGGATTGTGGAGTTTTGAATCCTATTTATGGTCCTAATACTGCTAGTCAAGATCAAGTTAAGCTTGCTCTCTCGAT
TTGCGAGTACTCCTTGGATCTATTTATGAGAGAATGGTTGAATGGAGCATCACTTGAGATCTATATCTGTGATAGTGACA
TGGAAATAGCAAATGACAGAAGACAAGCATTTCTCTCAAGACATCTTGCCTTTGTGTGTTGTTTAGCAGAGATAGCATCT
TTTGGACCAAATTTATTAAATCTAACATATCTAGAGAGACTTGATGAATTAAAACAATACTTAGATCTGAACATCAAAGA
AGATCCTACTCTTAAATATGTGCAAGTATCAGGACTGTTAATTAAATCATTCCCCTCAACTGTTACGTATGTAAGGAAAA
CTGCGATTAAGTATCTGAGGATTCGTGGTATTAATCCGCCTGAAACGATTGAAGATTGGGATCCCATAGAAGATGAGAAT
ATCTTAGACAATATTGTTAAAACTGTAAATGACAATTGCAGTGATAATCAAAAGAGAAATAAAAGTAGTTATTTCTGGGG
ATTAGCTCTAAAGAATTATCAAGTCGTGAAAATAAGATCCATAACGAGTGATTCTGAAGTTAATGAAGCTTCGAATGTTA
CTACACATGGAATGACACTTCCTCAGGGAGGAAGTTATCTATCACATCAGCTGAGGTTATTTGGAGTAAACAGTACAAGT
TGTCTTAAAGCTCTTGAATTATCACAAATCTTAATGAGGGAAGTTAAAAAAGATAAAGATAGACTCTTTTTAGGAGAAGG
AGCAGGAGCTATGTTAGCATGTTATGATGCTACACTCGGTCCTGCAATAAATTATTATAATTCTGGTTTAAATATTACAG
ATGTAATTGGTCAACGGGAATTAAAAATCTTCCCATCAGAAGTATCATTAGTAGGTAAAAAACTAGGAAATGTAACACAG
ATTCTTAATCGGGTGAGGGTGTTATTTAATGGGAATCCCAATTCAACATGGATAGGAAATATGGAATGTGAGAGTTTAAT
ATGGAGTGAATTAAATGATAAGTCAATTGGTTTAGTACATTGTGACATGGAGGGAGCGATAGGCAAATCAGAAGAAACTG
TTCTACATGAACATTATAGTATTATTAGGATTACATATTTAATCGGGGATGATGATGTTGTCCTAGTATCAAAAATTATA
CCAACTATTACTCCGAATTGGTCTAAAATACTCTATCTATACAAGTTGTATTGGAAGGATGTAAGTGTAGTGTCCCTTAA
AACATCCAATCCTGCCTCAACAGAGCTTTATTTAATTTCAAAAGATGCTTACTGTACTGTAATGGAACCCAGTAATCTTG
TTTTATCAAAACTTAAAAGGATATCATCAATAGAAGAAAATAATCTATTAAAGTGGATAATCTTATCAAAAAGGAAGAAT
AACGAGTGGTTACAGCATGAAATCAAAGAAGGAGAAAGGGATTATGGGATAATGAGGCCATATCATACAGCACTGCAAAT
TTTTGGATTCCAAATTAACTTAAATCACTTAGCTAGAGAATTTTTATCAACTCCTGATTTAACCAACATTAATAATATAA
TTCAAAGTTTTACAAGAACAATTAAAGATGTTATGTTCGAATGGGTCAATATCACTCATGACAATAAAAGACATAAATTA
GGAGGAAGATATAATCTATTCCCGCTTAAAAATAAGGGGAAATTAAGATTATTATCACGAAGATTAGTACTAAGCTGGAT
ATCATTATCCTTATCAACCAGATTACTGACGGGCCGTTTTCCAGATGAAAAATTTGAAAATAGGGCACAGACCGGATATG
TATCATTGGCTGATATTGATTTAGAATCCTTAAAGTTATTATCAAGAAATATTGTCAAAAATTACAAAGAACACATAGGA
TTAATATCATACTGGTTTTTGACCAAAGAGGTCAAAATACTAATGAAGCTTATAGGAGGAGTCAAACTACTAGGAATTCC
TAAACAGTACAAAGAGTTAGAGGATCGATCATCTCAGGGTTATGAATATGATAATGAATTTGATATTGATTAATACATAA
AAACAaAAAATAAAACACCTATTCCTCACCCATTCACTTCCAACAAAATGAAAAGTAAGAAAAACATGTAATATATATAT
ACCAAACAGAGTTTTTCTCTTGTTTGGT

In some embodiments, the genome of the rB/HPIV3-SARS-COV-2/S vector comprises an antigenomic cDNA sequence set forth as SEQ ID NO: 43.

rB/HPIV3/S-6P/B.1.529
(SEQ ID NO: 43)
ACCAAACAAGAGAAGAGACTGGTTTGGGAATATTAATTCAAATAAAAATTAACTTAGGATTAAAGAACTTTACCGAAAGG
TAAGGGGAAAGAAATCCTAAGAGCTTAGCCATGTTGAGTCTATTCGACACATTCAGTGCGCGTAGGCAGGAGAACATAAC
GAAATCAGCTGGTGGGGCTGTTATTCCCGGGCAAAAAAACACTGTGTCTATATTTGCTCTTGGACCATCAATAACAGATG
ACAATGATAAAATGACATTGGCTCTTCTCTTTTTGTCTCATTCTTTAGACAATGAAAAGCAGCATGCGCAAAGAGCTGGA
TTTTTAGTTTCTCTGTTATCAATGGCTTATGCCAACCCAGAATTATATTTAACATCAAATGGTAGTAATGCAGATGTTAA
ATATGTTATCTACATGATAGAGAAAGACCCAGGAAGACAGAAATATGGTGGGTTTGTCGTCAAGACTAGAGAGATGGTTT
ATGAAAAGACAACTGATTGGATGTTCGGGAGTGATCTTGAGTATGATCAAGACAATATGTTGCAAAATGGTAGAAGCACT
TCTACAATCGAGGATCTTGTTCATACTTTTGGATATCCATCGTGTCTTGGAGCCCTTATAATCCAAGTTTGGATAATACT
TGTTAAGGCTATAACCAGTATATCAGGATTGAGGAAAGGATTCTTTACTCGGTTAGAAGCATTTCGACAAGATGGAACAG
TTAAATCCAGTCTAGTGTTGAGCGGTGATGCAGTAGAACAAATTGGATCAATTATGAGGTCCCAACAGAGCTTGGTAACA
CTCATGGTTGAAACACTGATAACAATGAACACAGGCAGGAATGATCTGACAACAATAGAAAAGAATATACAGATTGTAGG
AAACTACATCAGAGATGCAGGTCTTGCTTCATTTTTCAACACAATCAGATATGGCATTGAGACTAGAATGGCAGCTCTAA
CTCTGTCTACCCTTAGACCGGATATCAACAGACTCAAGGCACTGATCGAGTTATATCTATCAAAGGGGCCACGTGCTCCT
TTTATATGCATTTTGAGAGATCCCGTGCATGGTGAGTTTGCACCAGGCAACTATCCTGCCCTCTGGAGTTATGCGATGGG
TGTAGCAGTTGTACAAAACAAGGCCATGCAACAGTATGTAACAGGAAGGTCTTATCTGGATATTGAAATGTTCCAACTTG
GTCAAGCAGTGGCACGTGATGCCGAGTCGCAGATGAGTTCAATATTAGAGGATGAACTGGGGGTCACACAAGAAGCCAAG
CAAAGCTTGAAGAAACACATGAAGAACATCAGCAGTTCAGATACAACCTTTCATAAGCCTACAGGGGGATCAGCCATAGA
AATGGCGATAGATGAAGAAGCAGGGCAGCCTGAATCCAGAGGAGATCAGGATCAAGGAGATGAGCCTCGGTCATCCATAG
TTCCTTATGCATGGGCAGACGAAACCGGGAATGACAATCAAACTGAATCAACTACAGAAATTGACAGCATCAAAACTGAA
CAAAGAAACATCAGAGACAGGCTGAACAAAAGACTCAACGAGAAAAGGAAACAGAGTGACCCGAGATCAACTGACATCAC
AAACAACACAAATCAAACTGAAATAGATGATTTGTTCAGTGCATTCGGAAGCAACTAGTCACAAAGAGATGACCAGGCGC
GCCAAGTAAGAAAAACTTAGGATTAATGGACCTGCAGGATGTTCGTGTTTCTGGTGCTGCTGCCTCTGGTGAGCTCCCAG
TGCGTGAACCTGACCACAAGGACCCAGCTGCCCCCTGCCTATACCAATTCCTTCACACGGGGCGTGTACTATCCCGACAA
GGTGTTTAGATCTAGCGTGCTGCACTCCACACAGGATCTGTTTCTGCCTTTCTTTTCTAACGTGACCTGGTTCCACGtgA
TCAGCGGCACCAATGGCACAAAGCGGTTCGACAATCCAGTGCTGCCCTTTAACGATGGCGTGTACTTCGCCTCCAtCGAG
AAGTCTAACATCATCAGAGGCTGGATCTTTGGCACCACACTGGACAGCAAGACACAGTCCCTGCTGATCGTGAACAATGC
CACCAACGTGGTCATCAAGGTGTGCGAGTTCCAGTTTTGTAATGATCCATTCCTGGaCCACAAGAACAATAAGTCTTGGA
TGGAGAGCGAGTTTCGCGTGTATTCCTCTGCCAACAATTGCACATTTGAGTACGTGTCCCAGCCCTTCCTGATGGACCTG
GAGGGCAAGCAGGGCAATTTCAAGAACCTGAGGGAGTTCGTGTTTAAGAATATCGATGGCTACTTCAAGATCTACTCCAA
GCACACCCCAATCaTcGTGCGCGAGCCAGAAGACCTGCCACAGGGCTTCTCTGCCCTGGAGCCACTGGTGGATCTGCCCA
TCGGCATCAACATCACCCGGTTTCAGACACTGCTGGCCCTGCACAGAAGCTACCTGACACCAGGCGACAGCTCCTCTGGA
TGGACCGCAGGAGCTGCCGCCTACTATGTGGGCTATCTGCAGCCCAGGACCTTCCTGCTGAAGTACAACGAGAATGGCAC
CATCACAGACGCCGTGGATTGCGCCCTGGATCCCCTGTCTGAGACCAAGTGTACACTGAAGAGCTTTACCGTGGAGAAGG
GCATCTATCAGACAAGCAATTTCAGGGTGCAGCCTACCGAGTCCATCGTGCGCTTTCCCAATATCACAAACCTGTGCCCT
TTTGaCGAGGTGTTCAACGCAACCCGCTTCGCCAGCGTGTACGCCTGGAATAGGAAGCGCATCTCCAACTGCGTGGCCGA
CTATTCTGTGCTGTACAACcttGCtccaTTCTtcACCTTTAAGTGCTATGGCGTGAGCCCCACAAAGCTGAATGACCTGT
GCTTTACCAACGTGTACGCCGATTCCTTCGTGATCAGGGGCGACGAGGTGCGCCAGATCGCCCCTGGCCAGACAGGCAAC
ATCGCCGACTACAATTATAAGCTGCCTGACGATTTCACCGGCTGCGTGATCGCCTGGAACTCTAACAAgCTGGATAGCAA
AGTGaGCGGCAACTACAATTATCTGTACCGGCTGTTTAGAAAGTCTAATCTGAAGCCATTCGAGAGGGACATCTCCACAG
AGATCTACCAGGCCGGCaacAagCCCTGCAATGGCGTGGccGGCTTTAACTGTTATTTCCCTCTGagGAGCTACaGCTTC
agGCCAACAtACGGCGTGGGacATCAGCCCTACCGCGTGGTGGTGCTGTCTTTTGAGCTGCTGCACGCACCTGCAACAGT
GTGCGGACCAAAGAAGAGCACCAATCTGGTGAAGAACAAGTGCGTGAACTTCAACTTCAACGGACTGAagGGCACAGGCG
TGCTGACCGAGTCCAACAAGAAGTTCCTGCCTTTTCAGCAGTTCGGCAGGGACATCGCAGATACCACAGACGCCGTGCGC
GACCCTCAGACCCTGGAGATCCTGGATATCACACCATGCTCCTTCGGCGGCGTGTCTGTGATCACACCAGGCACCAATAC
AAGCAACCAGGTGGCCGTGCTGTATCAGGgCGTGAATTGTACCGAGGTGCCCGTGGCAATCCACGCAGATCAGCTGACCC
CTACATGGCGGGTGTACTCTACCGGCAGCAACGTGTTCCAGACAAGAGCCGGATGCCTGATCGGAGCCGAGLACGTGAAC
AATAGCTATGAGTGCGACATCCCTATCGGCGCCGGCATCTGTGCCTCCTACCAGACCCAGACAAAgTCCCacgGGtctGC
CtccTCTGTGGCCAGCCAGTCCATCATCGCCTATACCATGAGCCTGGGCGCCGAGAATTCCGTGGCCTACTCCAACAATT
CTATCGCCATCCCTACCAACTTCACAATCTCCGTGACCACAGAGATCCTGCCAGTGAGCATGACCAAGACATCCGTGGAC
TGCACAATGTATATCTGTGGCGATTCCACCGAGTGCTCTAACCTGCTGCTGCAGTACGGCTCTTTTTGTACCCAGCTGAA
gAGAGCCCTGACAGGCATCGCCGTGGAGCAGGACAAGAACACACAGGAGGTGTTCGCCCAGGTGAAGCAGATCTACAAGA
CCCCACCCATCAAGtACTTTGGCGGCTTCAACTTCAGCCAGATCCTGCCCGATCCTAGCAAGCCATCCAAGCGGTCTCCT
ATCGAGGACCTGCTGTTCAACAAGGTGACCCTGGCCGATGCCGGCTTCATCAAGCAGTATGGCGATTGCCTGGGCGACAT
CGCCGCCAGAGACCTGATCTGTGCCCAGAAGTTTAAgGGCCTGACCGTGCTGCCTCCACTGCTGACAGATGAGATGATCG
CCCAGTACACATCTGCCCTGCTGGCCGGCACCATCACAAGCGGATGGACCTTCGGCGCAGGACCCGCCCTGCAGATCCCC
TTTCCCATGCAGATGGCCTATCGGTTCAACGGCATCGGCGTGACCCAGAATGTGCTGTACGAGAACCAGAAGCTGATCGC
CAATCAGTTTAACTCCGCCATCGGCAAGATCCAGGACTCTCTGAGCTCCACACCCAGCGCCCTGGGCAAGCTGCAGGATG
TGGTGAATCACAACGCCCAGGCCCTGAATACCCTGGTGAAGCAGCTGTCTAGCAAqTTCGGCGCCATCTCCTCTGTGCTG
AATGATATCtTcAGCAGGCTGGACcctccaGAGGCAGAGGTGCAGATCGACCGGCTGATCACAGGCAGACTGCAGTCCCT
GCAGACCTACGTGACACAGCAGCTGATCAGGGCAGCAGAGATCAGGGCCTCTGCCAATCTGGCCGCCACCAAGATGAGCG
AGTGCGTGCTGGGCCAGTCCAAGAGAGTGGACTTTTGTGGCAAGGGCTATCACCTGATGAGCTTCCCACAGTCCGCCCCT
CACGGAGTGGTGTTTCTGCACGTGACCTACGTGCCAGCCCAGGAGAAGAACTTCACCACAGCACCAGCAATCTGCCACGA
TGGCAAGGCACACTTTCCTAGGGAGGGCGTGTTCGTGAGCAACGGCACCCACTGGTTTGTGACACAGCGCAATTTCTACG
AGCCACAGATCATCACCACAGACAATACATTCGTGTCCGGCAACTGTGACGTGGTCATCGGCATCGTGAACAATACCGTG
TATGATCCTCTGCAGCCAGAGCTGGACTCTTTTAAGGAGGAGCTGGATAAGTACTTCAAGAATCACACCAGCCCCGACGT
GGATCTGGGCGACATCTCTGGCATCAATGCCAGCGTGGTGAACATCCAGAAGGAGATCGACAGGCTGAACGAGGTGGCCA
AGAATCTGAACGAGTCCCTGATCGATCTGCAGGAGCTGGGCAAGTATGAGCAGTACATCAAGTGGCCCTGGTATATCTGG
CTGGGCTTCATCGCCGGCCTGATCGCCATCGTGATGGTGACCATCATGCTGTGCTGTATGACAAGCTGCTGTTCCTGCCT
GAAGGGCTGCTGTTCTTGTGGCAGCTGCTGTAAGTTTGATGAGGACGATAGCGAGCCTGTGCTGAAGGGCGTGAAGCTGC
ACTACACCTGATAACTAGCGGCGCGCCAGCAACAAGTAAGAAAAACTTAGGATTAATGGAAATTATCCAATCCAGAGACG
GAAGGACAAATCCAGAATCCAACCACAACTCAATCAACCAAAGATTCATGGAAGACAATGTTCAAAACAATCAAATCATG
GATTCTTGGGAAGAGGGATCAGGAGATAAATCATCTGACATCTCATCGGCCCTCGACATCATTGAATTCATACTCAGCAC
CGACTCCCAAGAGAACACGGCAGACAGCAATGAAATCAACACAGGAACCACAAGACTTAGCACGACAATCTACCAACCTG
AATCCAAAACAACAGAAACAAGCAAGGAAAATAGTGGACCAGCTAACAAAAATCGACAGTTTGGGGCATCACACGAACGT
GCCACAGAGACAAAAGATAGAAATGTTAATCAGGAGACTGTACAGGGAGGATATAGGAGAGGAAGCAGCCCAGATAGTAG
AACTGAGACTATGGTCACTCGAAGAATCTCCAGAAGCAGCCCAGATCCTAACAATGGAACCCAAATCCAGGAAGATATTG
ATTACAATGAAGTTGGAGAGATGGATAAGGACTCTACTAAGAGGGAAATGCGACAATTTAAAGATGTTCCAGTCAAGGTA
TCAGGAAGTGATGCCATTCCTCCAACAAAACAAGATGGAGACGGTGATGATGGAAGAGGCCTGGAATCTATCAGTACATT
TGATTCAGGATATACCAGTATAGTGACTGCCGCAACACTAGATGACGAAGAAGAACTCCTTATGAAGAACAACAGGCCAA
GAAAGTATCAATCAACACCCCAGAACAGTGACAAGGGAATTAAAAAAGGGGTTGGAAGGCCAAAAGACACAGACAAACAA
TCATCAATATTGGACTACGAACTCAACTTCAAAGGATCGAAGAAGAGCCAGAAAATCCTCAAAGCCAGCACGAATACAGG
AGAACCAACAAGACCACAGAATGGATCCCAGGGGAAGAGAATCACATCCTGGAACATCCTCAACAGCGAGAGCGGCAATC
GAACAGAATCAACAAACCAAACCCATCAGACATCAACCTCGGGACAGAACCACACAATGGGACCAAGCAGAACAACCTCC
GAACCAAGGATCAAGACACAAAAGACGGATGGAAAGGAAAGAGAGGACACAGAAGAGAGCACTCGATTTACAGAAAGGGC
GATTACATTATTACAGAATCTTGGTGTAATCCAATCTGCAGCAAAATTAGACCTATACCAAGACAAGAGAGTTGTGTGTG
TGGCGAATGTCCTAAACAATGCAGATACTGCATCAAAGATAGACTTCCTAGCAGGTTTGATGATAGGAGTGTCAATGGAT
CATGATACCAAATTAAATCAGATTCAGAACGAGATATTAAGTTTGAAAACTGATCTTAAAAAGATGGATGAATCACATAG
AAGACTAATTGAGAATCAAAAAGAACAATTATCACTGATCACATCATTAATCTCAAATCTTAAAATTATGACAGAGAGAG
GAGGGAAGAAGGACCAACCAGAACCTAGCGGGAGGACATCCATGATCAAGACAAAAGCAAAAGAAGAGAAAATAAAGAAA
GTCAGGTTTGACCCTCTTATGGAAACACAGGGCATCGAGAAAAACATCCCTGACCTCTATAGATCAATAGAGAAAACACC
AGAAAACGACACACAGATCAAATCAGAAATAAACAGATTGAATGATGAATCCAATGCCACTAGATTAGTACCTAGAAGAA
TAAGCAGTACAATGAGATCATTAATAATAATCATTAACAACAGCAATTTATCATCAAAAGCAAAGCAATCATACATCAAC
GAACTCAAGCTCTGCAAGAGTGACGAGGAAGTGTCTGAGTTGATGGACATGTTCAATGAGGATGTCAGCTCCCAGTAAAC
CGCCAACCAAGGGTCAACACCAAGAAAACCAATAGCACAAAACAGCCAATCAGAGACCACCCCAATACACCAAACCAATC
AACACATAACAAAGATCGCGGCCGCATAGATGATTAAGAAAAACTTAGGATGAAAGGACTAATCAATCCTCCGAAACAAT
GAGCATCACCAACTCCACAATCTACACATTCCCAGAATCCTCTTTCTCCGAGAATGGCAACATAGAGCCGTTACCACTCA
AGGTCAATGAACAGAGAAAGGCCATACCTCATATTAGGGTTGTCAAGATAGGAGATCCGCCCAAACATGGATCCAGATAT
CTGGATGTCTTTTTACTGGGCTTCTTTGAGATGGAAAGGTCAAAAGACAGGTATGGGAGCATAAGTGATCTAGATGATGA
TCCAAGTTACAAGGTTTGTGGCTCTGGATCATTGCCACTTGGGTTGGCTAGATACACCGGAAATGATCAGGAACTCCTAC
AGGCTGCAACCAAGCTCGATATAGAAGTAAGAAGAACTGTAAAGGCTACGGAGATGATAGTTTACACTGTACAAAACATC
AAACCTGAACTATATCCATGGTCCAGTAGATTAAGAAAAGGGATGTTATTTGACGCTAATAAGGTTGCACTTGCTCCTCA
ATGTCTTCCACTAGATAGAGGGATAAAATTCAGGGTGATATTTGTGAACTGCACAGCAATTGGATCAATAACTCTATTCA
AAATCCCTAAGTCCATGGCATTGTTATCATTGCCTAATACAATATCAATAAATCTACAAGTACATATCAAAACAGGAGTT
CAGACAGATTCCAAAGGAGTAGTTCAGATTCTAGATGAAAAAGGTGAAAAATCACTAAATTTCATGGTTCATCTCGGGTT
GATCAAAAGGAAGATGGGCAGAATGTACTCAGTTGAATATTGTAAGCAGAAGATCGAGAAGATGAGATTATTATTCTCAT
TGGGATTAGTTGGAGGGATCAGCTTCCACGTCAACGCAACTGGCTCTATATCAAAGACATTAGCAAGTCAATTAGCATTC
AAAAGAGAAATCTGCTATCCCCTAATGGATCTGAATCCACACTTAAATTCAGTTATATGGGCATCATCAGTTGAAATTAC
AAGGGTAGATGCAGTTCTCCAGCCTTCATTACCTGGCGAATTCAGATACTACCCAAACATCATAGCAAAAGGGGTCGGGA
AAATCAGACAGTAAAATCAACAACCCTGATATCCACCGGTGTATTAAGCCGAAGCAAATAAAGGATAATCAAAAACTTAG
GACAAAAGAGGTCAATACCAACAACTATTAGCAGTCACACTCGCAAGAATAAGAGAGAAGGGACCAAAAAAGTCAAATAG
GAGAAATCAAAACAAAAGGTACAGAACACCAGAACAACAAAATCAAAACATCCAACTCACTCAAAACAAAAATTCCAAAA
GAGACCGGCAACACAACAAGCACTGAACACAATGCCAACTTCAATACTGCTAATTATTACAACCATGATCATGGCATCTT
TCTGCCAAATAGATATCACAAAACTACAGCACGTAGGTGTATTGGTCAACAGTCCCAAAGGGATGAAGATATCACAAAAC
TTTGAAACAAGATATCTAATTTTGAGCCTCATACCAAAAATAGAAGACTCTAACTCTTGTGGTGACCAACAGATCAAGCA
ATACAAGAAGTTATTGGATAGACTGATCATCCCTTTATATGATGGATTAAGATTACAGAAAGATGTGATAGTAACCAATC
AAGAATCCAATGAAAACACTGATCCCAGAACAAAACGATTCTTTGGAGGGGTAATTGGAACCATTGCTCTGGGAGTAGCA
ACCTCAGCACAAATTACAGCGGCAGTTGCTCTGGTTGAAGCCAAGCAGGCAAGATCAGACATCGAAAAACTCAAAGAAGC
AATTAGGGACACAAACAAAGCAGTGCAGTCAGTTCAGAGCTCCATAGGAAATTTAATAGTAGCAATTAAATCAGTCCAGG
ATTATGTTAACAAAGAAATCGTGCCATCGATTGCGAGGCTAGGTTGTGAAGCAGCAGGACTTCAATTAGGAATTGCATTA
ACACAGCATTACTCAGAATTAACAAACATATTTGGTGATAACATAGGATCGTTACAAGAAAAAGGAATAAAATTACAAGG
TATAGCATCATTATACCGCACAAATATCACAGAAATATTCACAACATCAACAGTTGATAAATATGATATCTATGATCTGT
TATTTACAGAATCAATAAAGGTGAGAGTTATAGATGTTGACTTGAATGATTACTCAATCACCCTCCAAGTCAGACTCCCT
TTATTAACTAGGCTGCTGAACACTCAGATCTACAAAGTAGATTCCATATCATATAACATCCAAAACAGAGAATGGTATAT
CCCTCTTCCCAGCCATATCATGACGAAAGGGGCATTTCTAGGTGGAGCAGACGTCAAAGAATGTATAGAAGCATTCAGCA
GCTATATATGCCCTTCTGATCCAGGATTTGTATTAAACCATGAAATAGAGAGCTGCTTATCAGGAAACATATCCCAATGT
CCAAGAACAACGGTCACATCAGACATTGTTCCAAGATATGCATTTGTCAATGGAGGAGTGGTTGCAAACTGTATAACAAC
CACCTGTACATGCAACGGAATTGGTAATAGAATCAATCAACCACCTGATCAAGGAGTAAAAATTATAACACATAAAGAAT
GTAGTACAATAGGTATCAACGGAATGCTGTTCAATACAAATAAAGAAGGAACTCTTGCATTCTATACACCAAATGATATA
ACACTAAACAATTCTGTTGCACTTGATCCAATTGACATATCAATCGAGCTCAACAAGGCCAAATCAGATCTAGAAGAATC
AAAAGAATGGATAAGAAGGTCAAATCAAAAACTAGATTCTATTGGAAATTGGCATCAATCTAGCACTACAATCATAATTA
TTTTGATAATGATCATTATATTGTTTATAATTAATATAACGATAATTACAATTGCAATTAAGTATTACAGAATTCAAAAG
AGAAATCGAGTGGATCAAAATGACAAGCCATATGTACTAACAAACAAATAACATATCTACAGATCATTAGATATTAAAAT
TATAAAAAACTTAGGAGTAAAGTTACGCAATCCAACTCTACTCATATAATTGAGGAAGGACCCAATAGACAAATCCAAAT
TCGAGATGGAATACTGGAAGCATACCAATCACGGAAAGGATGCTGGTAATGAGCTGGAGACGTCTATGGCTACTCATGGC
AACAAGCTCACTAATAAGATAATATACATATTATGGACAATAATCCTGGTGTTATTATCAATAGTCTTCATCATAGTGCT
AATTAATTCCATCAAAAGTGAAAAGGCCCACGAATCATTGCTGCAAGACATAAATAATGAGTTTATGGAAATTACAGAAA
AGATCCAAATGGCATCGGATAATACCAATGATCTAATACAGTCAGGAGTGAATACAAGGCTTCTTACAATTCAGAGTCAT
GTCCAGAATTACATACCAATATCATTGACACAACAGATGTCAGATCTTAGGAAATTCATTAGTGAAATTACAATTAGAAA
TGATAATCAAGAAGTGCTGCCACAAAGAATAACACATGATGTAGGTATAAAACCTTTAAATCCAGATGATTTTTGGAGAT
GCACGTCTGGTCTTCCATCTTTAATGAAAACTCCAAAAATAAGGTTAATGCCAGGGCCGGGATTATTAGCTATGCCAACG
ACTGTTGATGGCTGTGTTAGAACTCCGTCTTTAGTTATAAATGATCTGATTTATGCTTATACCTCAAATCTAATTACTCG
AGGTTGTCAGGATATAGGAAAATCATATCAAGTCTTACAGATAGGGATAATAACTGTAAACTCAGACTTGGTACCTGACT
TAAATCCTAGGATCTCTCATACCTTTAACATAAATGACAATAGGAAGTCATGTTCTCTAGCACTCCTAAATACAGATGTA
TATCAACTGTGTTCAACTCCCAAAGTTGATGAAAGATCAGATTATGCATCATCAGGCATAGAAGATATTGTACTTGATAT
TGTCAATTATGATGGTTCAATCTCAACAACAAGATTTAAGAATAATAACATAAGCTTTGATCAACCATATGCTGCACTAT
ACCCATCTGTTGGACCAGGGATATACTACAAAGGCAAAATAATATTTCTCGGGTATGGAGGTCTTGAACATCCAATAAAT
GAGAATGTAATCTGCAACACAACTGGGTGCCCCGGGAAAACACAGAGAGACTGTAATCAAGCATCTCATAGTcCaTGGTT
TTCAGATAGGAGGATGGTCAACTCCATCATTGTTGTTGACAAAGGCTTAAACTCAATTCCAAAATTGAAAGTATGGACGA
TATCTATGCGACAAAATTACTGGGGGTCAGAAGGAAGGTTACTTCTACTAGGTAACAAGATCTATATATATACAAGATCT
ACAAGTTGGCATAGCAAGTTACAATTAGGAATAATTGATATTACTGATTACAGTGATATAAGGATAAAATGGACATGGCA
TAATGTGCTATCAAGACCAGGAAACAATGAATGTCCATGGGGACATTCATGTCCAGATGGATGTATAACAGGAGTATATA
CTGATGCATATCCACTCAATCCCACAGGGAGCATTGTGTCATCTGTCATATTAGACTCACAAAAATCGAGAGTGAACCCA
GTCATAACTTACTCAACAGCAACCGAAAGAGTAAACGAGCTGGCCATCCTAAACAGAACACTCTCAGCTGGATATACAAC
AACAAGCTGCATTACACACTATAACAAAGGATATTGTTTTCATATAGTAGAAATAAATCATAAAAGCTTAAACACATTTC
AACCCATGTTGTTCAAAACAGAGATTCCAAAAAGCTGCAGTTAATCATAATTAACCATAATATGCATCAATCTATCTATA
ATACAAGTATATGATAAGTAATCAGCAATCAGACAATAGACGTACGGAAATAATAAAAAACTTAGGAGAAAAGTGTGCAA
GAAAAATGGACACCGAGTCCCACAGCGGCACAACATCTGACATTCTGTACCCTGAATGTCACCTCAATTCTCCTATAGTT
AAAGGAAAGATAGCACAACTGCATACAATAATGAGTTTGCCTCAGCCCTACGATATGGATGATGATTCAATACTGATTAT
TACTAGACAAAAAATTAAACTCAATAAATTAGATAAAAGACAACGGTCAATTAGGAAATTAAGATCAGTCTTAATGGAAA
GAGTAAGTGATCTAGGTAAATATACCTTTATCAGATATCCAGAGATGTCTAGTGAAATGTTCCAATTATGTATACCCGGA
ATTAATAATAAAATAAATGAATTGCTAAGTAAAGCAAGTAAAACATATAATCAAATGACTGATGGATTAAGAGATCTATG
GGTTACTATACTATCGAAGTTAGCATCGAAAAATGATGGAAGTAATTATGATATCAATGAAGATATTAGCAATATATCAA
ATGTTCACATGACTTATCAATCAGACAAATGGTATAATCCATTCAAGACATGGTTTACTATTAAGTATGACATGAGAAGA
TTACAAAAAGCCAAAAATGAGATTACATTCAATAGGCATAAAGATTATAATCTATTAGAAGACCAAAAGAATATATTGCT
GATACATCCAGAACTCGTCTTAATATTAGATAAACAAAATTACAATGGGTATATAATGACTCCTGAATTGGTACTAATGT
ATTGTGATGTAGTTGAAGGGAGGTGGAATATAAGTTCATGTGCAAAATTGGATCCTAAGTTACAATCAATGTATTATAAG
GGTAACAATTTATGGGAAATAATAGATGGACTATTCTCGACCTTAGGAGAAAGAACATTTGACATAATATCACTATTAGA
ACCACTTGCATTATCGCTCATTCAAACTTATGACCCGGTTAAACAGCTCAGGGGGGCTTTTTTAAATCACGTGTTATCAG
AAATGGAATTAATATTTGCAGCTGAGTGTACAACAGAGGAAATACCTAATGTGGATTATATAGATAAAATTTTAGATGTG
TTCAAAGAATCAACAATAGATGAAATAGCAGAAATTTTCTCTTTCTTCCGAACTTTTGGACACCCTCCATTAGAGGCGAG
TATAGCAGCAGAGAAAGTTAGAAAGTATATGTATACTGAGAAATGCTTGAAATTTGATACTATCAATAAATGTCATGCTA
TTTTTTGTACAATAATTATAAATGGATATAGAGAAAGACATGGTGGTCAATGGCCTCCAGTTACATTACCTGTCCATGCA
CATGAATTTATCATAAATGCATACGGATCAAATTCTGCCATATCATATGAGAATGCTGTAGATTATTATAAGAGCTTCAT
AGGAATAAAATTTGACAAGTTTATAGAGCCTCAATTGGATGAAGACTTAACTATTTATATGAAAGATAAAGCATTATCCC
CAAAGAAATCAAACTGGGACACAGTCTATCCAGCTTCAAACCTGTTATACCGCACTAATGTGTCTCATGATTCACGAAGA
TTGGTTGAAGTATTTATAGCAGATAGTAAATTTGATCCCCACCAAGTATTAGATTACGTAGAATCAGGATATTGGCTGGA
TGATCCTGAATTTAATATCTCATATAGTTTAAAAGAGAAAGAAATAAAACAAGAAGGTAGACTTTTTGCAAAAATGACAT
ACAAGATGAGGGCTACACAAGTATTATCAGAAACATTATTGGCGAATAATATAGGGAAATTCTTCCAAGAGAATGGGATG
GTTAAAGGAGAAATTGAATTACTCAAGAGACTAACAACAATATCTATGTCTGGAGTTCCGCGGTATAATGAGGTATACAA
TAATTCAAAAAGTCACACAGAAGAACTTCAAGCTTATAATGCAATTAGCAGTTCCAATTTATCTTCTAATCAGAAGTCAA
AGAAGTTTGAATTTAAATCTACAGATATATACAATGATGGATACGAAACCGTAAGCTGCTTCTTAACGACAGATCTTAAA
AAATATTGTTTAAATTGGAGGTATGAATCAACAGCTTTATTCGGTGATACTTGTAATCAGATATTTGGGTTAAAGGAATT
ATTTAATTGGCTGCACCCTCGCCTTGAAAAGAGTACAATATATGTTGGAGATCCTTATTGCCCGCCATCAGATATTGAAC
ATTTACCACTTGATGACCATCCTGATTCAGGATTTTATGTTCATAATCCTAAAGGAGGAATAGAAGGGTTTTGCCAAAAG
TTATGGACACTCATATCTATCAGTGCAATACATTTAGCAGCTGTCAAAATCGGTGTAAGAGTTACTGCAATGGTTCAAGG
GGATAATCAAGCCATAGCTGTTACCACAAGAGTACCTAATAATTATGATTATAAAGTTAAGAAAGAGATTGTTTATAAAG
ATGTGGTAAGATTTTTTGATTCCTTGAGAGAGGTGATGGATGATCTGGGTCATGAGCTCAAACTAAATGAAACTATAATA
AGTAGTAAAATGTTTATATATAGCAAAAGGATATACTATGACGGAAGAATCCTTCCTCAGGCATTAAAAGCATTGTCTAG
ATGTGTTTTTTGGTCTGAAACAATCATAGATGAGACAAGATCAGCATCCTCAAATCTGGCTACATCGTTTGCAAAGGCCA
TTGAGAATGGCTACTCACCTGTATTGGGATATGTATGCTCAATCTTCAAAAATATCCAACAGTTGTATATAGCGCTTGGA
ATGAATATAAACCCAACTATAACCCAAAATATTAAAGATCAATATTTCAGGAATATTCATTGGATGCAATATGCCTCCTT
AATCCCTGCTAGTGTCGGAGGATTTAATTATATGGCCATGTCAAGGTGTTTTGTCAGAAACATTGGAGATCCTACAGTCG
CTGCGTTAGCCGATATTAAAAGATTTATAAAAGCAAATTTGTTAGATCGAGGTGTCCTTTACAGAATTATGAATCAAGAA
CCAGGCGAGTCTTCTTTTTTAGACTGGGCCTCAGATCCCTATTCATGTAACTTACCACAATCTCAAAATATAACCACCAT
GATAAAGAATATAACTGCAAGAAATGTACTACAGGACTCACCAAACCCATTACTATCTGGATTATTTACAAGTACAATGA
TAGAAGAGGATGAGGAATTAGCTGAGTTCCTAATGGACAGGAGAATAATCCTCCCAAGAGTTGCACATGACATTTTAGAT
AATTCTCTTACTGGAATTAGGAATGCTATAGCTGGTATGTTGGATACAACAAAATCACTAATTCGAGTAGGGATAAGCAG
AGGAGGATTAACCTATAACTTATTAAGAAAGATAAGCAACTATGATCTTGTACAATATGAGACACTTAGTAAAACTTTAA
GACTAATAGTCAGTGACAAGATTAAGTATGAAGATATGTGCTCAGTAGACCTAGCCATATCATTAAGACAAAAAATGTGG
ATGCATTTATCAGGAGGAAGAATGATAAATGGACTTGAAACTCCAGATCCTTTAGAGTTACTGTCTGGAGTAATAATAAC
AGGATCTGAACATTGTAGGATATGTTATTCAACTGAAGGTGAAAGCCCATATACATGGATGTATTTACCAGGCAATCTTA
ATATAGGATCAGCTGAGACAGGAATAGCATCATTAAGGGTCCCTTACTTTGGATCAGTTACAGATGAGAGATCTGAAGCA
CAATTAGGGTATATCAAAAATCTAAGCAAACCAGCTAAGGCTGCTATAAGAATAGCAATGATATATACTTGGGCATTTGG
GAATGACGAAATATCTTGGATGGAAGCATCACAGATTGCACAAACACGTGCAAACTTTACATTGGATAGCTTAAAGATTT
TGACACCAGTGACAACATCAACAAATCTATCACACAGGTTAAAAGATACTGCTACTCAGATGAAATTTTCTAGTACATCA
CTTATTAGAGTAAGCAGGTTCATCACAATATCTAATGATAATATGTCTATTAAAGAAGCAAATGAAACTAAAGATACAAA
TCTTATTTATCAACAGGTAATGTTAACAGGATTAAGTGTATTTGAATATCTATTTAGGTTAGAGGAGAGTACAGGACATA
ACCCTATGGTCATGCATCTACATATAGAGGATGGATGTTGTATAAAAGAGAGTTACAATGATGAGCATATCAATCCGGAG
TCTACATTAGAGTTAATCAAATACCCTGAGAGTAATGAATTTATATATGATAAGGACCCTTTAAAGGATATAGATCTATC
AAAATTAATGGTTATAAGAGATCATTCTTATACAATTGACATGAATTACTGGGATGACACAGATATTGTACATGCAATAT
CAATATGTACTGCAGTTACAATAGCAGATACAATGTCGCAGCTAGATCGGGATAATCTTAAGGAGCTGGTTGTGATTGCA
AATGATGATGATATTAACAGTCTGATAACTGAATTTCTGACCCTAGATATACTAGTGTTTCTCAAAACATTTGGAGGGTT
ACTCGTGAATCAATTTGCATATACCCTTTATGGATTGAAAATAGAAGGAAGGGATCCCATTTGGGATTATATAATGAGAA
CATTAAAAGACACCTCACATTCAGTACTTAAAGTATTATCTAATGCACTATCTCATCCAAAAGTGTTTAAGAGATTTTGG
GATTGTGGAGTTTTGAATCCTATTTATGGTCCTAATACTGCTAGTCAAGATCAAGTTAAGCTTGCTCTCTCGATTTGCGA
GTACTCCTTGGATCTATTTATGAGAGAATGGTTGAATGGAGCATCACTTGAGATCTATATCTGTGATAGTGACATGGAAA
TAGCAAATGACAGAAGACAAGCATTTCTCTCAAGACATCTTGCCTTTGTGTGTTGTTTAGCAGAGATAGCATCTTTTGGA
CCAAATTTATTAAATCTAACATATCTAGAGAGACTTGATGAATTAAAACAATACTTAGATCTGAACATCAAAGAAGATCC
TACTCTTAAATATGTGCAAGTATCAGGACTGTTAATTAAATCATTCCCCTCAACTGTTACGTATGTAAGGAAAACTGCGA
TTAAGTATCTGAGGATTCGTGGTATTAATCCGCCTGAAACGATTGAAGATTGGGATCCCATAGAAGATGAGAATATCTTA
GACAATATTGTTAAAACTGTAAATGACAATTGCAGTGATAATCAAAAGAGAAATAAAAGTAGTTATTTCTGGGGATTAGC
TCTAAAGAATTATCAAGTCGTGAAAATAAGATCCATAACGAGTGATTCTGAAGTTAATGAAGCTTCGAATGTTACTACAC
ATGGAATGACACTTCCTCAGGGAGGAAGTTATCTATCACATCAGCTGAGGTTATTTGGAGTAAACAGTACAAGTTGTCTT
AAAGCTCTTGAATTATCACAAATCTTAATGAGGGAAGTTAAAAAAGATAAAGATAGACTCTTTTTAGGAGAAGGAGCAGG
AGCTATGTTAGCATGTTATGATGCTACACTCGGTCCTGCAATAAATTATTATAATTCTGGTTTAAATATTACAGATGTAA
TTGGTCAACGGGAATTAAAAATCTTCCCATCAGAAGTATCATTAGTAGGTAAAAAACTAGGAAATGTAACACAGATTCTT
AATCGGGTGAGGGTGTTATTTAATGGGAATCCCAATTCAACATGGATAGGAAATATGGAATGTGAGAGTTTAATATGGAG
TGAATTAAATGATAAGTCAATTGGTTTAGTACATTGTGACATGGAGGGAGCGATAGGCAAATCAGAAGAAACTGTTCTAC
ATGAACATTATAGTATTATTAGGATTACATATTTAATCGGGGATGATGATGTTGTCCTAGTATCAAAAATTATACCAACT
ATTACTCCGAATTGGTCTAAAATACTCTATCTATACAAGTTGTATTGGAAGGATGTAAGTGTAGTGTCCCTTAAAACATC
CAATCCTGCCTCAACAGAGCTTTATTTAATTTCAAAAGATGCTTACTGTACTGTAATGGAACCCAGTAATCTTGTTTTAT
CAAAACTTAAAAGGATATCATCAATAGAAGAAAATAATCTATTAAAGTGGATAATCTTATCAAAAAGGAAGAATAACGAG
TGGTTACAGCATGAAATCAAAGAAGGAGAAAGGGATTATGGGATAATGAGGCCATATCATACAGCACTGCAAATTTTTGG
ATTCCAAATTAACTTAAATCACTTAGCTAGAGAATTTTTATCAACTCCTGATTTAACCAACATTAATAATATAATTCAAA
GTTTTACAAGAACAATTAAAGATGTTATGTTCGAATGGGTCAATATCACTCATGACAATAAAAGACATAAATTAGGAGGA
AGATATAATCTATTCCCGCTTAAAAATAAGGGGAAATTAAGATTATTATCACGAAGATTAGTACTAAGCTGGATATCATT
ATCCTTATCAACCAGATTACTGACGGGCCGTTTTCCAGATGAAAAATTTGAAAATAGGGCACAGACCGGATATGTATCAT
TGGCTGATATTGATTTAGAATCCTTAAAGTTATTATCAAGAAATATTGTCAAAAATTACAAAGAACACATAGGATTAATA
TCATACTGGTTTTTGACCAAAGAGGTCAAAATACTAATGAAGCTTATAGGAGGAGTCAAACTACTAGGAATTCCTAAACA
GTACAAAGAGTTAGAGGATCGATCATCTCAGGGTTATGAATATGATAATGAATTTGATATTGATTAATACATAAAAACAa
AAAATAAAACACCTATTCCTCACCCATTCACTTCCAACAAAATGAAAAGTAAGAAAAACATGTAATATATATATACCAAA
CAGAGTTTTTCTCTTGTTTGGT

Non-limiting examples of methods of generating a recombinant parainfluenza virus (such as a rB/HPIV3) including a heterologous gene, methods of attenuating the viruses (e.g., by recombinant or chemical means), as well as viral sequences and reagents for use in such methods are provided in U.S. Patent Application Publication Nos. 2012/0045471, 2010/0119547, 2009/0263883, and 2009/0017517; U.S. Pat. Nos. 7,632,508, 7,622,123, 7,250,171, 7,208,161, 7,201,907, 7,192,593; PCT Publication No. WO 2016/118642; Liang et al. (J. Virol, 88(8): 4237-4250, 2014), and Tang et al. (J Virol, 77(20):10819-10828, 2003). In some embodiments, these methods can be modified as needed using the description provided herein to construct a disclosed rB/HPIV3-SARS-COV-2/S vector.

The genome of the rB/HPIV3-SARS-COV-2/S vector can include one or more variations (for example, mutations that cause an amino acid deletion, substitution, or insertion) as long as the resulting rB/HPIV3-SARS-COV-2/S retains the desired biological function, such as a level of attenuation or immunogenicity. These variations in sequence can be naturally occurring variations or they can be engineered through the use of genetic engineering technique.

Other mutations involve replacement of the 3′ end of genome with its counterpart from antigenome, which is associated with changes in RNA replication and transcription. In addition, the intergenic regions (Collins et al., Proc. Natl. Acad. Sci. USA 83:4594-4598 (1986)) can be shortened or lengthened or changed in sequence content, and the naturally-occurring gene overlap (Collins et al., Proc. Natl. Acad. Sci. USA 84:5134-5138 (1987)) can be removed or changed to a different intergenic region by the methods described herein.

In another embodiment, a sequence surrounding a translational start site (such as including a nucleotide in the -3 position) of a selected viral gene is modified, alone or in combination with introduction of an upstream start codon, to modulate gene expression by specifying up- or down-regulation of translation.

Alternatively, or in combination with other modifications disclosed herein, gene expression can be modulated by altering a transcriptional GS signal of a selected gene(s) of the virus. In additional embodiments, modifications to a transcriptional GE signal can be incorporated into the viral genome.

In addition to the above described modifications to rB/HPIV3-SARS-COV-2/S, different or additional modifications to the genome can be made to facilitate manipulations, such as the insertion of unique restriction sites in various intergenic regions (e.g., a unique Asc I site between the N and P genes) or elsewhere. Nontranslated gene sequences can be removed to increase capacity for inserting foreign sequences.

Introduction of the foregoing modifications into rB/HPIV3-SARS-COV-2/S can be achieved by a variety of well-known methods. Examples of such techniques are found in, e.g., Sambrook et al. (Molecular Cloning: A Laboratory Manual, 4^{th} ed., Cold Spring Harbor, New York, 2012) and Ausubel et al. (In Current Protocols in Molecular Biology, John Wiley & Sons, New York, through supplement 104, 2013). Thus, defined mutations can be introduced by conventional techniques (e.g., site-directed mutagenesis) into acDNA copy of the genome or antigenome. The use of antigenome or genome cDNA subfragments to assemble a complete antigenome or genome cDNA has the advantage that each region can be manipulated separately (smaller cDNAs are easier to manipulate than large ones) and then readily assembled into a complete cDNA. Thus, the complete antigenome or genome cDNA, or any subfragment thereof, can be used as template for oligonucleotide-directed mutagenesis. A mutated subfragment can then be assembled into the complete antigenome or genome cDNA. Mutations can vary from single nucleotide changes to replacement of large cDNA pieces containing one or more genes or genome regions.

The disclosed embodiments of rB/HPIV3-SARS-COV-2/S are self-replicating, that is they are capable of replicating following infection of an appropriate host cell, and have an attenuated phenotype, for example when administered to a human subject. In some examples, the rB/HPIV3-SARS-COV-2/S is attenuated about 3- to 500-fold or more in the upper respiratory tract and about 100- to 5000-fold or more in the lower respiratory tract in a mammal compared to control HPIV3. In some embodiments, the level of viral replication in vitro is sufficient to provide for production of virus for use on a wide-spread scale. In some embodiments, the level of viral replication of attenuated paramyxovirus in vitro is at least 10⁶, at least 10⁷, or at least 10⁸ per ml.

In some embodiments, the rB/HPIV3-SARS-COV-2/S vectors can be produced using the reverse genetics recombinant DNA-based technique (Collins, et al. 1995. Proc Natl Acad Sci USA 92:11563-11567). This system allows de novo recovery of infectious virus entirely from cDNA in a qualified cell substrate under defined conditions. Reverse genetics provides a means to introduce predetermined mutations into the rB/HPIV3-SARS-COV-2/S genome via the cDNA intermediate. Specific attenuating mutations were characterized in preclinical studies and combined to achieve the desired level of attenuation. Derivation of vaccine viruses from cDNA minimizes the risk of contamination with adventitious agents and helps to keep the passage history brief and well documented. Once recovered, the engineered virus strains propagate in the same manner as a biologically derived virus. As a result of passage and amplification, the virus does not contain recombinant DNA from the original recovery.

To propagate rB/HPIV3-SARS-COV-2/S vectors for immunization and other purposes, a number of cell lines which allow for viral growth may be used. Parainfluenza virus grows in a variety of human and animal cells. Exemplary cell lines for propagating attenuated rB/HPIV3-SARS-COV-2/S virus for immunization include HEp-2 cells, FRhL-DBS2 cells, LLC-MK2 cells, MRC-5 cells, and Vero cells. Highest virus yields are usually achieved with epithelial cell lines such as Vero cells. Cells can be inoculated with virus at a multiplicity of infection ranging from about 0.001 to 1.0, or more, and are cultivated under conditions permissive for replication of the virus, e.g., at about 30-37° C. and for about 3-10 days, or as long as necessary for virus to reach an adequate titer. Temperature-sensitive viruses often are grown using 32° C. as the “permissive temperature.” Virus is removed from cell culture and separated from cellular components, typically by standard clarification procedures, e.g., centrifugation, and may be further purified as desired using known procedures.

The rB/HPIV3-SARS-COV-2/S vectors can be tested in various well known and generally accepted in vitro and in vivo models to confirm adequate attenuation, resistance to phenotypic reversion, and immunogenicity. In in vitro assays, the modified virus is tested for temperature sensitivity of virus replication or “ts phenotype,” and for the small plaque phenotype. Modified virus also may be evaluated in an in vitro human airway epithelium (HAE) model, which provides a means of ranking viruses in the order of their relative attenuation in non-human primates and humans (Zhang et al., 2002 J Virol 76:5654-5666; Schaap-Nutt et al., 2010 Vaccine 28:2788-2798; Ilyushina et al., 2012 J Virol 86:11725-11734). Modified viruses are further tested in animal models of HPIV3 or SARS-COV-2 infection. A variety of animal models (e.g., murine, hamster, cotton rat, and primate) are available.

Immunogenicity of a rB/HPIV3-SARS-COV-2/S vector can be assessed in an animal model (such as a non-human primate, for example a rhesus macaque), for example, by determining the number of animals that form antibodies to SARS-COV-2 and HPIV3 after one immunization and after a second immunization, and by measuring the magnitude of that response. In some embodiments, a rB/HPIV3-SARS-COV-2/S has sufficient immunogenicity if about 60 to 80% of the animals develop antibodies after the first immunization and about 80 to 100% of the animals develop antibodies after the second immunization. In some instances, the immune response protects against infection by both SARS-COV-2 and HPIV3.

Also provided are isolated polynucleotides comprising or consisting of the genome or antigenome of a disclosed rB/HPIV3-SARS-COV-2/S vector, vectors comprising the polynucleotides, and host cells comprising the polynucleotides or vectors.

### IV. Immunogenic Compositions

Immunogenic compositions that include a disclosed rB/HPIV3-SARS-COV-2/S vector and a pharmaceutically acceptable carrier are also provided. Such compositions can be administered to a subject by a variety of modes, for example, by an intranasal route. Standard methods for preparing administrable immunogenic compositions are described, for example, in such publications as Remingtons Pharmaceutical Sciences, 19^{th} Ed., Mack Publishing Company, Easton, Pennsylvania, 1995.

Potential carriers include, but are not limited to, physiologically balanced culture medium, phosphate buffer saline solution, water, emulsions (e.g., oil/water or water/oil emulsions), various types of wetting agents, cryoprotective additives or stabilizers such as proteins, peptides or hydrolysates (e.g., albumin, gelatin), sugars (e.g., sucrose, lactose, sorbitol), amino acids (e.g., sodium glutamate), or other protective agents. The resulting aqueous solutions may be packaged for use as is or lyophilized. Lyophilized preparations are combined with a sterile solution prior to administration for either single or multiple dosing.

The immunogenic composition can contain a bacteriostat to prevent or minimize degradation during storage, including but not limited to effective concentrations (usually ≤1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The immunogenic composition can contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate.

The immunogenic composition may optionally include an adjuvant to enhance the immune response of the host. Suitable adjuvants are, for example, toll-like receptor agonists, alum, AIPO4, alhydrogel, Lipid-A and derivatives or variants thereof, oil-emulsions, saponins, neutral liposomes, liposomes containing the recombinant virus, and cytokines, non-ionic block copolymers, and chemokines. Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, IN) and IL-12 (Genetics Institute, Cambridge, MA), among many other suitable, well-known adjuvants may be used as an adjuvant (Newman et al., 1998, Critical Reviews in Therapeutic Drug Carrier Systems 15:89-142). These adjuvants have the advantage in that they help to stimulate the immune system in a non-specific way, thus enhancing the immune response to a pharmaceutical product.

In some instances, it may be desirable to combine the immunogenic composition including the rB/HPIV3- SARS-COV-2/S, with other pharmaceutical products (e.g., vaccines) which induce protective responses to other viral agents, particularly those causing other childhood illnesses. For example, a composition including a rB/HPIV3-SARS-COV-2/S as described herein can also include other vaccines recommended by the Advisory Committee on Immunization Practices (ACIP; cdc.gov/vaccines/acip) for the targeted age group (e.g., infants from approximately one to six months of age). These additional vaccines include, but are not limited to, IN-administered vaccines. As such, a rB/HPIV3-SARS-COV-2/S as described herein may be administered simultaneously with vaccines against, for example, hepatitis B (HepB), diphtheria, tetanus and pertussis (DTaP), pneumococcal bacteria (PCV), Haemophilus influenzae type b (Hib), polio, influenza and rotavirus.

In some embodiments, the immunogenic composition can be provided in unit dosage form to induce an immune response in a subject, for example, to prevent HPIV3 and/or SARS-COV-2 infection in the subject. A unit dosage form contains a suitable single preselected dosage for administration to a subject, or suitable marked or measured multiples of two or more preselected unit dosages, and/or a metering mechanism for administering the unit dose or multiples thereof.

### V. Methods of Eliciting an Immune Response

Provided herein are methods of eliciting an immune response in a subject by administering an immunogenic composition containing a disclosed rB/HPIV3-SARS-COV-2/S vector to the subject. Upon immunization, the subject responds by producing antibodies specific for one or more of SARS-COV-2 S protein and HPIV3 HN and F proteins. In addition, innate and cell-mediated immune responses are induced, which can provide antiviral effectors as well as regulating the immune response. As a result of the immunization the host becomes at least partially or completely immune to HPIV3 and/or SARS-COV-2 infection, or resistant to developing moderate or severe HPIV3 and/or SARS-COV-2 disease (such as COVID-19), particularly of the lower respiratory tract.

A subject who has or is at risk for developing a SARS-COV-2 infection and/or a HPIV3 infection, for example because of exposure or the possibility of exposure to the SARS-COV-2 and/or HPIV3, can be selected for immunization. Following administration of a disclosed immunogen, the subject can be monitored for infection or symptoms associated with SARS-COV-2 and/or HPIV3 infection.

Nearly all humans are infected with HPIV3 by the age of five and further are at risk of SARS-COV-2 infection. Therefore, the entire birth cohort is included as a relevant population for immunization. This could be done, for example, by beginning an immunization regimen anytime from birth to 6 months of age, from 6 months of age to 5 years of age, in pregnant women (or women of child-bearing age) to protect their infants by passive transfer of antibody, family members of newborn infants or those still in utero, and subjects greater than 50 years of age. The scope of this disclosure is meant to include maternal immunization. In several embodiments, the subject is a human subject that is seronegative for SARS-COV-2 and/or HPIV3 specific antibodies. In additional embodiments, the subject is no more than one year old, such as no more than 6 months old, no more than 3 months, or no more than 1 month old.

Subjects at greatest risk of SARS-COV-2 and/or HPIV infection with severe symptoms (e.g. requiring hospitalization) include children with prematurity, bronchopulmonary dysplasia, and congenital heart disease. During childhood and adulthood, disease is milder but can be associated with lower airway disease and is commonly complicated by sinusitis. Disease severity increases in the institutionalized elderly (e.g., humans over 65 years old). Severe disease also occurs in persons with severe combined immunodeficiency disease or following bone marrow or lung transplantation. In some embodiments, these subjects can be selected for administration of a disclosed rB/HPIV3/SARS-COV-2/S vector.

The immunogenic compositions containing the rB/HPIV3-SARS-COV-2/S are administered to a subject susceptible to or otherwise at risk of SARS-COV-2 and/or HPIV3 infection in an “effective amount” which is sufficient to induce or enhance the individual's immune response capabilities against SARS-COV-2 and/or HPIV3. The immunogenic composition may be administered by any suitable method, including but not limited to, via injection, aerosol delivery, nasal spray, nasal droplets, oral inoculation, or topical application. In a particular embodiment, the attenuated virus is administered according to established human intranasal administration protocols (e.g., as discussed in Karron et al., J Infect Dis 191:1093-104, 2005).

Briefly, adults or children are inoculated intranasally via droplet with an effective amount of the rB/HPIV3-SARS-COV-2/S, such as in a volume of 0.5 ml of a physiologically acceptable diluent or carrier. This has the advantage of simplicity and safety compared to parenteral immunization with a non-replicating virus. It also provides direct stimulation of local respiratory tract immunity, which plays a role in resistance to SARS-COV-2 and HPIV3. Further, this mode of vaccination effectively bypasses the immunosuppressive effects of HPIV3- and SARS-COV-2-specific maternally-derived serum antibodies, which are found in the very young.

In all subjects, the precise amount of rB/HPIV3-SARS-COV-2/S administered and the timing and repetition of administration will be determined by various factors, including the patient's state of health and weight, the mode of administration, the nature of the formulation, etc. Dosages will generally range from about 3.0 log₁₀ to about 6.0 log₁₀ plaque forming units (“PFU”) or more of virus per patient, more commonly from about 4.0 log₁₀ to 5.0 log₁₀ PFU virus per patient. In one embodiment, about 5.0 log₁₀ to 6.0 log₁₀ PFU per patient may be administered during infancy, such as between 1 and 6 months of age, and one or more additional booster doses could be given 2-6 months or more later. In another embodiment, young infants could be given a dose of about 5.0 log₁₀ to 6.0 log₁₀ PFU per patient at approximately 2, 4, and 6 months of age, which is the recommended time of administration of a number of other childhood vaccines. In yet another embodiment, an additional booster dose could be administered at approximately 10-15 months of age.

The embodiments of rB/HPIV3-SARS-COV-2/S described herein, and immunogenic compositions thereof, are administered to a subject in an amount effective to induce or enhance an immune response against the HPIV3 and SARS-COV-2 antigens included in the rB/HPIV3-SARS-COV-2/S in the subject. An effective amount will allow some growth and proliferation of the virus, in order to produce the desired immune response, but will not produce viral-associated symptoms or illnesses. Based on the guidance provided herein and knowledge in the art, the proper amount of rB/HPIV3-SARS-COV-2/S to use for immunization can be determined.

A desired immune response is to inhibit subsequent infection with SARS-COV-2 and/or HPIV3. The SARS-COV-2 and/or HPIV3 infection does not need to be completely inhibited for the method to be effective. For example, administration of an effective amount of a disclosed rB/HPIV3-SARS-COV-2/S can decrease subsequent SARS-COV-2 and/or HPIV3 infection (for example, as measured by infection of cells, or by number or percentage of subjects infected by SARS-COV-2 and/or HPIV3) by a desired amount, for example by at least 10%, at least 20%, at least 50%, at least 60%, at least 70%, at least 80%, at least 90%, at least 95%, at least 98%, or even at least 100% (prevention of detectable SARS-COV-2 and/or HPIV3infection), as compared to a suitable control.

Determination of effective dosages is typically based on animal model studies followed up by human clinical trials and is guided by administration protocols that significantly reduce the occurrence or severity of targeted disease symptoms or conditions in the subject, or that induce a desired response in the subject (such as a neutralizing immune response). Suitable models in this regard include, for example, murine, rat, hamster, cotton rat, bovine, ovine, porcine, feline, ferret, non-human primate, and other accepted animal model subjects known in the art. Alternatively, effective dosages can be determined using in vitro models (for example, immunologic and histopathologic assays). Using such models, only ordinary calculations and adjustments are needed to determine an appropriate concentration and dose to administer a therapeutically effective amount of the composition (for example, amounts that are effective to elicit a desired immune response or alleviate one or more symptoms of a targeted disease).

Administration of the rB/HPIV3-SARS-COV-2/S to a subject can elicit the production of an immune response that is protective against disease, such as COVID-19 and/or serious lower respiratory tract disease, such as pneumonia and bronchiolitis, or croup, when the subject is subsequently infected or re-infected with a wild-type SARS-COV-2 or HPIV3. While the naturally circulating virus is still capable of causing infection, particularly in the upper respiratory tract, there is a reduced possibility of rhinitis as a result of the immunization and a possible boosting of resistance by subsequent infection by wild-type virus. Following immunization, there are detectable levels of host engendered serum and secretory antibodies which are capable of neutralizing wild-type virus in vitro and in vivo.

An immunogenic composition including the disclosed rB/HPIV3-SARS-COV-2/S can be used in coordinate (or prime-boost) immunization protocols or combinatorial formulations. It is contemplated that there can be several boosts, and that each boost can be a different disclosed immunogen. It is also contemplated in some examples that the boost may be the same immunogen as another boost, or the prime. In certain embodiments, novel combinatorial immunogenic compositions and coordinate immunization protocols employ separate immunogens or formulations, each directed toward eliciting an anti-viral immune response, such as an immune response to SARS-COV-2 and HPIV3 proteins. Separate immunogenic compositions that elicit the anti-viral immune response can be combined in a polyvalent immunogenic composition administered to a subject in a single immunization step, or they can be administered separately (in monovalent immunogenic compositions) in a coordinate (or prime-boost) immunization protocol.

The resulting immune response can be characterized by a variety of methods. These include taking samples of nasal washes or sera for analysis of SARS-COV-2- or HPIV3-specific antibodies, which can be detected by tests including, but not limited to, complement fixation, plaque neutralization, enzyme-linked immunosorbent assay, luciferase-immunoprecipitation assay, and flow cytometry. In addition, immune responses can be detected by assay of cytokines in nasal washes or sera, ELISPOT of immune cells from either source, quantitative RT-PCR or microarray analysis of nasal wash or serum samples, and restimulation of immune cells from nasal washes or serum by re-exposure to viral antigen in vitro and analysis for the production or display of cytokines, surface markers, or other immune correlates measures by flow cytometry or for cytotoxic activity against indicator target cells displaying SARS-COV-2 or HPIV3 antigens. In this regard, individuals are also monitored for signs and symptoms of upper respiratory illness.

The following examples are provided to illustrate certain particular features and/or embodiments. These examples should not be construed to limit the disclosure to the particular features or embodiments described.

### EXAMPLES

Severe acute respiratory syndrome coronavirus 2 (SARS-COV-2) infects by the respiratory route, and single-dose vaccines with the ability to restrict SARS-COV-2 replication and shedding from the respiratory tract could reduce viral disease and transmission. The following examples describe the development of live-attenuated viral vector vaccines for intranasal immunization of infants and children against coronavirus disease 2019 (COVID-19) based on a replication-competent chimeric bovine/human parainfluenza virus type 3 vector (B/HPIV3) expressing from an added gene the native (S) or prefusion-stabilized (S-2P or S-6P) versions of the S spike protein, the major protective and neutralization antigen of SARS-COV-2. B/HPIV3/S, B/HPIV3/S-2P and B/HPIV3/S-6P replicated as efficiently as B/HPIV3 in Vero cells, while replication of S expressing versions in human lung epithelial A549 cells was slightly reduced compared to B/HPIV3. B/HPIV3/S, B/HPIV3/S-2P and B/HPIV3/S-6P stably expressed SARS-COV-2 S. Prefusion stabilization increased S expression by B/HPIV3 in vitro.

In hamsters, a single intranasal dose of B/HPIV3/S-2P induced serum antibodies with the broad functional ability to neutralize SARS-COV-2 of lineages A, B.1.1.7/Alpha and B.1.351/Beta, and levels of serum IgG to the SARS-COV-2 S protein or its receptor binding domain that were significantly higher than those induced by B/HPIV3/S; B/HPIV3/S-6P induced slightly higher IgG titers to the SARS-COV-2 receptor binding domain than B/HPIV3/S-2P. Intranasal immunization with B/HPIV3/S-2P or B/HPIV3/S-6P induced a serum IgA and IgG response to the SARS-COV-2 S protein of the vaccine-matched WA-1/2020 strain, and cross-reactive antibodies to B.1.1.7/Alpha and B.1.351/Beta, B.1.617.2/Delta, and B.1.1.529/Omicron. B/HPIV3/S-6P induced higher serum IgA and IgG titers to SARS-COV-2 S and its receptor binding domain in hamsters than B/HPIV3/S-2P. Four weeks after immunization, hamsters were challenged intranasally with 104.5 50 percent tissue culture infectious doses (TCID₅₀) of SARS-COV-2 isolate USA/WA-1/2020 (lineage A, S amino acid sequence identical to that of B/HPIV3/S). In B/HPIV3 control-immunized hamsters, SARS-COV-2 replicated to mean titers of 10^{6.6} TCID₅₀/g in lungs and 107 TCID₅₀/g in nasal tissues and induced moderate weight loss. Immunization with B/HPIV3/S, B/HPIV3/S-2P, or B/HPIV3/S-6P protected against weight loss after SARS-COV-2 challenge. In B/HPIV3/S-immunized hamsters, USA/WA-1/2020 challenge virus was reduced 20-fold in nasal tissues and undetectable in lungs. Immunization with B/HPIV3/S, B/HPIV3/S-2P or B/HPIV3/S-6P protected against weight loss after challenge. In B/HPIV3/S-2P-immunized hamsters, infectious USA/WA-1/2020 challenge virus was undetectable in nasal tissues and lungs, supporting the clinical evaluation of B/HPIV3/S-2P as a pediatric intranasal vaccine against HPIV3 and SARS-COV-2.

In a second study, B/HPIV3, B/HPIV3/S-2P or B/HPIV3/S-6P-immunized hamsters were challenged with USA/WA-1/2020 or with representatives of variants of concern of lineages B.1.1.7/Alpha, or B.1.351/Beta. All challenge viruses induced weight loss in B/HPIV3 control animals, but not in B/HPIV3/S-2P or B/HPIV3/S-6P immunized hamsters. In B/HPIV3/S-2P or B/HPIV3/S-6P immunized hamsters, challenge virus of all lineages was undetectable or significantly reduced in nasal turbinates and lungs on day 3 after challenge, and undetectable in nasal turbinates and lungs on day 5 post-challenge. Thus, B/HPIV3/S-2P and B/HPIV3-S6P are suitable for clinical development as an intranasal vaccine to protect infants and young children against HPIV3 and SARS-COV-2.

Additional studies were performed using rhesus macaques (RMs). A single intranasal/intratracheal immunization with B/HPIV3/S-6P efficiently induced mucosal IgA and IgG in the upper airway (UA) and lower airway (LA) of all immunized RMs, as well as strong serum IgM, IgA and IgG responses to SARS-CoV-2 S protein and its RBD. The anti S and anti-RBD IgG responses were comparable to those detected in human convalescent plasma of individuals with high levels of anti-S and anti-RBD IgG antibodies. The serum antibodies efficiently neutralized the vaccine-matched SARS-COV-2 WA1/2020 strain, as well as variants of concern (VoCs) of the B.1.1.7/Alpha and B.1.617.2/Delta lineages. B/HPIV3/S-6P also induced S-specific CD4 and CD8 T cells in the blood and the LA, including CD4+ and CD8+ T tissue-resident memory cells in the LA. Similarly to immunization with injectable SARS-COV-2 vaccines, intranasal/intratracheal immunization with B/HPIV3/S-6P induced S-specific Th1-biased CD4 T cells in the blood that expressed IFNy, TNFα and IL-2 (Corbett et al., Science 373:cabj0299, 2021; Joyce et al., Sci Transl Med 14(632): eabi5735, 2021; Corbett et al., N Engl J Med 383:1544-1555, 2020; Corbett et al., Nat Immunol 22:1306-1315, 2021). Furthermore, the B/HPIV3/S-6P-induced Th1-biased CD4 T cells expressed markers of cytotoxicity such as CD107ab and granzyme B, suggesting that they might also be directly involved in virus clearance. In addition, B/HPIV3/S-6P induced a stronger S-specific CD8 T cell response in the blood of RMs compared to injectable vaccines (Corbett et al., Science 373:eabj0299, 2021; Corbett et al., N Engl J Med 383: 1544-1555, 2020; Mercado et al., Nature 586:583-588, 2020). Moreover, RMs were fully protected from SARS-COV-2 challenge 1 month after immunization. No SARS-COV-2 challenge virus replication was detectable in the UA or LA or in lung tissues of immunized RMs. In summary, a single topical immunization with B/HPIV3/S-6P was highly immunogenic and protective against SARS-COV-2 in RMs. The data disclosed herein support the further development of this vaccine candidate for use as a stand-alone vaccine and/or in prime/boost combinations with an injectable mRNA-based vaccine for infants and young children.

### Example 1: Materials and Methods This example describes the material and experimental procedures for the studies described in Examples 2 and 3.

### Cells, Viruses and Reagents

African green monkey Vero cells (ATCC CCL-81), Vero E6 cells (ATCC CRL-1586), or LLC-MK2 rhesus monkey kidney cells were grown in OptiMEM (Thermo Fisher) with 5% fetal bovine serum. Human lung epithelial A549 cells (ATCC CCL-185) were grown in F12 medium (ATCC) with 5% FBS. Vero cells stably expressing TMPRSS2 were grown in DMEM with 10% FBS, 1% L-glutamine, and 250 μl/mL of hygromycin B Gold (Invivogen). The SARS-COV-2 USA-WA1/2020 challenge virus (lineage A; Genbank MN985325 and GISAID: EPI_ISL_404895; obtained from the Centers of Disease Control, Atlanta, GA) was passaged twice on Vero E6 cells. The USA/CA_CDC_5574/2020 isolate (lineage B.1.1.7/Alpha, GISAID: EPI_ISL_751801; provided by the Centers for Disease Control and Prevention) and the USA/MD-HP01542/2021 isolate (lineage B.1.351/Beta, GISAID: EPI_ISL_890360) were passaged on Vero E6 cells stably expressing TMPRSS2. Titration of SARS-COV-2 was performed by determination of the 50% tissue culture infectious dose (TCID50) in Vero E6 cells (5). Illumina sequence analysis confirmed that the complete genome sequences of the SARS-COV-2 challenge virus pools were identical to that of consensus sequences, except for minor backgrounds of reads. All experiments with SARS-COV-2 were conducted in Biosafety Level (BSL)-3 containment laboratories approved for use by the US Department of Agriculture and Centers for Disease Control and Prevention.

Virus stocks of recombinant B/HPIV3 vectors were propagated on Vero cells at 32° C. and titrated by dual-staining immunoplaque assay essentially as described (3), using a rabbit antiserum against sucrose gradient-purified HPIV3 virions described previously (6), and a goat hyperimmune antiserum N25-154 against a recombinantly-expressed secreted form (amino acids 1-1208) of the SARS-COV-2 S protein containing two proline substitutions (KV to PP, aa 986 and 987) and four amino acid substitutions (RRAR to GSAS, aa 682-685 with reference to SEQ ID NO: 22) that stabilize S in the prefusion conformation and ablate the furin cleavage site between SI and S2 (7). A plasmid encoding this secreted prefusion-stabilized uncleaved S protein (2019-nCOV S-2P_dFurin_F3CH2S) was transfected into 293Expi cells, and secreted S protein was purified to homogeneity from tissue culture supernatant by affinity chromatography and size-exclusion chromatography, and was used to immunize a goat. To perform the dual staining immunoplaque assay, Vero cell monolayers in 24-well plates were infected with 10-fold serially diluted samples. Infected monolayers were overlaid with culture medium containing 0.8% methylcellulose, and incubated at 32° C. for 6 days, fixed with 80% methanol, and immunostained with the HPIV3 specific rabbit hyperimmune serum to detect B/HPIV3 antigens, and the goat hyperimmune serum to secreted SARS-COV-2 S described above to detect co-expression of the S protein, followed by infrared-dye conjugated goat anti-rabbit IRDye680 IgG and donkey anti-goat IRDye800 IgG secondary antibodies. Plates were scanned with the Odyssey infrared imaging system (LiCor). Fluorescent staining for PIV3 proteins and SARS-COV-2 S was visualized in green and red, respectively, providing for yellow plaque staining when merged.

### Generation of Recombinant B/HPIV3 Expressing SARS-COV-2 Spike Protein

A cDNA clone encoding the B/HPIV3 antigenome was constructed previously (6) and also had previously been modified by two amino acid substitutions in the HN protein (1263T and T370P) that removed two sequence markers and restored the fully-wild-type sequence (8). The full-length cDNA encoding B/HPIV3 contains a unique AscI restriction site in the downstream noncoding region of the N gene. The 1,273 amino acid (aa) ORF encoding the wildtype SARS-COV-2 spike protein S was codon-optimized for human expression, and two versions were generated by DNA synthesis: (i) a version encoding the naturally occurring amino acid sequence, (ii) a version that was identical except that the encoded protein was stabilized in prefusion confirmation (S-2P) by two proline substitutions (KV to PP, aa 986, 987 of SEQ ID NO: 22) and the S1/S2 furin cleavage site was replaced by four amino acid substitutions (RRAR to GSAS, aa 682-685 of SEQ ID NO: 22), and (iii) a version that was identical except that the encoded protein was further stabilized in prefusion confirmation (S-6P) by four additional proline substitutions (F817P, A892P, A899P, A942P, of SEQ ID NO: 26). The sequences of the SARS-COV-2 S proteins used in the B/HPIV3/S-2P and B/HPIV3/S-6P vectors are provided herein as SEQ ID NO: 25 and SEQ ID NO: 26. In each case, the S ORF was preceded by a BPIV3 gene junction containing (in left-to-right order) a gene-end (AAGTAAGAAAAA; SEQ ID NO: 11), intergenic (CTT) and gene-start (AGGATTAATGGA; SEQ ID NO: 34) motif, followed by sequence preceding the ORF (CCTGCAGGATG; SEQ ID NO: 35) that contains the initiation ATG (underlined) in a context favorable for translation initiation (FIG. 1, (9)). Ascl sites were placed flanking each cDNA, and the synthetic DNA was inserted into the unique Ascl site present in the downstream noncoding region of the B/HPIV3 N gene in a cloned cDNA of the complete B/HPIV3 antigenome (FIG. 1). The sequence of the full-length antigenome plasmids was confirmed, and plasmids were used to transfect BHK BSR T7/5 cells as described previously (10) to produce B/HPIV3/S and B/HPIV3/S-2P recombinant viruses. Virus stocks were grown in Vero cells, and viral genomes purified from recovered virus were sequenced in their entirety by Sanger sequencing from overlapping uncloned RT-PCR fragments, confirming the absence of any adventitious mutations.

Multicycle Replication of rBPIV3 Vectors in Cell Culture

Vero cells in 6-well plates were infected in triplicate wells with indicated viruses at a multiplicity of infection (MOI) of 0.01 PFU per cell. After virus adsorption, the inoculum was removed, cells were washed, and 3 ml of fresh medium was added to each well followed by incubation at 32° C. for 7 days. At 24 hour intervals, 0.5 ml of culture medium was collected and flash-frozen, and 0.5 ml of fresh medium was added to each well. Virus aliquots were titrated together in Vero cells in 24-well plates by infrared fluorescent dual-staining immunoplaque assay described above.

### SDS-PAGE and Western Blot Analysis

Vero or A549 cells in 6-well plates were infected with B/HPIV3, B/HPIV3/S, B/HPIV3/S-2P, or B/HPIV3/S-6P at a MOI of 1 PFU per cell and incubated at 32° C. for 48 hours. Cells were washed once with cold PBS and lysed with 300 μl LDS lysis buffer (Thermo Fisher Scientific) containing NuPAGE reducing reagent (Thermo Fisher Scientific). Cell lysates were passed through a QIAshredder (Qiagen, Valencia CA), heated for 10 minutes at 95° C., separated on 4-12% Bis Tris NuPAGE gels (Thermo Fisher Scientific) in the presence of antioxidant (Thermo Fisher Scientific), and transferred to polyvinylidene difluoride (PVDF) membranes. Membranes were blocked with PBS blocking buffer (LiCor, Lincoln NE) and incubated with a goat hyperimmune serum to SARS-COV-2 S and rabbit polyclonal hyperimmune sera against HPIV3 (see cells, viruses and reagents above) primary antibodies in blocking buffer overnight at 4° C. A mouse monoclonal antibody to GAPDH (Sigma) was included to provide a loading control. Membranes were incubated with infrared dye-labeled secondary antibodies (goat anti-rabbit IgG IRDye 680, donkey anti-goat IRDye 800, and donkey anti-mouse IgG IRDye 800, LiCor). Images were acquired and the intensities of individual protein bands were quantified using Image Studio software (LiCor). The relative abundance of viral proteins was normalized by GAPDH, and presented as fold change compared to that of the B/HPIV3 vector.

To analyze the protein composition of virus particles, viruses were grown on Vero cells, purified from the supernatant by centrifugation through 30%/60% discontinuous sucrose gradients, and gently pelleted by centrifugation to remove sucrose as described previously (4). The protein concentration of the purified preparations was determined prior to the addition of lysis buffer, and 1 μg of protein per lane was used for SDS-PAGE and Western blotting.

### Replication, Immunogenicity, and Protective Efficacy Against SARS-COV-2 Challenge in Hamsters

In Experiment 1, Groups (n=30) of 5 to 6-week old female Golden Syrian hamsters (Envigo Laboratories, Frederick, MD), pre-screened to be HPIV3-seronegative, were anesthetized and inoculated intranasally (IN) with 100 μl of Leibovitz's L-15 medium (Thermo Fisher Scientific) containing 105 PFU of B/HPIV3, B/HPIV3/S, or B/HPIV3/S-2P viruses. On days 3 and day 5 post-inoculation, 6 hamsters per group were euthanized by CO2 inhalation, and nasal turbinates, lung, kidney, liver, spleen, intestine, brain, and blood were collected to evaluate virus replication. Lung tissue samples for histology were obtained from two additional hamsters per group on each day. For virus quantification, tissues were homogenized in Leibovitz 15 (L-15) medium, and virus titers of clarified homogenates were assessed by titration by dual-staining immunoplaque assay on Vero cells as described above. On day 28 post-immunization, sera were collected from the remaining 14 animals per group to evaluate the immunogenicity of the vaccine candidates to SARS-COV-2 and HPIV3. B/HPIV3 vector-specific neutralizing antibodies were detected by a 60% plaque reduction neutralization test (PRNT₆₀) on Vero cells in 24-well plates using a GFP expressing version of B/HPIV3. The neutralizing antibody response to SARS-COV-2 was evaluated in a 50% plaque reduction microneutralization assay as described for SARS-COV-1 (3, 5). Serum antibodies to SARS-COV-2 also were measured by ELISA using two different recombinantly-expressed purified forms of S: one was the secreted form of S-2P described above (plasmids generously provided by Drs. Barney Graham, Kizzmekia Corbett, and Jason McLellan), and the other was a fragment (amino acids 328-531) of the SARS-COV-2 S protein containing the receptor binding domain (RBD), obtained from David Veesler through BEI Resources, NIAID, NIH (11). The RBD fragment was expressed from a codon-optimized ORF in Expi293 cells and purified as described above for the secreted S-2P protein.

In Experiment 2, groups (n=10) of 6-week old female golden Syrian hamsters were immunized as described above. On day 30 after immunization, hamsters were challenged intranasally with 4.5 log₁₀ TCID 50 of SARS-COV-2 in 100 μl volumes. Five hamsters per group were euthanized by CO₂ inhalation on days 3 and 5 after challenge, and tissues were collected to evaluate challenge virus replication (n=5 per group). The presence of challenge virus in clarified tissue homogenates was evaluated later by TCID₅₀ assay. To detect serum antibodies specific to SARS-COV-2, twofold dilutions of heat-inactivated hamster sera were tested in a microneutralization assay for the presence of antibodies that neutralized the replication of 100 TCID₅₀ of SARS-COV-2 in Vero cells, with four wells per dilution on a 96-well plate. The presence of viral cytopathic effect was read on day 4. The dilution of serum that completely prevented cytopathic effect in 50% of the wells was calculated by the Reed and Muench formula (12).

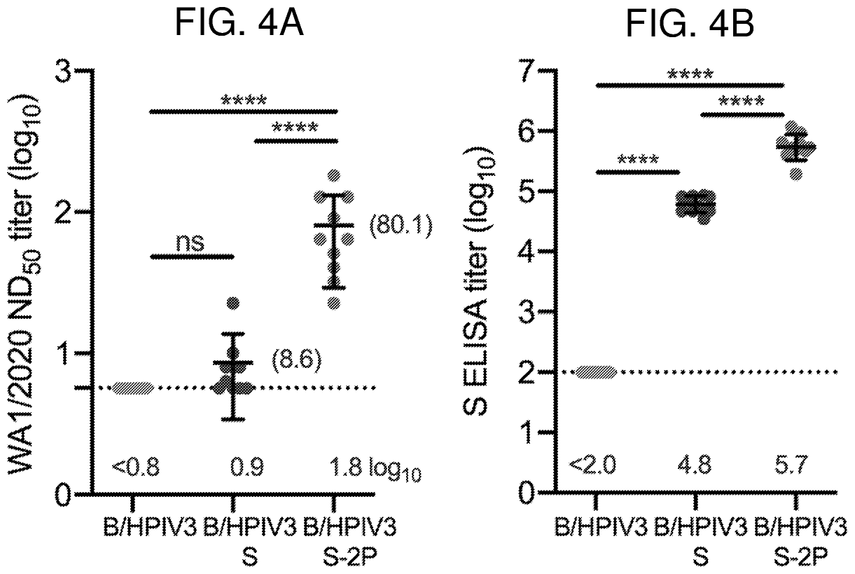

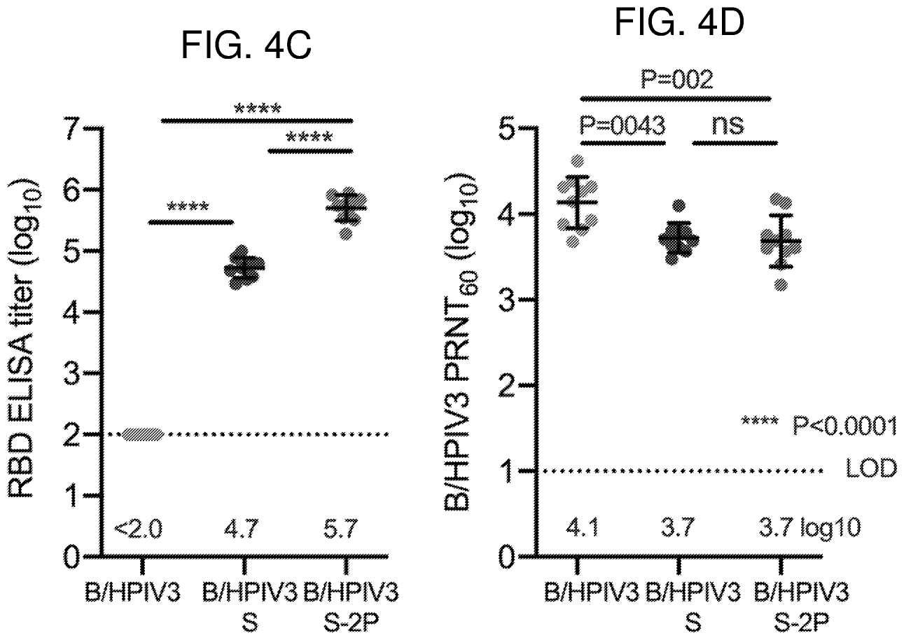

RT-qPCR analysis of gene expression in lung tissue. Total RNA was extracted from 0.125 ml of lung homogenates (0.1 mg/ml) using the TRIzol Reagent and Phasemaker Tubes Complete System (Thermo Fisher) along with the PureLink RNA Mini Kit (Thermo Fisher) following the manufacturer's instructions. Total RNA was also extracted from lung homogenates of three control hamsters (non-immunized and non-challenged) in the same manner. cDNA was synthesized from 350 ng of RNA by using the High-Capacity RNA-to-cDNA Kit (Thermo Fisher). Low-density Taqman gene array (Thermo Fisher) were configured to contain TaqMan primers and probes for 14 hamster (mesocritecus auratus) chemokine and cytokine genes, which were designed based on previous reports (13, 14). Hamster beta-actin was included as a housekeeping gene. A mixture of cDNA and 2× Fast Advanced Master Mix (Thermo Fisher) was added into each fill port of the array cards for real-time PCR with QuantStudio 7 Pro (Thermo Fisher). qPCR results were analyzed using the comparative threshold cycle (ΔΔC_{T}) method, normalized to beta-actin, and expressed as fold-change over the average of expression of three uninfected, unchallenged hamsters. Results in FIG. 4C are presented as heat maps using the Gene Expression Similarity Investigation Suite (GENESIS program, release 1.8.1, http://genome.tugraz.at).

### Immunohistopathology Analysis

Immunohistopathology analysis. Lung tissue samples from hamsters were fixed in 10% neutral buffered formalin, processed through a Leica ASP6025 tissue processor (Leica Biosystems), and embedded in paraffin. 5 um tissue sections were stained with hematoxylin and eosin (H&E) for routine histopathology. For immunohistochemical (IHC) evaluation, sections were deparaffinized and rehydrated. After epitope retrieval, sections were labeled with goat hyperimmune serum to SARS-COV-2 S (N25-154) at 1:1000, and rabbit polyclonal anti-HPIV3 serum (6) at 1:500. Chromogenic staining was carried out on the Bond RX platform (Leica Biosystems) according to manufacturer-supplied protocols. Detection with DAB chromogen was completed using the Bond Polymer Refine Detection kit (Leica Biosystems). The VisUCyte anti-goat HRP polymer (R&D Systems, VC004) replaced the standard Leica anti-rabbit HRP polymer from the kit to bind the SARS-COV-2 S goat antibodies. Slides were finally cleared through gradient alcohol and xylene washes prior to mounting. Sections were examined by a board-certified veterinary pathologist using an Olympus BX51 light microscope and photomicrographs were taken using an Olympus DP73 camera.

Replication and immunogenicity of B/HPIV3 and B/HPIV3/S-6P in rhesus macaques. Rhesus macaques (n=4 per group), seronegative for HPIV3 as determined by a 60% plaque reduction neutralization assay, were immunized intranasally and intratracheally with 6 log₁₀ PFU of B/HPIV3 or B/HPIV3/S-6P under light sedation. Serum was collected on days -3, 14, 21 and 28 post-inoculation for serology. Nasopharyngeal (NP) swabs were collected daily on days 0 through 10 and day 12, and tracheal lavage (TL) samples were collected on days 2, 4, 6, 8 10, and 12 to analyze vaccine virus shedding. Virus shedding was analyzed by dual-staining immunoplaque assay, and serum IgG titers to the SARS-COV-2 S protein were determined by ELISA. Human COVID-19 convalescent plasma sera (de-identified samples) were included in the ELISA assay for comparison and to provide benchmarks.

### Statistical Analysis

Data sets were assessed for significance using one-way ANOVA with Tukey's multiple comparison test using Prism 8 (GraphPad Software). Data were only considered significant at p≤0.05.

### Example 2: Intranasal Parainfluenza Vector Vaccine Protects Against SARS-COV-2 in Hamsters

This example describes the development and characterization of two recombinant bovine/human parainfluenza viruses (B/HPIV3) expressing a SARS-COV-2 spike protein (either WT or pre-fusion stabilized S protein) as candidate vaccines for SAR-COV-2.

Design, Recovery, and in-vitro Characterization of B/HPIV3 Vector Vaccine Candidates Expressing Wild-Type or Prefusion-Stabilized Versions of the SARS-COV-2 S Protein.

B/HPIV3 consists of BPIV3 in which the BPIV3 F and HN genes have been replaced, using reverse genetics, by those of HPIV3 [(15); FIG. 1A]. B/HPIV3 was used as a vector to express the SARS-COV-2 spike S protein, the major neutralization and protective antigen of SARS-COV-2, from an added gene. The 1,273 amino acid (aa) S ORF derived from the first available SARS-COV-2 genome sequence [Genbank MN908947; (16)] was codon-optimized for human expression, and placed under control of PIV3 gene start (transcription initiation) and gene end (transcription termination and polyadenylation) signals to direct its expression as a separate mRNA by the PIV3 transcriptional machinery (FIG. 1A). A second version of this gene (S-2P) was modified to contain two prefusion-stabilizing proline substitutions at aa positions K986P and V987P of S (S-2P), as well as four amino acid substitutions in the S1/S2 furin cleavage site (residues 682-685; RRAR-to-GSAS) that ablate cleavage (7). Each of the two S gene versions were inserted into full-length B/HPIV3 cDNAs between the N and P genes (FIG. 1A), which in previous studies provided efficient and stable expression of heterologous genes with minimal effect on B/HPIV3 vector replication (6). The resultant cDNAs were used to recover recombinant B/HPIV3/S and B/HPIV3/S-2P viruses by reverse genetics as described previously (10). Virus stocks were grown on Vero cells, a suitable substrate for vaccine manufacture, and viral genomes were sequenced in their entirety, confirming the absence of any adventitious mutations.

To determine the viral titers and evaluate the stability of expression of the S or S-2P proteins, dual-staining immunoplaque assays we performed on viral stocks with antibodies to PIV3 and SARS-COV-2 S. In stocks grown from 4 (B/HPIV3/S) or 8 (B/HPIV3/S-2P) independent recoveries, staining for both PIV3 and SARS-COV-2 was obtained in 99.4+1.3 and 94.9+3.4% of B/HPIV3/S and B/HPIV3/S-2P plaques (FIG. 1B), indicative of stable expression of the SARS-COV-2 S protein. Multicycle replication of B/HPIV3/S and B/HPIV3/S-2P in Vero cells was efficient and overall similar to that of B/HPIV3, indicating that the presence of the 3.8 kb S or S-2P inserts did not slow or reduce the replication of the B/HPIV3 vector in vitro (FIG. 1C).

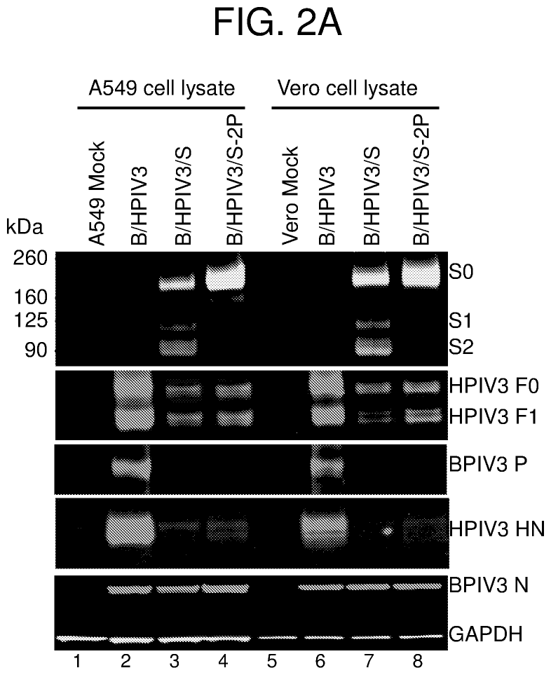

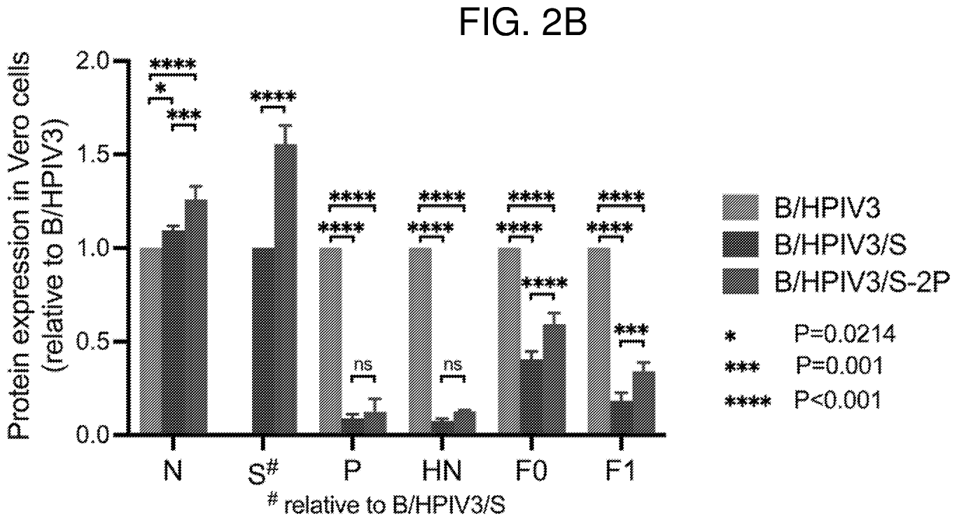

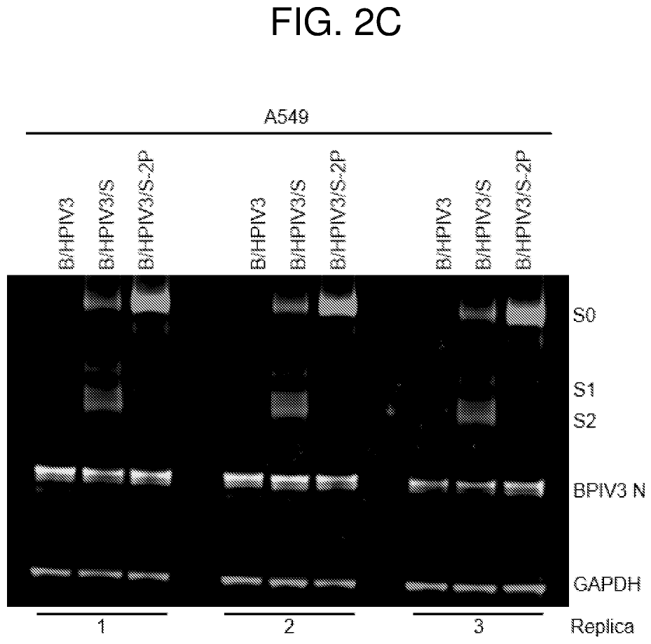

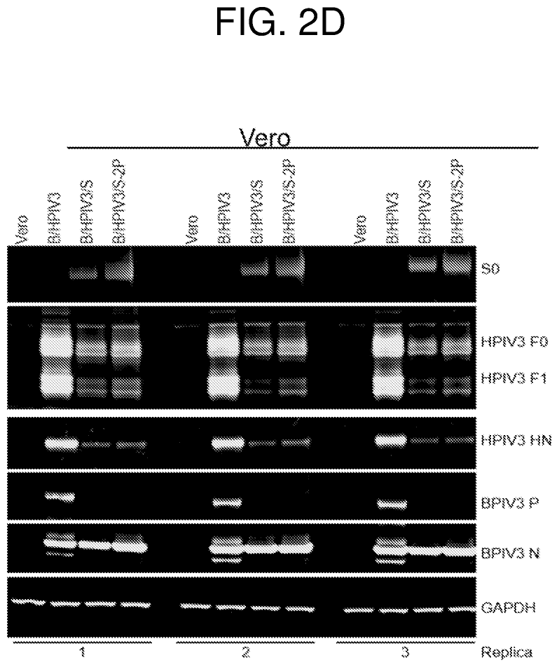

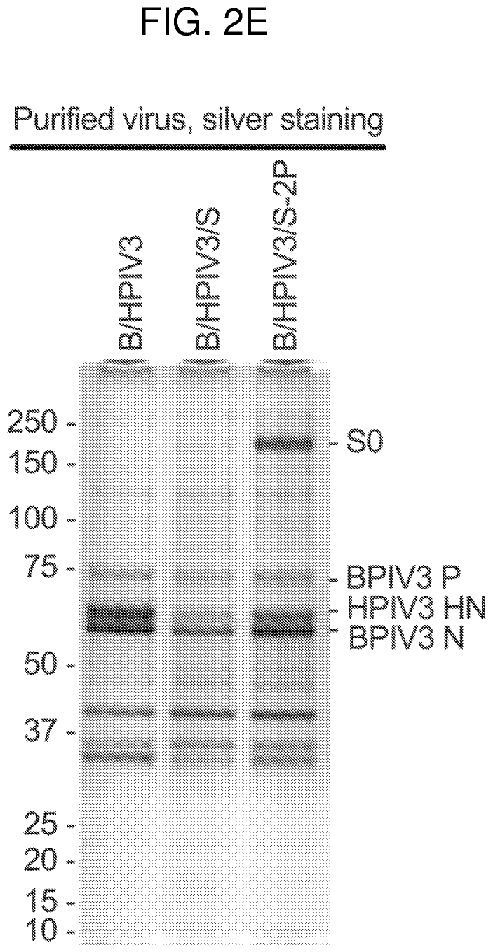

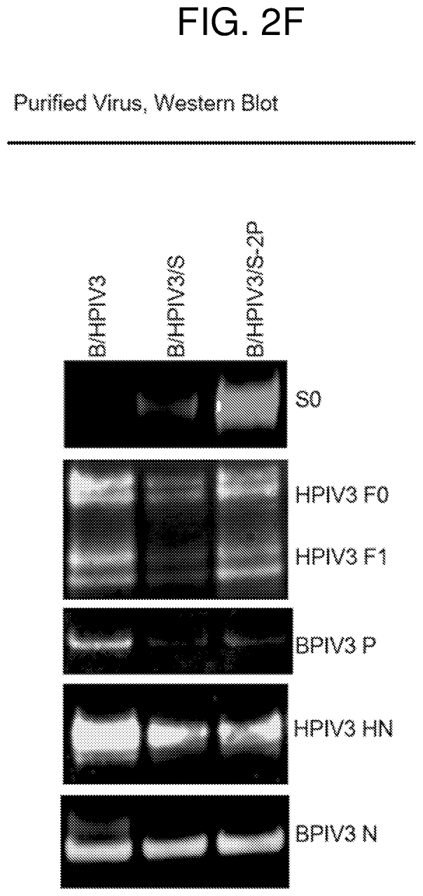

To characterize the expression of the SARS-COV-2 S and B/HPIV3 proteins in vitro, Vero and human lung epithelial A549 cells were infected with B/HPIV3, B/HPIV3/S, or B/HPIV3/S-2P at a multiplicity of infection (MOI) of 1 plaque forming unit (PFU) per cell. Cell lysates were prepared 48 hours after infection, and analyzed by SDS-PAGE and Western blotting, using antisera to detect SARS-COV-2 S or PIV3 proteins. (FIGS. 2A, 2B, 2C, 2D). In lysates from both cell lines, the S protein was detectable as a high-molecular band, consistent in size with uncleaved SO precursor protein (FIG. 2A, lanes 3, 4, 7, 8, and FIGS. 2C and 2D). In B/HPIV3/S infected A549 cells (FIG. 2A, lane 3, FIG. 2C) and Vero cells (FIG. 2A, lane 7), additional smaller products were present, consistent in size with cleavage products S1 and S2. The absence of these smaller bands in B/HPIV3/S-2P infected A549 and Vero cells confirmed the absence of the furin cleavage of the S-2P protein in which the cleavage site had been ablated (FIG. 2A, lanes 4 and 8, and FIG. 2C). Notably, prefusion stabilization increased the intensity of Western blot staining in both cell lines (FIG. 2A, compare B/HPIV3/S and B/HPIV3/S-2P; lanes 3 versus 4, and 7 versus 8; FIGS. 2C and 2D).

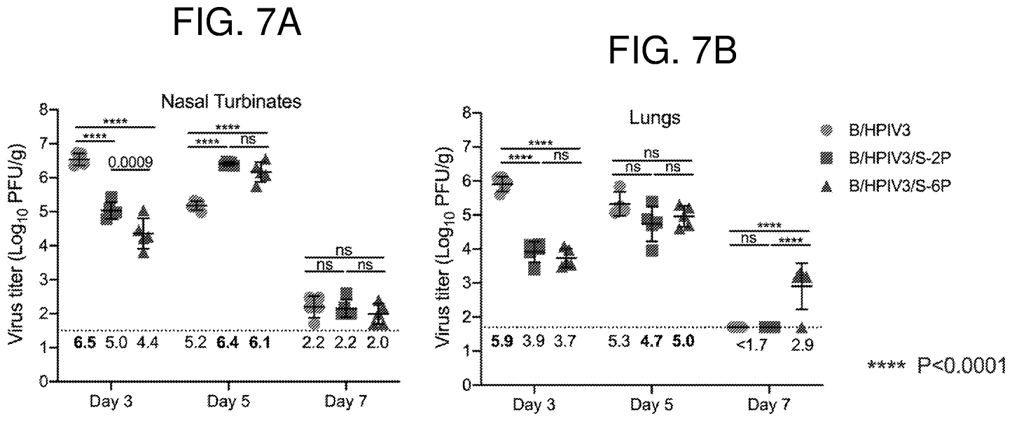

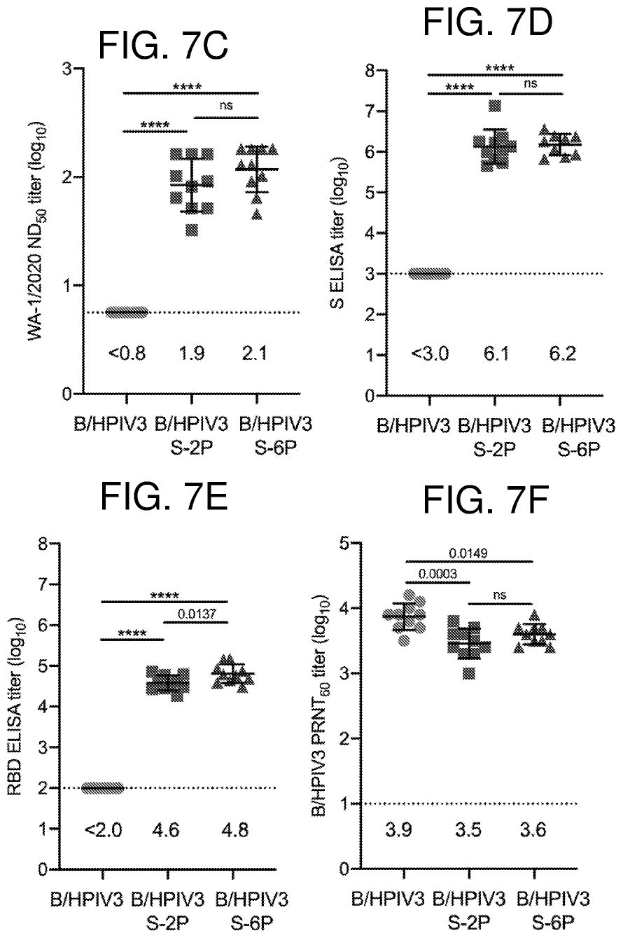

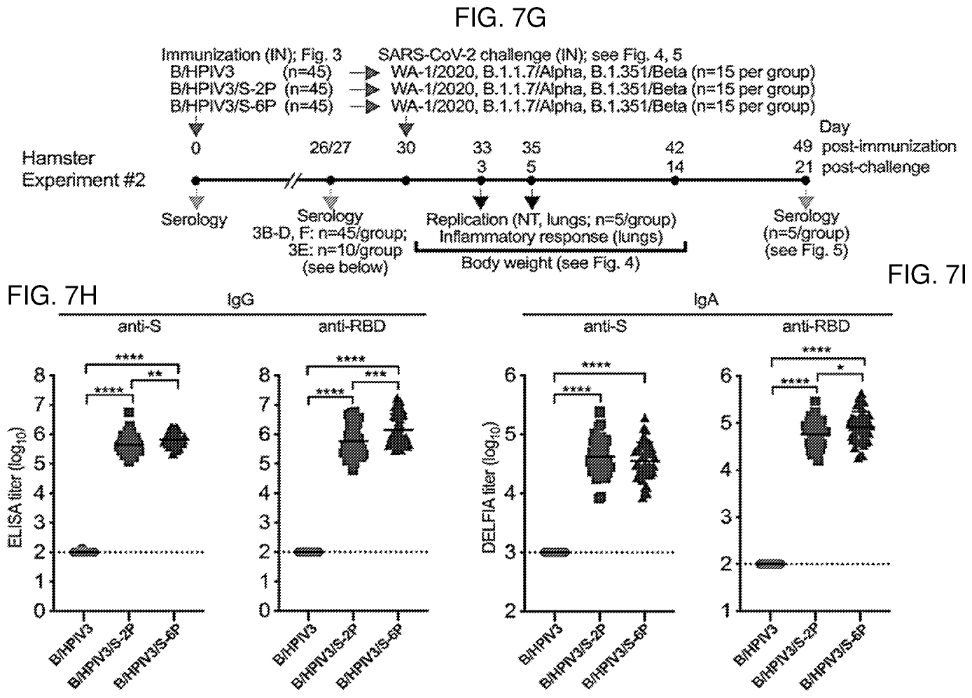

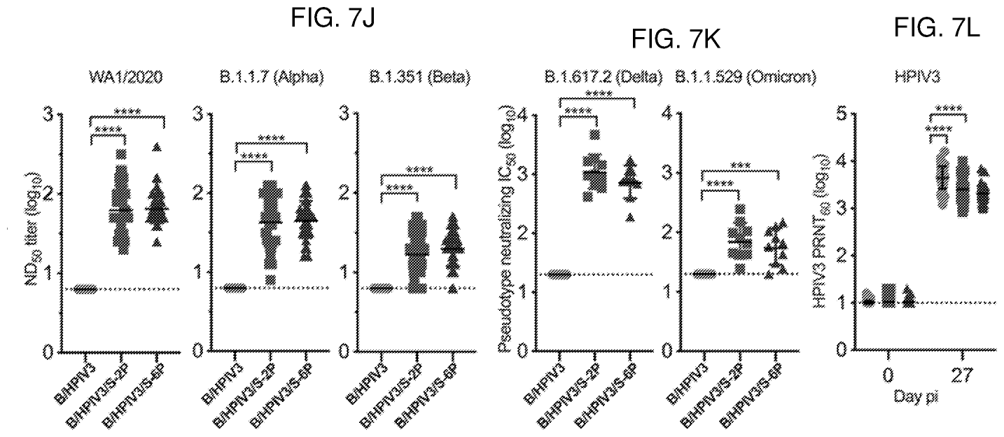

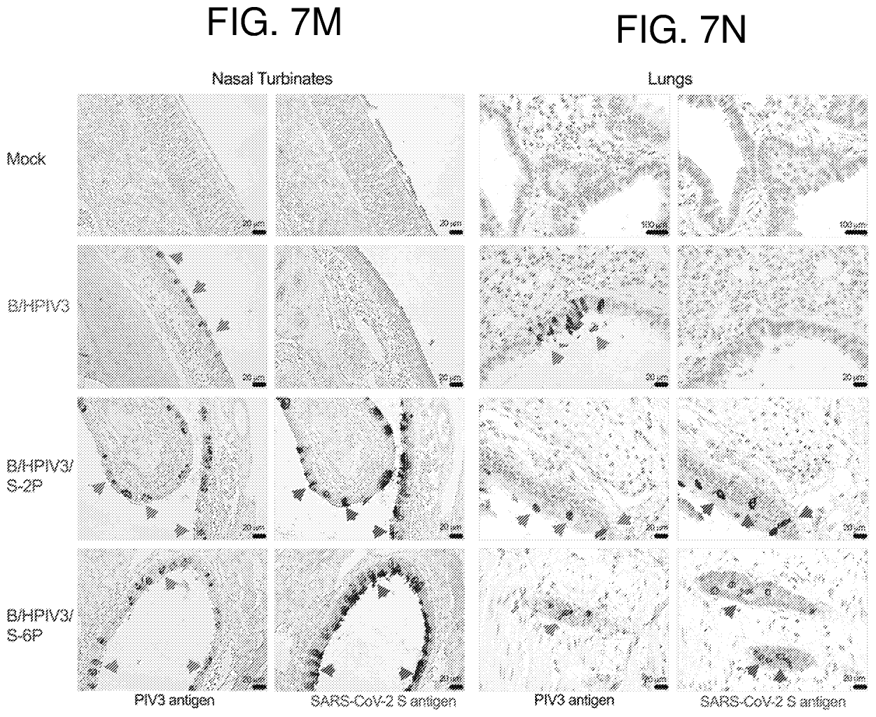

A quantitative comparison in Vero cells of protein expression by B/HPIV3/S and B/HPIV3/S-2P from 3 additional independent experiments showed that prefusion stabilization increased levels of vector-expressed SARS-COV-2 S protein by about twofold in Vero cells (FIGS. 2B, 2D), and 7 fold in A549 cells, respectively (FIG. 2C). We also investigated whether the insertion of the S gene cassettes between the vector N and P genes had any effect on the expression of the vector genes. The quantitative analysis in Vero cells revealed that the level of expression of the upstream N gene by B/HPIV3/S and B/HPIV3/S-2P was comparable to that of B/HPIV3, while the expression of downstream vector genes (BPIV3 P; HPIV3 F and HN) was strongly reduced by about 50-90% (FIGS. 2A, 2B, 2C, 2D).

To evaluate possible incorporation of the SARS-COV-2 S or S-2P protein into B/HPIV3 particles, Vero-grown viruses were purified by centrifugation through sucrose gradients, and the protein composition was analyzed by gel electrophoresis with silver staining and Western blotting (FIGS. 2E and 2F). In silver-stained gels, a high-molecular band consistent with SARS-COV-2 SO was visible in B/HPIV3/S-2P preparations, but not in B/HPIV3 or B/HPIV3/S preparations. Immunostaining identified this band as S0 (FIG. 2F), indicating that prefusion-stabilized version but not the wildtype version of the S protein was incorporated in the B/HPIV3 vector particles.

Immunization of Hamsters with the B/HPIV3/S Viruses

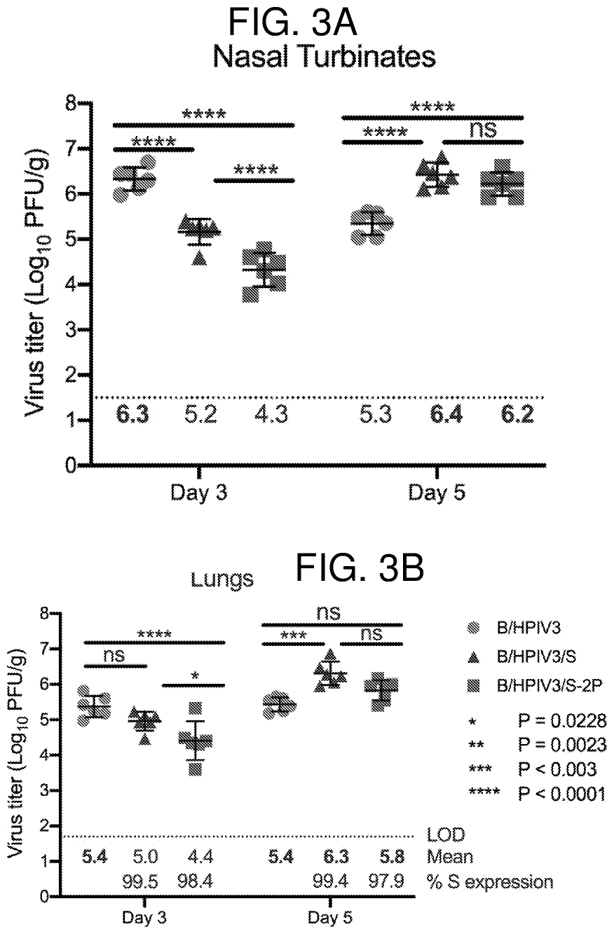

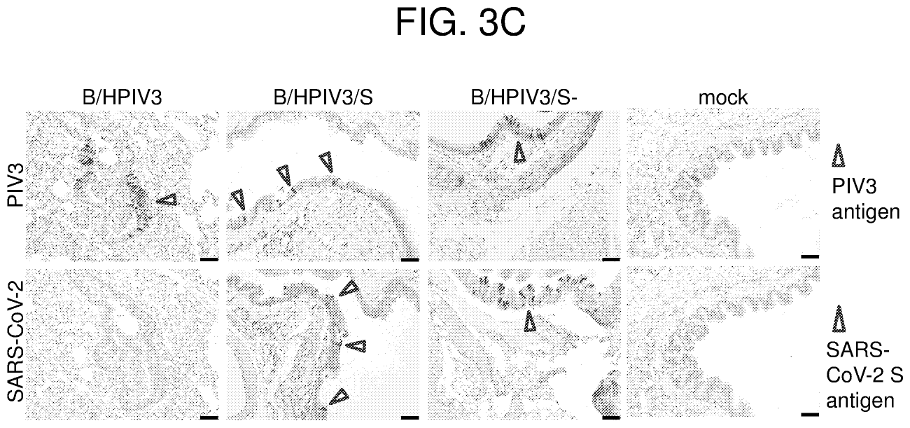

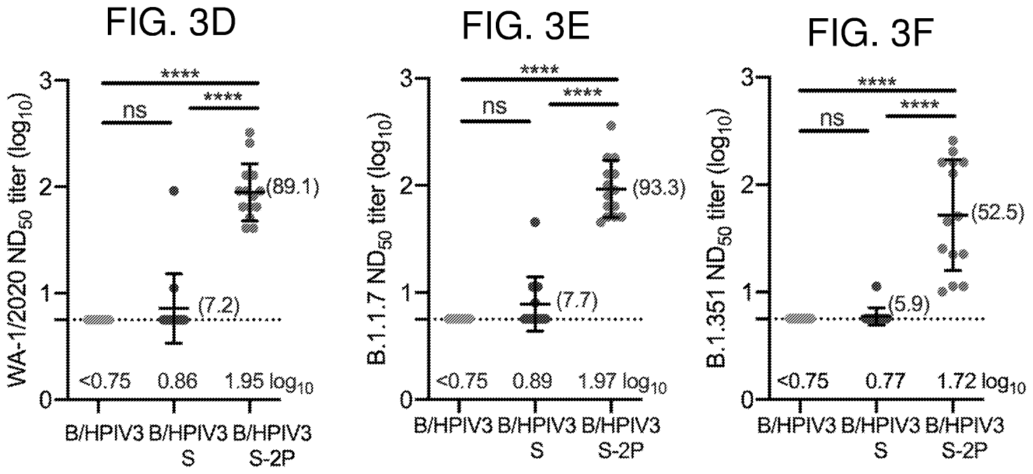

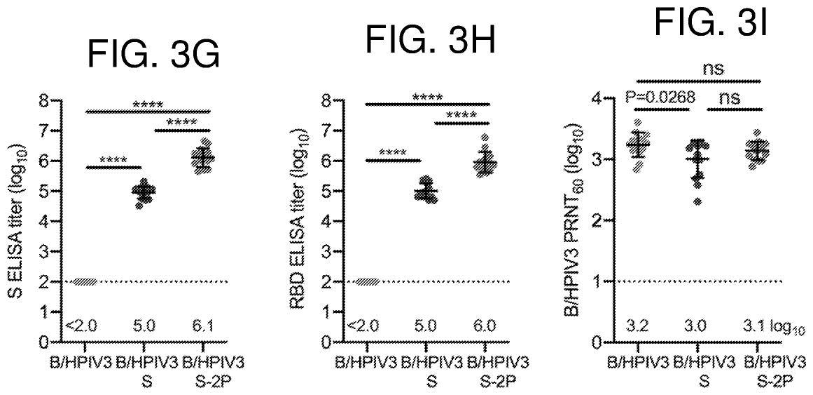

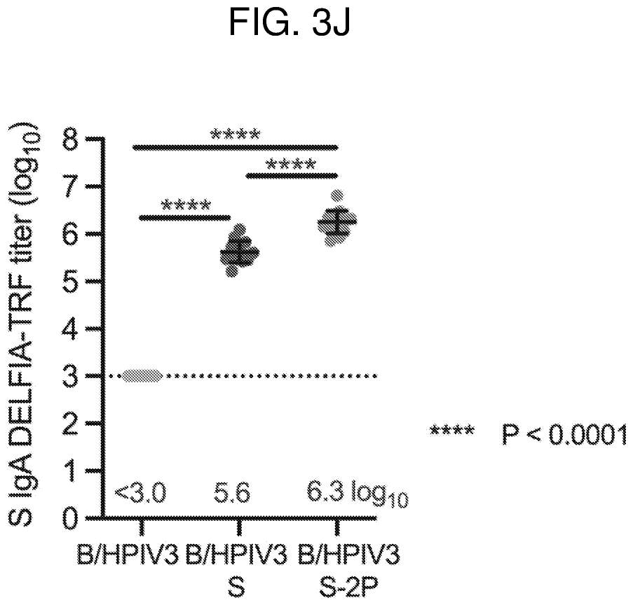

To evaluate the replication and immunogenicity of the vaccine candidates in a susceptible animal model, hamsters in groups of 30 were inoculated intranasally with 5 log₁₀ PFU of the B/HPIV3/S or B/HPIV3/S-2P vaccine candidates, or with B/HPIV3 empty vector control. On days 3 and 5 after inoculation, 8 hamsters per group were euthanized to evaluate vector replication in the respiratory tract: nasal turbinates and lungs were harvested from 6 animals and tissue homogenates were prepared and analyzed by immunoplaque assay (FIGS. 3A, B), and lungs were harvested from the remaining 2 animals and analyzed by immunohistochemistry (FIG. 3C).

B/HPIV3 replicated to high mean peak titers (6.3 and 5.4 log₁₀ PFU/g on day 3) in the nasal turbinates and lungs, respectively (FIGS. 3A, B), as typically observed. In the nasal turbinates, titers of the B/HPIV3 empty vector were higher on day 3 and decreased about ten-fold by day 5. In contrast, B/HPIV3/S and B/HPIV3/S-2P titers were 10- and 100-fold lower than that of the empty vector control in nasal turbinates on day 3 (5.2 and 4.3 log₁₀ PFU/g vs 6.3 log₁₀ PFU/g for B/HPIV3), but increased and were significantly higher than B/HPIV3 titers on day 5 (6.4 log₁₀ and 6.2 log₁₀ PFU/g for B/HPIV3/S and B/HPIV3/S-2P vs 5.3 log₁₀ PFU/g for B/HPIV3). This suggested that the presence of the large S insert in the B/HPIV3 genome delayed virus replication in the upper respiratory tract. However, mean nasal peak titers of all 3 viruses, independent of the study day (6.3 log₁₀ PFU/g for B/HPIV3; 6.4 log₁₀ and 6.2 log₁₀ PFU/g for B/HPIV3/S and B/HPIV3/S-2P) were not significantly different (FIG. 3A).

In the lungs, B/HPIV3 replication remained at a high level over both days (5.4 log₁₀ PFU/g). Similar to the findings in the nasal turbinates, mean titers of B/HPIV3/S and B/HPIV3/S-2P (5.0 log₁₀ and 4.4 log₁₀ PFU/g) were also lower than those of the B/HPIV3 empty vector on day 3, although the difference between B/HPIV3 and B/HPIV3/S in the lungs did not reach statistical significance. By day 5 post-immunization, B/HPIV3/S reached about 10-fold higher titers compared to the empty vector on either day, suggesting that the wild-type version of the S protein contributed to vector replication in the lungs. The peak titers of B/HPIV3/S-2P in lungs were also marginally higher than those of B/HPIV3, but this was not statistically significant (FIG. 3B).

The lung samples were also analyzed by dual-immunostaining plaque assay to determine the stability of expression of S and S-2P proteins in vivo. Specifically, 99.5% and 98.4% of B/HPIV3/S and B/HPIV3/S-2P plaques, respectively from lung samples obtained on day 3 after infection stably expressed the S protein, and 99.4% and 97.9% of B/HPIV3/S and B/HPIV3/S-2P plaques, respectively, obtained on day 5 expressed the S protein (FIG. 3B, bottom). Thus, vector expression of both versions of the S protein was stably maintained in vivo. In addition to lungs and nasal turbinates described above, brain, kidney, liver, spleen tissues and small intestine were also collected at both day 3 and day 5 post-inoculation, homogenized, and analyzed by immunostaining plaque assay. B/HPIV3, B/HPIV3/S and B/HPIV3/S-2P vaccine viruses were not detected in any of these non-respiratory tissues, showing that the presence of the S protein did not detectably alter the tropism for respiratory tissues of the B/HPIV3 vector.

Antigen expression in the lungs of immunized animals was analyzed by immunohistochemistry (IHC) on tissues from 2 animals per group on days 3 and 5 after immunization; representative IHC images are shown in FIG. 3C. B/HPIV3 antigen was detected in the lungs primarily in columnar bronchial epithelial cells lining the small airways, as shown in tissue from B/HPIV3, B/HPIV3/S, and B/HPIV3/S-2P immunized animals obtained on day 5 (arrowheads, FIG. 3C, top panel). SARS-COV-2 S antigen in animals immunized with B/HPIV3/S and B/HPIV3/S-2P similarly was detected in columnar bronchial epithelial cells lining the small airway (arrowheads, FIG. 3C, bottom panels). Overall, the B/HPIV3 and SARS-COV-2 S immunostaining pattern did not differ between the B/HPIV3/S and B/HPIV3/S-2P immunized animals. These results show that following intranasal immunization of hamsters, the B/HPIV3/S and B/HPIV3/S-2P vectors efficiently infected and expressed the SARS-COV-2 S protein in bronchial epithelial cells, with no obvious difference in tissue distribution between B/HPIV3 expressing the wildtype and the prefusion-stabilized versions of the S protein.

The serum antibody response was evaluated 28 days after intranasal immunization in the remaining animals (n=14 animals per group). SARS-COV-2-neutralizing antibody titers were measured by an ND₅₀ assay against SARS-COV-2, strain WA1/2020, a representative of the SARS-COV-2 lineage A with an S amino acid sequence identical to that expressed by B/HPIV3/S (FIG. 3D). As expected, SARS-COV-2 neutralizing antibodies were not detected in animals immunized with B/HPIV3 empty vector. B/HPIV3/S induced a very low response of SARS-COV-2 serum neutralizing antibodies [geometric mean reciprocal ND₅₀ titer: 0.86 log₁₀. (1:7.2)], whereas B/HPIV3/S-2P induced significantly higher (approximately 12-fold) titers of SARS-COV-2-neutralizing antibodies [geometric mean reciprocal ND₅₀ titer: 1.95 log₁₀ (1:89.1), FIG. 3D]. In addition, the ability of the serum antibodies induced by the B/HPIV3 vectors to neutralize SARS-COV-2 variants of concern was evaluated. Sera from immunized hamsters were evaluated in neutralization assays using isolates USA/CA_CDC_5574/2020 of lineage B.1.1.7 [United Kingdom (UK) variant, carrying the N501Y, A570D, D614G, P681H, T716I, S982A, and D1118H signature mutations in the S protein], and USA/MD-HP01542/2021 of lineage B.1.351/Beta [South Africa (SA) variant, carrying the signature mutations K417N, E484K, N501Y, D614G, and A701V in S] (17, 18). Remarkably, serum neutralizing antibody titers induced by B/HPIV3/S-2P to the B.1.1.7/Alpha representative [geometric mean reciprocal ND₅₀ titer: 1.97 log₁₀ (1:93.3), FIG. 3E] were comparable to those against WA1/2020 (lineage A). Using a representative of lineage B.1.351/Beta (FIG. 3F), we observed a greater animal-to-animal variability in neutralizing titers [geometric mean reciprocal ND₅₀ titer: 1.72 log₁₀ (1:52.2)]. Serum antibodies from B/HPIV3/S-immunized animals only exhibited very low neutralizing activities against these representatives of heterologous lineages, similarly to the low serum neutralizing antibody titers against WA1/2020 (lineage A).

In addition, SARS-COV-2-specific serum IgG was measured by ELISA using as antigen purified preparations of the secreted form of the S-2P protein (FIG. 3G) and a fragment (aa 319-591) of the S protein bearing the receptor-binding domain (RBD) (FIG. 3H). Moderate serum IgG titers to the secreted S-2P protein and to the RBD were detected in B/HPIV3/S-immunized animals, while significantly stronger IgG responses to the secreted S-2P (13-fold higher) and RBD (10-fold higher) antigens were induced by B/HPIV3/S-2P.

All three viruses also induced a strong neutralizing antibody response in a 60% plaque reduction assay against B/HPIV3 (FIG. 31). The antibody response to the B/HPIV3 vector induced by B/HPIV3/S-2P was similar to that induced by the empty B/HPIV3 vector control, while the antibody response induced by B/HPIV3/S was slightly lower than that of the empty vector.

Protection of B/HPIV3/S Vaccine Candidates Against Intranasal Challenge with SARS-COV-2

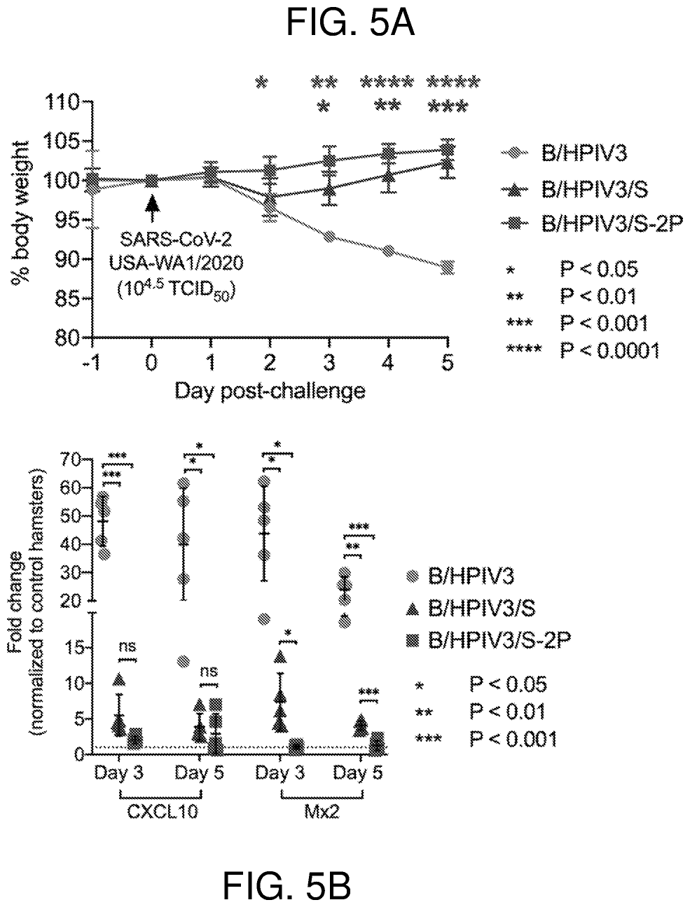

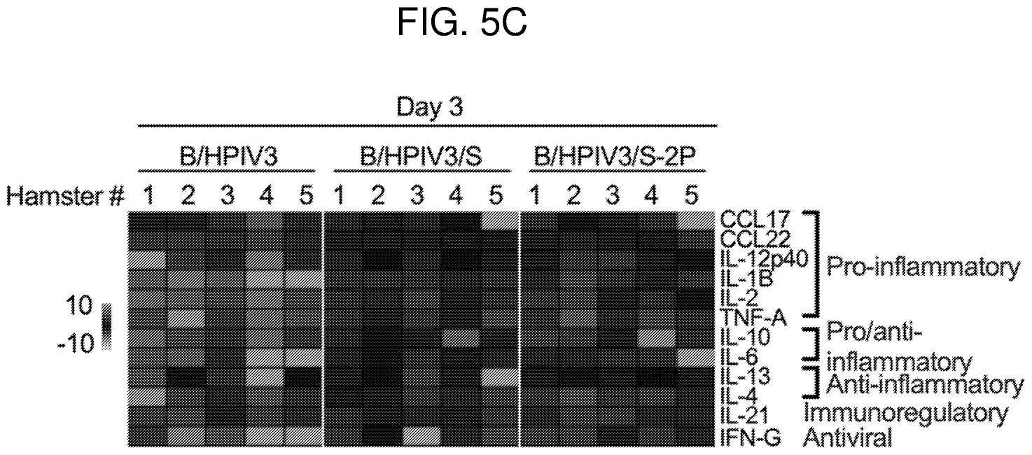

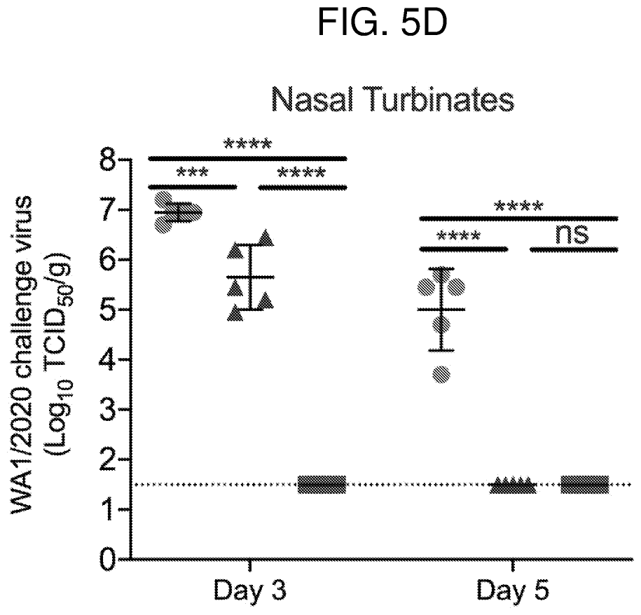

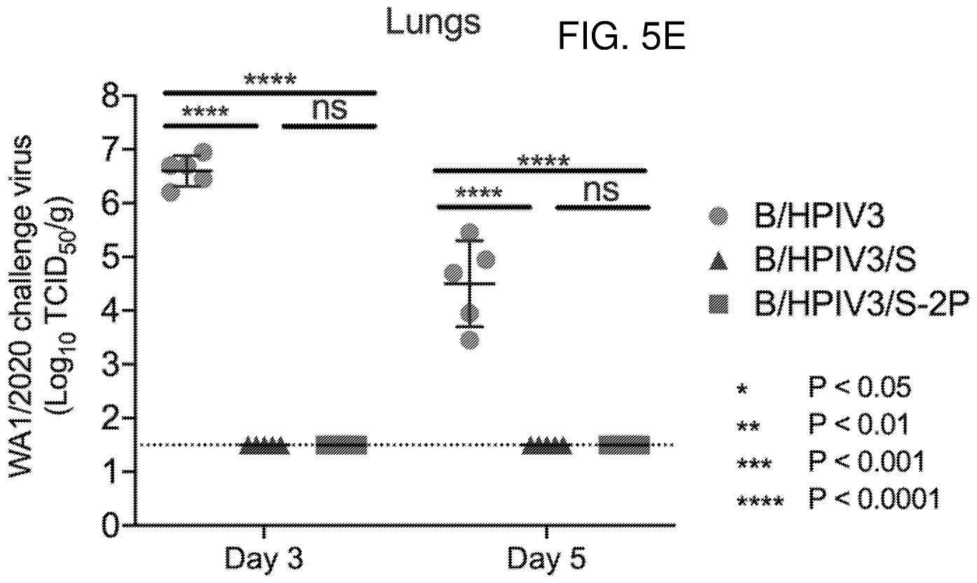

To evaluate protection against intranasal SARS-COV-2 challenge, hamsters in groups of 10 were immunized as described above. The serum antibody response 27 days after immunization (FIGS. 4A-4D) was comparable to that in the previous study. Animals were challenged intranasally on day 30 after immunization with 4.5 log₁₀ TCID₅₀ of SARS-COV-2, isolate WA1-USA/2020 from a preparation that had been subjected to complete-genome deep sequencing to confirm its integrity. Animals were observed for clinical symptoms and monitored for weight loss (FIG. 5A). During the first five days following SARS-CoV-2 challenge, animals immunized with the empty B/HPIV3 vector exhibited moderate weight loss, representing the only clinical symptom after challenge (10% average loss by day 5 post-challenge), while animals immunized with B/HPIV3/S and B/HPIV3/S-2P generally continued to gain body weight. The weight loss in the empty B/HPIV3 vector-immunized group reached significant levels compared to the B/HPIV3/S-2P-immunized animals on day 2 and the B/HPIV3/S-immunized animals on day 3. Five animals per group were euthanized on days 3 and 5 post-challenge, and tissues were collected. RNA was extracted from lung homogenates, and the expression of inflammatory cytokine genes after SARS-COV-2 challenge was assayed by taqman assay (FIG. 5B). In FIG. 5B, results are shown for the two genes that were most strongly expressed in the B/HPIV3 control immunized animals, namely C-X-C motif chemokine ligand 10 (CXCL10) and myxovirus resistance protein 2 (Mx2). CXCL10 is an interferon-inducible cytokine which stands out as a biomarker of the SARS-COV-2 cytokine storm, serving as a correlate of COVID-19 disease severity in COVID-19 patients (19). Mx2 is a type I interferon stimulated gene. On both study days, expression of these two marker genes was significantly lower in B/HPIV3/S and B/HPIV3/S-2P immunized animals, indicating that both vaccine candidates protected against inflammatory responses after challenge. Moreover, in B/HPIV3/S-2P immunized animals, Mx2 expression remained at baseline, while in B/HPIV3/S immunized animals low but significantly higher levels were present, suggesting a stronger efficacy of protection of B/HPIV3/S-2P. In addition, we evaluated a panel of 12 additional immune response genes, including pro-inflammatory cytokines C-C-ligand (CCL) 17, CCL22, interleukine (IL)-12p40, IL-1B, IL2, and tumor necrosis factor alpha (TNF-A); immunoregulatory factors IL-10 and IL-6, anti-inflammatory factors IL-13 and IL-4, and IL-21 and interferon (IFN)-G (FIG. 4C). Most genes were expressed at a higher level in B/HPIV3 vector control immunized animals compared to B/HPIV3/S and B/HPIV3/S-2P immunized animals on day 3 after challenge. Thus, SARS-COV-2 challenge induced a strong inflammatory cytokine response in B/HPIV3 immunized animals, but not in B/HPIV3/S and B/HPIV3/S-2P immunized animals.

Lungs and nasal turbinates obtained on days 3 and 5 post-challenge were homogenized, and assayed by limiting dilution on Vero E6 cells to quantify SARS-COV-2 challenge virus replication (FIGS. 5C, D). In the nasal turbinates, animals immunized with the empty vector had high mean titers of 7.0 log₁₀ and 5.0 log₁₀ TCID₅₀/g of challenge SARS-COV-2 on days 3 and 5 (FIG. 5C). In animals immunized with B/HPIV3/S, the mean challenge virus titers in the nasal turbinates were about 20-fold lower on day 3 and undetectable on day 5. In animals immunized with B/HPIV3/S-2P, challenge virus was undetectable in nasal turbinates on both days. In the lungs, mean titers of 6.6 log₁₀ TCID₅₀/g and 4.5 log₁₀ TCID₅₀/g of SARS-COV-2 were detected on days 3 and 5 in animals immunized with the empty vector (FIG. 5D). Remarkably, challenge virus was undetectable in the lungs of B/HPIV3/S- and B/HPIV3/S-2P-immunized hamsters. Thus, B/HPIV3 expressing SARS-COV-2 S is highly protective against SARS-COV-2 challenge, and the prefusion stabilization substantially enhanced immunogenicity and protective efficacy.

### Example 3: Intranasal Parainfluenza Vector Vaccines Expressing Prefusion-Stabilized Versions of the SARS-COV-2 S Protein Protect Against SARS-COV-2 Derived from Three Major Genetic Lineages in Hamsters

This example describes the side-by-side characterization of two recombinant bovine/human parainfluenza viruses B/HPIV3/S-2P and B/HPIV3/S-6P (FIG. 1A), expressing prefusion-stabilized SARS-CoV-2 spike proteins, as candidate vaccines for SAR-COV-2.

### In-Vitro Characterization of B/HPIV3 Vector Vaccine Candidates B/HPIV3/S-2P and B/HPIV3/S-6P Expressing Prefusion-Stabilized Versions of the SARS-COV-2 S Protein.

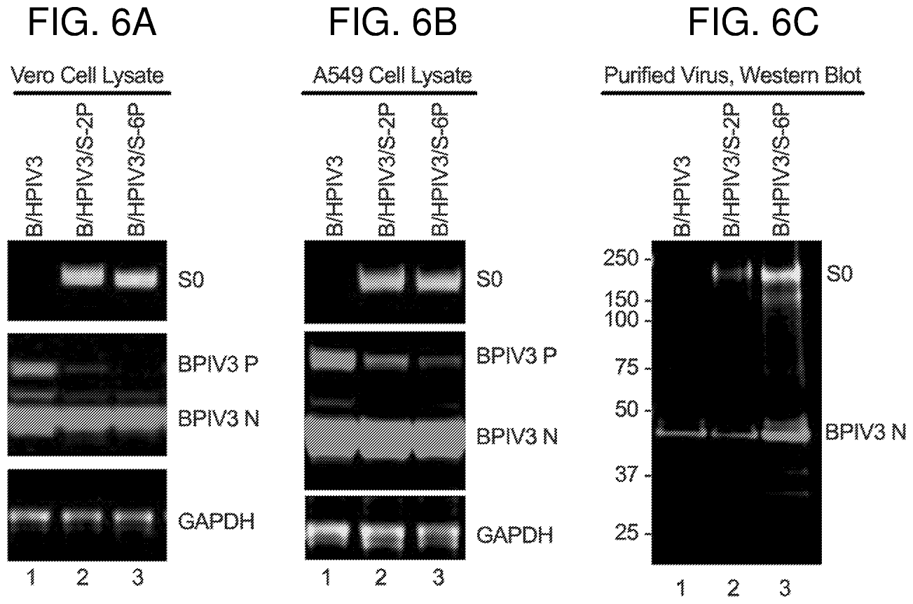

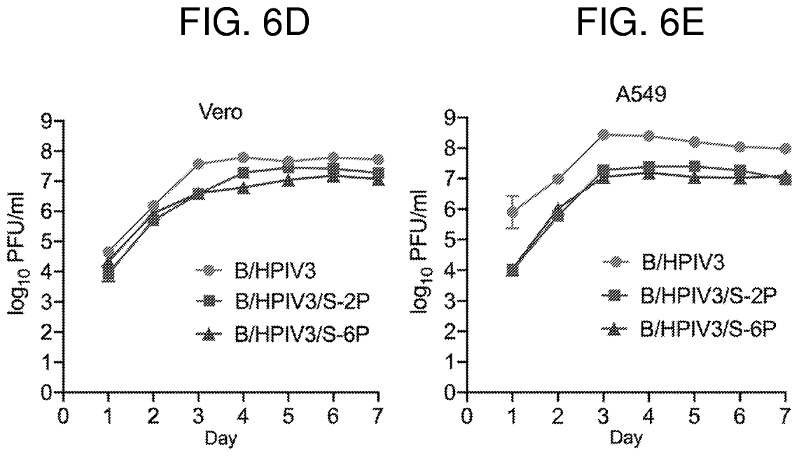

To characterize and compare the expression of the prefusion-stabilized versions of the SARS-COV-2 S protein by B/HPIV3 proteins in vitro, Vero and human lung epithelial A549 cells were infected with B/HPIV3, B/HPIV3/S-2P, or B/HPIV3/S-6P at an MOI of 1 plaque forming unit (PFU) per cell. Cell lysates were prepared 48 hours after infection, and analyzed by SDS-PAGE and Western blotting, using antisera to detect SARS-COV-2 S or PIV3 proteins. (FIGS. 6A, 6B). In lysates from both cell lines, the S protein was detectable as a high-molecular band, consistent in size with uncleaved SO precursor protein (FIGS. 6A and 6B, lanes 2, 3).

To evaluate possible incorporation of the SARS-COV-2 S or S-2P protein into B/HPIV3 particles, Vero-grown viruses were purified by centrifugation through sucrose gradients, and the protein composition was analyzed by gel electrophoresis with Western blotting (FIG. 6C). Immunostaining identified this band as SO (FIG. 6C), indicating that the prefusion-stabilized versions were incorporated in the B/HPIV3 vector particles. Multicycle replication of B/HPIV3/S-2P and B/HPIV3/S-6P in Vero cells was efficient and overall similar to that of B/HPIV3, confirming that the presence of the 3.8 kb S-2P or S-6P inserts did not slow or reduce the replication of the B/HPIV3 vector in vitro in Vero cells. However, in human airway epithelial A549 cells, replication of B/HPIV3/S-2P and B/HPIV3/S-6P was reduced compared to the B/HPIV3 empty vector by about 10-fold at all time points (FIGS. 6D, 6E).

Immunization of Hamsters with the B/HPIV3/S Viruses Expressing Prefusion-Stabilized Versions of the SARS-COV-2 S Protein.

To evaluate the replication and immunogenicity of the B/HPIV3/S-2P and B/HPIV3/S-6P vaccine candidates, hamsters in groups of 27 were inoculated intranasally with 5 log₁₀ PFU of the B/HPIV3/S-2P and B/HPIV3/S-6P vaccine candidates, or with B/HPIV3 empty vector control. On days 3, 5, and 7 after inoculation, 5 hamsters per group were euthanized to evaluate vector replication in the respiratory tract: nasal turbinates and lungs were harvested from 5 animals and tissue homogenates were prepared and analyzed by immunoplaque assay (FIGS. 7A, B).

As typically observed, including in the first hamster study described above, B/HPIV3 replicated to high mean peak titers (6.5 and 5.9 log₁₀ PFU/g on day 3) in the nasal turbinates and lungs, respectively (FIGS. 7A, B). In the nasal turbinates, titers of the B/HPIV3 empty vector were higher on day 3 and decreased about ten-fold by day 5, and further decreased by about 5 log₁₀ by day 7. In contrast, B/HPIV3/S-2P and B/HPIV3/S-6P titers were about 30- and 100-fold lower than that of the empty vector control in nasal turbinates on day 3 (5.0 and 4.4 log₁₀ PFU/g vs 6.5 log₁₀ PFU/g for B/HPIV3), but increased and were significantly higher than B/HPIV3 titers on day 5 (6.4 log₁₀ and 6.1 log₁₀ PFU/g for B/HPIV3/S-2P and B/HPIV3/S-6P vs 5.2 log₁₀ PFU/g for B/HPIV3). This confirmed the previous observation that the presence of the large S insert in the B/HPIV3 genome delayed virus replication in the upper respiratory tract of hamsters. However, mean nasal peak titers of all 3 viruses, independent of the study day (6.5 log₁₀ PFU/g for B/HPIV3; 6.4 log₁₀ and 6.1 log₁₀ PFU/g for B/HPIV3/S-2 and B/HPIV3/S-6P) were not significantly different.

In the lungs, B/HPIV3 replication remained at a high level over both days (5.9 log₁₀ PFU/g and 5.3 log₁₀ PFU/g on days 3 and 5). Similar to the findings in the nasal turbinates, mean titers of B/HPIV3/S-2P and B/HPIV3/S-6P (4.7 log₁₀ and 5.0 log₁₀ PFU/g) were also lower than those of the B/HPIV3 empty vector on day 3. By day 7, a low level of B/HPIV3/S-6P was still detectable in four of 5 hamsters, but undetectable in the other groups.

The serum antibody response was evaluated 28 days after intranasal immunization in the remaining animals (n=12 animals per group). SARS-COV-2-neutralizing antibody titers were measured by an ND₅₀ assay against SARS-COV-2, strain WA1/2020, a representative of lineage A with a with an S amino acid sequence identical to that expressed by B/HPIV3/S (FIG. 7C). As expected, SARS-COV-2 neutralizing antibodies were not detected in animals immunized with B/HPIV3 empty vector. B/HPIV3/S-2P and B/HPIV3/S-6P induced a robust response of SARS-COV-2 serum neutralizing antibodies [geometric mean reciprocal ND₅₀ titers: 1.9 log₁₀ (1:79) and 2.1 log₁₀ (1:126) for B/HPIV3/S-2P and B/HPIV3/S-6P; FIG. 7C].

In addition, SARS-COV-2-specific serum IgG was measured by ELISA using as antigen purified preparations of the secreted form of the S-2P protein (FIG. 7D) and a fragment (aa 319-591) of the SARS-CoV-2 S protein bearing the RBD (FIG. 7E). Both B/HPIV3/S-2P and B/HPIV3/S-6P induced a very robust serum IgG response to the S antigen. Interestingly, the RBD IgG response induced by B/HPIV3/S-6P was significantly stronger than that induced by B/HPIV3/S-2P.

B/HPIV3/S-2P and B/HPIV3/S-6P Vaccine Candidates Expressing Prefusion-Stabilized Versions of the SARS-COV-2 S Protein Protect Against Intranasal Challenge with SARS-COV-2 Isolates of Three Major Genetic Lineages.

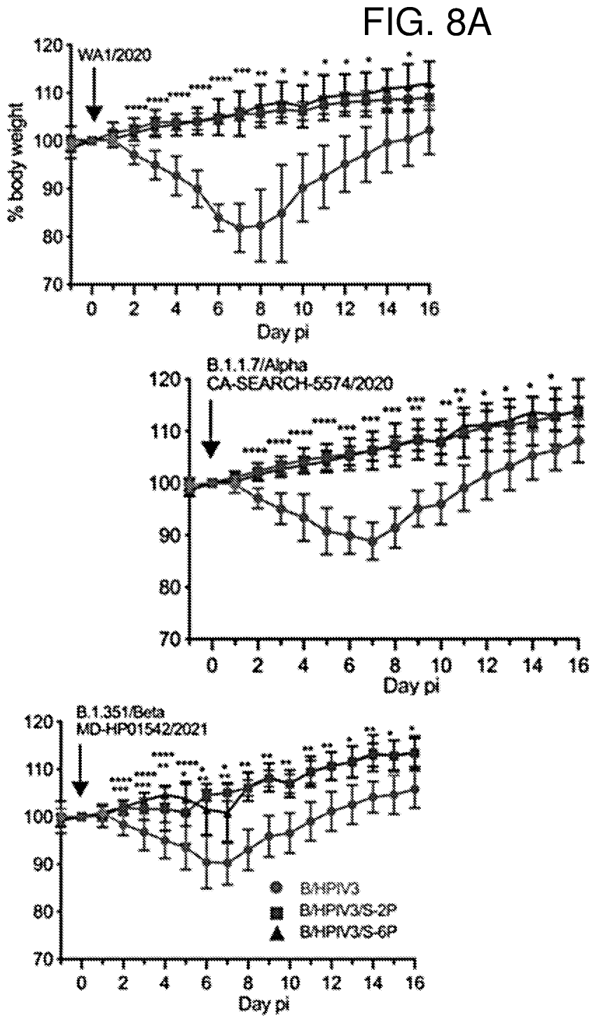

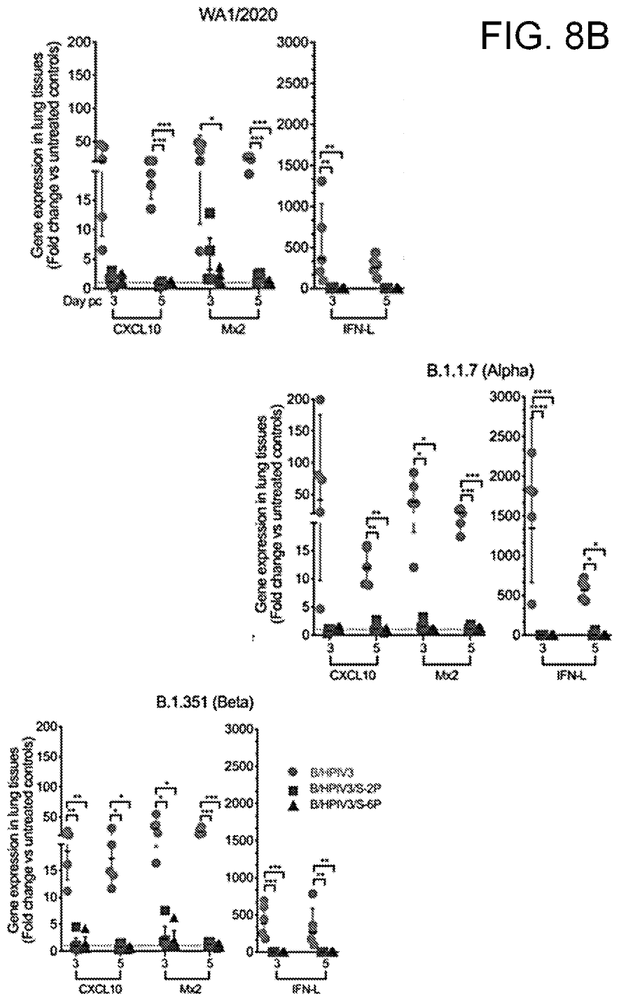

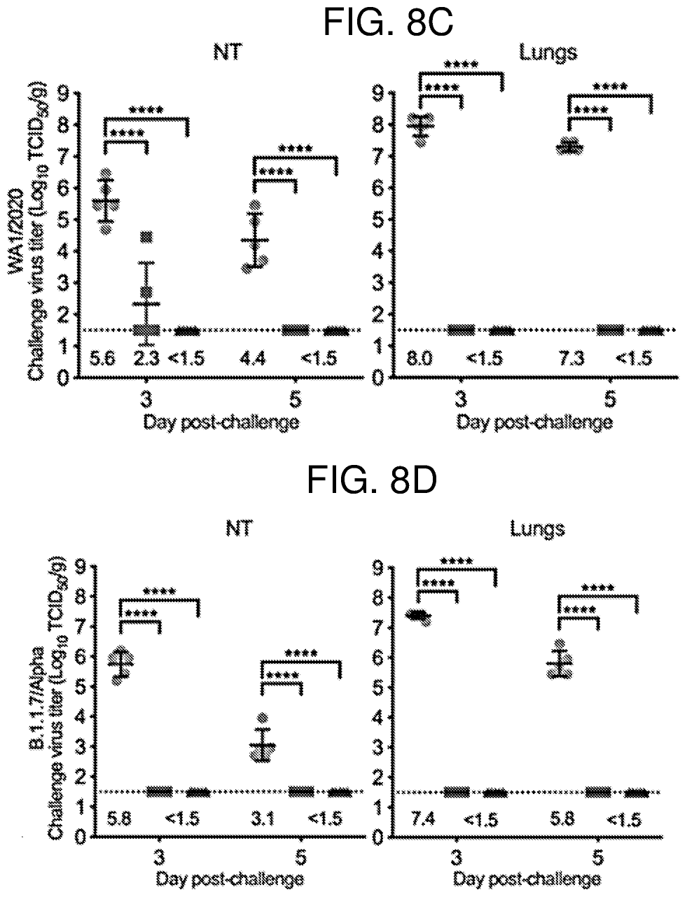

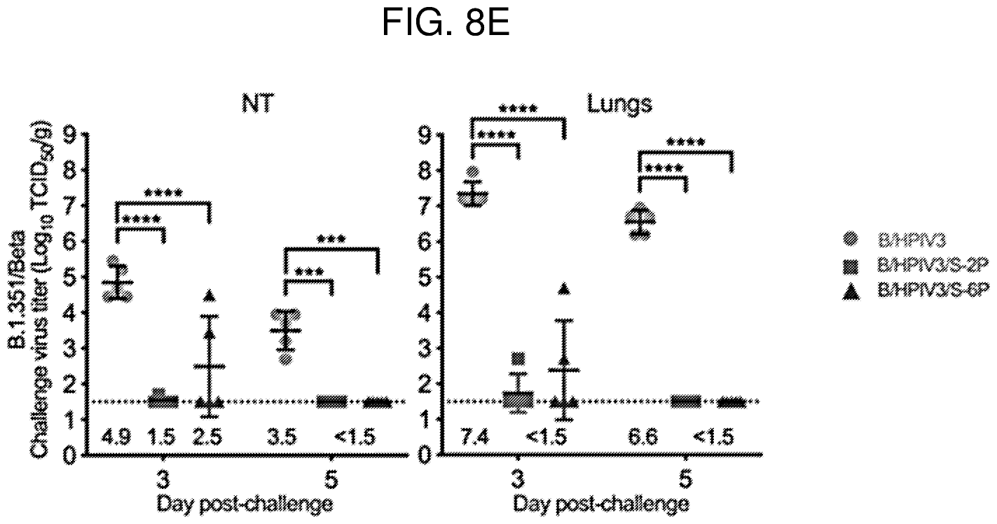

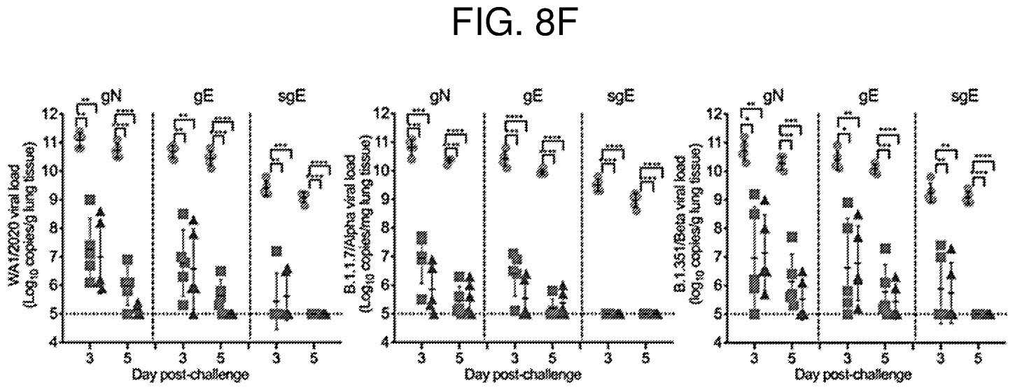

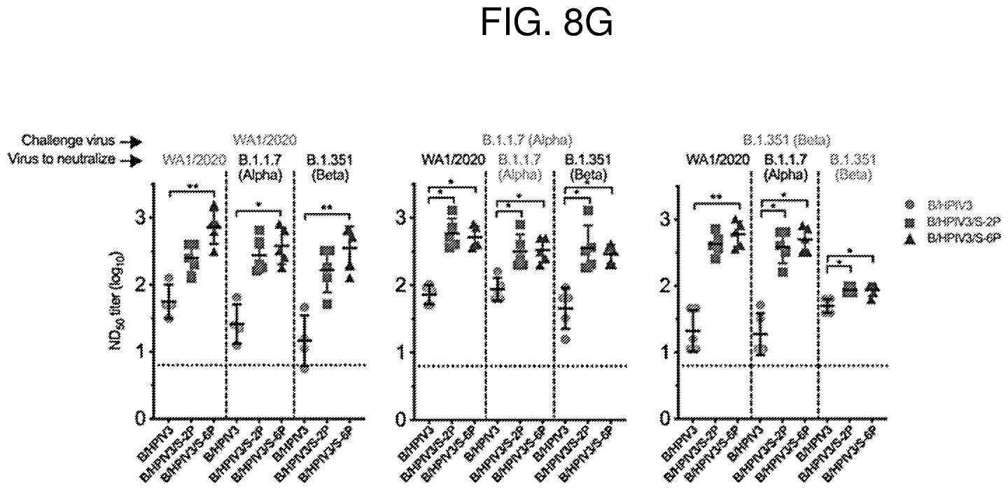

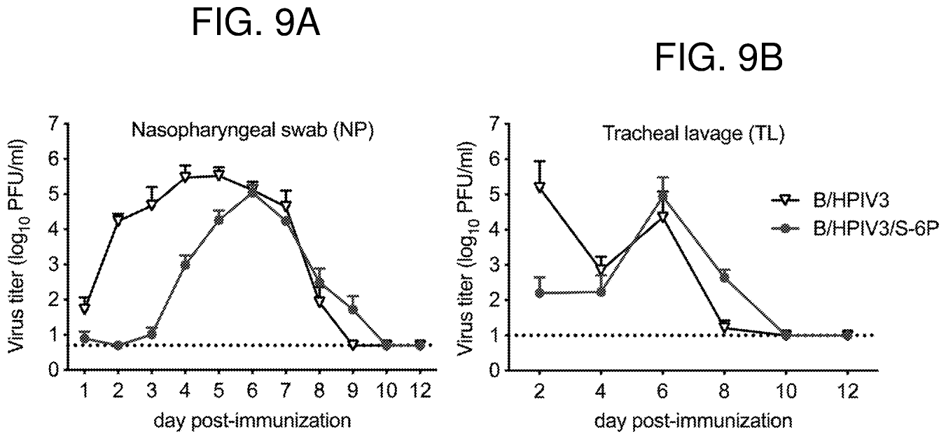

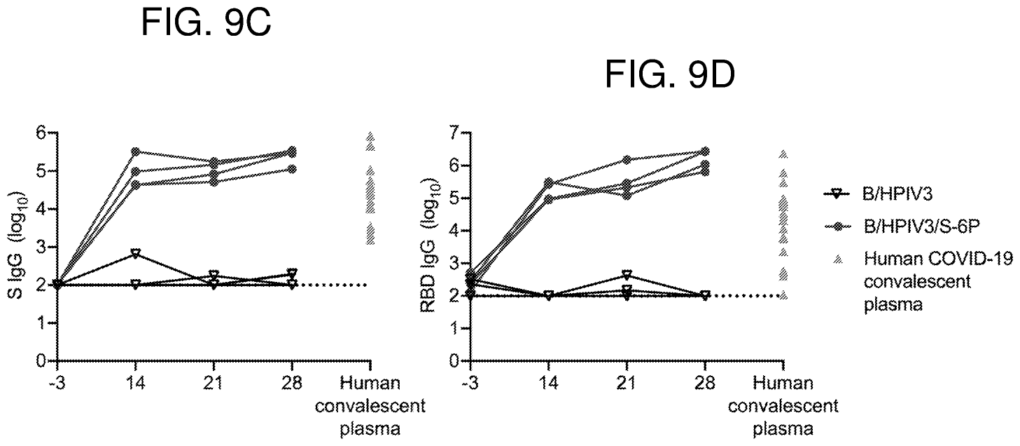

To evaluate the breadth of protection against major SARS-COV-2 variants of concern, an additional experiment was performed. Hamsters in groups of 45 were immunized intranasally with a single 5 log₁₀ PFU dose of B/HPIV3 vector control, B/HPIV3/S-2P or B/HPIV3/S-6P as described above. On day 33 after immunization, each immunized group was divided into 3 groups of 15 animals, and challenged intranasally with 4.5 log₁₀ TCID₅₀ per animal of SARS-COV-2, isolate WA1-USA/2020 (lineage A), isolate USA/CA_CDC_5574/2020 (lineage B.1.1.7/Alpha), or USA/MD-HP01542/2021 (lineage B.1.351/Beta) from preparations that had been subjected to complete-genome deep sequencing to confirm their integrity. Animals were observed for clinical symptoms and monitored for weight loss (FIG. 8A). In all three challenge groups, animals immunized with the empty B/HPIV3 vector exhibited weight loss over the first 7 days after challenge [18%, 11%, and 10% average loss by day 7 post-challenge with WA1/2020 (lineage A), or isolates of lineages B.1.1.7/Alpha or B.1.351/Beta, respectively], while animals immunized with B/HPIV3/S-2P and B/HPIV3/S-6P generally continued to gain body weight. Five animals from each group were euthanized on days 3 and 5 post-challenge, and lungs and nasal turbinates were collected to evaluate SARS-COV-2 challenge virus replication (FIG. 8B).

In the nasal turbinates, animals immunized with the empty B/HPIV3 vector had high mean peak titers of 5.6 log 10, 5.8 log_{10,} and 4.9 log₁₀ TCID5/g of lineage A, B.1.1.7/Alpha or B.1.351/Beta SARS-COV-2 challenge virus on day 3. On day 5, challenge virus titers in nasal turbinates were generally lower by about 1.2-1.7 log₁₀ TCID₅₀ compared to day 3 (FIG. 8B). Among the B/HPIV3/S-2P immunized animals, only two of five had low levels of WA1/2020 (lineage A) detectable in nasal turbinates on day 3, and a single animal had virus of the B.1.351/Beta lineage detectable on day 3; among the B/HPIV3/S-6P-immunized animals, only two had virus of the B.1.351/Beta lineage detectable in the nasal turbinates on day 3. On day 5, challenge viruses from all three lineages were undetectable in nasal turbinates of B/HPIV3/S-2P and B/HPIV3/S-6P immunized animals.

In the lungs of empty B/HPIV3 vector immunized animals, high titers of 7.4 log₁₀ or 8.0 log₁₀ TCID₅₀/g of the challenge viruses were detectable. Remarkably, B/HPIV3/S-2P and B/HPIV3/S-6P immunized animals had no lineage A and B.1.1.7/Alpha virus detectable in the lungs on either day, while virus of the B.1.351/Beta lineage was detectable at low titers in 3 and 2 of five B/HPIV3/S-2P and B/HPIV3/S-6P immunized hamsters on day 3 after challenge (FIG. 8B). Thus, B/HPIV3 vectors expressing prefusion-stabilized versions of SARS-COV-2 S are highly protective against SARS-COV-2 challenge of three major lineages.

B/HPIV3/S-6P Expressing Prefusion-Stabilized Versions of the SARS-COV-2 S Protein Replicates in Nonhuman Primates after Intranasal/Intratracheal Immunization, and Induce Serum IgG Titers to SARS-COV-2 S Comparable to those in Human Convalescent Plasma Samples.

B/HPIV3/S-6P was further evaluated in rhesus macaques. Rhesus macaques were immunized intranasally and intratracheally with 6 log₁₀ PFU of B/HPIV3/S-6P or B/HPIV3 control. To evaluate virus replication, nasopharyngeal swabs and tracheal lavages were performed over 12 days after immunization. Sera were collected before immunization and on days 14, 21, and 28 to evaluate the immune response to the SARS-COV-2 S protein by IgG ELISA. Replication of B/HPIV3 in rhesus macaques was very robust, as previously observed [see for example (20)], and reached peak titers on day 5 in the upper respiratory tract, and on day 6 in the lower respiratory tract. Replication of B/HPIV3/S-6P was also robust. In the upper respiratory tract, B/HPIV3/S-6P peak titers were detected on day 7, about two days after the peak of replication of the empty B/HPIV3 vector control. In the lower respiratory tract, high titers were detectable on day 2 after immunization, and again on day 6 post-immunization. Thus, the presence of the additional gene expressing S-6P did not seem to substantially affect the ability of B/HPIV3 to replicate in primate hosts.

Serum IgG titers to the S protein and to the S RBD were determined by ELISA, using a soluble form of the S protein as antigen, or a fragment (aa 319-591) of the S protein bearing the RBD. No S or RBD specific IgG was detectable in B/HPIV3 immunized animals, while in B/HPIV3/S-6P immunized animals, a strong serum IgG response to both antigens was detected as early as 14 days after intranasal/intratracheal immunization. By day 28 after immunization, the immune response was very uniform and at a level comparable with highest quartile of S-or RBD-specific IgG titers detected in human plasma samples from de-identified donors with past COVID-19 infection. Based on the robust replication and strong immunogenicity in nonhuman primates, B/HPIV3/S-6P is a suitable candidate for clinical evaluation as a pediatric intranasal vaccine against HPIV3 and SARS-COV-2.

### Discussion

To gain more complete control of SARS-COV-2, safe and effective vaccines are needed for all age groups. Even though SARS-COV-2 infections in children are generally milder than in adults, SARS-COV-2 causes clinical disease and replicates to high titers in pediatric patients, and viral loads seem to correlate well with disease severity in this population (21-24). A pediatric vaccine that directly induces a robust local respiratory tract immune response in addition to a systemic response has the potential to strongly restrict SARS-COV-2 at its primary site of infection and shedding, which should enhance protection and restrict community transmission.

B/HPIV3 was used to express three versions of the SARS-COV-2 S protein: namely, the unmodified wild-type S protein, and the stabilized prefusion versions S-2P and S-6P (both with ablated S1/S2 cleavage site), resulting in the viruses B/HPIV3/S, B/HPIV3/S-2P, and B/HPIV3/S-6P.

To evaluate the effects of prefusion stabilization by 2 P mutations and ablation of the S1/S2 cleavage site on expression and immunogenicity of full-length S protein of SARS-COV-2, B/HPIV3/S was included as a control, which expressed the unmodified wild-type S protein. When B/HPIV3/S-2P and B/HPIV3/S were compared in side-by-side studies, it was found that in vitro expression of the prefusion-stabilized noncleaved S-2P version was increased. Since antigens had been denatured and reduced prior to analysis, ablating conformational epitopes, the quantitative differences detected by Western blot should reflect differences in protein expression, rather than differences in antibody reactivity with S-2P compared to S.

Prefusion stabilization and lack of cleavage was associated with significantly better immunogenicity in the hamster model: compared to B/HPIV3/S, B/HPIV3/S-2P replicated to similar or lower titers in the respiratory tract of hamsters while inducing significantly higher serum ELISA IgG titers to prefusion-stabilized S (13-fold higher) and the RBD (10-fold), as well as higher (9-fold) titers of SARS-COV-2-neutralizing serum antibodies to the SARS-COV-2 isolate WA1/2020, a representative of the SARS-COV-2 lineage A with an S amino acid sequence identical to that expressed by B/HPIV3/S. Thus, prefusion stabilization and lack of cleavage of the full-length S protein with complete cytoplasmic/transmembrane domain resulted in increased immunogenicity and provided for a broad neutralizing activity against major SARS-COV-2 variants. In a comparison in hamsters of the protective efficacy of B/HPIV3/S and B/HPIV3/S-2P against high-dose intranasal SARS-COV-2 challenge with strain WA1/2020 (lineage A), infectious challenge virus was not detected in respiratory tissues of B/HPIV3/S-2P-immunized hamsters, whereas protection in the upper respiratory tract of animals immunized with B/HPIV3/S, bearing the non- stabilized version of S, was less than complete, at least on day 3 after challenge. Even though immunization with B/HPIV3/S did not entirely protect the animals from challenge virus infection, it reduced challenge virus replication substantially in magnitude and duration, prevented weight loss and pulmonary induction of inflammatory cytokines in hamsters after challenge, highlighting the overall potency of the B/HPIV3 vector platform. Notably, serum antibodies induced in hamsters by the prefusion-stabilized version expressed by B/HPIV3/S-2P also were functional in neutralizing variants of concern of lineages B.1.1.7 (UK lineage) and B.1.351/Beta (South Africa lineage). Moreover, immunization with B/HPIV3/S-2P or B/HPIV3/S-6P was protective against challenge with the SARS-COV-2 lineage A strain WA1/2020, with an amino acid sequence of the S protein identical to that of the nonstabilized version expressed by B/HPIV3/S; immunization with B/HPIV3/S-2P or B/HPIV3/S-6P also induced complete protection in the hamster model against challenge with an isolate of lineage B.1.1.7/Alpha (UK lineage), and substantial protection against a B.1.351/Beta isolate (South Africa lineage).

Unexpectedly, the S-2P and the S-6P versions, but not the wild-type S version, were efficiently packaged into the B/HPIV3 vector particles. Why prefusion stabilization and/or ablation of the furin cleavage site resulted in efficient incorporation is not known. In the case of RSV F protein, the unmodified wild-type protein was not packaged significantly into the vector particle, and required substitution of its transmembrane and cytoplasmic tail domains with those of the vector F protein. For RSV F, packaging into the vector particle resulted in a large increase in the amount and neutralizing capability of serum antibodies induced by immunization, an effect that was similar in quality and magnitude to that of stabilization of RSV F in the prefusion conformation (20, 28). It similarly may be that the packaging of the S-2P and S-6P proteins into the B/HPIV3 particle contributed, in addition to prefusion stabilization, to their greater immunogenicity compared to wild-type S protein.

Although expression of the S, S-2P, or S-6P proteins by B/HPIV3 had little or no effect on the efficiency of vector replication in vitro, B/HPIV3/S replicated to a 10-fold higher titer in the hamster lungs compared to B/HPIV3 and B/HPIV3/S-2P. In the case of B/HPIV3/S, the SARS-COV-2 S protein was unmodified and would have retained its functions, raising the possibly that it might have contributed to infection. However, the unmodified S protein was packaged into vector particles only in trace amounts. Therefore, it seems unlikely that it would have made a significant contribution to infection by vector particles. The possibility remains that unmodified S protein accumulating on the cell surface might have contributed to cell-to-cell spread. In the case of B/HPIV3/S-2P and B/HPIV3/S-6P, prefusion stabilization and ablation of the cleavage site in S-2P and S-6P would render these proteins functionally inactive for viral entry and fusion, precluding any contribution to vector tropism. Importantly, for both B/HPIV3/S and B/HPIV/S-2P, no vector replication was detected outside the respiratory tract in the hamster model, indicating that the tropism of the B/HPIV3 vector was unchanged in either case.

Based on the very promising results in the hamster challenge model provided herein, B/HPIV3/S-2P and B/HPIV3/S-6P are candidates for being advanced to a Phase 1 pediatric clinical studies, and are expected to be safe and efficacious against both SARS-COV-2 and HPIV3 in infants and young children.

### REFERENCES FOR EXAMPLES 1-3

1. Wrapp D, Wang N, Corbett K S, Goldsmith J A, Hsieh C L, Abiona O, Graham B S, Mclellan JS . 2020. Cryo-EM Structure of the 2019-nCOV Spike in the Prefusion Conformation. bioRxiv doi: 10.1101/2020.02.11.944462.
2. Hsich C L, Goldsmith J A, Schaub J M, DiVenere A M, Kuo H C, Javanmardi K, Le K C, Wrapp D, Lee A G, Liu Y, Chou C W, Byrne P O, Hjorth C K, Johnson N V, Ludes-Meyers J, Nguyen A W, Park J, Wang N, Amengor D, Lavinder J J, Ippolito G C, Maynard J A, Finkelstein I J, Mclellan J S. 2020. Structure-based design of prefusion-stabilized SARS-COV-2 spikes. Science 369:1501-1505.
3. Liang B, Surman S, Amaro-Carambot E, Kabatova B, Mackow N, Lingemann M, Yang L, McLellan J S, Graham B S, Kwong P D, Schaap-Nutt A, Collins PL, Munir S. 2015. Enhanced Neutralizing Antibody Response Induced by Respiratory Syncytial Virus Prefusion F Protein Expressed by a Vaccine Candidate. J Virol 89:9499-510.
4. Munir S, Le Nouen C, Luongo C, Buchholz U J, Collins P L, Bukreyev A. 2008. Nonstructural proteins 1 and 2 of respiratory syncytial virus suppress maturation of human dendritic cells. J Virol 82:8780-96.
5. Subbarao K, Mcauliffe J, Vogel L, Fahle G, Fischer S, Tatti K, Packard M, Shich W J, Zaki S, Murphy B. 2004. Prior infection and passive transfer of neutralizing antibody prevent replication of severe acute respiratory syndrome coronavirus in the respiratory tract of mice. J Virol 78:3572-7.
6. Liang B, Munir S, Amaro-Carambot E, Surman S, Mackow N, Yang L, Buchholz U J, Collins P L, 6. Schaap-Nutt A. 2014. Chimeric bovine/human parainfluenza virus type 3 expressing respiratory syncytial virus (RSV) F glycoprotein: effect of insert position on expression, replication, immunogenicity, stability, and protection against RSV infection. J Virol 88:4237-50.
7. Wrapp D, Wang N, Corbett K S, Goldsmith J A, Hsich C L, Abiona O, Graham B S, Mclellan J S. 2020. Cryo-EM structure of the 2019-nCOV spike in the prefusion conformation. Science 367:1260-1263.
8. Durbin A P, Hall S L, Siew J W, Whitehead S S, Collins P L, Murphy B R. 1997. Recovery of infectious human parainfluenza virus type 3 from cDNA. Virology 235:323-32.
9. Kozak M. 1987. An analysis of 5′-noncoding sequences from 699 vertebrate messenger RNAs. Nucleic Acids Res 15:8125-48.
10. Buchholz U J, Bukreyev A, Yang L, Lamirande E W, Murphy B R, Subbarao K, Collins P L. 2004. Contributions of the structural proteins of severe acute respiratory syndrome coronavirus to protective immunity. Proc Natl Acad Sci U S A 101:9804-9.
11. Walls A C, Park Y J, Tortorici M A, Wall A, McGuire A T, Veesler D. 2020. Structure, Function, and Antigenicity of the SARS-COV-2 Spike Glycoprotein. Cell 181:281-292 e6.
12. Reed LJaM, H. 1938. A simple method of estimating fifty per cent endpoints. Am J Epidemiol 27:493-497.
13. Sanchez-Felipe L, Vercruysse T, Sharma S, Ma J, Lemmens V, Van Looveren D, Arkalagud Javarappa M P, Boudewijns R, Malengier-Devlies B, Liesenborghs L, Kaptein SJF, De Keyzer C, Bervoets L, Debaveye S, Rasulova M, Seldeslachts L, Li L H, Jansen S, Yakass M B, Verstrepen B E, Boszormenyi KP, Kiemenyi-Kayere G, van Driel N, Quaye O, Zhang X, Ter Horst S, Mishra N, Deboutte W, Matthijnssens J, Coelmont L, Vandermeulen C, Heylen E, Vergote V, Schols D, Wang Z, Bogers W, Kuiken T, Verschoor E, Cawthorne C, Van Laere K, Opdenakker G, Vande Velde G, Weynand B, Teuwen D E, Matthys P, Neyts J, Jan Thibaut H, Dallmeier K. 2021. A single-dose live-attenuated YF17D-vectored SARS-COV-2 vaccine candidate. Nature 590:320-325.
14. Espitia C M, Zhao W, Saldarriaga O, Osorio Y, Harrison L M, Cappello M, Travi BL, Melby P C. 2010. Duplex real-time reverse transcriptase PCR to determine cytokine mRNA expression in a hamster model of New World cutaneous leishmaniasis. BMC Immunol 11:31.
15. Schmidt A C, McAuliffe J M, Huang A, Surman S R, Bailly J E, Elkins W R, Collins P L, Murphy B R, Skiadopoulos M H. 2000. Bovine parainfluenza virus type 3 (BPIV3) fusion and hemagglutinin-neuraminidase glycoproteins make an important contribution to the restricted replication of BPIV3 in primates. J Virol 74:8922-9.
16. Wu F, Zhao S, Yu B, Chen Y M, Wang W, Song Z G, Hu Y, Tao Z W, Tian J H, Pei Y Y, Yuan M L, Zhang Y L, Dai F H, Liu Y, Wang Q M, Zheng J J, Xu L, Holmes E C, Zhang Y Z. 2020. A new coronavirus associated with human respiratory disease in China. Nature 579:265-269.
17. Rambaut A, Holmes E C, O'Toole A, Hill V, McCrone J T, Ruis C, du Plessis L, Pybus O G. 2020. A dynamic nomenclature proposal for SARS-COV-2 lineages to assist genomic epidemiology. Nat Microbiol 5:1403-1407.
18. Galloway S E, Paul P, MacCannell D R, Johansson M A, Brooks J T, MacNeil A, Slayton R B, Tong S, Silk B J, Armstrong G L, Biggerstaff M, Dugan V G. 2021. Emergence of SARS-COV-2 B.1.1.7 Lineage - United States, December 29, 2020-January 12, 2021. MMWR Morb Mortal Wkly Rep 70:95-99.
19. Yang Y, Shen C, Li J, Yuan J, Wei J, Huang F, Wang F, Li G, Li Y, Xing L, Peng L, Yang M, Cao M, Zheng H, Wu W, Zou R, Li D, Xu Z, Wang H, Zhang M, Zhang Z, Gao GF, Jiang C, Liu L, Liu Y. 2020. Plasma IP-10 and MCP-3 levels are highly associated with disease severity and predict the progression of COVID-19. J Allergy Clin Immunol 146:119-127 e4.
20. Liang B, Ngwuta JO, Surman S, Kabatova B, Liu X, Lingemann M, Liu X, Yang L, Herbert R, Swerczek J, Chen M, Moin S M, Kumar A, Mclellan J S, Kwong P D, Graham B S, Collins P L, Munir S. 2017. Improved Prefusion Stability, Optimized Codon Usage, and Augmented Virion Packaging Enhance the Immunogenicity of Respiratory Syncytial Virus Fusion Protein in a Vectored-Vaccine Candidate. J Virol 91.
21. Zachariah P, Johnson C L, Halabi K C, Ahn D, Sen A I, Fischer A, Banker S L, Giordano M, Manice C S, Diamond R, Sewell T B, Schweickert A J, Babineau J R, Carter R C, Fenster D B, Orange J S, McCann T A, Kernie S G, Saiman L, Columbia Pediatric C-MG. 2020. Epidemiology, Clinical Features, and Disease Severity in Patients With Coronavirus Disease 2019 (COVID-19) in a Children's Hospital in New York City, New York. JAMA Pediatr 174:e202430.
22. Zachariah P, Halabi K C, Johnson C L, Whitter S, Sepulveda J, Green D A. 2020. Symptomatic Infants Have Higher Nasopharyngeal SARS-COV-2 Viral Loads but Less Severe Disease Than Older Children. Clin Infect Dis 71:2305-2306.
23. Heald-Sargent T, Muller W J, Zheng X, Rippe J, Patel A B, Kociolek L K. 2020. Age-Related Differences in Nasopharyngeal Severe Acute Respiratory Syndrome Coronavirus 2 (SARS-COV-2) Levels in Patients With Mild to Moderate Coronavirus Disease 2019 (COVID-19). JAMA Pediatr 174:902-903.
24. Liguoro I, Pilotto C, Bonanni M, Ferrari ME, Pusiol A, Nocerino A, Vidal E, Cogo P. 2020. SARS-COV-2 infection in children and newborns: a systematic review. Eur J Pediatr 179:1029-1046.
25. Karron RA, Thumar B, Schappell E, Surman S, Murphy BR, Collins PL, Schmidt AC. 2012. 25. Evaluation of two chimeric bovine-human parainfluenza virus type 3 vaccines in infants and young children. Vaccine 30:3975-81.
26. Bernstein DI, Malkin E, Abughali N, Falloon J, Yi T, Dubovsky F, Investigators M-C. 2012. Phase I study of the safety and immunogenicity of a live, attenuated respiratory syncytial virus and parainfluenza virus type 3 vaccine in seronegative children. Pediatr Infect Dis J 31:109-14.
27. Liang B, Matsuoka Y, Le Nouen C, Liu X, Herbert R, Swerczek J, Santos C, Paneru M, Collins P L, Buchholz U J, Munir S. 2020. A parainfluenza virus vector expressing the respiratory syncytial virus (RSV) prefusion F protein is more effective than RSV for boosting a primary immunization with RSV. J Virol doi: 10.1128/JVI.01512-20.
28. Liang B, Ngwuta J O, Herbert R, Swerczek J, Dorward D W, Amaro-Carambot E, Mackow N, Kabatova B, Lingemann M, Surman S, Yang L, Chen M, Moin S M, Kumar A, Mclellan J S, Kwong P D, Graham B S, Schaap-Nutt A, Collins PL, Munir S. 2016. Packaging and Prefusion Stabilization Separately and Additively Increase the Quantity and Quality of Respiratory Syncytial Virus (RSV)-Neutralizing Antibodies Induced by an RSV Fusion Protein Expressed by a Parainfluenza Virus Vector. J Virol 90:10022-10038.
29. Kirchdoerfer R N, Wang N, Pallesen J, Wrapp D, Turner H L, Cottrell C A, Corbett K S, Graham B S, Mclellan J S, Ward A B. 2018. Stabilized coronavirus spikes are resistant to conformational changes induced by receptor recognition or proteolysis. Sci Rep 8:15701.
30. Pallesen J, Wang N, Corbett K S, Wrapp D, Kirchdoerfer R N, Turner H L, Cottrell C A, Becker M M, Wang L, Shi W, Kong W P, Andres E L, Kettenbach A N, Denison M R, Chappell J D, Graham B S, Ward A B, Mclellan J S. 2017. Immunogenicity and structures of a rationally designed prefusion MERS-COV spike antigen. Proc Natl Acad Sci U S A 114:E7348-E7357.
31. Corbett K S, Edwards D K, Leist S R, Abiona O M, Boyoglu-Barnum S, Gillespie R A, Himansu S, Schafer A, Ziwawo C T, DiPiazza A T, Dinnon K H, Elbashir S M, Shaw C A, Woods A, Fritch E J, Martinez D R, Bock K W, Minai M, Nagata B M, Hutchinson G B, Wu K, Henry C, Bahi K, Garcia-Dominguez D, Ma L, Renzi I, Kong W P, Schmidt S D, Wang L, Zhang Y, Phung E, Chang L A, Loomis R J, Altaras N E, Narayanan E, Metkar M, Presnyak V, Liu C, Louder M K, Shi W, Leung K, Yang E S, West A, Gully K L, Stevens L J, Wang N, Wrapp D, Doria-Rose N A, Stewart-Jones G, Bennett H, et al. 2020. SARS-COV-2 mRNA vaccine design enabled by prototype pathogen preparedness. Nature doi: 10.1038/s41586-020-2622-0.
32. Hoffmann M, Kleine-Weber H, Schroeder S, Kruger N, Herrler T, Erichsen S, Schiergens T S, Herrler G, Wu N H, Nitsche A, Muller M A, Drosten C, Pohlmann S. 2020. SARS-COV-2 Cell Entry Depends on ACE2 and TMPRSS2 and Is Blocked by a Clinically Proven Protease Inhibitor. Cell 181:271-280 e8.
33. Wrobel A G, Benton D J, Xu P, Roustan C, Martin S R, Rosenthal P B, Skehel J J, Gamblin S J. 2020. SARS-COV-2 and bat RaTG13 spike glycoprotein structures inform on virus evolution and furin-cleavage effects. Nat Struct Mol Biol 27:763-767.
34. Bos R, Rutten L, van der Lubbe J E M, Bakkers M J G, Hardenberg G, Wegmann F, Zuijdgeest D, de Wilde A H, Koornneef A, Verwilligen A, van Manen D, Kwaks T, Vogels R, Dalebout T J, Myeni SK, Kikkert M, Snijder E J, Li Z, Barouch D H, Vellinga J, Langedijk J P M, Zahn R C, Custers J, Schuitemaker H. 2020. Ad26 vector-based COVID-19 vaccine encoding a prefusion-stabilized SARS-COV-2 Spike immunogen induces potent humoral and cellular immune responses. NPJ Vaccines 5:91.
35. Papa G, Mallery D L, Albecka A, Welch L G, Cattin-Ortola J, Luptak J, Paul D, McMahon H T, Goodfellow I G, Carter A, Munro S, James L C. 2021. Furin cleavage of SARS-COV-2 Spike promotes but is not essential for infection and cell-cell fusion. PLOS Pathog 17:e1009246.
36. Bukreyev A, Lamirande E W, Buchholz U J, Vogel L N, Elkins W R, St Claire M, Murphy B R, Subbarao K, Collins P L. 2004. Mucosal immunisation of African green monkeys (Cercopithecus aethiops) with an attenuated parainfluenza virus expressing the SARS coronavirus spike protein for the prevention of SARS. Lancet 363:2122-7.
37. Bukreyev A, Lamirande E W, Buchholz U J, Vogel L N, Elkins W R, St. Clair M, Murphy B M, Subbarao K, Collins P L. 2004. Mucosal immunization protects monkeys against SARS coronavirus infection. Lancet submitted.
38. Huff HV, Singh A. 2020. Asymptomatic transmission during the COVID-19 pandemic and implications for public health strategies. Clin Infect Dis doi: 10.1093/cid/ciaa654.
39. He X, Lau EHY, Wu P, Deng X, Wang J, Hao X, Lau Y C, Wong J Y, Guan Y, Tan X, Mo X, Chen Y, Liao B, Chen W, Hu F, Zhang Q, Zhong M, Wu Y, Zhao L, Zhang F, Cowling BJ, Li F, Leung GM. 2020. Temporal dynamics in viral shedding and transmissibility of COVID-19. Nat Med 26:672-675.
40. Meyer M, Garron T, Lubaki N M, Mire C E, Fenton K A, Klages C, Olinger G G, Geisbert T W, Collins P L, Bukreyev A. 2015. Aerosolized Ebola vaccine protects primates and elicits lung-resident T cell responses. J Clin Invest 125:3241-55.
41. Acosta P L, Caballero M T, Polack F P. 2015. Brief History and Characterization of Enhanced Respiratory Syncytial Virus Disease. Clin Vaccine Immunol 23:189-95.
42. Karron R A, Atwell J E, McFarland E J, Cunningham C K, Muresan P, Perlowski C, Libous J, Spector S A, Yogev R, Aziz M, Woods S, Wanionek K, Collins P L, Buchholz U J. 2021. Live-attenuated Vaccines Prevent Respiratory Syncytial Virus-associated Illness in Young Children. Am J Respir Crit Care Med 203:594-603.
43. Wright P F, Karron R A, Belshe R B, Shi J R, Randolph V B, Collins PL, O'Shea AF, Gruber W C, Murphy B R. 2007. The absence of enhanced disease with wild type respiratory syncytial virus infection occurring after receipt of live, attenuated, respiratory syncytial virus vaccines. Vaccine 25:7372-8.
44. Bolles M, Deming D, Long K, Agnihothram S, Whitmore A, Ferris M, Funkhouser W, Gralinski L, Totura A, Heise M, Baric R S. 2011. A double-inactivated severe acute respiratory syndrome coronavirus vaccine provides incomplete protection in mice and induces increased eosinophilic proinflammatory pulmonary response upon challenge. J Virol 85:12201-15.
45. Tseng C T, Sbrana E, Iwata-Yoshikawa N, Newman P C, Garron T, Atmar R L, Peters C J, Couch R B. 2012. Immunization with SARS coronavirus vaccines leads to pulmonary immunopathology on challenge with the SARS virus. PLOS One 7:e35421.
46. Agrawal A S, Tao X, Algaissi A, Garron T, Narayanan K, Peng B H, Couch R B, Tseng C T. 2016. Immunization with inactivated Middle East Respiratory Syndrome coronavirus vaccine leads to lung immunopathology on challenge with live virus. Hum Vaccin Immunother 12:2351-6.

### Example 4: Materials and Methods

This example describes the materials and experimental procedures for the studies described in Examples 5-10.

### Generation of B/HPIV3/S-6P Vaccine

The B/HPIV3/S-2P cDNA was created previously as follows (4). The ORF encoding the full-length 1,273 aa SARS-COV-2 S protein from the first available sequence (GenBank MN908947) was codon-optimized for human expression, and a cDNA clone was synthesized commercially (BioBasic). Two proline substitutions (aa positions 986 and 987) and four aa substitutions (RRAR to GSAS, aa 682-685, with reference to SEQ ID NO: 22) that stabilize S in the prefusion conformation and ablate the furin cleavage site between S1 and S2 (7) were introduced by site-directed mutagenesis (Agilent) to generate the S-2P cDNA (4). This S-2P ORF was then inserted into a cDNA clone encoding the B/HPIV3 antigenome between the N and P ORFs to create the B/HPIV3/S-2P cDNA (22). This cDNA was then modified by the introduction of 4 additional proline substitutions (aa position 817, 892, 899, and 942 for a total of 6 proline substitutions) to create the B/HPIV3/S-6P cDNA. The 4 additional proline substitutions confer increased stability to a soluble version of the prefusion-stabilized S protein (8). The sequence of the B/HPIV3/S-6P cDNA was confirmed by Sanger sequencing and used to transfect BHK21 cells (clone BSR T7/5) with helper plasmids encoding the N, P and L proteins as described previously (4, 23) to produce the B/HPIV3/S-6P recombinant virus. The empty control virus B/HPIV3 was rescued in parallel using the same protocol. Virus stocks were grown in Vero cells, and viral genomes purified from recovered virus were completely sequenced by Sanger sequencing using overlapping uncloned RT-PCR fragments, confirming the absence of any adventitious mutations.

### Immunization and Challenge of Rhesus Macaques and Sample Collection

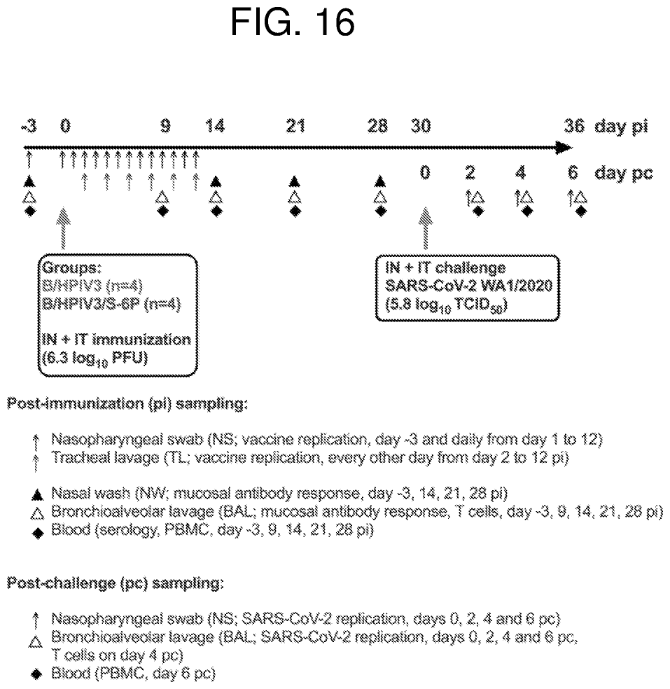

All animal studies were approved by the NIAID Animal Care and Use Committee. The timeline of the experiment and sampling is summarized in FIG. 16. Eight juvenile to young adult male rhesus macaques (Macaca mulatta) confirmed to be seronegative for HPIV3 and SARS-COV-2 were immunized intranasally (0.5 ml per nostril) and intratracheally (1 ml) with 6.3 log₁₀ plaque-forming units (PFU) total of B/HPIV3/S-6P or the empty vector control B/HPIV3. Animals were observed daily from day -3 until the end of the study. Each time they were sedated, animals were weighed, their rectal temperature was taken, as well as the pulse in beats per minute and the respiratory rate in breaths per minute. In addition, the blood oxygen levels were determined by pulse oximetry.

Blood for analysis of serum antibodies and peripheral blood mononuclear cells (PBMC) was collected on days -3, 4, 9, 14, 21, and 28 pi. A fraction was used to collect PBMC and the other fraction was allowed to clot for serum collection. Nasopharyngeal swabs (NS) for vaccine virus quantification in the URT were performed daily from day -3 to day 10 pi and on days 12 and 14 pi using cotton-tipped applicators. Swabs were placed in 2 ml Leibovitz (L-15) medium with 1× sucrose phosphate (SP) used as stabilizer, and vortexed for 10 seconds. Aliquots were then snap frozen in dry ice before being stored at −80° C. Nasal washes (NWs) for analysis of mucosal IgA and IgG were performed using 1 ml of Lactated Ringer's solution per nostril (2 ml total) on days -3, 14, 21 and 28 pi and aliquots were snap frozen in dry ice and stored at −80° C. until further analysis. Tracheal lavages (TL) for virus quantification in the LRT were done every other day from day 2 to 8 pi and on day 12 pi using 3 ml PBS. The samples were mixed 1:1 with L-15 medium containing 2x SP and aliquots were snap frozen in dry ice and stored at −80° C. for further analysis. Bronchoalveolar lavages (BALs) for analysis of mucosal IgA and IgG and airway immune cells were done on days -3, 9, 14 and 28 pi using 30 ml PBS (3 times 10 ml). For analysis of mononuclear cells, BAL was filtered through a 100 μm filter, and centrifuged at 1,600 rpm for 15 min at 4° C. The cell pellet was resuspended at 2×10⁷ cells/ml in X-VIVO 15 media supplemented with 10% FBS for subsequent analysis. The cell-free BAL was aliquoted, snap frozen in dry ice and stored at -80° C. for further analysis. Rectal swabs were done on day -3 and then every other day from day 2 to 14 following the same procedure than NS.

On day 30 pi, animals were transferred to BSL3 and challenged intranasally and intratracheally with 10^{5.8} TCID₅₀ of SARS-COV-2, USA-WA-1/2020 that was entirely sequenced and free of any prominent adventitious mutations. Sample collections were done following the same procedures as during the immunization phase. Briefly, blood was collected before challenge and on day 6 post-challenge (pc). NS were performed every other day from day 0 to day 6 pc. NWs were done on day 6 pc, BAL on days 2, 4 and 6 pc and rectal swabs on days 0, 2, 4 and 6 pc. Animals were necropsied on day 6 pc, and tissues were collected. In particular, 6 samples per animal from individual lung lobes were collected, and snap frozen in dry ice for further analysis. Lung tissues were fixed in 10% phosphate-buffered formalin.

Immunoplaque Assay for Titration of B/HPIV3 and B/HPIV3/S-6P from RM Samples

Titers of B/HPIV3 and B/HPIV3/S-6P from NS and TLs were determined by dual-staining immunoplaque assay as described previously (4). Briefly, Vero cell monolayers in 24-well plates were infected in duplicate with 10-fold serially diluted samples. Infected monolayers were overlaid with culture medium containing 0.8% methylcellulose, and incubated at 32° C. for 6 days, fixed with 80% methanol, and immunostained with a rabbit hyperimmune serum raised against purified HPIV3 virions to detect B/HPIV3 antigens, and a goat hyperimmune serum to the secreted SARS-COV-2 S to detect co-expression of the S protein, followed by infrared-dye conjugated donkey anti-rabbit IRDye680 IgG and donkey anti-goat IRDye800 IgG secondary antibodies. Plates were scanned with the Odyssey infrared imaging system (LiCor). Fluorescent staining for PIV3 proteins and SARS-COV-2 S was visualized in green and red, respectively, providing for yellow plaque staining when merged.

### Dissociation-Enhanced Lanthanide Fluorescent (DELFIA) Time Resolved Fluorescence (TRF) Immunoassay, ELISA and Live HPIV3 and SARS-COV-2 Neutralization Assays

Levels of anti-SARS-COV-2 S antibodies induced by B/HPIV3/S-6P were determined by DELFIA-TRF from NW or BAL, and from serum samples by ELISA, using two different recombinantly-expressed purified forms of S as described previously (4): one was the secreted form of S-2P, and the other was a fragment (aa 328-531) of the SARS-COV-2 S protein containing the RBD. Mucosal antibody titers were determined as described previously (4) by DELFIA-TRF (Perkin Elmer) following the supplier's protocol. Serum antibody titers were determined by ELISA as described previously (4). The secondary anti-monkey antibodies used in both assays were goat anti-monkey IgG(H+L)-HRP (Thermofisher, Cat #PA1-84631), goat anti-monkey IgA (alpha chain)-biotin (Alpha Diagnostic International, Cat #70049), and goat anti-monkey IgM-biotin (Brookwoodbiomedical, Cat#1152).

The B/HPIV3 vector-specific neutralizing antibodies titers were measured by a 60% plaque reduction neutralization test (PRNT₆₀) as described previously (4). The serum neutralizing antibody assays using live SARS-COV-2 virus was performed in a BSL3 laboratory as described previously. Results were expressed as neutralizing dose 50 (ND₅₀) (4).

### Lentivirus-Based Pseudotype Virus Neutralization Assay

The SARS-COV-2 pseudovirus neutralization studies were performed as previously reported (13). Briefly, the single-round luciferase-expressing pseudoviruses were generated by co-transfection of encoding SARS-COV-2 S (Wuhan-1, GenBank accession number, MN908947.3 or, B.1.351/Beta South Africa, B.1.1.7/Alpha UK, B.1617.2. Delta), luciferase reporter (pHR' CMV Luc), lentivirus backbone (pCMV AR8.2), and human transmembrane protease serine 2 (TMPRSS2) at a ratio of 1:20:20:0.3 into HEK293T/17 cells (ATCC) with transfection reagent LiFect293™. The pseudoviruses were harvested at 72 h post transfection. The supernatants were collected after centrifugation at 1500 rpm for 10 minutes to remove gross cell debris, then filtered through 0.45 mm filter, aliquoted and titrated before neutralization assay. For the antibody neutralization assay, the 6-point, 5-fold dilution series were prepared in culture medium

(DMEM medium with 10% FBS, 1% Pen/Strep and 3 μg/ml puromycin.). Fifty ul antibody dilution were mixed with 50 μl of diluted pseudoviruses in the 96-well plate and incubated for 30 min at 37° C. Ten thousand ACE-2 expressing 293T cells (293T-hACE2.MF stable cell line cells) were added in a final volume of 200 μl. Seventy-two hours later, after carefully removing all the supernatants, cells were lysed with Bright-Glo™ Luciferase Assay substrate (Promega), and luciferase activity (relative light units, RLU) was measured. Percent neutralization was normalized relative to uninfected cells as 100% neutralization and cells infected with only pseudoviruses as 0% neutralization. IC₅₀ titers were determined using a log (agonist) vs. normalized response (variable slope) nonlinear function in Prism v8 (GraphPad).

### Evaluation of the T Cell Response in the Blood and Lower Airways of RMs

Blood and BAL collection procedures followed ACUC approved standard operating procedures and limits. Blood that was collected in EDTA tubes was diluted 1:1 with 1× PBS. Fifteen ml of Ficoll-Paque density gradient (GE Healthcare) was added to Leucosep PBMC isolation tubes (Greiner bio-one) and centrifuged at 1,000 g for 1 min at 22° C., to collect Ficoll below the separation filter. The blood and PBS mixture was added to the Leucosep tubes with Ficoll-Paque and centrifuged at 1,200 g for 10 min at 22° C. The upper layer was poured into a 50 ml conical and brought to 50 ml with PBS, and then centrifuged at 1,600 rpm for 5 min at 4° C. The cell pellet was resuspended at 2×10⁷ cell/ml in 90% FBS and 10% DMSO for storage at −80° C. overnight before being transferred into liquid nitrogen.

Single cell suspensions of PBMCs that had been rested overnight or freshly collected BAL cells were plated at 2×10⁷ cells/ml in 200 μl in 96 well plates with X-VIVO 15 media, with 10% FBS, Brefeldin 1000× (Thermofisher Cat#00-4506-51) and Monensin 1000 × (Thermofisher Cat#00-4505-51), CD107a APC 1:50, CD107b APC 1:50, and the indicated peptide pools at 1 μg/ml. Replica wells were not stimulated. Spike peptide pools consisted of Peptivator SARS-COV-2 Prot_S1 (Miltenyi Cat#130-127-048), Peptivator SARS-COV-2 Prot_S+(Miltenyi Cat# 130-127-312), and Peptivator SARS-COV-2 Prot_S (Miltenyi Cat#130-127-953) covering the whole spike protein. Nucleocapsid peptide pool consisted of Peptivator SARS-COV-2 Prot_N (Miltenyi Cat# 130-126-699). Cells were stimulated for 6 h at 37° C. with 5% CO₂. After stimulation, cells were centrifuged at 1,600 rpm for 5 min at 4° C., and further processed by surface staining.

Cells were resuspended in 50 μl surface stain antibodies diluted in PBS with 1% FBS and incubated for 20 min at 4° C. Cells were washed 3 times with PBS with 1% FBS before fixation with eBioscience Intracellular Fixation & Permeabilization Buffer Set (Thermo Cat# 88-8824-00) for 16 h at 4° C. After fixation, cells were centrifuged at 2,200 rpm for 5 min at 4° C. without brake and washed once with eBioscience Permeabilization Buffer. Cells were resuspended in 50 μl intracellular stains diluted in eBioscience Permeabilization Buffer, and stained for 30 min at 4° C. The antibodies used for extracellular and intracellular staining were: CD69 (FITC, clone FN50, Biolegend), granzyme B (BV421, clone GB11, BD Biosciences), CD8a (eFluor 506, clone RPA-T8, Thermofisher), IL-2 (BV605, 17H12, Biolegend), IFNγ (BV711, clone 4S.B3, Biolegend), IL-17 (BV785, clone BL168, Biolegend), TNFα (BUV395, clone Mab11, BD Biosciences), CD4 (BUV496, clone SK3, BD Biosciences), CD95 (BUV737, clone DX2, BD Biosciences), CD3 (BUV805, clone SP34-2, BD Biosciences), CD107a (AF647, clone H4A3, Biolegend), CD107b (AF647, clone H4B4, Biolegend), viability Dye eFluor780 (Thermofisher), CD103 (PE, clone B-Ly7, ebioscience), CD28 (PE/Dazzle 594, clone CD28.2, Biolegend), Ki-67 (PE-Cy7, clone B56, BD Biosciences), Foxp3 (AF700, clone PCH101, Thermofisher). After staining, cells were washed with eBioscience permeabilization buffer 2x and resuspended in PBS supplemented with 1% FBS and 0.05% sodium azide for flow cytometry analysis on the BD Symphony platform. Data were analyzed using FlowJo version 10.

### Quantification of SARS-COV-2 Genomic and Subgenomic RNA

One hundred ul each of NS, NW and BAL fluid collected on day 2, 4 and 6 pc and rectal swabs collected on day 6 pc were inactivated in a BSL3 laboratory using 400 μl buffer AVL (Qiagen) and 500 μl ethanol, and RNA was extracted using the QIAamp Viral RNA Mini Kit (Qiagen) according to the manufacturer's protocol. To extract total RNA from lung homogenates harvested on day 6 pc, 300 μl of each lung homogenate (at a concentration of 0.1 g of tissue/ml) was mixed with 900 μl TRIzol LS (Thermo Fisher) using Phasemaker Tubes (Thermo Fisher) and RNA was extracted using the PureLink RNA Mini Kit (Thermo Fisher) following the manufacturer's instructions. Then, the SARS-COV-2 genomic N RNA and subgenomic E mRNA were quantified in triplicate using the TaqMan RNA-to-Ct 1-Step Kit (ThermoFisher) using previously reported TaqMan primers/probes (24-26) on the QuantStudio 7 Pro (ThermoFisher). Standard curves were generated using serially diluted pcDNA3.1 plasmids encoding gN, gE, or sgE sequences. The limit of detection was 2.57 log₁₀ copies per ml of NP, nasal wash, BAL fluid, or rectal swabs and 3.32 log₁₀ copies per g of lung tissue.

### Statistical Analysis

Data sets were assessed for significance using two-way ANOVA with Sidak's multiple comparison test using Prism 8 (GraphPad Software). Data were only considered significant at p ≤ 0.05.

### Example 5: B/HPIV3/S-6P replicates efficiently in the upper and lower airways of rhesus macaques

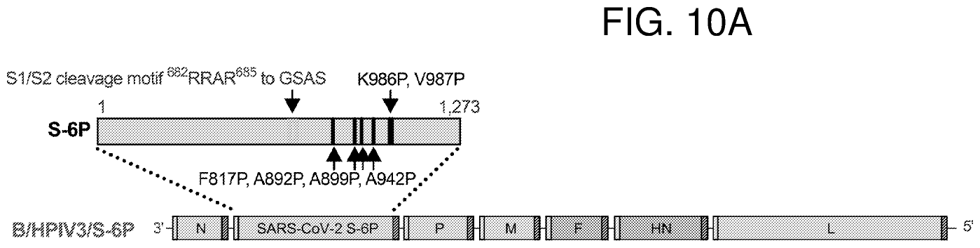

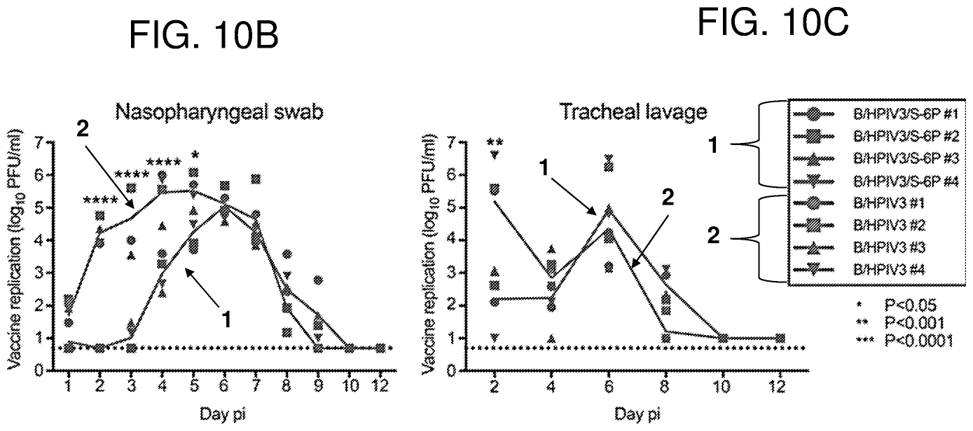

B/HPIV3 was used to express a prefusion-stabilized version of the SARS-COV-2 S protein. B/HPIV3 is a cDNA-derived version of bovine PIV3 (BPIV3) strain Kansas in which the BPIV3 hemagglutinin-neuraminidase (HN) and F glycoproteins (the two PIV3 neutralization antigens) have been replaced by those of the human PIV3 strain JS (4, 7) (FIG. 10A). The BPIV3 backbone provides host-range restriction of replication in humans, resulting in stable attenuation (4, 5). B/HPIV3/S-6P expresses a full-length prefusion-stabilized version (S-6P) of the SARS-COV-2 S protein (1,273 aa) from a supernumerary gene, inserted between the N and P genes (FIG. 10A). The S-6P version of the S-protein contains 6 proline substitutions (25) that stabilize S in its trimeric prefusion form and increase expression and immunogenicity. The S1/S2 polybasic furin cleavage motif “RRAR” was ablated by amino acid substitutions (RRAR-to-GSAS) (4) (FIG. 1A), rendering S-6P non-functional for virus entry, which eliminates the possibility of S altering the tissue tropism of the B/HPIV3 vector.

To evaluate vaccine replication and immunogenicity, RMs were immunized in 2 groups of 4 with a single dose of 6.3 log₁₀ plaque-forming units (PFU) of B/HPIV3/S-6P or the B/HPIV3 vector control, administered by the combined intranasal and intratracheal route (IN/IT) (FIG. 16). Nasopharyngeal swabs (NS) and tracheal lavages (TL) were performed daily and every other day, respectively, from day 0 to 12 pi to evaluate vaccine replication in the upper and lower airways (UA and LA, respectively; FIGS. 10B, 10C, FIG. 16). Replication of B/HPIV3/S-6P and the B/HPIV3 control was detectable through days 8 or 9 in the UA and LA. In the UA, peak replication of B/HPIV3/S-6P and B/HPIV3 control was detected between study days 4 and 6 (medians independent of study day: 4.9 log₁₀ PFU/mL vs 5.9 log₁₀ PFU/mL, respectively; P=0.1429 by two-tailed Mann-Whitney test); replication of B/HPIV3/S-6P was delayed by 1-2 days compared to that of the empty vector (p<0.0001 on days 2 to 4) (FIG. 10B). In the LA, B/HPIV3/S-6P replicated with similar kinetics as B/HPIV3, reaching median peak titers of 4.6 log₁₀ PFU/mL and 4.0 log₁₀ PFU/mL on day 6 pi (FIG. 10C). Thus, despite its supernumerary S gene, B/HPIV3/S-6P replicated efficiently in the UA or LA of RMs.

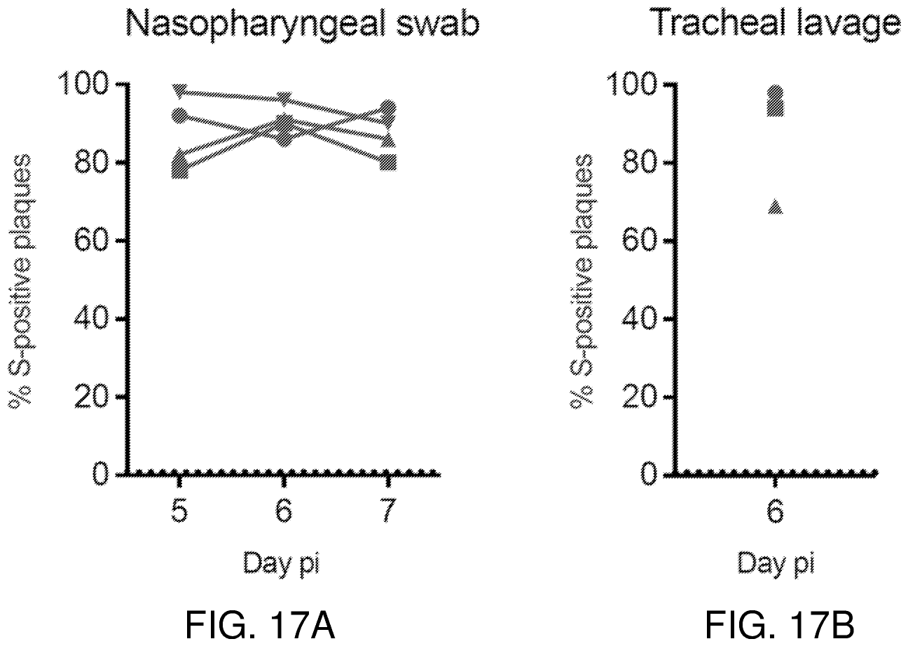

To evaluate the stability of S expression during vector replication, NS and TL specimens positive for B/HPIV3/S-6P were evaluated by a dual-staining immunoplaque assay, which detects the expression of S and vector proteins. On average, 89% of the B/HPIV3/S-6P plaques recovered between days 5 and 7 from NS were positive for S expression (FIG. 17), suggesting stable S-6P expression in the UA. In TL specimens collected on day 6 pi, S expression was stable in 3 of 4 RM, with on average 88% of the plaques positive for S expression. In TL samples from one B/HPIV3/S-6P-immunized RM (B/HPIV3/S-6P #3), plaques were negative for S expression on day 6 pi. Sanger sequencing of the S gene revealed 13 cytidine-to-thymidine mutations in a 430-nucleotide region, suggestive of deaminase activity in the LA of this animal. Eleven were missense mutations resulting in amino acid substitutions, including 7 proline substitutions which might affect S protein folding.

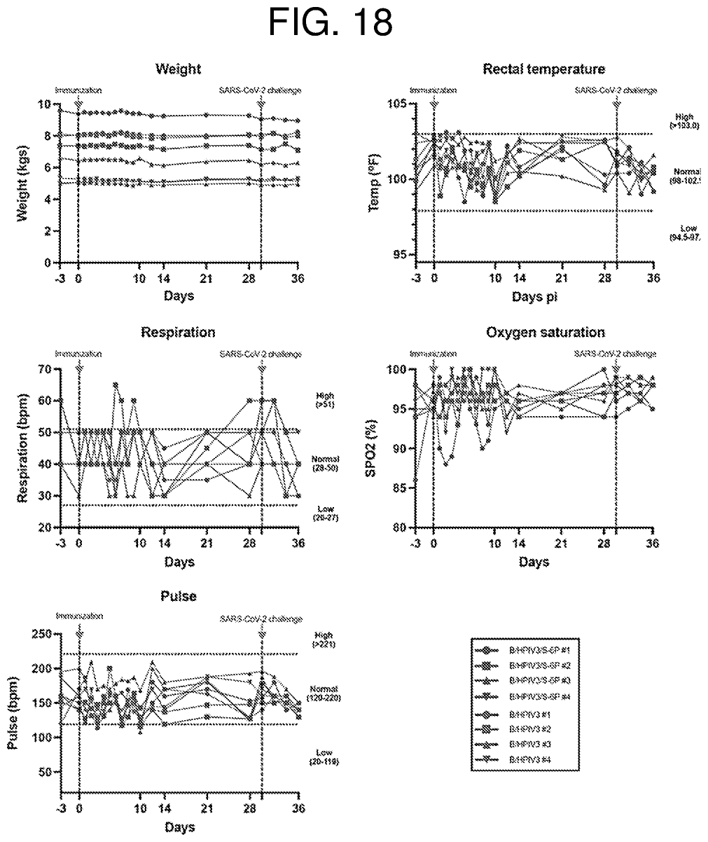

No changes in body weight, rectal temperature, respiration, oxygen saturation or pulse were detected following immunization of RMs with B/HPIV3 or B/HPIV3/S-6P (FIG. 18). Thus, B/HPIV3/S-6P replicates efficiently in the UA and LA of RMs, leads to S protein gene expression, causes no apparent symptoms, and is cleared in approximately ten days.

Example 6: B/HPIV3/S-6P Induces Anti-SARS-COV-2 S Mucosal Antibodies in the Upper and Lower Airways

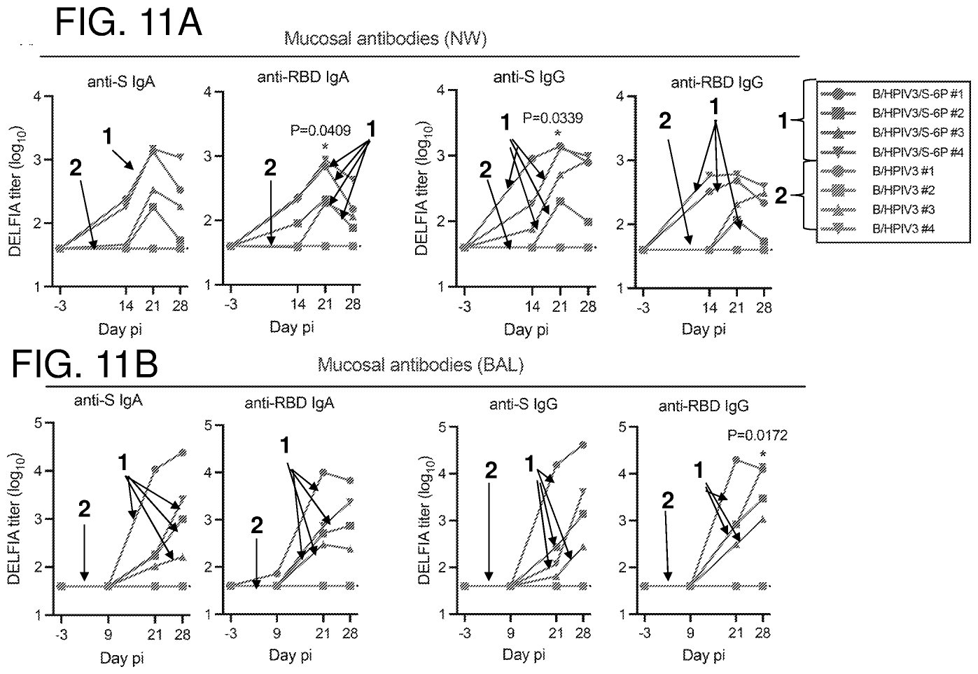

To assess the kinetics of airway mucosal antibody responses to the SARS-COV-2 S protein or to a fragment (aa 328-531) containing its receptor binding domain (RBD) in the UA and LA, nasal washes (NW) were collected 3 days before immunization and on days 14, 21, and 28 after immunization, and bronchoalveolar lavage fluid (BAL) were collected on days 9, 21, and 28 after immunization (FIG. 16). IgA and IgG binding antibodies were evaluated using a soluble S-2P prefusion stabilized version of the vaccine-matched S protein (10) or its receptor binding domain (RBD) (10) in a highly-sensitive dissociation-enhanced lanthanide fluorescence (DELFIA) immunoassay (FIGS. 16, 11A and 11B).

In B/HPIV3/S-6P-immunized animals, mucosal anti-S (2/4 animals) and anti-RBD IgA (3/4 animals) were detected in the UA as early as 14 days pi (FIG. 11A). By day 21 pi, all 4 B/HPIV3/S-6P immunized RMs exhibited anti-S and anti-RBD IgA (DELFIA geometric mean titers (GMT) between 2.3 and 3.2 log₁₀. P=0.0409 for anti-RBD IgA on day 21 pi). B/HPIV3/S-6P also induced mucosal anti-S and anti-RBD IgG responses in the UA on day 14 pi in 3/4 and 2/4 RMs, respectively, with responses observed in all animals by day 21 (DELFIA anti-S titers between 2.1 and 3.1 log₁₀, P=0.0339).

B/HPIV3/S-6P also induced mucosal anti-S and anti-RBD IgA and IgG in the LA (FIG. 11B). On day 21 pi, anti-S and anti-RBD IgA titers between 2.0 and 4.0 log₁₀ were detectable in the LA of all 4 B/HPIV3/S-6P-immunized RMs (IgA). Anti-S IgA titers continued to rise in all RMs until day 28 after immunization. Anti-RBD IgA titers also continued to rise in 2 RMs, but modestly declined in the other 2 RMs. On day 21 pi, all B/HPIV3/S-6P-immunized RMs also had anti-S and anti-RBD IgG detectable (DELFIA titers between 1.8 and 4.2 log₁₀), and titers continued to rise between days 21 and 28 after immunization. Similarly, anti-RBD IgG titers continued to rise until day 28 pi in 3 RMs, but modestly declined in 1 RM. None of the RMs immunized with the empty B/HPIV3 vector had anti-S or anti-RBD IgA or IgG antibodies detectable in the UA or LA.

### Example 7: B/HPIV3/S-6P Immunization Induces Serum Antibodies Against SARS-COV-2 S that Neutralize SARS-COV-2 WA1/2020 and Variants of Concern (VoCs)

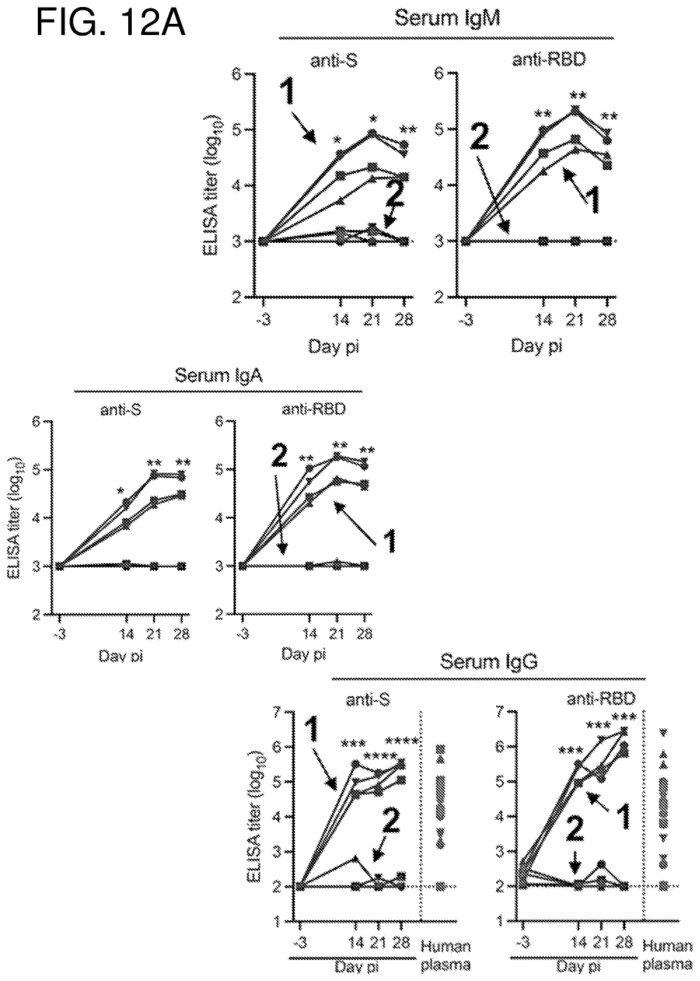

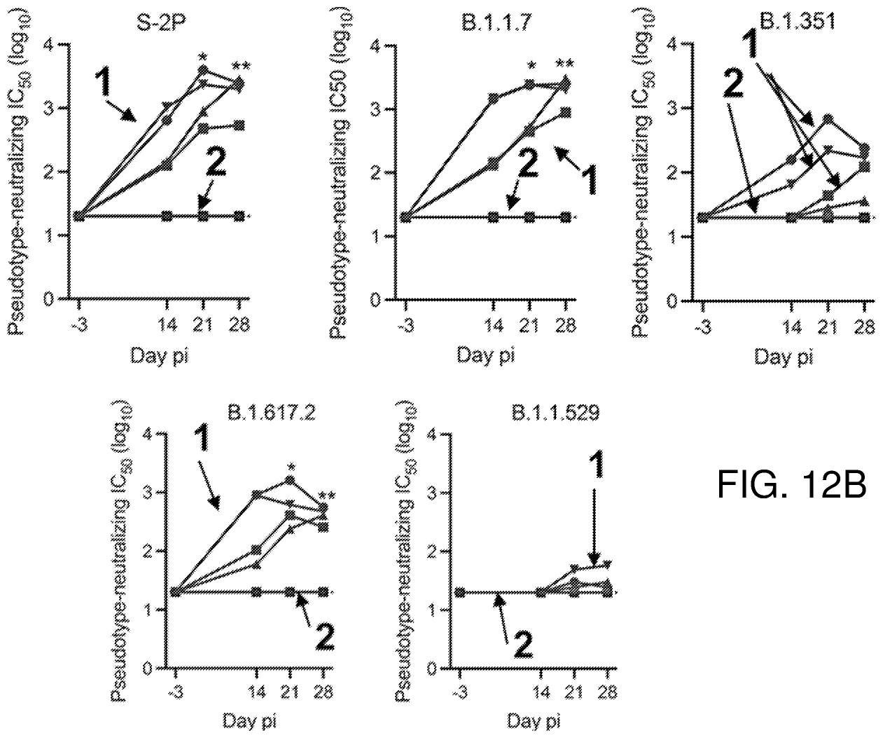

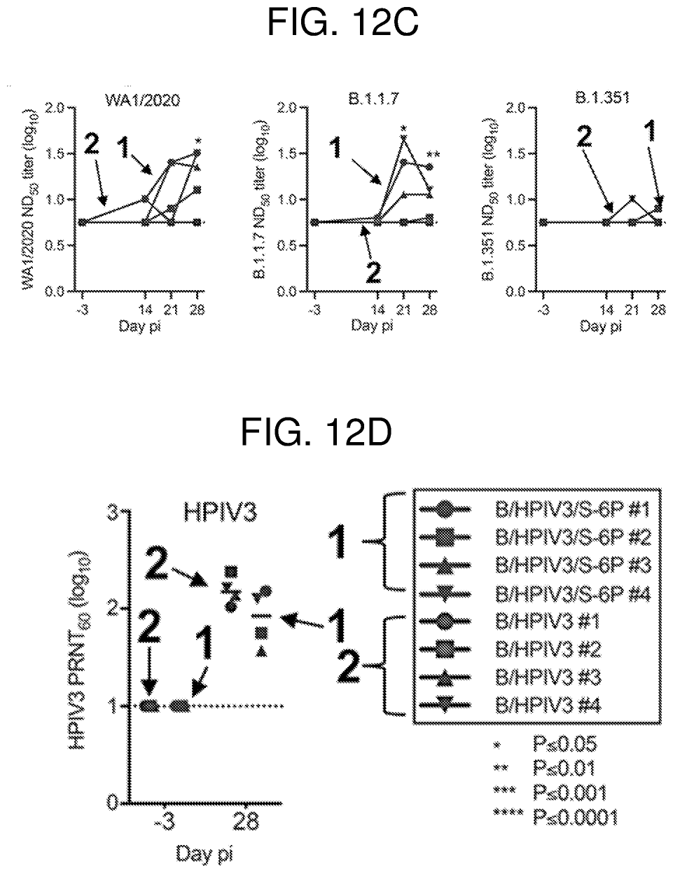

Next, the kinetics and breadth of the serum antibody response to B/HPIV3/S-6P (FIG. 12) was assessed. Robust serum IgM, IgA and IgG binding antibody responses to the S protein and RBD were detected by ELISAs in 4/4 B/HPIV3/S-6P-immunized RMs as early as 14 days pi (FIG. 12A). Serum anti-S and anti-RBD IgM titers peaked on day 21 pi in all 4 RMs (ELISA titers between 4.1 and 5.3 log₁₀, p<0.05), and declined towards day 28 pi. Serum anti-S IgA ELISA titers peaked on day 21 pi in 2 RMs and remained steady, while they continued to rise until day 28 pi in the other 2 RMs (peak ELISA titers between 4.3 and 4.9 log₁₀, p<0.01). Serum anti-RBD IgA titers peaked on day 21 in all 4 RMs (ELISA titers between 4.8 and 5.3 log₁₀, p<0.01) and modestly declined by day 28 pi. High levels of serum anti-S and anti-RBD IgG were also measured in all B/HPIV3/S-6P-immunized RMs on day 14 pi, continuing to rise in all RMs until day 28 pi (ELISA GMTs between 5.8 to 6.4 log₁₀ on day 28 pi, p<0.0001). These levels of anti-S and anti-RBD IgG antibodies were 16-fold and 180-fold higher than the mean anti-S and anti-RBD IgG titers, respectively, detected in plasma obtained from 23 SARS-COV-2-convalescent humans. As expected, 0/4 RMs immunized with empty B/HPIV3 control had serum anti-S or anti-RBD IgM, IgA or IgG antibodies detectable at any time.

The kinetics and breadth of the serum neutralizing antibody response to the vaccine-matched SARS-CoV-2 strain WA-1 and to 4 VoCs (B.1.1.7/Alpha, B.1.351/Beta, B.1.617.2/Delta, and B.1.1.529/Omicron BA.I sublineage) were evaluated using a lentivirus pseudotype neutralization assay (11) (FIG. 12B). The sera efficiently and similarly neutralized lentiviruses pseudotyped with the vaccine-matched WAI S protein (in the stabilized S-2P prefusion form; IC₅₀ on day 28 between 2.7 and 3.5 log₁₀, p≤0.01) or with S from the Alpha lineage (IC₅₀ between 3.0 and 3.5 log₁₀, p<0.01). The sera also neutralized the Beta S-pseudotyped lentivirus, although the titer was reduced compared to the vaccine match (IC₅₀ between 1.6 and 2.4 log₁₀). Day-14 sera from all 4 RMs efficiently neutralized the Delta S-pseudotyped lentivirus; titers further increased, but, on day 28, were about 5-fold reduced compared to the vaccine match (IC₅₀ between 2.4 and 2.8 log₁₀, p<0.01). A low neutralizing activity against Omicron BA.I S-pseudotyped lentivirus was detected in day-28 sera from 3 of 4 RMs (IC₅₀ between 1.4 and 1.8 log₁₀) that was 59-fold reduced compared to the vaccine match.

The serum neutralizing antibody titers also were assessed by a live virus neutralization assay using the vaccine-matched WA1/2020 isolate or an isolate of the Alpha or Beta lineages (FIG. 12C). Results were overall comparable with those of the pseudotyped lentivirus neutralization assays, although the sensitivity and the dynamic range of the live virus neutralization assays were much lower than those of the pseudotype neutralization assays. As expected, neutralizing antibodies against the various SARS-COV-2 lineages were undetectable in sera from B/HPIV3-control immunized RMs by pseudotype or live virus SARS-COV-2 neutralization assay. Additionally, all 8 RMs developed neutralizing serum antibodies against the HPIV3 vector (PRNT50 titers between 1.6 and 2.4 log₁₀, FIG. 12D).

### Example 8: B/HPIV3/S-6P Immunization Induces High Frequencies of SARS-COV-2 S-Specific CD4+ and CD8+ T Cells in the Blood and the Airways

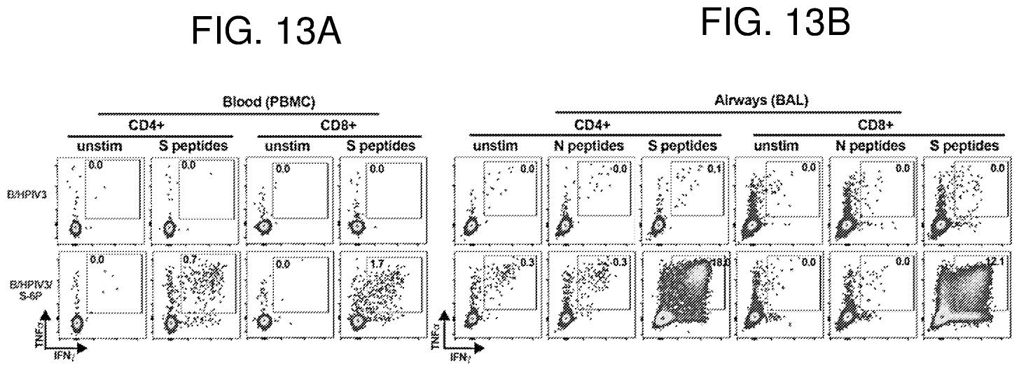

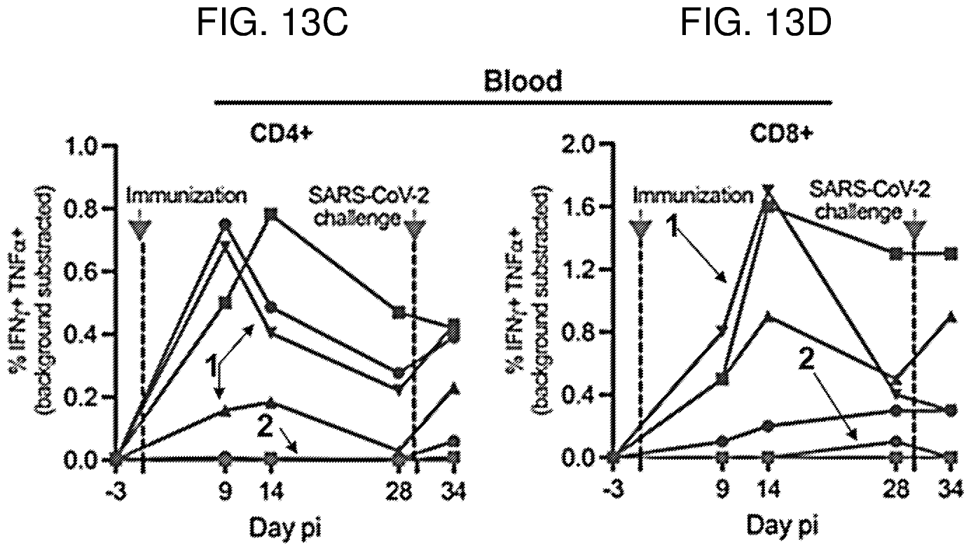

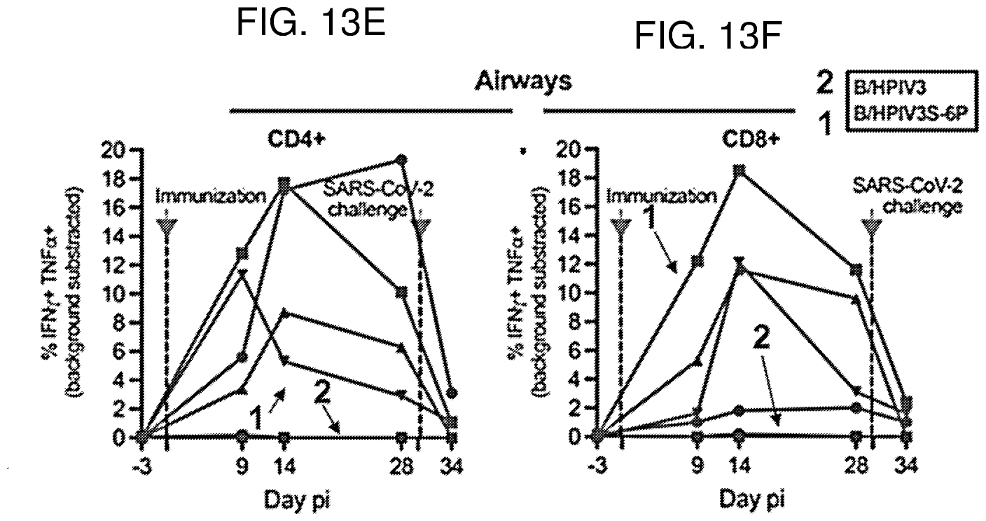

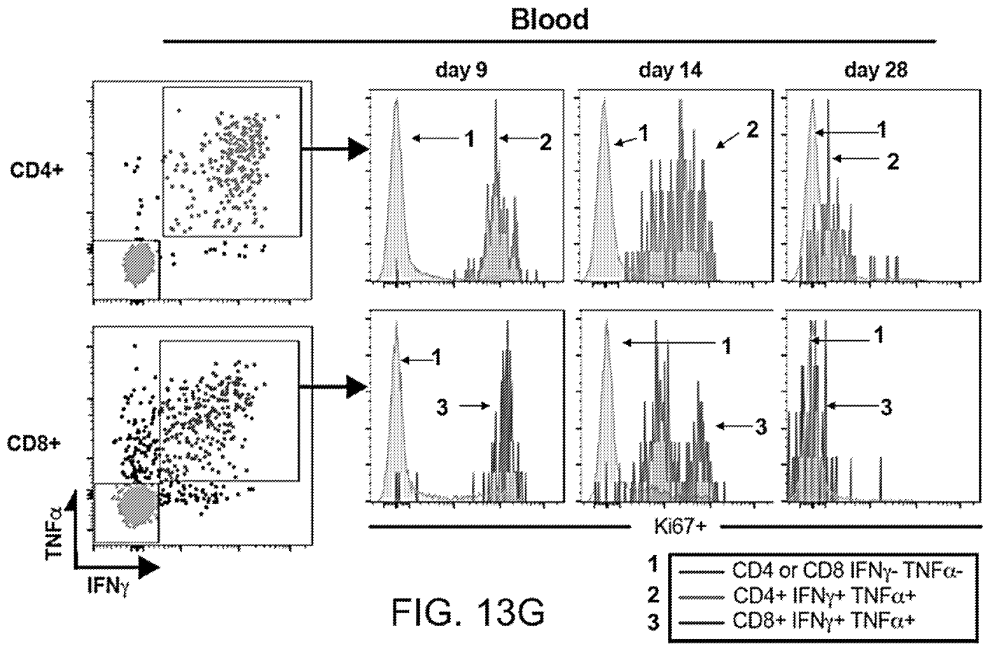

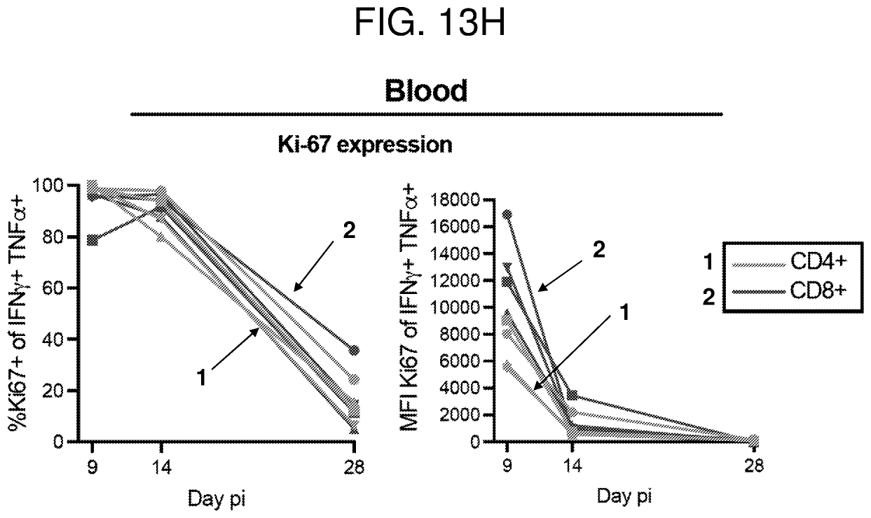

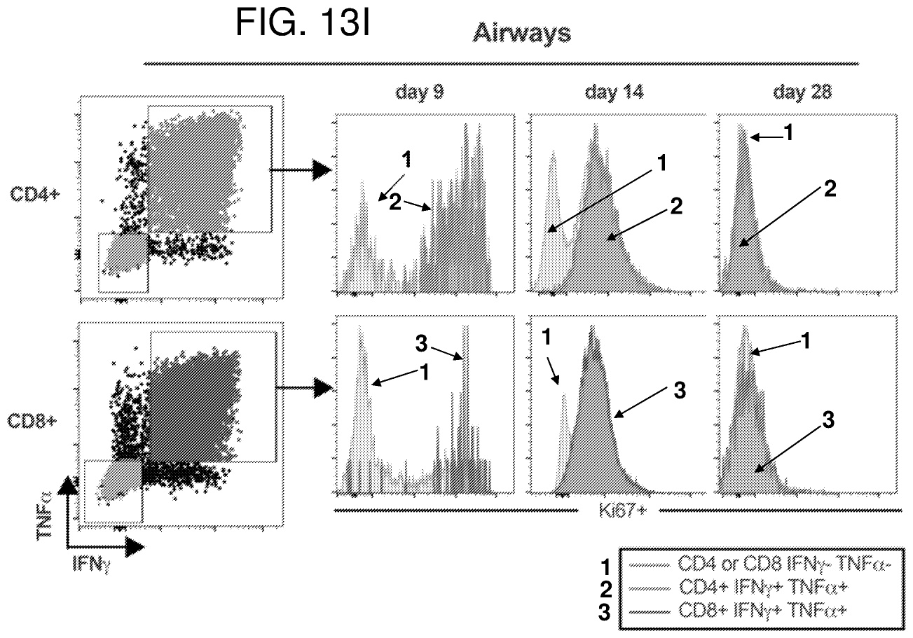

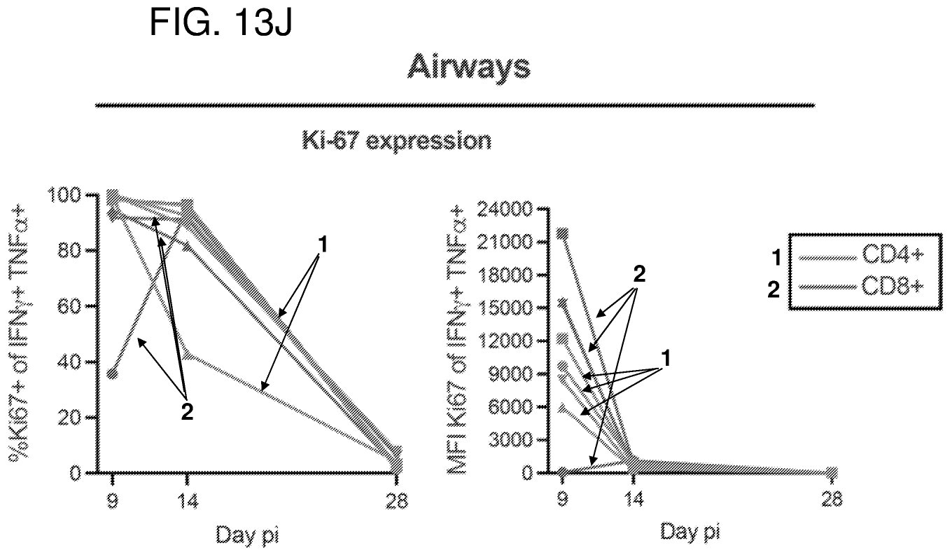

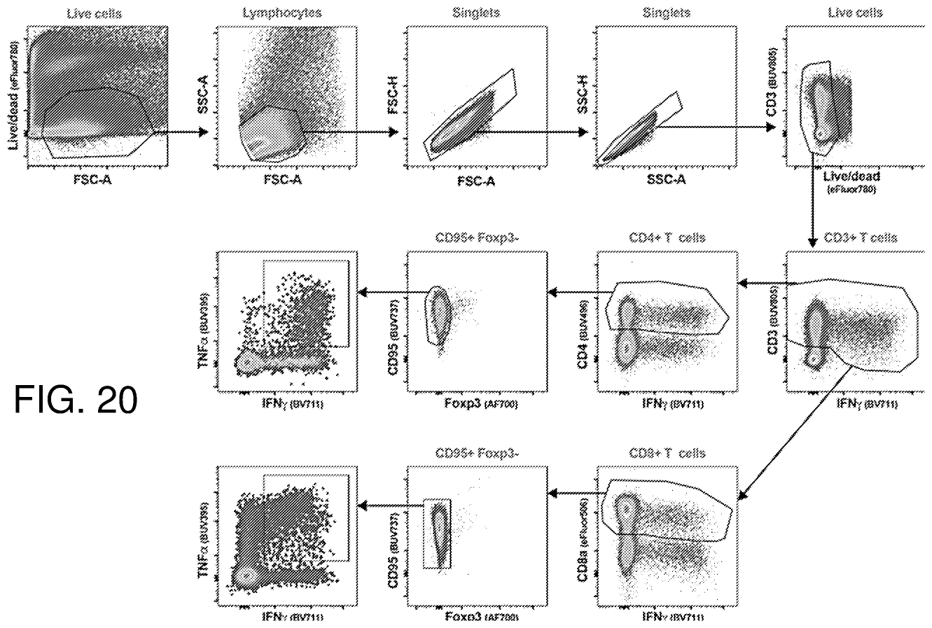

SARS-COV-2 S-specific CD4+ and CD8+ T cell responses were evaluated using peripheral blood mononuclear cells (PBMCs) and cells recovered from the LA by BAL (see FIG. 20 for gating strategy) at the indicated time points following immunization with B/HPIV3/S-6P (FIGS. 13, 14 and 19) and SARS-CoV-2 challenge (FIG. 16). S and N-specific CD4 and CD8 T cells were identified as IFNγ⁺/TNFα⁺ double-positive cells after stimulation with pools of overlapping 15-mer peptides covering the entire length of these proteins. S-specific CD4 T cells were present in the blood of all B/HPIV3/S-6P-immunized RMs by day 9 pi (FIG. 13A left panels; kinetics are shown in FIG. 13C); frequencies peaked on day 9 (2 RMs) or day 14 pi (2 RMs; average peak % of S-specific CD4 T cells irrespective of the peak day =0.6%), and then steadily declined until day 28 pi. S-specific CD8 T cells were also detectable in the blood of B/HPIV3/S-6P-immunized RMs on day 9 pi, and their frequencies peaked on day 14 pi in 3 of 4 RMs (average peak % of S-specific CD8 T cells irrespective of the peak day =1.1%, FIG. 13A, right panels and FIG. 13D).

In the LA of B/HPIV3/S-6P-immunized animals, S-specific IFNγ⁺/TNFα⁺ CD4+ and CD8+ T cells were abundant by day 9 pi (FIGS. 13B, 13E and 13F). Remarkably, the average peak % of S-specific IFNγ⁺/TNFα⁺ CD4+ T cells recovered from BAL irrespective of day pi reached 14.3% (FIG. 13B, left panels and FIG. 13E). In 3 of 4 animals, their frequency declined between day 14 and 28 pi. S-specific IFNγ⁺/TNFα⁺ CD8+ T cells in BAL also peaked on day 14 pi in 3 of 4 RMs (average peak % of S-specific IFNγ⁺/TNFα⁺ CD8+ T cells irrespective of the peak day=11.1%, FIG. 13B right panels and FIG. 13F). No S-specific CD4+ or CD8+ T cells were detected in the blood or BAL of RMs immunized with B/HPIV3 (FIGS. 13A-13F). Lastly, stimulation of CD4+ or CD8+ T cells isolated from BAL with N peptides, which were included as negative controls, did not reveal IFNγ⁺/TNFα⁺ positive cells above the background present in unstimulated cells (FIG. 13B).

On day 9 pi, close to 100% of the S-specific CD4+ and CD8+ T cells in the blood (FIGS. 13G-13H) and in the lungs (FIGS. 13I-13J) of the B/HPIV3/S-6P-immunized RMs expressed high level of Ki-67, confirming active proliferation. While most of these cells still expressed Ki-67 on day 14, the level of expression was strongly reduced, and the majority of cells were Ki-67 and had ceased to proliferate by day 28 pi.

### Example 9: B/HPIV3/S-6P Immunization Induces Highly Functional SARS-COV-2-Specific Memory CD4 T Cells and Cytotoxic CD8 T Cells in the Airways Which Transition to Tissue-Resident Memory Phenotypes

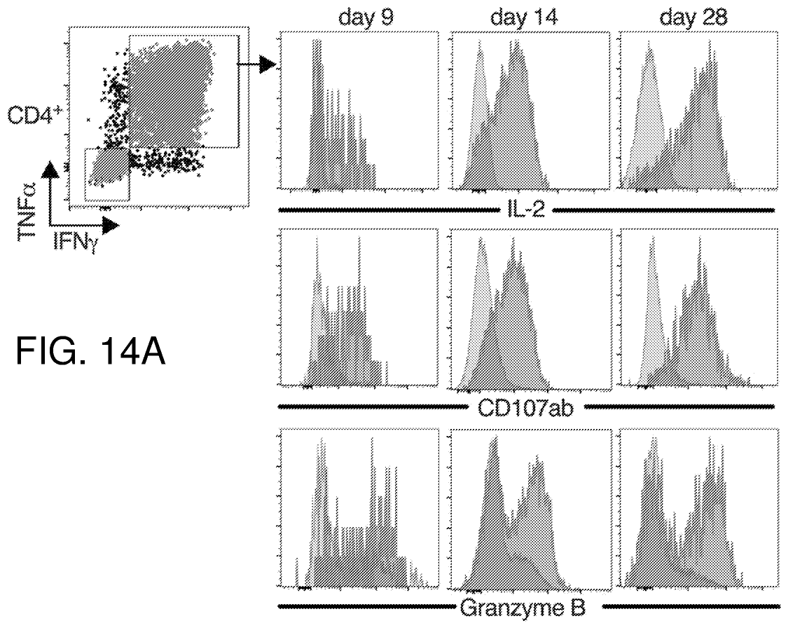

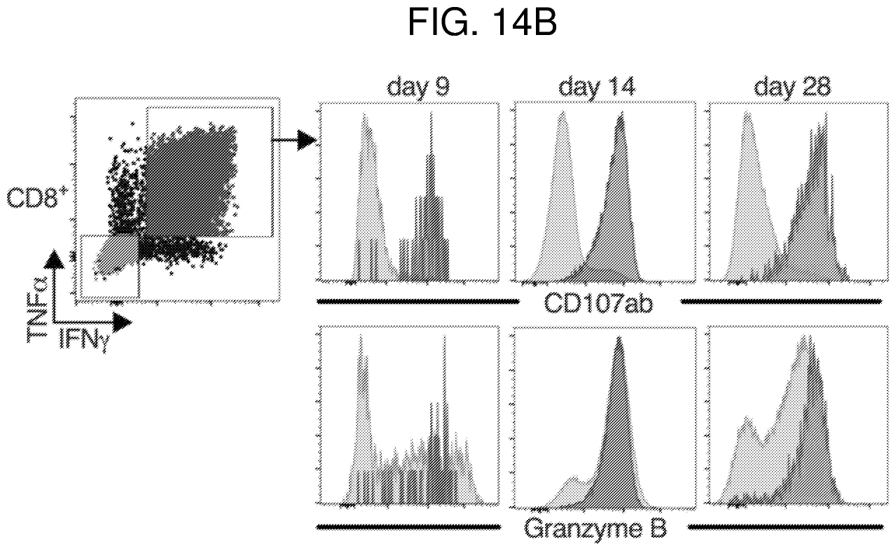

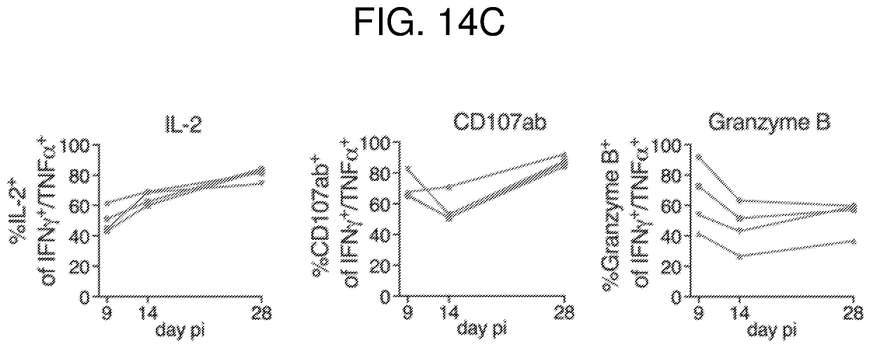

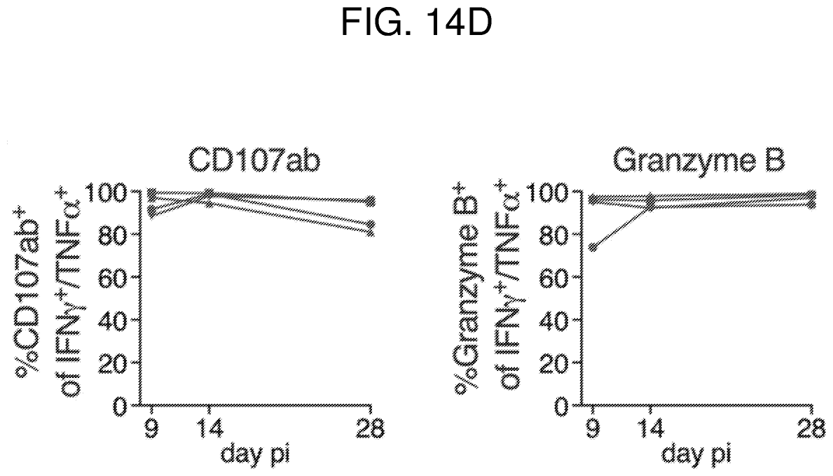

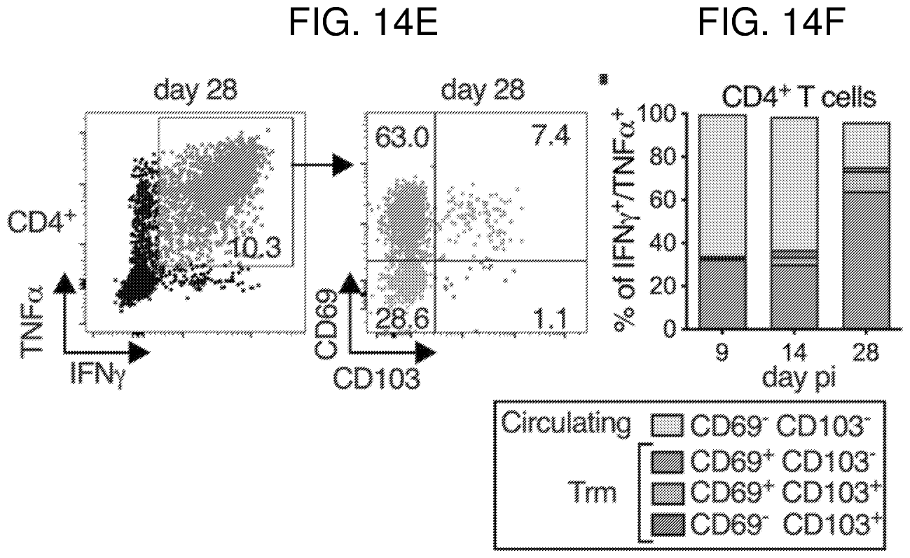

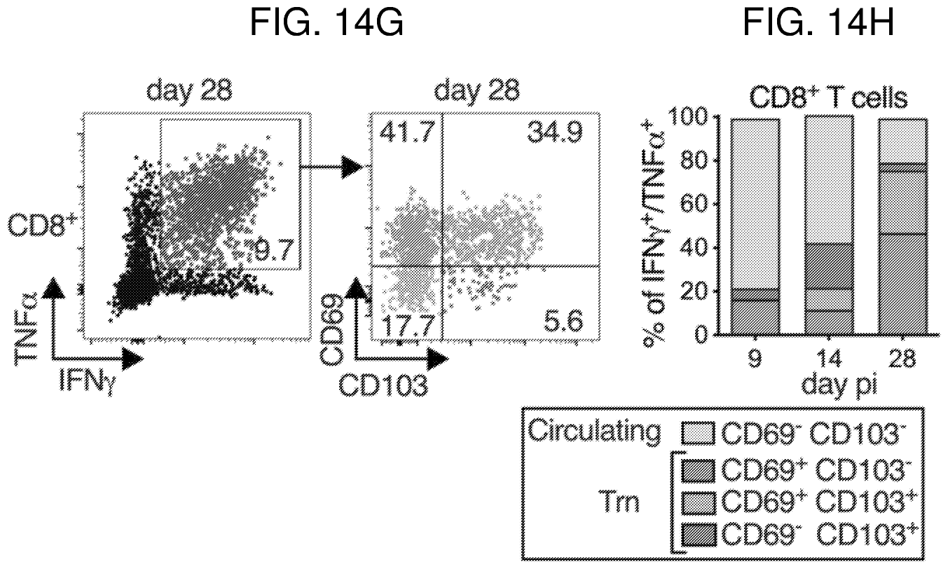

A more comprehensive phenotypic analysis of the lung-derived S-specific CD4+ T cells revealed that, in addition to expressing IFNγ and TNFα, a proportion of these cells (about 40 to 80% from day 9 to 28 pi) also expressed IL-2, characteristic for a type 1 helper (Th1)-biased phenotype (FIGS. 14A-14B). Furthermore, a fraction of these Th1-biased S-specific CD4+ T cells also expressed markers of cytotoxicity such as the degranulation markers CD107ab and granzyme B.

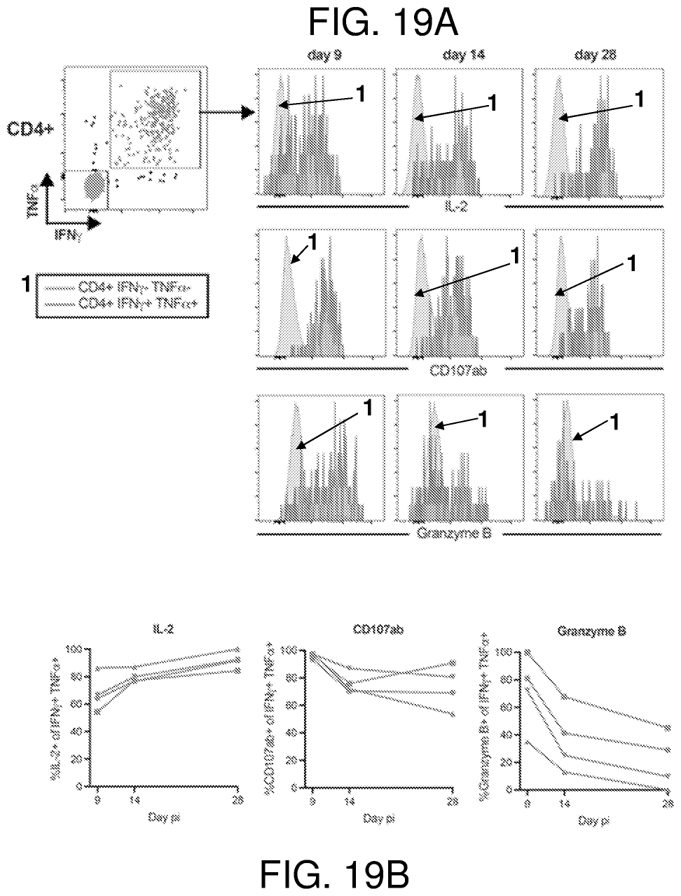

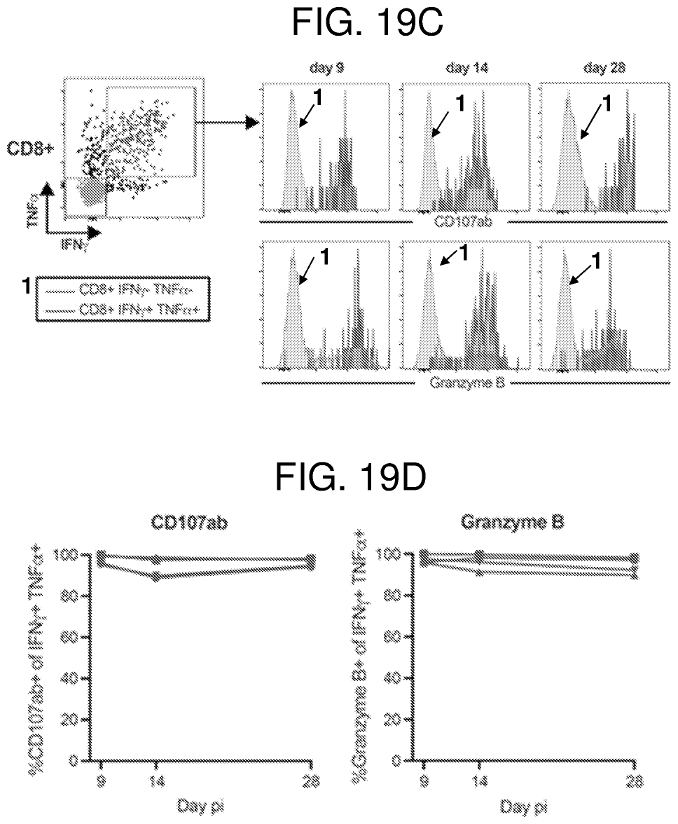

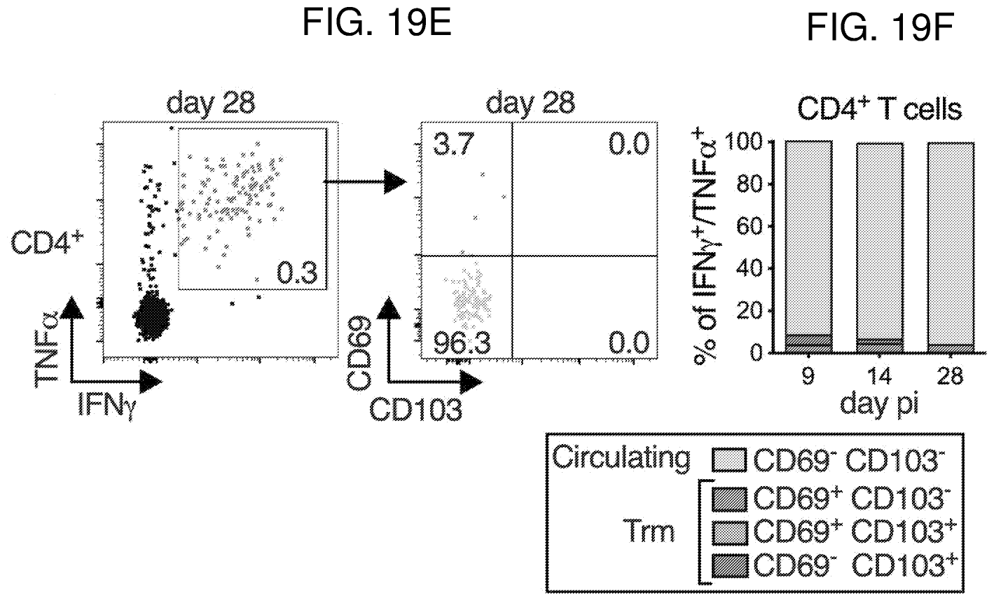

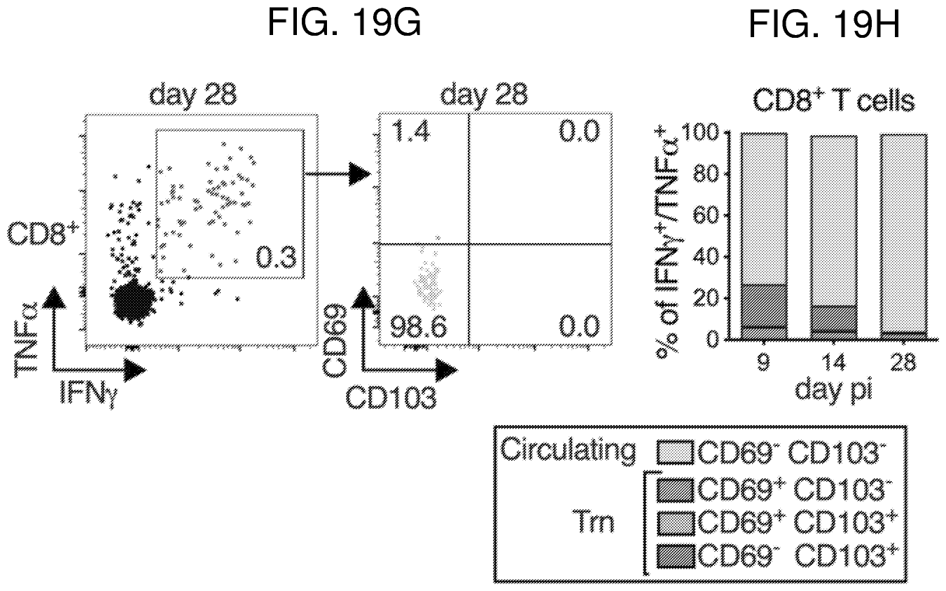

Thus, the memory CD4 T cells induced by this vaccine displayed typical Th1-biased phenotype, similar to those generated after natural SARS-COV-2 infection (33-35). S-specific CD8⁺ T-cells, in addition to expressing IFNγ and TNFα, also expressed high levels of degranulation markers CD107ab and granzyme B from day 9 to 28 pi, suggesting that they were highly functional (FIGS. 14B, 14D). The phenotype of the blood-derived S-specific CD4 and CD8 T cells was overall comparable to that of the airway-derived S-specific T cells (FIG. 19).

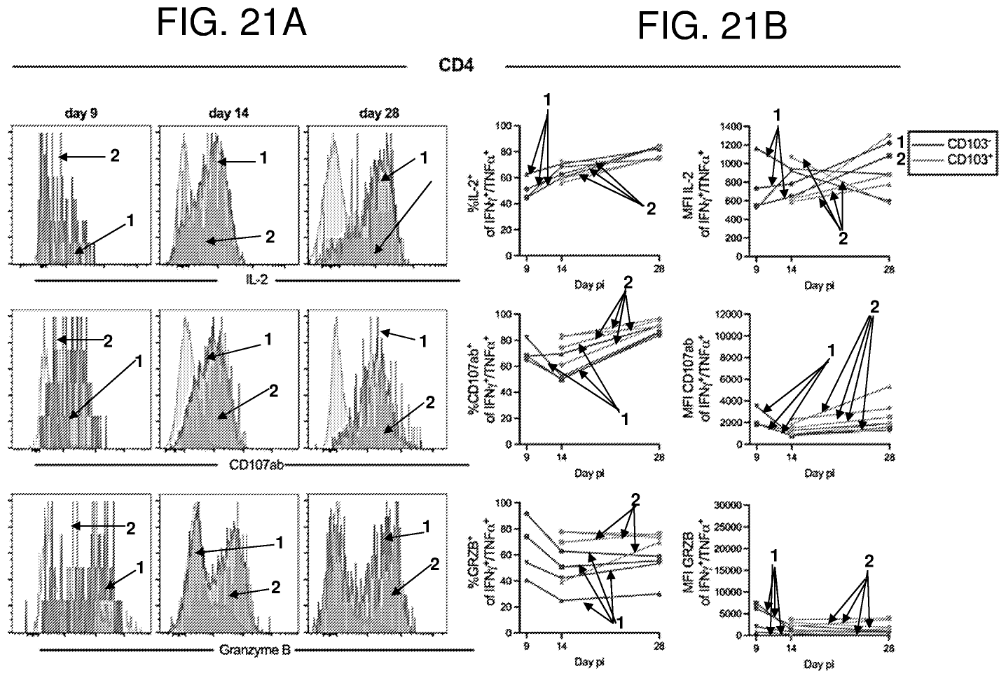

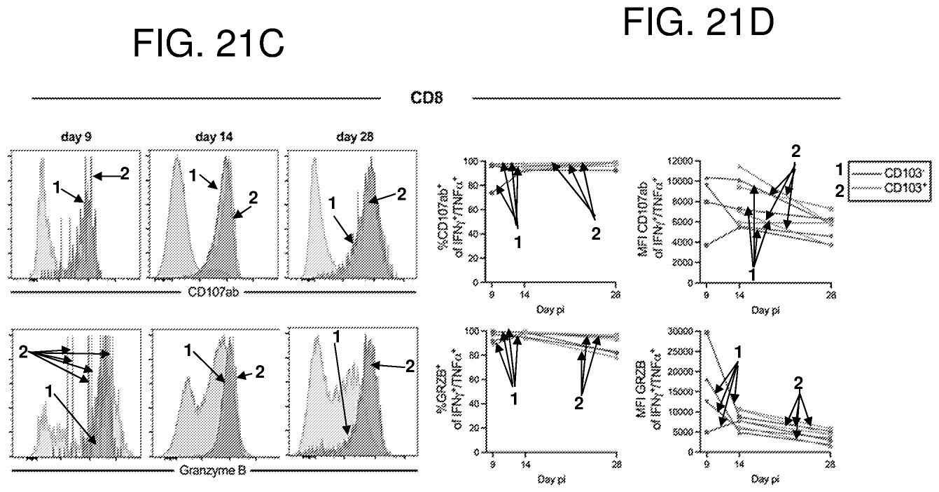

Furthermore, S-specific (IFNY*/TNFa+) CD4⁺ and CD8⁺ T-cells from BAL could be separated into circulating CD69⁻ CD103⁻ and tissue-resident memory (Trm) CD69⁺ CD103^{+/-} subsets (36) (FIGS. 14E and 14G, for CD4⁺ and CD8⁺ cells, respectively). An additional subset of presumably tissue-resident S-specific CD4⁺ and CD8⁺ T-cells was identified as CD69⁻ CD103⁺ and has been previously detected in SARS-COV-2-infected RMs (37). Circulating CD69⁻ CD103⁻ S-specific CD4+ and CD8+ T-cells were detectable in BAL on day 9 pi and were prominent until day 14, representing about 60% of the S-specific T-cells (FIGS. 13F and 13H). Lung-resident S-specific CD69⁺ CD103⁻ CD4+ and CD8+ T-cells were detectable from day 9 pi (FIGS. 13F and 13H) and their proportion increased through day 28 pi. On day 14, a fraction of these CD69⁺ S-specific T-cells had acquired CD103, and the proportion of CD69⁺ CD103⁺ Trm CD4⁺ and CD8⁺ T-cells increased from day 14 to 28 pi; by day 28 pi, about 80% of the S-specific T cells in the airways were positive for CD103 and/or CD69, indicating transition of antigen specific T cells to Trm phenotypes (FIGS. 13F and 13H). Following antigen stimulation, the S-specific circulating and tissue-resident CD4⁺ or CD8⁺ T-cells recovered from the airways on days 9, 14, or 28 were phenotypically comparable with respect to expression of CD107ab and granzyme B (FIG. 21), suggesting that they were highly functional. S-specific (IFNγ⁺/TNFα⁺) CD4⁺ and CD8⁺ T-cells in the blood mostly retained a circulating (CD69 CD103-) phenotype throughout day 28 post-immunization (FIGS. 19E-19G).

### Example 10: B/HPIV3/S-6P Immunization Protects RM Against SARS-COV-2 Challenge Virus Replication in the Upper and Lower Airways

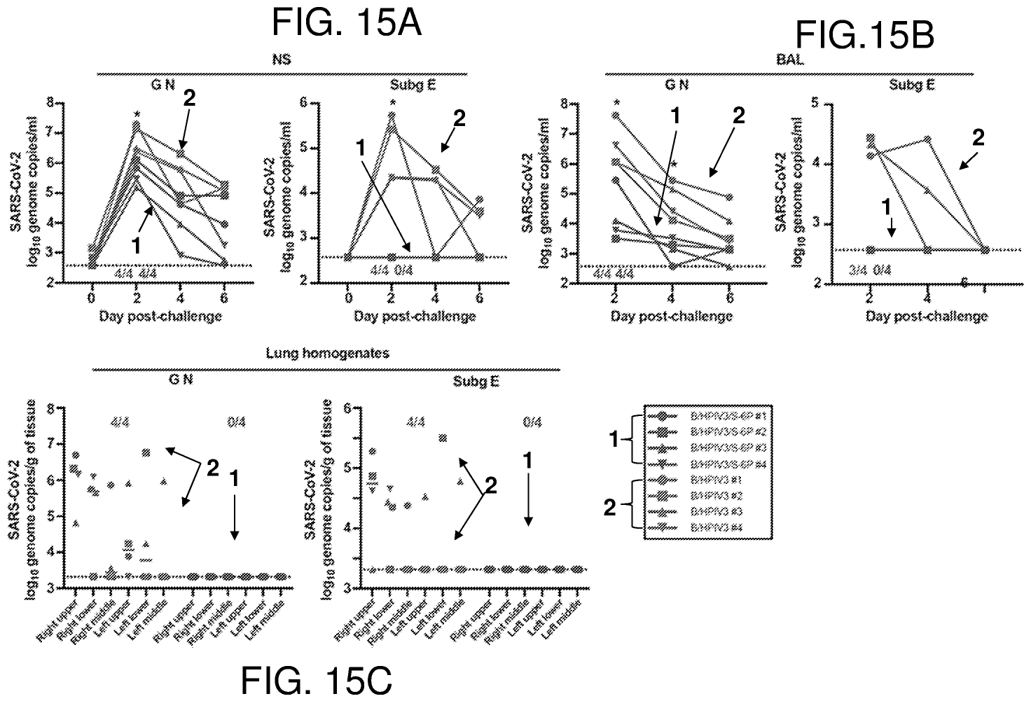

To assess protective efficacy of intranasal/intratracheal immunization with B/HPIV3/S-6P, RMs from both groups were challenged intranasally and intratracheally with 5.8 log₁₀ TCID₅₀ of SARS-COV-2 WA1/2020 on day 30 after immunization (FIG. 16). NS and BAL specimens were collected before challenge and on days 2, 4, and 6 after challenge. Viral RNA was extracted from these specimens, and the SARS-CoV-2 virus load was evaluated by RT-qPCR (FIGS. 15A-15B). The copy number of genomic N RNA/ml was maximal on day 2 pi in the UA and LA of all 8 RMs, and then steadily decreased over time. In the UA, RMs immunized with B/HPIV3/S-6P exhibited on average 16-fold less genomic N RNA copies/ml than the RMs immunized with B/HPIV3 empty vector control (6.8 log₁₀ and 5.6 log₁₀ copies/ml in the B/HPIV3- and the B/HPIV3/S-6P-immunized RMs, respectively, p<0.05). In the LA, B/HPIV3/S-6P-immunized RMs exhibited 240-fold lower levels of genomic N RNA copies/ml than RMs immunized with B/HPIV3 (6.6 log₁₀ and 4.2 log₁₀ copies in the B/HPIV3- and the B/HPIV3/S-6P-immunized RMs, respectively, p<0.05).

From the same samples, subgenomic E (sgE) mRNA, indicative of SARS-COV-2 mRNA synthesis and active virus replication, was also quantified. In B/HPIV3 empty-vector immunized RMs, sgE mRNA was detected in the UA and LA of 4 and 3 of 4 RMs immunized with B/HPIV3, and was maximal on day 2 post-challenge (pc; mean 5.0 log₁₀ copies/ml in the UA, and 4.3 log₁₀ copies/ml in the LA), and decreased until day 6 pc. In all 4 B/HPIV3/S-6P-immunized RMs sgE RNA was below the limit of detection in the UA and LA at all time points (p<0.05), showing that intranasal/intratracheal immunization with a single dose of B/HPIV3/S-6P induces robust protection against high levels of challenge virus replication.

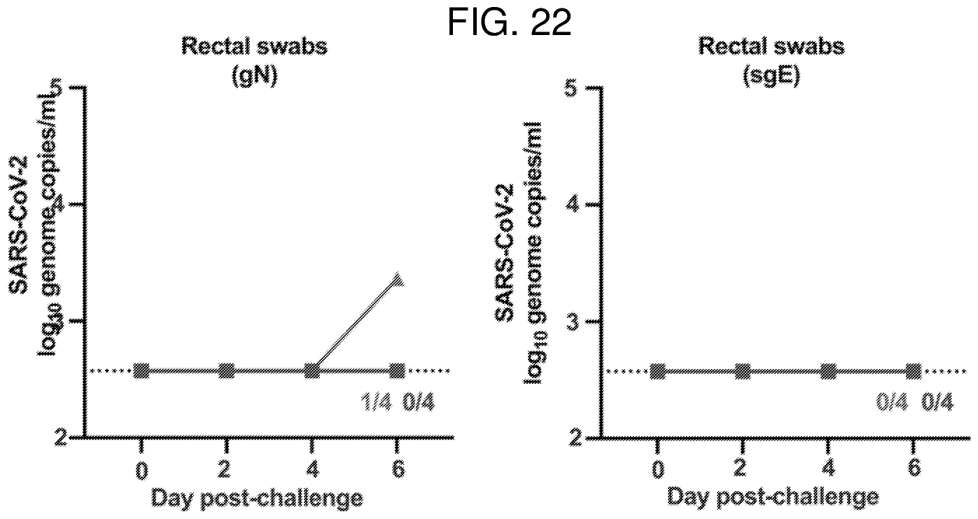

Quantification of genomic N (gN) RNA and sgE mRNA was also performed for lung tissues from different areas obtained on day 6 pc (FIG. 15C). Genomic N RNA and subgenomic E mRNA were detected in all 4 B/HPIV3-immunized RMs, mostly in the right upper and right lower lobes of the lungs. No genomic N RNA or subgenomic E mRNA were detected in the lungs of any B/HPIV3/S-6P-immunized RMs, confirming robust protection induced by B/HPIV3/S-6P. Furthermore, no active SARS-COV-2 replication was detected from rectal swabs samples (FIG. 22).

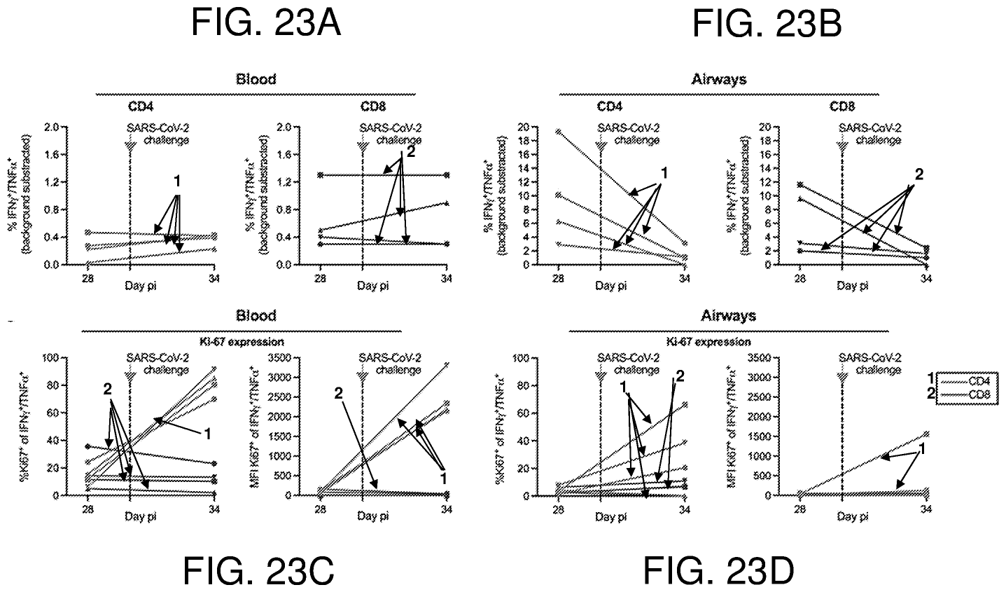

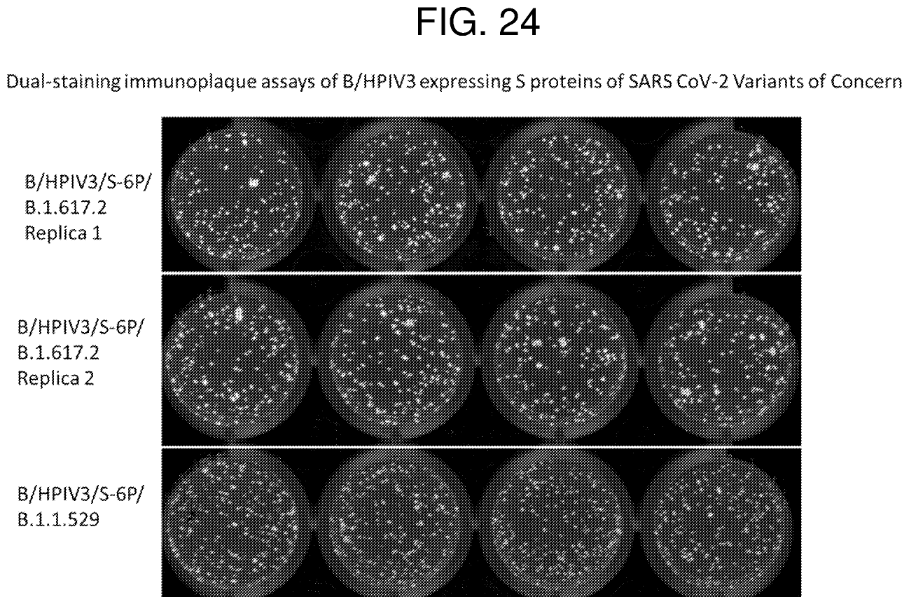

An additional study assessed the CD4+ and CD8+ T cell response in the blood (FIGS. 13C-13D) and lower airways (FIGS. 13E-13F) of immunized RMs, 4 days after challenge with SARS-COV-2. In the blood, an increase of S-specific IFNγ⁺/TNFα⁺ CD4+ and CD8+T cells was detected in 3 and 1 of 4 B/HPIV3/S-6P-immunized RMs, respectively that correlated well with the increased expression of Ki-67 by the S-specific CD4 T cells (FIG. 23C). A modest increase of S-specific IFNγ⁺/TNFα⁺ CD4+ T cells was also detected in 1 B/HPIV3-immunized RM (FIG. 13C). However, in the lower airways, a decrease rather than an increase of the S-specific CD4+ and CD8+T cells was detected in the B/HPIV3/S6-P-immunized RMs (FIG. 23D).

### REFERENCES FOR EXAMPLES 4-10

1. D. A. Rankin, R. Talj, L. M. Howard, N. B. Halasa, Epidemiologic trends and characteristics of SARS-COV-2 infections among children in the United States. Curr Opin Pediatr 33, 114-121 (2021).
2. A. L. Funk et al., Outcomes of SARS-COV-2-Positive Youths Tested in Emergency Departments: The Global PERN-COVID-19 Study. JAMA Netw Open 5, e2142322 (2022).
3. J. S. Gerber, P. A. Offit, COVID-19 vaccines for children. Science 374, 913 (2021).
4. L. Verdoni et al., An outbreak of severe Kawasaki-like disease at the Italian epicentre of the SARS-CoV-2 epidemic: an observational cohort study. Lancet 395, 1771-1778 (2020).
5. S. Riphagen, X. Gomez, C. Gonzalez-Martinez, N. Wilkinson, P. Theocharis, Hyperinflammatory shock in children during COVID-19 pandemic. Lancet 395, 1607-1608 (2020).
6. A. T. DiPiazza, B. S. Graham, T. J. Ruckwardt, T cell immunity to SARS-COV-2 following natural infection and vaccination. Biochem Biophys Res Commun 538, 211-217 (2021).
7. R. A. Karron et al., Evaluation of two chimeric bovine-human parainfluenza virus type 3 vaccines in infants and young children. Vaccine 30, 3975-3981 (2012).
8. D. I. Bernstein et al., Phase I study of the safety and immunogenicity of a live, attenuated respiratory syncytial virus and parainfluenza virus type 3 vaccine in seronegative children. Pediatr Infect Dis J 31, 109-114 (2012).
9. X. Liu et al., A single intranasal dose of a live-attenuated parainfluenza virus-vectored SARS-COV-2 vaccine is protective in hamsters. Proc Natl Acad Sci U S A 118, (2021).
10. A. C. Schmidt et al., Bovine parainfluenza virus type 3 (BPIV3) fusion and hemagglutinin-neuraminidase glycoproteins make an important contribution to the restricted replication of BPIV3 in primates. J Virol 74, 8922-8929 (2000).
11. B. Liang et al., Chimeric bovine/human parainfluenza virus type 3 expressing respiratory syncytial virus (RSV) F glycoprotein: effect of insert position on expression, replication, immunogenicity, stability, and protection against RSV infection. J Virol 88, 4237-4250 (2014).
12. C. L. Hsich et al., Structure-based design of prefusion-stabilized SARS-COV-2 spikes. Science 369, 1501-1505 (2020).
13. D. Wrapp et al., Cryo-EM structure of the 2019-nCOV spike in the prefusion conformation. Science 367, 1260-1263 (2020).
14. K. S. Corbett et al., SARS-COV-2 mRNA vaccine design enabled by prototype pathogen preparedness. Nature, (2020).
15. N. N. Jarjour, D. Masopust, S. C. Jameson, T Cell Memory: Understanding COVID-19. Immunity 54, 14-18 (2021).
16. P. A. Szabo et al., Longitudinal profiling of respiratory and systemic immune responses reveals myeloid cell-driven lung inflammation in severe COVID-19. Immunity 54, 797-814 e796 (2021).
17. J. E. Oh et al., Intranasal priming induces local lung-resident B cell populations that secrete protective mucosal antiviral IgA. Sci Immunol 6, cabj5129 (2021).
18. Z. Wang et al., Enhanced SARS-COV-2 neutralization by dimeric IgA. Sci Transl Med 13, (2021).
19. K. S. Corbett et al., Immune correlates of protection by mRNA-1273 vaccine against SARS-COV-2 in nonhuman primates. Science 373, cabj0299 (2021).
20. M. G. Joyce et al., A SARS-COV-2 ferritin nanoparticle vaccine elicits protective immune responses in nonhuman primates. Sci Transl Med, cabi5735 (2021).
21. K. S. Corbett et al., Evaluation of the mRNA-1273 Vaccine against SARS-COV-2 in Nonhuman Primates. N Engl J Med 383, 1544-1555 (2020).
22. K. S. Corbett et al., mRNA-1273 protects against SARS-COV-2 beta infection in nonhuman primates. Nat Immunol 22, 1306-1315 (2021).
23. N. B. Mercado et al., Single-shot Ad26 vaccine protects against SARS-COV-2 in rhesus macaques. Nature 586, 583-588 (2020).
24. A. Sette, S. Crotty, Adaptive immunity to SARS-COV-2 and COVID-19. Cell 184, 861-880 (2021).
25. K. McMahan et al., Correlates of protection against SARS-COV-2 in rhesus macaques. Nature 590, 630-634 (2021).
26. A. T. Tan et al., Early induction of functional SARS-COV-2-specific T cells associates with rapid viral clearance and mild disease in COVID-19 patients. Cell reports 34, 108728 (2021).
27. T. Nomura et al., Subacute SARS-COV-2 replication can be controlled in the absence of CD8+ T cells in cynomolgus macaques. PLOS Pathog 17, e1009668 (2021).
28. K. J. Hasenkrug et al., Recovery from Acute SARS-COV-2 Infection and Development of Anamnestic Immune Responses in T Cell-Depleted Rhesus Macaques. mBio 12, e0150321 (2021). J. E. Kohlmeier, S. C. Miller, D. L. Woodland, Cutting edge: Antigen is not required for the 29. activation and maintenance of virus-specific memory CD8+ T cells in the lung airways. J Immunol 178, 4721-4725 (2007).
30. H. X. Tan et al., Lung-resident memory B cells established after pulmonary influenza infection display distinct transcriptional and phenotypic profiles. Sci Immunol 7, eabf5314 (2022).
31. M. Auladell et al., Recalling the Future: Immunological Memory Toward Unpredictable Influenza Viruses. Front Immunol 10, 1400 (2019).
32. A. Tarke et al., Impact of SARS-COV-2 variants on the total CD4(+) and CD8(+) T cell reactivity in infected or vaccinated individuals. Cell Rep Med 2, 100355 (2021).
33. R. Keeton et al., T cell responses to SARS-COV-2 spike cross-recognize Omicron. Nature, (2022). A. Chandrashekar et al., Vaccine Protection Against the SARS-COV-2 Omicron Variant in 34. Macaques. bioRxiv, (2022).
35. A. C. Schmidt, J. M. Mcauliffe, B. R. Murphy, P. L. Collins, Recombinant bovine/human parainfluenza virus type 3 (B/HPIV3) expressing the respiratory syncytial virus (RSV) G and F proteins can be used to achieve simultaneous mucosal immunization against RSV and HPIV3. J Virol 75, 4594-4603 (2001).

In view of the many possible embodiments to which the principles of the disclosure may be applied, it should be recognized that the illustrated embodiments are only examples and should not be taken as limiting the scope of the disclosure. Rather, the scope of the disclosure is defined by the following claims.

1. A recombinant chimeric bovine/human parainfluenza virus 3 (rB/HPIV3), comprising:
a genome comprising, in a 3′ to 5′ order, a 3′ leader region, a BPIV3 N gene, a heterologous gene, BPIV3 P and M genes, HPIV3 F and HN genes, a BPIV3 L gene, and a 5′ trailer region;
wherein the heterologous gene encodes a recombinant SARS-COV-2 S protein comprising K986P and V987P substitutions and an amino acid sequence at least 90% identical to SEQ ID NO: 22;
wherein the HPIV3 HN gene encodes a HPIV3 HN protein comprising threonine and proline residues at positions 263 and 370, respectively, with reference to SEQ ID NO: 7; and
wherein the recombinant B/HPIV3 is infectious, attenuated, and self-replicating.

2. The rB/HPIV3 of claim 1, wherein the SARS-COV-2 S protein further comprises F817P, A892P, A899P, and A942P substitutions.

3. The rB/HPIV3 of claim 1, wherein the SARS-CoV-2 S protein further comprises one or more modifications selected from L18F, T19R, T20N, P26S, A67V, codon deletions 69-70, D80A, T95I, D138Y, G142D, codon deletions 142-144 or 143-145, Y145D, codon deletions 156-157, R158G, R190S, N211I, L212V, L212I, codon deletions 1213-214, codon insertions 213-214RE, D215G, R216E, G339D, S373P, S375F, K417N, N439K, N440K, G446S, L452R, S477G, S477N, T478K, E484K, E484A, E484Q, Q493R, S494P, G496S, Q498R, N501Y, Y505H, T547K, A570D, D614G, H655Y, N679K, P681H, P681R, A701V, T716K, N764K, D796Y, N856K, D950N, Q954H, N969K, L981F, S982A, T1027I, and D1118H.

4. The rB/HPIV3 of claim 1, wherein the SARS-COV-2 S protein further comprises K417N, E484K, N501Y, D614G, and A701V substitutions.

5. The rB/HPIV3 of claim 1, wherein the SARS-COV-2 S protein comprises or further comprises:
one or more deletions of amino acids H69, V70, Y144, L242, A243, and L244;
one or more of T19R, E156G, a F157 deletion, a R158 codon deletion, L452R, T478K, D614G, P681R, D950N; or one or more of A67V, a H69 deletion, V70 deletion, T95I, a N211 deletion, L212I, an insertion of 3 codons 214EPE, G142D, a 3-codon deletion V143, Y144, Y145, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F.

6. The rB/HPIV3 of claim 1, wherein a S1/S2 protease cleavage site of the S protein is mutated by amino acid substitution to inhibit S1/S2 protease cleavage.

7. The rB/HPIV3 of claim 6, wherein the mutation is a RRAR(682-685) GSAS substitution of the SARS-COV-2 S protein.

8. The rB/HPIV3 of claim 1, wherein the recombinant SARS-COV-2 protein comprises the amino acid substitutions and a sequence at least 95% identical to SEQ ID NO: 22.

9. The rB/HPIV3 of claim 8, wherein the recombinant SARS-COV-2 protein comprises the amino acid substitutions and a sequence at least 99% identical to SEQ ID NO: 22.

10. The rB/HPIV3 of claim 1, wherein the SARS-COV-2 S protein comprises or consists of the amino acid sequence set forth as any one of SEQ ID NOs: 23-26 or an amino acid sequence at least 90% identical thereto. JLC1/GKS:gh 10/12/23 E-239-2020-0-US-02 FILED VIA EFS ON OCTOBER 12, 2023

11. The rB/HPIV3 of any one of claims 1 10claim 1, wherein:
the BPIV3 N gene encodes an N protein comprising or consisting of the amino acid sequence set forth as SEQ ID NO: 1, or an amino acid sequence at least 90% identical thereto;
the BPIV3 P gene encodes P, C, and V proteins comprising or consisting of the amino acid sequences set forth as SEQ ID NOs: 2, 3, and 4, respectively, or amino acid sequences at least 90% identical thereto;
the BPIV3 M gene encodes an M protein comprising or consisting of the amino acid sequence set forth as SEQ ID NO: 5, or an amino acid sequence at least 90% identical thereto;
the HPIV3 F gene encodes an F protein comprising or consisting of the amino acid sequence set forth as SEQ ID NO: 6, or an amino acid sequence at least 90% identical thereto;
the HPIV3 HN gene encodes an HN protein comprising or consisting of the amino acid sequence set forth as SEQ ID NO: 7, or an amino acid sequence at least 90% identical thereto;
and/or the BPIV3 L gene encodes an L protein comprising or consisting of the amino acid sequence set forth as SEQ ID NO: 10, or an amino acid sequence at least 90% identical thereto.

12. The rB/HPIV3 of claim 1, wherein the heterologous gene is codon-optimized for expression in human cells.

13. The rB/HPIV3 of claim 12, wherein the heterologous gene that is codon optimized for human expression comprises an antigenomic cDNA sequence set forth as SEQ ID NO: 28, SEQ ID NO: 29, SEQ ID NO: 40 or SEQ ID NO: 41.

14. The rB/HPIV3 of claim 1, wherein the genome comprises an antigenomic cDNA sequence set forth as SEQ ID NO: 30, SEQ ID NO: 31, SEQ ID NO: 42 or SEQ ID NO: 43.

15. The rB/HPIV3 of claim 1, wherein the rB/HPIV3 induces an immune response to SARS-COV-2 S protein, HPIV3 F protein, and HPIV3 HN protein.

16. The rB/HPIV3 of claim 1, wherein the rB/HPIV3 induces an immune response that neutralizes SARS-COV-2 and HPIV3.

17. A nucleic acid molecule comprising the nucleotide sequence of the genome of the rB/HPIV3 of claim 1, or an antigenomic cDNA or RNA sequence of the genome.

18. A vector comprising the nucleic acid molecule of claim 17.

19. A host cell comprising the vector claim 18.

20. A method of producing a rB/HPIV3, comprising:
transfecting a permissive cell culture with the vector of claim 18;
incubating the cell culture for a sufficient period of time to allow for viral replication; and
purifying the replicated virus to produce the rB/HPIV3.

21. A rB/HPIV3 produced by the method of claim 20.

22. An immunogenic composition comprising a pharmaceutically acceptable carrier and the rB/HPIV3 of claim 1.

23. A method of eliciting an immune response to SARS-COV-2 and human parainfluenza virus 3 (HPIV3) in a subject, comprising administering the immunogenic composition of claim 22 to the subject to generate the immune response.

24. The method of claim 23, comprising intranasal administration of the immunogenic composition.

25. The method of claim 23, wherein the subject is a human.

26. The method of claim 23, wherein the subject is less than one year old.

27. The method of claim 23, wherein the immune response is a protective immune response.

28. The method of claim 27, wherein the protective immune response is elicited after a single dose of the immunogenic composition.

29. (canceled)


SEQUENCE LISTING (43 sequences)

SEQ ID NO: 1 - protein, 515 aa, Bovine parainfluenza virus type 3
MLSLFDTFSARRQENITKSAGGAVIPGQKNTVSIFALGPSITDDNDKMTLALLFLSHSLDNEKQHAQRAGFLVSLLSMAYANPELYLTSNGSNADVKYVIYMIEKDPGRQKYGGFVVKTREMVYEKTTDWMFGSDLEYDQDNMLQNGRSTSTIEDLVHTFGYPSCLGALIIQVWIILVKAITSISGLRKGFFTRLEAFRQDGTVKSSLVLSGDAVEQIGSIMRSQQSLVTLMVETLITMNTGRNDLTTIEKNIQIVGNYIRDAGLASFFNTIRYGIETRMAALTLSTLRPDINRLKALIELYLSKGPRAPFICILRDPVHGEFAPGNYPALWSYAMGVAVVQNKAMQQYVTGRSYLDIEMFQLGQAVARDAESQMSSILEDELGVTQEAKQSLKKHMKNISSSDTTFHKPTGGSAIEMAIDEEAGQPESRGDQDQGDEPRSSIVPYAWADETGNDNQTESTTEIDSIKTEQRNIRDRLNKRLNEKRKQSDPRSTDITNNTNQTEIDDLFSAFGSN

SEQ ID NO: 2 - protein, 596 aa, Bovine parainfluenza virus type 3
MEDNVQNNQIMDSWEEGSGDKSSDISSALDIIEFILSTDSQENTADSNEINTGTTRLSTTIYQPESKTTETSKENSGPANKNRQFGASHERATETKDRNVNQETVQGGYRRGSSPDSRTETMVTRRISRSSPDPNNGTQIQEDIDYNEVGEMDKDSTKREMRQFKDVPVKVSGSDAIPPTKQDGDGDDGRGLESISTFDSGYTSIVTAATLDDEEELLMKNNRPRKYQSTPQNSDKGIKKGVGRPKDTDKQSSILDYELNFKGSKKSQKILKASTNTGEPTRPQNGSQGKRITSWNILNSESGNRTESTNQTHQTSTSGQNHTMGPSRTTSEPRIKTQKTDGKEREDTEESTRFTERAITLLQNLGVIQSAAKLDLYQDKRVVCVANVLNNADTASKIDFLAGLMIGVSMDHDTKLNQIQNEILSLKTDLKKMDESHRRLIENQKEQLSLITSLISNLKIMTERGGKKDQPEPSGRTSMIKTKAKEEKIKKVRFDPLMETQGIEKNIPDLYRSIEKTPENDTQIKSEINRLNDESNATRLVPRRISSTMRSLIIIINNSNLSSKAKQSYINELKLCKSDEEVSELMDMFNEDVSSQ

SEQ ID NO: 3 - protein, 201 aa, Bovine parainfluenza virus type 3
MFKTIKSWILGKRDQEINHLTSHRPSTSLNSYSAPTPKRTRQTAMKSTQEPQDLARQSTNLNPKQQKQARKIVDQLTKIDSLGHHTNVPQRQKIEMLIRRLYREDIGEEAAQIVELRLWSLEESPEAAQILTMEPKSRKILITMKLERWIRTLLRGKCDNLKMFQSRYQEVMPFLQQNKMETVMMEEAWNLSVHLIQDIPV

SEQ ID NO: 4 - protein, 412 aa, Bovine parainfluenza virus type 3
MEDNVQNNQIMDSWEEGSGDKSSDISSALDIIEFILSTDSQENTADSNEINTGTTRLSTTIYQPESKTTETSKENSGPANKNRQFGASHERATETKDRNVNQETVQGGYRRGSSPDSRTETMVTRRISRSSPDPNNGTQIQEDIDYNEVGEMDKDSTKREMRQFKDVPVKVSGSDAIPPTKQDGDGDDGRGLESISTFDSGYTSIVTAATLDDEEELLMKNNRPRKYQSTPQNSDKGIKKGGWKAKRHRQTIINIGLRTQLQRIEEEPENPQSQHEYRRTNKTTEWIPGEENHILEHPQQRERQSNRINKPNPSDINLGTEPHNGTKQNNLRTKDQDTKDGWKGKRGHRREHSIYRKGDYIITESWCNPICSKIRPIPRQESCVCGECPKQCRYCIKDRLPSRFDDRSVNGS

SEQ ID NO: 5 - protein, 351 aa, Bovine parainfluenza virus type 3
MSITNSTIYTFPESSFSENGNIEPLPLKVNEQRKAIPHIRVVKIGDPPKHGSRYLDVFLLGFFEMERSKDRYGSISDLDDDPSYKVCGSGSLPLGLARYTGNDQELLQAATKLDIEVRRTVKATEMIVYTVQNIKPELYPWSSRLRKGMLFDANKVALAPQCLPLDRGIKFRVIFVNCTAIGSITLFKIPKSMALLSLPNTISINLQVHIKTGVQTDSKGVVQILDEKGEKSLNFMVHLGLIKRKMGRMYSVEYCKQKIEKMRLLFSLGLVGGISFHVNATGSISKTLASQLAFKREICYPLMDLNPHLNSVIWASSVEITRVDAVLQPSLPGEFRYYPNIIAKGVGKIRQ

SEQ ID NO: 6 - protein, 539 aa, Human parainfluenza virus type 3
MPTSILLIITTMIMASFCQIDITKLQHVGVLVNSPKGMKISQNFETRYLILSLIPKIEDSNSCGDQQIKQYKKLLDRLIIPLYDGLRLQKDVIVTNQESNENTDPRTKRFFGGVIGTIALGVATSAQITAAVALVEAKQARSDIEKLKEAIRDTNKAVQSVQSSIGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEIESCLSGNISQCPRTTVTSDIVPRYAFVNGGVVANCITTTCTCNGIGNRINQPPDQGVKIITHKECSTIGINGMLFNTNKEGTLAFYTPNDITLNNSVALDPIDISIELNKAKSDLEESKEWIRRSNQKLDSIGNWHQSSTTIIIILIMIIILFIINITIITIAIKYYRIQKRNRVDQNDKPYVLTNK

SEQ ID NO: 7 - protein, 572 aa, Human parainfluenza virus type 3
MEYWKHTNHGKDAGNELETSMATHGNKLTNKIIYILWTIILVLLSIVFIIVLINSIKSEKAHESLLQDINNEFMEITEKIQMASDNTNDLIQSGVNTRLLTIQSHVQNYIPISLTQQMSDLRKFISEITIRNDNQEVLPQRITHDVGIKPLNPDDFWRCTSGLPSLMKTPKIRLMPGPGLLAMPTTVDGCVRTPSLVINDLIYAYTSNLITRGCQDIGKSYQVLQIGIITVNSDLVPDLNPRISHTFNINDNRKSCSLALLNTDVYQLCSTPKVDERSDYASSGIEDIVLDIVNYDGSISTTRFKNNNISFDQPYAALYPSVGPGIYYKGKIIFLGYGGLEHPINENVICNTTGCPGKTQRDCNQASHSPWFSDRRMVNSIIVVDKGLNSIPKLKVWTISMRQNYWGSEGRLLLLGNKIYIYTRSTSWHSKLQLGIIDITDYSDIRIKWTWHNVLSRPGNNECPWGHSCPDGCITGVYTDAYPLNPTGSIVSSVILDSQKSRVNPVITYSTATERVNELAILNRTLSAGYTTTSCITHYNKGYCFHIVEINHKSLNTFQPMLFKTEIPKSCS

SEQ ID NO: 8 - protein, 572 aa, Human parainfluenza virus type 3
MEYWKHTNHGKDAGNELETSMATHGNKLTNKIIYILWTIILVLLSIVFIIVLINSIKSEKAHESLLQDINNEFMEITEKIQMASDNTNDLIQSGVNTRLLTIQSHVQNYIPISLTQQMSDLRKFISEITIRNDNQEVLPQRITHDVGIKPLNPDDFWRCTSGLPSLMKTPKIRLMPGPGLLAMPTTVDGCVRTPSLVINDLIYAYTSNLITRGCQDIGKSYQVLQIGIITVNSDLVPDLNPRISHTFNINDNRKSCSLALLNIDVYQLCSTPKVDERSDYASSGIEDIVLDIVNYDGSISTTRFKNNNISFDQPYAALYPSVGPGIYYKGKIIFLGYGGLEHPINENVICNTTGCPGKTQRDCNQASHSTWFSDRRMVNSIIVVDKGLNSIPKLKVWTISMRQNYWGSEGRLLLLGNKIYIYTRSTSWHSKLQLGIIDITDYSDIRIKWTWHNVLSRPGNNECPWGHSCPDGCITGVYTDAYPLNPTGSIVSSVILDSQKSRVNPVITYSTATERVNELAILNRTLSAGYTTTSCITHYNKGYCFHIVEINHKSLNTFQPMLFKTEIPKSCS

SEQ ID NO: 9 - DNA, 1719 bp, Human parainfluenza virus type 3
atggaatactggaagcataccaatcacggaaaggatgctggtaatgagctggagacgtctatggctactcatggcaacaagctcactaataagataatatacatattatggacaataatcctggtgttattatcaatagtcttcatcatagtgctaattaattccatcaaaagtgaaaaggcccacgaatcattgctgcaagacataaataatgagtttatggaaattacagaaaagatccaaatggcatcggataataccaatgatctaatacagtcaggagtgaatacaaggcttcttacaattcagagtcatgtccagaattacataccaatatcattgacacaacagatgtcagatcttaggaaattcattagtgaaattacaattagaaatgataatcaagaagtgctgccacaaagaataacacatgatgtaggtataaaacctttaaatccagatgatttttggagatgcacgtctggtcttccatctttaatgaaaactccaaaaataaggttaatgccagggccgggattattagctatgccaacgactgttgatggctgtgttagaactccgtctttagttataaatgatctgatttatgcttatacctcaaatctaattactcgaggttgtcaggatataggaaaatcatatcaagtcttacagatagggataataactgtaaactcagacttggtacctgacttaaatcctaggatctctcatacctttaacataaatgacaataggaagtcatgttctctagcactcctaaatatagatgtatatcaactgtgttcaactcccaaagttgatgaaagatcagattatgcatcatcaggcatagaagatattgtacttgatattgtcaattatgatggttcaatctcaacaacaagatttaagaataataacataagctttgatcaaccatatgctgcactatacccatctgttggaccagggatatactacaaaggcaaaataatatttctcgggtatggaggtcttgaacatccaataaatgagaatgtaatctgcaacacaactgggtgccccgggaaaacacagagagactgtaatcaagcatctcatagtacttggttttcagataggaggatggtcaactccatcattgttgttgacaaaggcttaaactcaattccaaaattgaaagtatggacgatatctatgcgacaaaattactgggggtcagaaggaaggttacttctactaggtaacaagatctatatatatacaagatctacaagttggcatagcaagttacaattaggaataattgatattactgattacagtgatataaggataaaatggacatggcataatgtgctatcaagaccaggaaacaatgaatgtccatggggacattcatgtccagatggatgtataacaggagtatatactgatgcatatccactcaatcccacagggagcattgtgtcatctgtcatattagactcacaaaaatcgagagtgaacccagtcataacttactcaacagcaaccgaaagagtaaacgagctggccatcctaaacagaacactctcagctggatatacaacaacaagctgcattacacactataacaaaggatattgttttcatatagtagaaataaatcataaaagcttaaacacatttcaacccatgttgttcaaaacagagattccaaaaagctgcagttaa

SEQ ID NO: 10 - protein, 2233 aa, Bovine parainfluenza virus type 3
MDTESHSGTTSDILYPECHLNSPIVKGKIAQLHTIMSLPQPYDMDDDSILIITRQKIKLNKLDKRQRSIRKLRSVLMERVSDLGKYTFIRYPEMSSEMFQLCIPGINNKINELLSKASKTYNQMTDGLRDLWVTILSKLASKNDGSNYDINEDISNISNVHMTYQSDKWYNPFKTWFTIKYDMRRLQKAKNEITFNRHKDYNLLEDQKNILLIHPELVLILDKQNYNGYIMTPELVLMYCDVVEGRWNISSCAKLDPKLQSMYYKGNNLWEIIDGLFSTLGERTFDIISLLEPLALSLIQTYDPVKQLRGAFLNHVLSEMELIFAAECTTEEIPNVDYIDKILDVFKESTIDEIAEIFSFFRTFGHPPLEASIAAEKVRKYMYTEKCLKFDTINKCHAIFCTIIINGYRERHGGQWPPVTLPVHAHEFIINAYGSNSAISYENAVDYYKSFIGIKFDKFIEPQLDEDLTIYMKDKALSPKKSNWDTVYPASNLLYRTNVSHDSRRLVEVFIADSKFDPHQVLDYVESGYWLDDPEFNISYSLKEKEIKQEGRLFAKMTYKMRATQVLSETLLANNIGKFFQENGMVKGEIELLKRLTTISMSGVPRYNEVYNNSKSHTEELQAYNAISSSNLSSNQKSKKFEFKSTDIYNDGYETVSCFLTTDLKKYCLNWRYESTALFGDTCNQIFGLKELFNWLHPRLEKSTIYVGDPYCPPSDIEHLPLDDHPDSGFYVHNPKGGIEGFCQKLWTLISISAIHLAAVKIGVRVTAMVQGDNQAIAVTTRVPNNYDYKVKKEIVYKDVVRFFDSLREVMDDLGHELKLNETIISSKMFIYSKRIYYDGRILPQALKALSRCVFWSETIIDETRSASSNLATSFAKAIENGYSPVLGYVCSIFKNIQQLYIALGMNINPTITQNIKDQYFRNIHWMQYASLIPASVGGFNYMAMSRCFVRNIGDPTVAALADIKRFIKANLLDRGVLYRIMNQEPGESSFLDWASDPYSCNLPQSQNITTMIKNITARNVLQDSPNPLLSGLFTSTMIEEDEELAEFLMDRRIILPRVAHDILDNSLTGIRNAIAGMLDTTKSLIRVGISRGGLTYNLLRKISNYDLVQYETLSKTLRLIVSDKIKYEDMCSVDLAISLRQKMWMHLSGGRMINGLETPDPLELLSGVIITGSEHCRICYSTEGESPYTWMYLPGNLNIGSAETGIASLRVPYFGSVTDERSEAQLGYIKNLSKPAKAAIRIAMIYTWAFGNDEISWMEASQIAQTRANFTLDSLKILTPVTTSTNLSHRLKDTATQMKFSSTSLIRVSRFITISNDNMSIKEANETKDTNLIYQQVMLTGLSVFEYLFRLEESTGHNPMVMHLHIEDGCCIKESYNDEHINPESTLELIKYPESNEFIYDKDPLKDIDLSKLMVIRDHSYTIDMNYWDDTDIVHAISICTAVTIADTMSQLDRDNLKELVVIANDDDINSLITEFLTLDILVFLKTFGGLLVNQFAYTLYGLKIEGRDPIWDYIMRTLKDTSHSVLKVLSNALSHPKVFKRFWDCGVLNPIYGPNTASQDQVKLALSICEYSLDLFMREWLNGASLEIYICDSDMEIANDRRQAFLSRHLAFVCCLAEIASFGPNLLNLTYLERLDELKQYLDLNIKEDPTLKYVQVSGLLIKSFPSTVTYVRKTAIKYLRIRGINPPETIEDWDPIEDENILDNIVKTVNDNCSDNQKRNKSSYFWGLALKNYQVVKIRSITSDSEVNEASNVTTHGMTLPQGGSYLSHQLRLFGVNSTSCLKALELSQILMREVKKDKDRLFLGEGAGAMLACYDATLGPAINYYNSGLNITDVIGQRELKIFPSEVSLVGKKLGNVTQILNRVRVLFNGNPNSTWIGNMECESLIWSELNDKSIGLVHCDMEGAIGKSEETVLHEHYSIIRITYLIGDDDVVLVSKIIPTITPNWSKILYLYKLYWKDVSVVSLKTSNPASTELYLISKDAYCTVMEPSNLVLSKLKRISSIEENNLLKWIILSKRKNNEWLQHEIKEGERDYGIMRPYHTALQIFGFQINLNHLAREFLSTPDLTNINNIIQSFTRTIKDVMFEWVNITHDNKRHKLGGRYNLFPLKNKGKLRLLSRRLVLSWISLSLSTRLLTGRFPDEKFENRAQTGYVSLADIDLESLKLLSRNIVKNYKEHIGLISYWFLTKEVKILMKLIGGVKLLGIPKQYKELEDRSSQGYEYDNEFDID

SEQ ID NO: 11 - DNA, 12 bp, Artificial Sequence
  Synthetic nucleic acid
aagtaagaaaaa

SEQ ID NO: 12 - DNA, 10 bp, Artificial Sequence
  Synthetic nucleic acid
aggattaaag

SEQ ID NO: 13 - DNA, 10 bp, Artificial Sequence
  Synthetic nucleic acid
aggacaaaag

SEQ ID NO: 14 - DNA, 10 bp, Artificial Sequence
  Synthetic nucleic acid
aggagtaaag

SEQ ID NO: 15 - DNA, 10 bp, Artificial Sequence
  Synthetic nucleic acid
aggagcaaag

SEQ ID NO: 16 - DNA, 12 bp, Artificial Sequence
  Synthetic nucleic acid
aaataagaaaaa

SEQ ID NO: 17 - DNA, 12 bp, Artificial Sequence
  Synthetic nucleic acid
aaataagaaaaa

SEQ ID NO: 18 - DNA, 20 bp, Artificial Sequence
  Synthetic nucleic acid
aaataaaggataatcaaaaa

SEQ ID NO: 19 - DNA, 12 bp, Artificial Sequence
  Synthetic nucleic acid
aattataaaaaa

SEQ ID NO: 20 - DNA, 12 bp, Artificial Sequence
  Synthetic nucleic acid
aaatataaaaaa

SEQ ID NO: 21 - DNA, 13 bp, Artificial Sequence
  Synthetic nucleic acid
aaagtaagaaaaa

SEQ ID NO: 22 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 23 - protein, 1273 aa, Artificial Sequence
  Recombinant protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 24 - protein, 1273 aa, Artificial Sequence
  Recombinant protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 25 - protein, 1273 aa, Artificial Sequence
  Recombinant protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 26 - protein, 1273 aa, Artificial Sequence
  Recombinant protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 27 - DNA, 3822 bp, Artificial Sequence
  Recombinant nucleic acid
atgttcgtgtttctggtgctgctgcctctggtgagctcccagtgcgtgaacctgaccacaaggacccagctgccccctgcctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagcggttcgacaatccagtgctgccctttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcgagtttcgcgtgtattcctctgccaacaattgcacatttgagtacgtgtcccagcccttcctgatggacctggagggcaagcagggcaatttcaagaacctgagggagttcgtgtttaagaatatcgatggctacttcaagatctactccaagcacaccccaatcaacctggtgcgcgacctgccacagggcttctctgccctggagccactggtggatctgcccatcggcatcaacatcacccggtttcagacactgctggccctgcacagaagctacctgacaccaggcgacagctcctctggatggaccgcaggagctgccgcctactatgtgggctatctgcagcccaggaccttcctgctgaagtacaacgagaatggcaccatcacagacgccgtggattgcgccctggatcccctgtctgagaccaagtgtacactgaagagctttaccgtggagaagggcatctatcagacaagcaatttcagggtgcagcctaccgagtccatcgtgcgctttcccaatatcacaaacctgtgcccttttggcgaggtgttcaacgcaacccgcttcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcccctggccagacaggcaagatcgccgactacaattataagctgcctgacgatttcaccggctgcgtgatcgcctggaactctaacaatctggatagcaaagtgggcggcaactacaattatctgtaccggctgtttagaaagtctaatctgaagccattcgagagggacatctccacagagatctaccaggccggctctaccccctgcaatggcgtggagggctttaactgttatttccctctgcagagctacggcttccagccaacaaacggcgtgggctatcagccctaccgcgtggtggtgctgtcttttgagctgctgcacgcacctgcaacagtgtgcggaccaaagaagagcaccaatctggtgaagaacaagtgcgtgaacttcaacttcaacggactgaccggcacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcaggacgtgaattgtaccgaggtgcccgtggcaatccacgcagatcagctgacccctacatggcgggtgtactctaccggcagcaacgtgttccagacaagagccggatgcctgatcggagccgagcacgtgaacaatagctatgagtgcgacatccctatcggcgccggcatctgtgcctcctaccagacccagacaaactccccacggagagcccggtctgtggccagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggctctttttgtacccagctgaatagagccctgacaggcatcgccgtggagcaggacaagaacacacaggaggtgttcgcccaggtgaagcagatctacaagaccccacccatcaaggactttggcggcttcaacttcagccagatcctgcccgatcctagcaagccatccaagcggtcttttatcgaggacctgctgttcaacaaggtgaccctggccgatgccggcttcatcaagcagtatggcgattgcctgggcgacatcgccgccagagacctgatctgtgcccagaagtttaatggcctgaccgtgctgcctccactgctgacagatgagatgatcgcccagtacacatctgccctgctggccggcaccatcacaagcggatggaccttcggcgcaggagccgccctgcagatcccctttgccatgcagatggcctatcggttcaacggcatcggcgtgacccagaatgtgctgtacgagaaccagaagctgatcgccaatcagtttaactccgccatcggcaagatccaggactctctgagctccacagccagcgccctgggcaagctgcaggatgtggtgaatcagaacgcccaggccctgaataccctggtgaagcagctgtctagcaacttcggcgccatctcctctgtgctgaatgatatcctgagcaggctggacaaggtggaggcagaggtgcagatcgaccggctgatcacaggcagactgcagtccctgcagacctacgtgacacagcagctgatcagggcagcagagatcagggcctctgccaatctggccgccaccaagatgagcgagtgcgtgctgggccagtccaagagagtggacttttgtggcaagggctatcacctgatgagcttcccacagtccgcccctcacggagtggtgtttctgcacgtgacctacgtgccagcccaggagaagaacttcaccacagcaccagcaatctgccacgatggcaaggcacactttcctagggagggcgtgttcgtgagcaacggcacccactggtttgtgacacagcgcaatttctacgagccacagatcatcaccacagacaatacattcgtgtccggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgacaggctgaacgaggtggccaagaatctgaacgagtccctgatcgatctgcaggagctgggcaagtatgagcagtacatcaagtggccctggtatatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactacacctga

SEQ ID NO: 28 - DNA, 3822 bp, Artificial Sequence
  Recombinant nucleic acid
atgttcgtgtttctggtgctgctgcctctggtgagctcccagtgcgtgaacctgaccacaaggacccagctgccccctgcctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagcggttcgacaatccagtgctgccctttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcgagtttcgcgtgtattcctctgccaacaattgcacatttgagtacgtgtcccagcccttcctgatggacctggagggcaagcagggcaatttcaagaacctgagggagttcgtgtttaagaatatcgatggctacttcaagatctactccaagcacaccccaatcaacctggtgcgcgacctgccacagggcttctctgccctggagccactggtggatctgcccatcggcatcaacatcacccggtttcagacactgctggccctgcacagaagctacctgacaccaggcgacagctcctctggatggaccgcaggagctgccgcctactatgtgggctatctgcagcccaggaccttcctgctgaagtacaacgagaatggcaccatcacagacgccgtggattgcgccctggatcccctgtctgagaccaagtgtacactgaagagctttaccgtggagaagggcatctatcagacaagcaatttcagggtgcagcctaccgagtccatcgtgcgctttcccaatatcacaaacctgtgcccttttggcgaggtgttcaacgcaacccgcttcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcccctggccagacaggcaagatcgccgactacaattataagctgcctgacgatttcaccggctgcgtgatcgcctggaactctaacaatctggatagcaaagtgggcggcaactacaattatctgtaccggctgtttagaaagtctaatctgaagccattcgagagggacatctccacagagatctaccaggccggctctaccccctgcaatggcgtggagggctttaactgttatttccctctgcagagctacggcttccagccaacaaacggcgtgggctatcagccctaccgcgtggtggtgctgtcttttgagctgctgcacgcacctgcaacagtgtgcggaccaaagaagagcaccaatctggtgaagaacaagtgcgtgaacttcaacttcaacggactgaccggcacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcaggacgtgaattgtaccgaggtgcccgtggcaatccacgcagatcagctgacccctacatggcgggtgtactctaccggcagcaacgtgttccagacaagagccggatgcctgatcggagccgagcacgtgaacaatagctatgagtgcgacatccctatcggcgccggcatctgtgcctcctaccagacccagacaaactccccagggtctgcctcctctgtggccagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggctctttttgtacccagctgaatagagccctgacaggcatcgccgtggagcaggacaagaacacacaggaggtgttcgcccaggtgaagcagatctacaagaccccacccatcaaggactttggcggcttcaacttcagccagatcctgcccgatcctagcaagccatccaagcggtcttttatcgaggacctgctgttcaacaaggtgaccctggccgatgccggcttcatcaagcagtatggcgattgcctgggcgacatcgccgccagagacctgatctgtgcccagaagtttaatggcctgaccgtgctgcctccactgctgacagatgagatgatcgcccagtacacatctgccctgctggccggcaccatcacaagcggatggaccttcggcgcaggagccgccctgcagatcccctttgccatgcagatggcctatcggttcaacggcatcggcgtgacccagaatgtgctgtacgagaaccagaagctgatcgccaatcagtttaactccgccatcggcaagatccaggactctctgagctccacagccagcgccctgggcaagctgcaggatgtggtgaatcagaacgcccaggccctgaataccctggtgaagcagctgtctagcaacttcggcgccatctcctctgtgctgaatgatatcctgagcaggctggaccctccagaggcagaggtgcagatcgaccggctgatcacaggcagactgcagtccctgcagacctacgtgacacagcagctgatcagggcagcagagatcagggcctctgccaatctggccgccaccaagatgagcgagtgcgtgctgggccagtccaagagagtggacttttgtggcaagggctatcacctgatgagcttcccacagtccgcccctcacggagtggtgtttctgcacgtgacctacgtgccagcccaggagaagaacttcaccacagcaccagcaatctgccacgatggcaaggcacactttcctagggagggcgtgttcgtgagcaacggcacccactggtttgtgacacagcgcaatttctacgagccacagatcatcaccacagacaatacattcgtgtccggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgacaggctgaacgaggtggccaagaatctgaacgagtccctgatcgatctgcaggagctgggcaagtatgagcagtacatcaagtggccctggtatatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactacacctga

SEQ ID NO: 29 - DNA, 3822 bp, Artificial Sequence
  Recombinant nucleic acid
atgttcgtgtttctggtgctgctgcctctggtgagctcccagtgcgtgaacctgaccacaaggacccagctgccccctgcctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagcggttcgacaatccagtgctgccctttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcgagtttcgcgtgtattcctctgccaacaattgcacatttgagtacgtgtcccagcccttcctgatggacctggagggcaagcagggcaatttcaagaacctgagggagttcgtgtttaagaatatcgatggctacttcaagatctactccaagcacaccccaatcaacctggtgcgcgacctgccacagggcttctctgccctggagccactggtggatctgcccatcggcatcaacatcacccggtttcagacactgctggccctgcacagaagctacctgacaccaggcgacagctcctctggatggaccgcaggagctgccgcctactatgtgggctatctgcagcccaggaccttcctgctgaagtacaacgagaatggcaccatcacagacgccgtggattgcgccctggatcccctgtctgagaccaagtgtacactgaagagctttaccgtggagaagggcatctatcagacaagcaatttcagggtgcagcctaccgagtccatcgtgcgctttcccaatatcacaaacctgtgcccttttggcgaggtgttcaacgcaacccgcttcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcccctggccagacaggcaagatcgccgactacaattataagctgcctgacgatttcaccggctgcgtgatcgcctggaactctaacaatctggatagcaaagtgggcggcaactacaattatctgtaccggctgtttagaaagtctaatctgaagccattcgagagggacatctccacagagatctaccaggccggctctaccccctgcaatggcgtggagggctttaactgttatttccctctgcagagctacggcttccagccaacaaacggcgtgggctatcagccctaccgcgtggtggtgctgtcttttgagctgctgcacgcacctgcaacagtgtgcggaccaaagaagagcaccaatctggtgaagaacaagtgcgtgaacttcaacttcaacggactgaccggcacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcaggacgtgaattgtaccgaggtgcccgtggcaatccacgcagatcagctgacccctacatggcgggtgtactctaccggcagcaacgtgttccagacaagagccggatgcctgatcggagccgagcacgtgaacaatagctatgagtgcgacatccctatcggcgccggcatctgtgcctcctaccagacccagacaaactccccagggtctgcctcctctgtggccagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggctctttttgtacccagctgaatagagccctgacaggcatcgccgtggagcaggacaagaacacacaggaggtgttcgcccaggtgaagcagatctacaagaccccacccatcaaggactttggcggcttcaacttcagccagatcctgcccgatcctagcaagccatccaagcggtctcctatcgaggacctgctgttcaacaaggtgaccctggccgatgccggcttcatcaagcagtatggcgattgcctgggcgacatcgccgccagagacctgatctgtgcccagaagtttaatggcctgaccgtgctgcctccactgctgacagatgagatgatcgcccagtacacatctgccctgctggccggcaccatcacaagcggatggaccttcggcgcaggacccgccctgcagatcccctttcccatgcagatggcctatcggttcaacggcatcggcgtgacccagaatgtgctgtacgagaaccagaagctgatcgccaatcagtttaactccgccatcggcaagatccaggactctctgagctccacacccagcgccctgggcaagctgcaggatgtggtgaatcagaacgcccaggccctgaataccctggtgaagcagctgtctagcaacttcggcgccatctcctctgtgctgaatgatatcctgagcaggctggaccctccagaggcagaggtgcagatcgaccggctgatcacaggcagactgcagtccctgcagacctacgtgacacagcagctgatcagggcagcagagatcagggcctctgccaatctggccgccaccaagatgagcgagtgcgtgctgggccagtccaagagagtggacttttgtggcaagggctatcacctgatgagcttcccacagtccgcccctcacggagtggtgtttctgcacgtgacctacgtgccagcccaggagaagaacttcaccacagcaccagcaatctgccacgatggcaaggcacactttcctagggagggcgtgttcgtgagcaacggcacccactggtttgtgacacagcgcaatttctacgagccacagatcatcaccacagacaatacattcgtgtccggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgacaggctgaacgaggtggccaagaatctgaacgagtccctgatcgatctgcaggagctgggcaagtatgagcagtacatcaagtggccctggtatatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactacacctga

SEQ ID NO: 30 - DNA, 19314 bp, Artificial Sequence
  Recombinant nucleic acid
accaaacaagagaagagactggtttgggaatattaattcaaataaaaattaacttaggattaaagaactttaccgaaaggtaaggggaaagaaatcctaagagcttagccatgttgagtctattcgacacattcagtgcgcgtaggcaggagaacataacgaaatcagctggtggggctgttattcccgggcaaaaaaacactgtgtctatatttgctcttggaccatcaataacagatgacaatgataaaatgacattggctcttctctttttgtctcattctttagacaatgaaaagcagcatgcgcaaagagctggatttttagtttctctgttatcaatggcttatgccaacccagaattatatttaacatcaaatggtagtaatgcagatgttaaatatgttatctacatgatagagaaagacccaggaagacagaaatatggtgggtttgtcgtcaagactagagagatggtttatgaaaagacaactgattggatgttcgggagtgatcttgagtatgatcaagacaatatgttgcaaaatggtagaagcacttctacaatcgaggatcttgttcatacttttggatatccatcgtgtcttggagcccttataatccaagtttggataatacttgttaaggctataaccagtatatcaggattgaggaaaggattctttactcggttagaagcatttcgacaagatggaacagttaaatccagtctagtgttgagcggtgatgcagtagaacaaattggatcaattatgaggtcccaacagagcttggtaacactcatggttgaaacactgataacaatgaacacaggcaggaatgatctgacaacaatagaaaagaatatacagattgtaggaaactacatcagagatgcaggtcttgcttcatttttcaacacaatcagatatggcattgagactagaatggcagctctaactctgtctacccttagaccggatatcaacagactcaaggcactgatcgagttatatctatcaaaggggccacgtgctccttttatatgcattttgagagatcccgtgcatggtgagtttgcaccaggcaactatcctgccctctggagttatgcgatgggtgtagcagttgtacaaaacaaggccatgcaacagtatgtaacaggaaggtcttatctggatattgaaatgttccaacttggtcaagcagtggcacgtgatgccgagtcgcagatgagttcaatattagaggatgaactgggggtcacacaagaagccaagcaaagcttgaagaaacacatgaagaacatcagcagttcagatacaacctttcataagcctacagggggatcagccatagaaatggcgatagatgaagaagcagggcagcctgaatccagaggagatcaggatcaaggagatgagcctcggtcatccatagttccttatgcatgggcagacgaaaccgggaatgacaatcaaactgaatcaactacagaaattgacagcatcaaaactgaacaaagaaacatcagagacaggctgaacaaaagactcaacgagaaaaggaaacagagtgacccgagatcaactgacatcacaaacaacacaaatcaaactgaaatagatgatttgttcagtgcattcggaagcaactagtcacaaagagatgaccaggcgcgccaagtaagaaaaacttaggattaatggacctgcaggatgttcgtgtttctggtgctgctgcctctggtgagctcccagtgcgtgaacctgaccacaaggacccagctgccccctgcctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagcggttcgacaatccagtgctgccctttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcgagtttcgcgtgtattcctctgccaacaattgcacatttgagtacgtgtcccagcccttcctgatggacctggagggcaagcagggcaatttcaagaacctgagggagttcgtgtttaagaatatcgatggctacttcaagatctactccaagcacaccccaatcaacctggtgcgcgacctgccacagggcttctctgccctggagccactggtggatctgcccatcggcatcaacatcacccggtttcagacactgctggccctgcacagaagctacctgacaccaggcgacagctcctctggatggaccgcaggagctgccgcctactatgtgggctatctgcagcccaggaccttcctgctgaagtacaacgagaatggcaccatcacagacgccgtggattgcgccctggatcccctgtctgagaccaagtgtacactgaagagctttaccgtggagaagggcatctatcagacaagcaatttcagggtgcagcctaccgagtccatcgtgcgctttcccaatatcacaaacctgtgcccttttggcgaggtgttcaacgcaacccgcttcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcccctggccagacaggcaagatcgccgactacaattataagctgcctgacgatttcaccggctgcgtgatcgcctggaactctaacaatctggatagcaaagtgggcggcaactacaattatctgtaccggctgtttagaaagtctaatctgaagccattcgagagggacatctccacagagatctaccaggccggctctaccccctgcaatggcgtggagggctttaactgttatttccctctgcagagctacggcttccagccaacaaacggcgtgggctatcagccctaccgcgtggtggtgctgtcttttgagctgctgcacgcacctgcaacagtgtgcggaccaaagaagagcaccaatctggtgaagaacaagtgcgtgaacttcaacttcaacggactgaccggcacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcaggacgtgaattgtaccgaggtgcccgtggcaatccacgcagatcagctgacccctacatggcgggtgtactctaccggcagcaacgtgttccagacaagagccggatgcctgatcggagccgagcacgtgaacaatagctatgagtgcgacatccctatcggcgccggcatctgtgcctcctaccagacccagacaaactccccagggtctgcctcctctgtggccagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggctctttttgtacccagctgaatagagccctgacaggcatcgccgtggagcaggacaagaacacacaggaggtgttcgcccaggtgaagcagatctacaagaccccacccatcaaggactttggcggcttcaacttcagccagatcctgcccgatcctagcaagccatccaagcggtcttttatcgaggacctgctgttcaacaaggtgaccctggccgatgccggcttcatcaagcagtatggcgattgcctgggcgacatcgccgccagagacctgatctgtgcccagaagtttaatggcctgaccgtgctgcctccactgctgacagatgagatgatcgcccagtacacatctgccctgctggccggcaccatcacaagcggatggaccttcggcgcaggagccgccctgcagatcccctttgccatgcagatggcctatcggttcaacggcatcggcgtgacccagaatgtgctgtacgagaaccagaagctgatcgccaatcagtttaactccgccatcggcaagatccaggactctctgagctccacagccagcgccctgggcaagctgcaggatgtggtgaatcagaacgcccaggccctgaataccctggtgaagcagctgtctagcaacttcggcgccatctcctctgtgctgaatgatatcctgagcaggctggaccctccagaggcagaggtgcagatcgaccggctgatcacaggcagactgcagtccctgcagacctacgtgacacagcagctgatcagggcagcagagatcagggcctctgccaatctggccgccaccaagatgagcgagtgcgtgctgggccagtccaagagagtggacttttgtggcaagggctatcacctgatgagcttcccacagtccgcccctcacggagtggtgtttctgcacgtgacctacgtgccagcccaggagaagaacttcaccacagcaccagcaatctgccacgatggcaaggcacactttcctagggagggcgtgttcgtgagcaacggcacccactggtttgtgacacagcgcaatttctacgagccacagatcatcaccacagacaatacattcgtgtccggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgacaggctgaacgaggtggccaagaatctgaacgagtccctgatcgatctgcaggagctgggcaagtatgagcagtacatcaagtggccctggtatatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactacacctgatagtaactagcggcgcgccagcaacaagtaagaaaaacttaggattaatggaaattatccaatccagagacggaaggacaaatccagaatccaaccacaactcaatcaaccaaagattcatggaagacaatgttcaaaacaatcaaatcatggattcttgggaagagggatcaggagataaatcatctgacatctcatcggccctcgacatcattgaattcatactcagcaccgactcccaagagaacacggcagacagcaatgaaatcaacacaggaaccacaagacttagcacgacaatctaccaacctgaatccaaaacaacagaaacaagcaaggaaaatagtggaccagctaacaaaaatcgacagtttggggcatcacacgaacgtgccacagagacaaaagatagaaatgttaatcaggagactgtacagggaggatataggagaggaagcagcccagatagtagaactgagactatggtcactcgaagaatctccagaagcagcccagatcctaacaatggaacccaaatccaggaagatattgattacaatgaagttggagagatggataaggactctactaagagggaaatgcgacaatttaaagatgttccagtcaaggtatcaggaagtgatgccattcctccaacaaaacaagatggagacggtgatgatggaagaggcctggaatctatcagtacatttgattcaggatataccagtatagtgactgccgcaacactagatgacgaagaagaactccttatgaagaacaacaggccaagaaagtatcaatcaacaccccagaacagtgacaagggaattaaaaaaggggttggaaggccaaaagacacagacaaacaatcatcaatattggactacgaactcaacttcaaaggatcgaagaagagccagaaaatcctcaaagccagcacgaatacaggagaaccaacaagaccacagaatggatcccaggggaagagaatcacatcctggaacatcctcaacagcgagagcggcaatcgaacagaatcaacaaaccaaacccatcagacatcaacctcgggacagaaccacacaatgggaccaagcagaacaacctccgaaccaaggatcaagacacaaaagacggatggaaaggaaagagaggacacagaagagagcactcgatttacagaaagggcgattacattattacagaatcttggtgtaatccaatctgcagcaaaattagacctataccaagacaagagagttgtgtgtgtggcgaatgtcctaaacaatgcagatactgcatcaaagatagacttcctagcaggtttgatgataggagtgtcaatggatcatgataccaaattaaatcagattcagaacgagatattaagtttgaaaactgatcttaaaaagatggatgaatcacatagaagactaattgagaatcaaaaagaacaattatcactgatcacatcattaatctcaaatcttaaaattatgacagagagaggagggaagaaggaccaaccagaacctagcgggaggacatccatgatcaagacaaaagcaaaagaagagaaaataaagaaagtcaggtttgaccctcttatggaaacacagggcatcgagaaaaacatccctgacctctatagatcaatagagaaaacaccagaaaacgacacacagatcaaatcagaaataaacagattgaatgatgaatccaatgccactagattagtacctagaagaataagcagtacaatgagatcattaataataatcattaacaacagcaatttatcatcaaaagcaaagcaatcatacatcaacgaactcaagctctgcaagagtgacgaggaagtgtctgagttgatggacatgttcaatgaggatgtcagctcccagtaaaccgccaaccaagggtcaacaccaagaaaaccaatagcacaaaacagccaatcagagaccaccccaatacaccaaaccaatcaacacataacaaagatcgcggccgcatagatgattaagaaaaacttaggatgaaaggactaatcaatcctccgaaacaatgagcatcaccaactccacaatctacacattcccagaatcctctttctccgagaatggcaacatagagccgttaccactcaaggtcaatgaacagagaaaggccatacctcatattagggttgtcaagataggagatccgcccaaacatggatccagatatctggatgtctttttactgggcttctttgagatggaaaggtcaaaagacaggtatgggagcataagtgatctagatgatgatccaagttacaaggtttgtggctctggatcattgccacttgggttggctagatacaccggaaatgatcaggaactcctacaggctgcaaccaagctcgatatagaagtaagaagaactgtaaaggctacggagatgatagtttacactgtacaaaacatcaaacctgaactatatccatggtccagtagattaagaaaagggatgttatttgacgctaataaggttgcacttgctcctcaatgtcttccactagatagagggataaaattcagggtgatatttgtgaactgcacagcaattggatcaataactctattcaaaatccctaagtccatggcattgttatcattgcctaatacaatatcaataaatctacaagtacatatcaaaacaggagttcagacagattccaaaggagtagttcagattctagatgaaaaaggtgaaaaatcactaaatttcatggttcatctcgggttgatcaaaaggaagatgggcagaatgtactcagttgaatattgtaagcagaagatcgagaagatgagattattattctcattgggattagttggagggatcagcttccacgtcaacgcaactggctctatatcaaagacattagcaagtcaattagcattcaaaagagaaatctgctatcccctaatggatctgaatccacacttaaattcagttatatgggcatcatcagttgaaattacaagggtagatgcagttctccagccttcattacctggcgaattcagatactacccaaacatcatagcaaaaggggtcgggaaaatcagacagtaaaatcaacaaccctgatatccaccggtgtattaagccgaagcaaataaaggataatcaaaaacttaggacaaaagaggtcaataccaacaactattagcagtcacactcgcaagaataagagagaagggaccaaaaaagtcaaataggagaaatcaaaacaaaaggtacagaacaccagaacaacaaaatcaaaacatccaactcactcaaaacaaaaattccaaaagagaccggcaacacaacaagcactgaacacaatgccaacttcaatactgctaattattacaaccatgatcatggcatctttctgccaaatagatatcacaaaactacagcacgtaggtgtattggtcaacagtcccaaagggatgaagatatcacaaaactttgaaacaagatatctaattttgagcctcataccaaaaatagaagactctaactcttgtggtgaccaacagatcaagcaatacaagaagttattggatagactgatcatccctttatatgatggattaagattacagaaagatgtgatagtaaccaatcaagaatccaatgaaaacactgatcccagaacaaaacgattctttggaggggtaattggaaccattgctctgggagtagcaacctcagcacaaattacagcggcagttgctctggttgaagccaagcaggcaagatcagacatcgaaaaactcaaagaagcaattagggacacaaacaaagcagtgcagtcagttcagagctccataggaaatttaatagtagcaattaaatcagtccaggattatgttaacaaagaaatcgtgccatcgattgcgaggctaggttgtgaagcagcaggacttcaattaggaattgcattaacacagcattactcagaattaacaaacatatttggtgataacataggatcgttacaagaaaaaggaataaaattacaaggtatagcatcattataccgcacaaatatcacagaaatattcacaacatcaacagttgataaatatgatatctatgatctgttatttacagaatcaataaaggtgagagttatagatgttgacttgaatgattactcaatcaccctccaagtcagactccctttattaactaggctgctgaacactcagatctacaaagtagattccatatcatataacatccaaaacagagaatggtatatccctcttcccagccatatcatgacgaaaggggcatttctaggtggagcagacgtcaaagaatgtatagaagcattcagcagctatatatgcccttctgatccaggatttgtattaaaccatgaaatagagagctgcttatcaggaaacatatcccaatgtccaagaacaacggtcacatcagacattgttccaagatatgcatttgtcaatggaggagtggttgcaaactgtataacaaccacctgtacatgcaacggaattggtaatagaatcaatcaaccacctgatcaaggagtaaaaattataacacataaagaatgtagtacaataggtatcaacggaatgctgttcaatacaaataaagaaggaactcttgcattctatacaccaaatgatataacactaaacaattctgttgcacttgatccaattgacatatcaatcgagctcaacaaggccaaatcagatctagaagaatcaaaagaatggataagaaggtcaaatcaaaaactagattctattggaaattggcatcaatctagcactacaatcataattattttgataatgatcattatattgtttataattaatataacgataattacaattgcaattaagtattacagaattcaaaagagaaatcgagtggatcaaaatgacaagccatatgtactaacaaacaaataacatatctacagatcattagatattaaaattataaaaaacttaggagtaaagttacgcaatccaactctactcatataattgaggaaggacccaatagacaaatccaaattcgagatggaatactggaagcataccaatcacggaaaggatgctggtaatgagctggagacgtctatggctactcatggcaacaagctcactaataagataatatacatattatggacaataatcctggtgttattatcaatagtcttcatcatagtgctaattaattccatcaaaagtgaaaaggcccacgaatcattgctgcaagacataaataatgagtttatggaaattacagaaaagatccaaatggcatcggataataccaatgatctaatacagtcaggagtgaatacaaggcttcttacaattcagagtcatgtccagaattacataccaatatcattgacacaacagatgtcagatcttaggaaattcattagtgaaattacaattagaaatgataatcaagaagtgctgccacaaagaataacacatgatgtaggtataaaacctttaaatccagatgatttttggagatgcacgtctggtcttccatctttaatgaaaactccaaaaataaggttaatgccagggccgggattattagctatgccaacgactgttgatggctgtgttagaactccgtctttagttataaatgatctgatttatgcttatacctcaaatctaattactcgaggttgtcaggatataggaaaatcatatcaagtcttacagatagggataataactgtaaactcagacttggtacctgacttaaatcctaggatctctcatacctttaacataaatgacaataggaagtcatgttctctagcactcctaaatacagatgtatatcaactgtgttcaactcccaaagttgatgaaagatcagattatgcatcatcaggcatagaagatattgtacttgatattgtcaattatgatggttcaatctcaacaacaagatttaagaataataacataagctttgatcaaccatatgctgcactatacccatctgttggaccagggatatactacaaaggcaaaataatatttctcgggtatggaggtcttgaacatccaataaatgagaatgtaatctgcaacacaactgggtgccccgggaaaacacagagagactgtaatcaagcatctcatagtccatggttttcagataggaggatggtcaactccatcattgttgttgacaaaggcttaaactcaattccaaaattgaaagtatggacgatatctatgcgacaaaattactgggggtcagaaggaaggttacttctactaggtaacaagatctatatatatacaagatctacaagttggcatagcaagttacaattaggaataattgatattactgattacagtgatataaggataaaatggacatggcataatgtgctatcaagaccaggaaacaatgaatgtccatggggacattcatgtccagatggatgtataacaggagtatatactgatgcatatccactcaatcccacagggagcattgtgtcatctgtcatattagactcacaaaaatcgagagtgaacccagtcataacttactcaacagcaaccgaaagagtaaacgagctggccatcctaaacagaacactctcagctggatatacaacaacaagctgcattacacactataacaaaggatattgttttcatatagtagaaataaatcataaaagcttaaacacatttcaacccatgttgttcaaaacagagattccaaaaagctgcagttaatcataattaaccataatatgcatcaatctatctataatacaagtatatgataagtaatcagcaatcagacaatagacgtacggaaataataaaaaacttaggagaaaagtgtgcaagaaaaatggacaccgagtcccacagcggcacaacatctgacattctgtaccctgaatgtcacctcaattctcctatagttaaaggaaagatagcacaactgcatacaataatgagtttgcctcagccctacgatatggatgatgattcaatactgattattactagacaaaaaattaaactcaataaattagataaaagacaacggtcaattaggaaattaagatcagtcttaatggaaagagtaagtgatctaggtaaatatacctttatcagatatccagagatgtctagtgaaatgttccaattatgtatacccggaattaataataaaataaatgaattgctaagtaaagcaagtaaaacatataatcaaatgactgatggattaagagatctatgggttactatactatcgaagttagcatcgaaaaatgatggaagtaattatgatatcaatgaagatattagcaatatatcaaatgttcacatgacttatcaatcagacaaatggtataatccattcaagacatggtttactattaagtatgacatgagaagattacaaaaagccaaaaatgagattacattcaataggcataaagattataatctattagaagaccaaaagaatatattgctgatacatccagaactcgtcttaatattagataaacaaaattacaatgggtatataatgactcctgaattggtactaatgtattgtgatgtagttgaagggaggtggaatataagttcatgtgcaaaattggatcctaagttacaatcaatgtattataagggtaacaatttatgggaaataatagatggactattctcgaccttaggagaaagaacatttgacataatatcactattagaaccacttgcattatcgctcattcaaacttatgacccggttaaacagctcaggggggcttttttaaatcacgtgttatcagaaatggaattaatatttgcagctgagtgtacaacagaggaaatacctaatgtggattatatagataaaattttagatgtgttcaaagaatcaacaatagatgaaatagcagaaattttctctttcttccgaacttttggacaccctccattagaggcgagtatagcagcagagaaagttagaaagtatatgtatactgagaaatgcttgaaatttgatactatcaataaatgtcatgctattttttgtacaataattataaatggatatagagaaagacatggtggtcaatggcctccagttacattacctgtccatgcacatgaatttatcataaatgcatacggatcaaattctgccatatcatatgagaatgctgtagattattataagagcttcataggaataaaatttgacaagtttatagagcctcaattggatgaagacttaactatttatatgaaagataaagcattatccccaaagaaatcaaactgggacacagtctatccagcttcaaacctgttataccgcactaatgtgtctcatgattcacgaagattggttgaagtatttatagcagatagtaaatttgatccccaccaagtattagattacgtagaatcaggatattggctggatgatcctgaatttaatatctcatatagtttaaaagagaaagaaataaaacaagaaggtagactttttgcaaaaatgacatacaagatgagggctacacaagtattatcagaaacattattggcgaataatatagggaaattcttccaagagaatgggatggttaaaggagaaattgaattactcaagagactaacaacaatatctatgtctggagttccgcggtataatgaggtatacaataattcaaaaagtcacacagaagaacttcaagcttataatgcaattagcagttccaatttatcttctaatcagaagtcaaagaagtttgaatttaaatctacagatatatacaatgatggatacgaaaccgtaagctgcttcttaacgacagatcttaaaaaatattgtttaaattggaggtatgaatcaacagctttattcggtgatacttgtaatcagatatttgggttaaaggaattatttaattggctgcaccctcgccttgaaaagagtacaatatatgttggagatccttattgcccgccatcagatattgaacatttaccacttgatgaccatcctgattcaggattttatgttcataatcctaaaggaggaatagaagggttttgccaaaagttatggacactcatatctatcagtgcaatacatttagcagctgtcaaaatcggtgtaagagttactgcaatggttcaaggggataatcaagccatagctgttaccacaagagtacctaataattatgattataaagttaagaaagagattgtttataaagatgtggtaagattttttgattccttgagagaggtgatggatgatctgggtcatgagctcaaactaaatgaaactataataagtagtaaaatgtttatatatagcaaaaggatatactatgacggaagaatccttcctcaggcattaaaagcattgtctagatgtgttttttggtctgaaacaatcatagatgagacaagatcagcatcctcaaatctggctacatcgtttgcaaaggccattgagaatggctactcacctgtattgggatatgtatgctcaatcttcaaaaatatccaacagttgtatatagcgcttggaatgaatataaacccaactataacccaaaatattaaagatcaatatttcaggaatattcattggatgcaatatgcctccttaatccctgctagtgtcggaggatttaattatatggccatgtcaaggtgttttgtcagaaacattggagatcctacagtcgctgcgttagccgatattaaaagatttataaaagcaaatttgttagatcgaggtgtcctttacagaattatgaatcaagaaccaggcgagtcttcttttttagactgggcctcagatccctattcatgtaacttaccacaatctcaaaatataaccaccatgataaagaatataactgcaagaaatgtactacaggactcaccaaacccattactatctggattatttacaagtacaatgatagaagaggatgaggaattagctgagttcctaatggacaggagaataatcctcccaagagttgcacatgacattttagataattctcttactggaattaggaatgctatagctggtatgttggatacaacaaaatcactaattcgagtagggataagcagaggaggattaacctataacttattaagaaagataagcaactatgatcttgtacaatatgagacacttagtaaaactttaagactaatagtcagtgacaagattaagtatgaagatatgtgctcagtagacctagccatatcattaagacaaaaaatgtggatgcatttatcaggaggaagaatgataaatggacttgaaactccagatcctttagagttactgtctggagtaataataacaggatctgaacattgtaggatatgttattcaactgaaggtgaaagcccatatacatggatgtatttaccaggcaatcttaatataggatcagctgagacaggaatagcatcattaagggtcccttactttggatcagttacagatgagagatctgaagcacaattagggtatatcaaaaatctaagcaaaccagctaaggctgctataagaatagcaatgatatatacttgggcatttgggaatgacgaaatatcttggatggaagcatcacagattgcacaaacacgtgcaaactttacattggatagcttaaagattttgacaccagtgacaacatcaacaaatctatcacacaggttaaaagatactgctactcagatgaaattttctagtacatcacttattagagtaagcaggttcatcacaatatctaatgataatatgtctattaaagaagcaaatgaaactaaagatacaaatcttatttatcaacaggtaatgttaacaggattaagtgtatttgaatatctatttaggttagaggagagtacaggacataaccctatggtcatgcatctacatatagaggatggatgttgtataaaagagagttacaatgatgagcatatcaatccggagtctacattagagttaatcaaataccctgagagtaatgaatttatatatgataaggaccctttaaaggatatagatctatcaaaattaatggttataagagatcattcttatacaattgacatgaattactgggatgacacagatattgtacatgcaatatcaatatgtactgcagttacaatagcagatacaatgtcgcagctagatcgggataatcttaaggagctggttgtgattgcaaatgatgatgatattaacagtctgataactgaatttctgaccctagatatactagtgtttctcaaaacatttggagggttactcgtgaatcaatttgcatataccctttatggattgaaaatagaaggaagggatcccatttgggattatataatgagaacattaaaagacacctcacattcagtacttaaagtattatctaatgcactatctcatccaaaagtgtttaagagattttgggattgtggagttttgaatcctatttatggtcctaatactgctagtcaagatcaagttaagcttgctctctcgatttgcgagtactccttggatctatttatgagagaatggttgaatggagcatcacttgagatctatatctgtgatagtgacatggaaatagcaaatgacagaagacaagcatttctctcaagacatcttgcctttgtgtgttgtttagcagagatagcatcttttggaccaaatttattaaatctaacatatctagagagacttgatgaattaaaacaatacttagatctgaacatcaaagaagatcctactcttaaatatgtgcaagtatcaggactgttaattaaatcattcccctcaactgttacgtatgtaaggaaaactgcgattaagtatctgaggattcgtggtattaatccgcctgaaacgattgaagattgggatcccatagaagatgagaatatcttagacaatattgttaaaactgtaaatgacaattgcagtgataatcaaaagagaaataaaagtagttatttctggggattagctctaaagaattatcaagtcgtgaaaataagatccataacgagtgattctgaagttaatgaagcttcgaatgttactacacatggaatgacacttcctcagggaggaagttatctatcacatcagctgaggttatttggagtaaacagtacaagttgtcttaaagctcttgaattatcacaaatcttaatgagggaagttaaaaaagataaagatagactctttttaggagaaggagcaggagctatgttagcatgttatgatgctacactcggtcctgcaataaattattataattctggtttaaatattacagatgtaattggtcaacgggaattaaaaatcttcccatcagaagtatcattagtaggtaaaaaactaggaaatgtaacacagattcttaatcgggtgagggtgttatttaatgggaatcccaattcaacatggataggaaatatggaatgtgagagtttaatatggagtgaattaaatgataagtcaattggtttagtacattgtgacatggagggagcgataggcaaatcagaagaaactgttctacatgaacattatagtattattaggattacatatttaatcggggatgatgatgttgtcctagtatcaaaaattataccaactattactccgaattggtctaaaatactctatctatacaagttgtattggaaggatgtaagtgtagtgtcccttaaaacatccaatcctgcctcaacagagctttatttaatttcaaaagatgcttactgtactgtaatggaacccagtaatcttgttttatcaaaacttaaaaggatatcatcaatagaagaaaataatctattaaagtggataatcttatcaaaaaggaagaataacgagtggttacagcatgaaatcaaagaaggagaaagggattatgggataatgaggccatatcatacagcactgcaaatttttggattccaaattaacttaaatcacttagctagagaatttttatcaactcctgatttaaccaacattaataatataattcaaagttttacaagaacaattaaagatgttatgttcgaatgggtcaatatcactcatgacaataaaagacataaattaggaggaagatataatctattcccgcttaaaaataaggggaaattaagattattatcacgaagattagtactaagctggatatcattatccttatcaaccagattactgacgggccgttttccagatgaaaaatttgaaaatagggcacagaccggatatgtatcattggctgatattgatttagaatccttaaagttattatcaagaaatattgtcaaaaattacaaagaacacataggattaatatcatactggtttttgaccaaagaggtcaaaatactaatgaagcttataggaggagtcaaactactaggaattcctaaacagtacaaagagttagaggatcgatcatctcagggttatgaatatgataatgaatttgatattgattaatacataaaaacaaaaaataaaacacctattcctcacccattcacttccaacaaaatgaaaagtaagaaaaacatgtaatatatatataccaaacagagtttttctcttgtttggt

SEQ ID NO: 31 - DNA, 19314 bp, Artificial Sequence
  Recombinant nucleic acid
accaaacaagagaagagactggtttgggaatattaattcaaataaaaattaacttaggattaaagaactttaccgaaaggtaaggggaaagaaatcctaagagcttagccatgttgagtctattcgacacattcagtgcgcgtaggcaggagaacataacgaaatcagctggtggggctgttattcccgggcaaaaaaacactgtgtctatatttgctcttggaccatcaataacagatgacaatgataaaatgacattggctcttctctttttgtctcattctttagacaatgaaaagcagcatgcgcaaagagctggatttttagtttctctgttatcaatggcttatgccaacccagaattatatttaacatcaaatggtagtaatgcagatgttaaatatgttatctacatgatagagaaagacccaggaagacagaaatatggtgggtttgtcgtcaagactagagagatggtttatgaaaagacaactgattggatgttcgggagtgatcttgagtatgatcaagacaatatgttgcaaaatggtagaagcacttctacaatcgaggatcttgttcatacttttggatatccatcgtgtcttggagcccttataatccaagtttggataatacttgttaaggctataaccagtatatcaggattgaggaaaggattctttactcggttagaagcatttcgacaagatggaacagttaaatccagtctagtgttgagcggtgatgcagtagaacaaattggatcaattatgaggtcccaacagagcttggtaacactcatggttgaaacactgataacaatgaacacaggcaggaatgatctgacaacaatagaaaagaatatacagattgtaggaaactacatcagagatgcaggtcttgcttcatttttcaacacaatcagatatggcattgagactagaatggcagctctaactctgtctacccttagaccggatatcaacagactcaaggcactgatcgagttatatctatcaaaggggccacgtgctccttttatatgcattttgagagatcccgtgcatggtgagtttgcaccaggcaactatcctgccctctggagttatgcgatgggtgtagcagttgtacaaaacaaggccatgcaacagtatgtaacaggaaggtcttatctggatattgaaatgttccaacttggtcaagcagtggcacgtgatgccgagtcgcagatgagttcaatattagaggatgaactgggggtcacacaagaagccaagcaaagcttgaagaaacacatgaagaacatcagcagttcagatacaacctttcataagcctacagggggatcagccatagaaatggcgatagatgaagaagcagggcagcctgaatccagaggagatcaggatcaaggagatgagcctcggtcatccatagttccttatgcatgggcagacgaaaccgggaatgacaatcaaactgaatcaactacagaaattgacagcatcaaaactgaacaaagaaacatcagagacaggctgaacaaaagactcaacgagaaaaggaaacagagtgacccgagatcaactgacatcacaaacaacacaaatcaaactgaaatagatgatttgttcagtgcattcggaagcaactagtcacaaagagatgaccaggcgcgccaagtaagaaaaacttaggattaatggacctgcaggatgttcgtgtttctggtgctgctgcctctggtgagctcccagtgcgtgaacctgaccacaaggacccagctgccccctgcctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagcggttcgacaatccagtgctgccctttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcgagtttcgcgtgtattcctctgccaacaattgcacatttgagtacgtgtcccagcccttcctgatggacctggagggcaagcagggcaatttcaagaacctgagggagttcgtgtttaagaatatcgatggctacttcaagatctactccaagcacaccccaatcaacctggtgcgcgacctgccacagggcttctctgccctggagccactggtggatctgcccatcggcatcaacatcacccggtttcagacactgctggccctgcacagaagctacctgacaccaggcgacagctcctctggatggaccgcaggagctgccgcctactatgtgggctatctgcagcccaggaccttcctgctgaagtacaacgagaatggcaccatcacagacgccgtggattgcgccctggatcccctgtctgagaccaagtgtacactgaagagctttaccgtggagaagggcatctatcagacaagcaatttcagggtgcagcctaccgagtccatcgtgcgctttcccaatatcacaaacctgtgcccttttggcgaggtgttcaacgcaacccgcttcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcccctggccagacaggcaagatcgccgactacaattataagctgcctgacgatttcaccggctgcgtgatcgcctggaactctaacaatctggatagcaaagtgggcggcaactacaattatctgtaccggctgtttagaaagtctaatctgaagccattcgagagggacatctccacagagatctaccaggccggctctaccccctgcaatggcgtggagggctttaactgttatttccctctgcagagctacggcttccagccaacaaacggcgtgggctatcagccctaccgcgtggtggtgctgtcttttgagctgctgcacgcacctgcaacagtgtgcggaccaaagaagagcaccaatctggtgaagaacaagtgcgtgaacttcaacttcaacggactgaccggcacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcaggacgtgaattgtaccgaggtgcccgtggcaatccacgcagatcagctgacccctacatggcgggtgtactctaccggcagcaacgtgttccagacaagagccggatgcctgatcggagccgagcacgtgaacaatagctatgagtgcgacatccctatcggcgccggcatctgtgcctcctaccagacccagacaaactccccagggtctgcctcctctgtggccagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggctctttttgtacccagctgaatagagccctgacaggcatcgccgtggagcaggacaagaacacacaggaggtgttcgcccaggtgaagcagatctacaagaccccacccatcaaggactttggcggcttcaacttcagccagatcctgcccgatcctagcaagccatccaagcggtctcctatcgaggacctgctgttcaacaaggtgaccctggccgatgccggcttcatcaagcagtatggcgattgcctgggcgacatcgccgccagagacctgatctgtgcccagaagtttaatggcctgaccgtgctgcctccactgctgacagatgagatgatcgcccagtacacatctgccctgctggccggcaccatcacaagcggatggaccttcggcgcaggacccgccctgcagatcccctttcccatgcagatggcctatcggttcaacggcatcggcgtgacccagaatgtgctgtacgagaaccagaagctgatcgccaatcagtttaactccgccatcggcaagatccaggactctctgagctccacacccagcgccctgggcaagctgcaggatgtggtgaatcagaacgcccaggccctgaataccctggtgaagcagctgtctagcaacttcggcgccatctcctctgtgctgaatgatatcctgagcaggctggaccctccagaggcagaggtgcagatcgaccggctgatcacaggcagactgcagtccctgcagacctacgtgacacagcagctgatcagggcagcagagatcagggcctctgccaatctggccgccaccaagatgagcgagtgcgtgctgggccagtccaagagagtggacttttgtggcaagggctatcacctgatgagcttcccacagtccgcccctcacggagtggtgtttctgcacgtgacctacgtgccagcccaggagaagaacttcaccacagcaccagcaatctgccacgatggcaaggcacactttcctagggagggcgtgttcgtgagcaacggcacccactggtttgtgacacagcgcaatttctacgagccacagatcatcaccacagacaatacattcgtgtccggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgacaggctgaacgaggtggccaagaatctgaacgagtccctgatcgatctgcaggagctgggcaagtatgagcagtacatcaagtggccctggtatatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactacacctgatagtaactagcggcgcgccagcaacaagtaagaaaaacttaggattaatggaaattatccaatccagagacggaaggacaaatccagaatccaaccacaactcaatcaaccaaagattcatggaagacaatgttcaaaacaatcaaatcatggattcttgggaagagggatcaggagataaatcatctgacatctcatcggccctcgacatcattgaattcatactcagcaccgactcccaagagaacacggcagacagcaatgaaatcaacacaggaaccacaagacttagcacgacaatctaccaacctgaatccaaaacaacagaaacaagcaaggaaaatagtggaccagctaacaaaaatcgacagtttggggcatcacacgaacgtgccacagagacaaaagatagaaatgttaatcaggagactgtacagggaggatataggagaggaagcagcccagatagtagaactgagactatggtcactcgaagaatctccagaagcagcccagatcctaacaatggaacccaaatccaggaagatattgattacaatgaagttggagagatggataaggactctactaagagggaaatgcgacaatttaaagatgttccagtcaaggtatcaggaagtgatgccattcctccaacaaaacaagatggagacggtgatgatggaagaggcctggaatctatcagtacatttgattcaggatataccagtatagtgactgccgcaacactagatgacgaagaagaactccttatgaagaacaacaggccaagaaagtatcaatcaacaccccagaacagtgacaagggaattaaaaaaggggttggaaggccaaaagacacagacaaacaatcatcaatattggactacgaactcaacttcaaaggatcgaagaagagccagaaaatcctcaaagccagcacgaatacaggagaaccaacaagaccacagaatggatcccaggggaagagaatcacatcctggaacatcctcaacagcgagagcggcaatcgaacagaatcaacaaaccaaacccatcagacatcaacctcgggacagaaccacacaatgggaccaagcagaacaacctccgaaccaaggatcaagacacaaaagacggatggaaaggaaagagaggacacagaagagagcactcgatttacagaaagggcgattacattattacagaatcttggtgtaatccaatctgcagcaaaattagacctataccaagacaagagagttgtgtgtgtggcgaatgtcctaaacaatgcagatactgcatcaaagatagacttcctagcaggtttgatgataggagtgtcaatggatcatgataccaaattaaatcagattcagaacgagatattaagtttgaaaactgatcttaaaaagatggatgaatcacatagaagactaattgagaatcaaaaagaacaattatcactgatcacatcattaatctcaaatcttaaaattatgacagagagaggagggaagaaggaccaaccagaacctagcgggaggacatccatgatcaagacaaaagcaaaagaagagaaaataaagaaagtcaggtttgaccctcttatggaaacacagggcatcgagaaaaacatccctgacctctatagatcaatagagaaaacaccagaaaacgacacacagatcaaatcagaaataaacagattgaatgatgaatccaatgccactagattagtacctagaagaataagcagtacaatgagatcattaataataatcattaacaacagcaatttatcatcaaaagcaaagcaatcatacatcaacgaactcaagctctgcaagagtgacgaggaagtgtctgagttgatggacatgttcaatgaggatgtcagctcccagtaaaccgccaaccaagggtcaacaccaagaaaaccaatagcacaaaacagccaatcagagaccaccccaatacaccaaaccaatcaacacataacaaagatcgcggccgcatagatgattaagaaaaacttaggatgaaaggactaatcaatcctccgaaacaatgagcatcaccaactccacaatctacacattcccagaatcctctttctccgagaatggcaacatagagccgttaccactcaaggtcaatgaacagagaaaggccatacctcatattagggttgtcaagataggagatccgcccaaacatggatccagatatctggatgtctttttactgggcttctttgagatggaaaggtcaaaagacaggtatgggagcataagtgatctagatgatgatccaagttacaaggtttgtggctctggatcattgccacttgggttggctagatacaccggaaatgatcaggaactcctacaggctgcaaccaagctcgatatagaagtaagaagaactgtaaaggctacggagatgatagtttacactgtacaaaacatcaaacctgaactatatccatggtccagtagattaagaaaagggatgttatttgacgctaataaggttgcacttgctcctcaatgtcttccactagatagagggataaaattcagggtgatatttgtgaactgcacagcaattggatcaataactctattcaaaatccctaagtccatggcattgttatcattgcctaatacaatatcaataaatctacaagtacatatcaaaacaggagttcagacagattccaaaggagtagttcagattctagatgaaaaaggtgaaaaatcactaaatttcatggttcatctcgggttgatcaaaaggaagatgggcagaatgtactcagttgaatattgtaagcagaagatcgagaagatgagattattattctcattgggattagttggagggatcagcttccacgtcaacgcaactggctctatatcaaagacattagcaagtcaattagcattcaaaagagaaatctgctatcccctaatggatctgaatccacacttaaattcagttatatgggcatcatcagttgaaattacaagggtagatgcagttctccagccttcattacctggcgaattcagatactacccaaacatcatagcaaaaggggtcgggaaaatcagacagtaaaatcaacaaccctgatatccaccggtgtattaagccgaagcaaataaaggataatcaaaaacttaggacaaaagaggtcaataccaacaactattagcagtcacactcgcaagaataagagagaagggaccaaaaaagtcaaataggagaaatcaaaacaaaaggtacagaacaccagaacaacaaaatcaaaacatccaactcactcaaaacaaaaattccaaaagagaccggcaacacaacaagcactgaacacaatgccaacttcaatactgctaattattacaaccatgatcatggcatctttctgccaaatagatatcacaaaactacagcacgtaggtgtattggtcaacagtcccaaagggatgaagatatcacaaaactttgaaacaagatatctaattttgagcctcataccaaaaatagaagactctaactcttgtggtgaccaacagatcaagcaatacaagaagttattggatagactgatcatccctttatatgatggattaagattacagaaagatgtgatagtaaccaatcaagaatccaatgaaaacactgatcccagaacaaaacgattctttggaggggtaattggaaccattgctctgggagtagcaacctcagcacaaattacagcggcagttgctctggttgaagccaagcaggcaagatcagacatcgaaaaactcaaagaagcaattagggacacaaacaaagcagtgcagtcagttcagagctccataggaaatttaatagtagcaattaaatcagtccaggattatgttaacaaagaaatcgtgccatcgattgcgaggctaggttgtgaagcagcaggacttcaattaggaattgcattaacacagcattactcagaattaacaaacatatttggtgataacataggatcgttacaagaaaaaggaataaaattacaaggtatagcatcattataccgcacaaatatcacagaaatattcacaacatcaacagttgataaatatgatatctatgatctgttatttacagaatcaataaaggtgagagttatagatgttgacttgaatgattactcaatcaccctccaagtcagactccctttattaactaggctgctgaacactcagatctacaaagtagattccatatcatataacatccaaaacagagaatggtatatccctcttcccagccatatcatgacgaaaggggcatttctaggtggagcagacgtcaaagaatgtatagaagcattcagcagctatatatgcccttctgatccaggatttgtattaaaccatgaaatagagagctgcttatcaggaaacatatcccaatgtccaagaacaacggtcacatcagacattgttccaagatatgcatttgtcaatggaggagtggttgcaaactgtataacaaccacctgtacatgcaacggaattggtaatagaatcaatcaaccacctgatcaaggagtaaaaattataacacataaagaatgtagtacaataggtatcaacggaatgctgttcaatacaaataaagaaggaactcttgcattctatacaccaaatgatataacactaaacaattctgttgcacttgatccaattgacatatcaatcgagctcaacaaggccaaatcagatctagaagaatcaaaagaatggataagaaggtcaaatcaaaaactagattctattggaaattggcatcaatctagcactacaatcataattattttgataatgatcattatattgtttataattaatataacgataattacaattgcaattaagtattacagaattcaaaagagaaatcgagtggatcaaaatgacaagccatatgtactaacaaacaaataacatatctacagatcattagatattaaaattataaaaaacttaggagtaaagttacgcaatccaactctactcatataattgaggaaggacccaatagacaaatccaaattcgagatggaatactggaagcataccaatcacggaaaggatgctggtaatgagctggagacgtctatggctactcatggcaacaagctcactaataagataatatacatattatggacaataatcctggtgttattatcaatagtcttcatcatagtgctaattaattccatcaaaagtgaaaaggcccacgaatcattgctgcaagacataaataatgagtttatggaaattacagaaaagatccaaatggcatcggataataccaatgatctaatacagtcaggagtgaatacaaggcttcttacaattcagagtcatgtccagaattacataccaatatcattgacacaacagatgtcagatcttaggaaattcattagtgaaattacaattagaaatgataatcaagaagtgctgccacaaagaataacacatgatgtaggtataaaacctttaaatccagatgatttttggagatgcacgtctggtcttccatctttaatgaaaactccaaaaataaggttaatgccagggccgggattattagctatgccaacgactgttgatggctgtgttagaactccgtctttagttataaatgatctgatttatgcttatacctcaaatctaattactcgaggttgtcaggatataggaaaatcatatcaagtcttacagatagggataataactgtaaactcagacttggtacctgacttaaatcctaggatctctcatacctttaacataaatgacaataggaagtcatgttctctagcactcctaaatacagatgtatatcaactgtgttcaactcccaaagttgatgaaagatcagattatgcatcatcaggcatagaagatattgtacttgatattgtcaattatgatggttcaatctcaacaacaagatttaagaataataacataagctttgatcaaccatatgctgcactatacccatctgttggaccagggatatactacaaaggcaaaataatatttctcgggtatggaggtcttgaacatccaataaatgagaatgtaatctgcaacacaactgggtgccccgggaaaacacagagagactgtaatcaagcatctcatagtccatggttttcagataggaggatggtcaactccatcattgttgttgacaaaggcttaaactcaattccaaaattgaaagtatggacgatatctatgcgacaaaattactgggggtcagaaggaaggttacttctactaggtaacaagatctatatatatacaagatctacaagttggcatagcaagttacaattaggaataattgatattactgattacagtgatataaggataaaatggacatggcataatgtgctatcaagaccaggaaacaatgaatgtccatggggacattcatgtccagatggatgtataacaggagtatatactgatgcatatccactcaatcccacagggagcattgtgtcatctgtcatattagactcacaaaaatcgagagtgaacccagtcataacttactcaacagcaaccgaaagagtaaacgagctggccatcctaaacagaacactctcagctggatatacaacaacaagctgcattacacactataacaaaggatattgttttcatatagtagaaataaatcataaaagcttaaacacatttcaacccatgttgttcaaaacagagattccaaaaagctgcagttaatcataattaaccataatatgcatcaatctatctataatacaagtatatgataagtaatcagcaatcagacaatagacgtacggaaataataaaaaacttaggagaaaagtgtgcaagaaaaatggacaccgagtcccacagcggcacaacatctgacattctgtaccctgaatgtcacctcaattctcctatagttaaaggaaagatagcacaactgcatacaataatgagtttgcctcagccctacgatatggatgatgattcaatactgattattactagacaaaaaattaaactcaataaattagataaaagacaacggtcaattaggaaattaagatcagtcttaatggaaagagtaagtgatctaggtaaatatacctttatcagatatccagagatgtctagtgaaatgttccaattatgtatacccggaattaataataaaataaatgaattgctaagtaaagcaagtaaaacatataatcaaatgactgatggattaagagatctatgggttactatactatcgaagttagcatcgaaaaatgatggaagtaattatgatatcaatgaagatattagcaatatatcaaatgttcacatgacttatcaatcagacaaatggtataatccattcaagacatggtttactattaagtatgacatgagaagattacaaaaagccaaaaatgagattacattcaataggcataaagattataatctattagaagaccaaaagaatatattgctgatacatccagaactcgtcttaatattagataaacaaaattacaatgggtatataatgactcctgaattggtactaatgtattgtgatgtagttgaagggaggtggaatataagttcatgtgcaaaattggatcctaagttacaatcaatgtattataagggtaacaatttatgggaaataatagatggactattctcgaccttaggagaaagaacatttgacataatatcactattagaaccacttgcattatcgctcattcaaacttatgacccggttaaacagctcaggggggcttttttaaatcacgtgttatcagaaatggaattaatatttgcagctgagtgtacaacagaggaaatacctaatgtggattatatagataaaattttagatgtgttcaaagaatcaacaatagatgaaatagcagaaattttctctttcttccgaacttttggacaccctccattagaggcgagtatagcagcagagaaagttagaaagtatatgtatactgagaaatgcttgaaatttgatactatcaataaatgtcatgctattttttgtacaataattataaatggatatagagaaagacatggtggtcaatggcctccagttacattacctgtccatgcacatgaatttatcataaatgcatacggatcaaattctgccatatcatatgagaatgctgtagattattataagagcttcataggaataaaatttgacaagtttatagagcctcaattggatgaagacttaactatttatatgaaagataaagcattatccccaaagaaatcaaactgggacacagtctatccagcttcaaacctgttataccgcactaatgtgtctcatgattcacgaagattggttgaagtatttatagcagatagtaaatttgatccccaccaagtattagattacgtagaatcaggatattggctggatgatcctgaatttaatatctcatatagtttaaaagagaaagaaataaaacaagaaggtagactttttgcaaaaatgacatacaagatgagggctacacaagtattatcagaaacattattggcgaataatatagggaaattcttccaagagaatgggatggttaaaggagaaattgaattactcaagagactaacaacaatatctatgtctggagttccgcggtataatgaggtatacaataattcaaaaagtcacacagaagaacttcaagcttataatgcaattagcagttccaatttatcttctaatcagaagtcaaagaagtttgaatttaaatctacagatatatacaatgatggatacgaaaccgtaagctgcttcttaacgacagatcttaaaaaatattgtttaaattggaggtatgaatcaacagctttattcggtgatacttgtaatcagatatttgggttaaaggaattatttaattggctgcaccctcgccttgaaaagagtacaatatatgttggagatccttattgcccgccatcagatattgaacatttaccacttgatgaccatcctgattcaggattttatgttcataatcctaaaggaggaatagaagggttttgccaaaagttatggacactcatatctatcagtgcaatacatttagcagctgtcaaaatcggtgtaagagttactgcaatggttcaaggggataatcaagccatagctgttaccacaagagtacctaataattatgattataaagttaagaaagagattgtttataaagatgtggtaagattttttgattccttgagagaggtgatggatgatctgggtcatgagctcaaactaaatgaaactataataagtagtaaaatgtttatatatagcaaaaggatatactatgacggaagaatccttcctcaggcattaaaagcattgtctagatgtgttttttggtctgaaacaatcatagatgagacaagatcagcatcctcaaatctggctacatcgtttgcaaaggccattgagaatggctactcacctgtattgggatatgtatgctcaatcttcaaaaatatccaacagttgtatatagcgcttggaatgaatataaacccaactataacccaaaatattaaagatcaatatttcaggaatattcattggatgcaatatgcctccttaatccctgctagtgtcggaggatttaattatatggccatgtcaaggtgttttgtcagaaacattggagatcctacagtcgctgcgttagccgatattaaaagatttataaaagcaaatttgttagatcgaggtgtcctttacagaattatgaatcaagaaccaggcgagtcttcttttttagactgggcctcagatccctattcatgtaacttaccacaatctcaaaatataaccaccatgataaagaatataactgcaagaaatgtactacaggactcaccaaacccattactatctggattatttacaagtacaatgatagaagaggatgaggaattagctgagttcctaatggacaggagaataatcctcccaagagttgcacatgacattttagataattctcttactggaattaggaatgctatagctggtatgttggatacaacaaaatcactaattcgagtagggataagcagaggaggattaacctataacttattaagaaagataagcaactatgatcttgtacaatatgagacacttagtaaaactttaagactaatagtcagtgacaagattaagtatgaagatatgtgctcagtagacctagccatatcattaagacaaaaaatgtggatgcatttatcaggaggaagaatgataaatggacttgaaactccagatcctttagagttactgtctggagtaataataacaggatctgaacattgtaggatatgttattcaactgaaggtgaaagcccatatacatggatgtatttaccaggcaatcttaatataggatcagctgagacaggaatagcatcattaagggtcccttactttggatcagttacagatgagagatctgaagcacaattagggtatatcaaaaatctaagcaaaccagctaaggctgctataagaatagcaatgatatatacttgggcatttgggaatgacgaaatatcttggatggaagcatcacagattgcacaaacacgtgcaaactttacattggatagcttaaagattttgacaccagtgacaacatcaacaaatctatcacacaggttaaaagatactgctactcagatgaaattttctagtacatcacttattagagtaagcaggttcatcacaatatctaatgataatatgtctattaaagaagcaaatgaaactaaagatacaaatcttatttatcaacaggtaatgttaacaggattaagtgtatttgaatatctatttaggttagaggagagtacaggacataaccctatggtcatgcatctacatatagaggatggatgttgtataaaagagagttacaatgatgagcatatcaatccggagtctacattagagttaatcaaataccctgagagtaatgaatttatatatgataaggaccctttaaaggatatagatctatcaaaattaatggttataagagatcattcttatacaattgacatgaattactgggatgacacagatattgtacatgcaatatcaatatgtactgcagttacaatagcagatacaatgtcgcagctagatcgggataatcttaaggagctggttgtgattgcaaatgatgatgatattaacagtctgataactgaatttctgaccctagatatactagtgtttctcaaaacatttggagggttactcgtgaatcaatttgcatataccctttatggattgaaaatagaaggaagggatcccatttgggattatataatgagaacattaaaagacacctcacattcagtacttaaagtattatctaatgcactatctcatccaaaagtgtttaagagattttgggattgtggagttttgaatcctatttatggtcctaatactgctagtcaagatcaagttaagcttgctctctcgatttgcgagtactccttggatctatttatgagagaatggttgaatggagcatcacttgagatctatatctgtgatagtgacatggaaatagcaaatgacagaagacaagcatttctctcaagacatcttgcctttgtgtgttgtttagcagagatagcatcttttggaccaaatttattaaatctaacatatctagagagacttgatgaattaaaacaatacttagatctgaacatcaaagaagatcctactcttaaatatgtgcaagtatcaggactgttaattaaatcattcccctcaactgttacgtatgtaaggaaaactgcgattaagtatctgaggattcgtggtattaatccgcctgaaacgattgaagattgggatcccatagaagatgagaatatcttagacaatattgttaaaactgtaaatgacaattgcagtgataatcaaaagagaaataaaagtagttatttctggggattagctctaaagaattatcaagtcgtgaaaataagatccataacgagtgattctgaagttaatgaagcttcgaatgttactacacatggaatgacacttcctcagggaggaagttatctatcacatcagctgaggttatttggagtaaacagtacaagttgtcttaaagctcttgaattatcacaaatcttaatgagggaagttaaaaaagataaagatagactctttttaggagaaggagcaggagctatgttagcatgttatgatgctacactcggtcctgcaataaattattataattctggtttaaatattacagatgtaattggtcaacgggaattaaaaatcttcccatcagaagtatcattagtaggtaaaaaactaggaaatgtaacacagattcttaatcgggtgagggtgttatttaatgggaatcccaattcaacatggataggaaatatggaatgtgagagtttaatatggagtgaattaaatgataagtcaattggtttagtacattgtgacatggagggagcgataggcaaatcagaagaaactgttctacatgaacattatagtattattaggattacatatttaatcggggatgatgatgttgtcctagtatcaaaaattataccaactattactccgaattggtctaaaatactctatctatacaagttgtattggaaggatgtaagtgtagtgtcccttaaaacatccaatcctgcctcaacagagctttatttaatttcaaaagatgcttactgtactgtaatggaacccagtaatcttgttttatcaaaacttaaaaggatatcatcaatagaagaaaataatctattaaagtggataatcttatcaaaaaggaagaataacgagtggttacagcatgaaatcaaagaaggagaaagggattatgggataatgaggccatatcatacagcactgcaaatttttggattccaaattaacttaaatcacttagctagagaatttttatcaactcctgatttaaccaacattaataatataattcaaagttttacaagaacaattaaagatgttatgttcgaatgggtcaatatcactcatgacaataaaagacataaattaggaggaagatataatctattcccgcttaaaaataaggggaaattaagattattatcacgaagattagtactaagctggatatcattatccttatcaaccagattactgacgggccgttttccagatgaaaaatttgaaaatagggcacagaccggatatgtatcattggctgatattgatttagaatccttaaagttattatcaagaaatattgtcaaaaattacaaagaacacataggattaatatcatactggtttttgaccaaagaggtcaaaatactaatgaagcttataggaggagtcaaactactaggaattcctaaacagtacaaagagttagaggatcgatcatctcagggttatgaatatgataatgaatttgatattgattaatacataaaaacaaaaaataaaacacctattcctcacccattcacttccaacaaaatgaaaagtaagaaaaacatgtaatatatatataccaaacagagtttttctcttgtttggt

SEQ ID NO: 32 - DNA, 43 bp, Artificial Sequence
  Synthetic nucleic acid
ggcgcgccaagtaagaaaaacttaggattaatggacctgcagg

SEQ ID NO: 33 - DNA, 34 bp, Artificial Sequence
  Synthetic nucleic acid
tagctagcggcgcgccagcaacaagtaagaaaaa

SEQ ID NO: 34 - DNA, 12 bp, Artificial Sequence
  Synthetic nucleic acid
aggattaatgga

SEQ ID NO: 35 - DNA, 11 bp, Artificial Sequence
  Synthetic nucleic acid
cctgcaggatg

SEQ ID NO: 36 - DNA, 15456 bp, Bovine parainfluenza virus 3
accaaacaagagaagagacttgcttgggaatattaattcaaataaaaattaacttaggattaaagaactttaccgaaaggtaaggggaaagaaatcctaagactgtaatcatgttgagtctattcgacacattcagtgcgcgtaggcaggagaacataacgaaatcagctggtggggctgttattcccgggcaaaaaaacactgtgtctatatttgctcttggaccatcaataacagatgacaatgataaaatgacattggctcttctctttttgtctcattctttagacaatgaaaagcagcatgcgcaaagagctggatttttagtttctctgttatcaatggcttatgccaacccagaattatatttaacatcaaatggtagtaatgcagatgttaaatatgttatctacatgatagagaaagacccaggaagacagaaatatggtgggtttgtcgtcaagactagagagatggtttatgaaaagacaactgattggatgttcgggagtgatcttgagtatgatcaagacaatatgttgcaaaatggtagaagcacttctacaatcgaggatcttgttcatacttttggatatccatcgtgtcttggagcccttataatccaagtttggataatacttgttaaggctataaccagtatatcaggattgaggaaaggattctttactcggttagaagcatttcgacaagatggaacagttaaatccagtctagtgttgagcggtgatgcagtagaacaaattggatcaattatgaggtcccaacagagcttggtaacactcatggttgaaacactgataacaatgaacacaggcaggaatgatctgacaacaatagaaaagaatatacagattgtaggaaactacatcagagatgcaggtcttgcttcatttttcaacacaatcagatatggcattgagactagaatggcagctctaactctgtctacccttagaccggatatcaacagactcaaggcactgatcgagttatatctatcaaaggggccacgtgctccttttatatgcattttgagagatcccgtgcatggtgagtttgcaccaggcaactatcctgccctctggagttatgcgatgggtgtagcagttgtacaaaacaaggccatgcaacagtatgtaacaggaaggtcttatctggatattgaaatgttccaacttggtcaagcagtggcacgtgatgccgagtcgcagatgagttcaatattagaggatgaactgggggtcacacaagaagccaagcaaagcttgaagaaacacatgaagaacatcagcagttcagatacaacctttcataagcctacagggggatcagccatagaaatggcgatagatgaagaagcagggcagcctgaatccagaggagatcaggatcaaggagatgagcctcggtcatccatagttccttatgcatgggcagacgaaaccgggaatgacaatcaaactgaatcaactacagaaattgacagcatcaaaactgaacaaagaaacatcagagacaggctgaacaaaagactcaacgagaaaaggaaacagagtgacccgagatcaactgacatcacaaacaacacaaatcaaactgaaatagatgatttgttcagtgcattcggaagcaactagtcacaaagagatgaccactatcaccagcaacaagtaagaaaaacttaggattaatggaaattatccaatccagagacggaaggacaaatccagaatccaaccacaactcaatcaaccaaagattcatggaagacaatgttcaaaacaatcaaatcatggattcttgggaagagggatcaggagataaatcatctgacatctcatcggccctcgacatcattgaattcatactcagcaccgactcccaagagaacacggcagacagcaatgaaatcaacacaggaaccacaagacttagcacgacaatctaccaacctgaatccaaaacaacagaaacaagcaaggaaaatagtggaccagctaacaaaaatcgacagtttggggcatcacacgaacgtgccacagagacaaaagatagaaatgttaatcaggagactgtacagggaggatataggagaggaagcagcccagatagtagaactgagactatggtcactcgaagaatctccagaagcagcccagatcctaacaatggaacccaaatccaggaagatattgattacaatgaagttggagagatggataaggactctactaagagggaaatgcgacaatttaaagatgttccagtcaaggtatcaggaagtgatgccattcctccaacaaaacaagatggagacggtgatgatggaagaggcctggaatctatcagtacatttgattcaggatataccagtatagtgactgccgcaacactagatgacgaagaagaactccttatgaagaacaacaggccaagaaagtatcaatcaacaccccagaacagtgacaagggaattaaaaaaggggttggaaggccaaaagacacagacaaacaatcatcaatattggactacgaactcaacttcaaaggatcgaagaagagccagaaaatcctcaaagccagcacgaatacaggagaaccaacaagaccacagaatggatcccaggggaagagaatcacatcctggaacatcctcaacagcgagagcggcaatcgaacagaatcaacaaaccaaacccatcagacatcaacctcgggacagaaccacacaatgggaccaagcagaacaacctccgaaccaaggatcaagacacaaaagacggatggaaaggaaagagaggacacagaagagagcactcgatttacagaaagggcgattacattattacagaatcttggtgtaatccaatctgcagcaaaattagacctataccaagacaagagagttgtgtgtgtggcgaatgtcctaaacaatgcagatactgcatcaaagatagacttcctagcaggtttgatgataggagtgtcaatggatcatgataccaaattaaatcagattcagaacgagatattaagtttgaaaactgatcttaaaaagatggatgaatcacatagaagactaattgagaatcaaaaagaacaattatcactgatcacatcattaatctcaaatcttaaaattatgacagagagaggagggaagaaggaccaaccagaacctagcgggaggacatccatgatcaagacaaaagcaaaagaagagaaaataaagaaagtcaggtttgaccctcttatggaaacacagggcatcgagaaaaacatccctgacctctatagatcaatagagaaaacaccagaaaacgacacacagatcaaatcagaaataaacagattgaatgatgaatccaatgccactagattagtacctagaagaataagcagtacaatgagatcattaataataatcattaacaacagcaatttatcatcaaaagcaaagcaatcatacatcaacgaactcaagctctgcaagagtgacgaggaagtgtctgagttgatggacatgttcaatgaggatgtcagctcccagtaaaccgccaaccaagggtcaacaccaagaaaaccaatagcacaaaacagccaatcagagaccaccccaatacaccaaaccaatcaacacataacaaagatctccagatcatagatgattaagaaaaacttaggatgaaaggactaatcaatcctccgaaacaatgagcatcaccaactccacaatctacacattcccagaatcctctttctccgagaatggcaacatagagccgttaccactcaaggtcaatgaacagagaaaggccatacctcatattagggttgtcaagataggagatccgcccaaacatggatccagatatctggatgtctttttactgggcttctttgagatggaaaggtcaaaagacaggtatgggagcataagtgatctagatgatgatccaagttacaaggtttgtggctctggatcattgccacttgggttggctagatacaccggaaatgatcaggaactcctacaggctgcaaccaagctcgatatagaagtaagaagaactgtaaaggctacggagatgatagtttacactgtacaaaacatcaaacctgaactatatccatggtccagtagattaagaaaagggatgttatttgacgctaataaggttgcacttgctcctcaatgtcttccactagatagagggataaaattcagggtgatatttgtgaactgcacagcaattggatcaataactctattcaaaatccctaagtccatggcattgttatcattgcctaatacaatatcaataaatctacaagtacatatcaaaacaggagttcagacagattccaaaggagtagttcagattctagatgaaaaaggtgaaaaatcactaaatttcatggttcatctcgggttgatcaaaaggaagatgggcagaatgtactcagttgaatattgtaagcagaagatcgagaagatgagattattattctcattgggattagttggagggatcagcttccacgtcaacgcaactggctctatatcaaagacattagcaagtcaattagcattcaaaagagaaatctgctatcccctaatggatctgaatccacacttaaattcagttatatgggcatcatcagttgaaattacaagggtagatgcagttctccagccttcattacctggcgaattcagatactacccaaacatcatagcaaaaggggtcgggaaaatcagacagtaaaatcaacaaccctgatatccaacattgcaaatcaggctacccacaggagaaaaatcaaaaacttaggatcaaagggatcaccacgaaccccggaaaacagccaaacaaaccaacacacaaatcacagacaaaaaggagaaggcactgcaaagaccgagaaaaaacagaacgcacacaaccaagcagagaaaagccaaagcccgccattcacaaacacaccaacaatcctgcaaacaagcaccaaaacagaggtcaaaagacaaagagcaccagatatgaccatcacaaccacaatcatagccatattactaatacccccatcattttgtcaaatagacataacaaaactgcaacgtgtaggtgtgttagtcaacaatcctaaaggcatgaagatttcacaaaatttcgaaacgagatacctgatattaagtttgatacccaaaatagagaattcacactcatgtggggatcaacagataaaccaatacaagaagttattggatagattgataattcctctatatgatggattaaaattacaaaaagatgtaatagtagtaagtcatgaaacccacaacaatactaatcttaggacaaaacgattctttggagagataattgggacaattgcgatagggatagccacttcagcacaaatcaccgcagcagtcgctcttgtcgaagctaaacaggcaaagtcagacatagaaaaactcaaagaggctataagagacacaaacaaggcagtacaatcgattcaaagttctgtaggtaacctaattgttgcagttaaatcagttcaagactatgtcaacaatgaaattataccttcaatcacaagattaggctgtgaagcagcagggttacaattgggaattgcattgacacaacattactcagaattaacaaatatatttggtgataatataggaacactgaaagaaaaagggataaaattacaagggatagcatcattatatcacacaaacataacggaaatatttactacttcaacagttgaccaatatgatatttatgacctattattcactgagtcaatcaagatgagagtgatagatgttgatttgagtgattactcaattactcttcaagttagacttcctttattaactaaactatcaaatactcaaatttataaagtagattctatatcatacaacatccagggcaaagagtggtatattcctcttcccaatcacatcatgacaaaaggggcttttctaggtggtgctgatattaaagaatgcatagaggcattcagcagttatatatgtccttctgatccaggttacatattaaatcacgagatagagaattgtttatcagggaacataacacagtgtcctaagactgttgttacatcagatgtggtaccacgatacgcgtttgtgaatggtggattaattgcaaactgcataacaactacatgtacatgcaatggaattgacaatagaattaatcaatcacctgatcaaggaattaagatcataacacataaagaatgccaggtaataggtataaacggaatgttattcaatactaatagagaagggacattagcaacttatacatttgatgacatcatattaaataactctgttgcacttaatccaattgatatatctatggaactcaacaaggcaaaactagaattagaagaatcgaaggaatggataaagaaatcaaatcaaaagttagattccgttggaagttggtatcaatctagtgcaacaatcaccataatcatagtgatgataataattctagttataatcaatataacaattattgtagtcataatcaaattccatagaattcaggggaaagatcaaaacgacaaaaacagtgagccgtatatactgacaaatagacaataagactatacacgatcaaatataaaaagtacaaaaaacttaggaacaaagttgttcaacacagcagcaccgaatagaccaaaaggcagcgcagaggcgacaccaaactcaaaaatggaatattggaaacacacaaacagcataaataacaccaacaatgaaaccgaaacagccagaggcaaacatagtagcaaggttacaaatatcataatgtacaccttctggacaataacattaacaatattatcagtcatttttataatgatattgacaaacttaattcaagagaacaatcataataaattaatgttgcaggaaataagaaaagaattcgcggcaatagacaccaagattcagaggacttcggatgacattggaacctcaatacagtcaggaataaatacaagacttctcacaattcagagtcatgttcaaaactatatcccactatcattaacacaacaaatgtcagatctcagaaaatttatcaatgatctaacaaataaaagagaacatcaagaagtgccaatacagagaatgactcatgatagaggtatagaacccctaaatccaaacaagttctggaggtgtacatctggtaacccatctctaacaagtagtcctaagataaggttaataccaggaccaggtttattagcaacatctactacagtaaatggctgtattagaattccatcgttagtaatcaatcatctaatctatgcttacacctctaatcttattacccagggctgtcaagatatagggaaatcttaccaagtactacaaatagggataattactataaattcggacctagtacctgatttaaaccccagagtcacacatacatttaatattgatgataatagaagatcttgctctctggcactattgaatacagatgtttatcagttatgctcaacaccaaaagttgatgaaagatccgattatgcatcaacaggtattgaggatattgtacttgacattgtcactaataatggattaattataacaacaaggtttacaaataataatataacttttgataaaccgtatgcagcattgtatccatcagtgggaccaggaatctattataaggataaagttatatttctcggatatggaggtctagagcatgaagaaaacggagacgtaatatgtaatacaactggttgtcctggcaaaacacagagagactgtaatcaggcttcttatagcccatggttctcaaataggagaatggtaaactctattattgttgttgataaaggcatagatgcaacttttagcttgagggtgtggactattccaatgagccaaaattattggggatcagaaggaagattacttttattaggtgacagaatatacatatatactagatccacaagttggcacagtaaattacagttaggggtaattgatatttctgattatactaatataagaataaattggacttggcataatgtactatcacggccagggaatgatgaatgtccatggggtcattcatgcccagacggatgtataacaggagtttacactgatgcatatccgctaaacccatcggggagtgttgtatcatcagtaattcttgattcacaaaagtctagagaaaacccaatcattacttactcaacagctacaaatagaataaatgaattagctatatataacagaacacttccagctgcatatacaacaacaaattgtatcacacattatgataaagggtattgttttcatatagtagaaataaatcacagaagtttgaatacgtttcaacctatgttattcaaaacagaagttccaaaaaactgcagctaaattgatcatcgcatatcggatgcaagatgacattaaaagagaccaccagacagacaacacaggagacgatgcaagatataaagaaataataaaaaacttaggagaaaagtgtgcaagaaaaatggacaccgagtcccacagcggcacaacatctgacattctgtaccctgaatgtcacctcaattctcctatagttaaaggaaagatagcacaactgcatacaataatgagtttgcctcagccctacgatatggatgatgattcaatactgattattactagacaaaaaattaaactcaataaattagataaaagacaacggtcaattaggaaattaagatcagtcttaatggaaagagtaagtgatctaggtaaatatacctttatcagatatccagagatgtctagtgaaatgttccaattatgtatacccggaattaataataaaataaatgaattgctaagtaaagcaagtaaaacatataatcaaatgactgatggattaagagatctatgggttactatactatcgaagttagcatcgaaaaatgatggaagtaattatgatatcaatgaagatattagcaatatatcaaatgttcacatgacttatcaatcagacaaatggtataatccattcaagacatggtttactattaagtatgacatgagaagattacaaaaagccaaaaatgagattacattcaataggcataaagattataatctattagaagaccaaaagaatatattgctgatacatccagaactcgtcttaatattagataaacaaaattacaatgggtatataatgactcctgaattggtactaatgtattgtgatgtagttgaagggaggtggaatataagttcatgtgcaaaattggatcctaagttacaatcaatgtattataagggtaacaatttatgggaaataatagatggactattctcgaccttaggagaaagaacatttgacataatatcactattagaaccacttgcattatcgctcattcaaacttatgacccggttaaacagctcaggggggcttttttaaatcacgtgttatcagaaatggaattaatatttgcagctgagtgtacaacagaggaaatacctaatgtggattatatagataaaattttagatgtgttcaaagaatcaacaatagatgaaatagcagaaattttctctttcttccgaacttttggacaccctccattagaggcgagtatagcagcagagaaagttagaaagtatatgtatactgagaaatgcttgaaatttgatactatcaataaatgtcatgctattttttgtacaataattataaatggatatagagaaagacatggtggtcaatggcctccagttacattacctgtccatgcacatgaatttatcataaatgcatacggatcaaattctgccatatcatatgagaatgctgtagattattataagagcttcataggaataaaatttgacaagtttatagagcctcaattggatgaagacttaactatttatatgaaagataaagcattatccccaaagaaatcaaactgggacacagtctatccagcttcaaacctgttataccgcactaatgtgtctcatgattcacgaagattggttgaagtatttatagcagatagtaaatttgatccccaccaagtattagattacgtagaatcaggatattggctggatgatcctgaatttaatatctcatatagtttaaaagagaaagaaataaaacaagaaggtagactttttgcaaaaatgacatacaagatgagggctacacaagtattatcagaaacattattggcgaataatatagggaaattcttccaagagaatgggatggttaaaggagaaattgaattactcaagagactaacaacaatatctatgtctggagttccgcggtataatgaggtatacaataattcaaaaagtcacacagaagaacttcaagcttataatgcaattagcagttccaatttatcttctaatcagaagtcaaagaagtttgaatttaaatctacagatatatacaatgatggatacgaaaccgtaagctgcttcttaacgacagatcttaaaaaatattgtttaaattggaggtatgaatcaacagctttattcggtgatacttgtaatcagatatttgggttaaaggaattatttaattggctgcaccctcgccttgaaaagagtacaatatatgttggagatccttattgcccgccatcagatattgaacatttaccacttgatgaccatcctgattcaggattttatgttcataatcctaaaggaggaatagaagggttttgccaaaagttatggacactcatatctatcagtgcaatacatttagcagctgtcaaaatcggtgtaagagttactgcaatggttcaaggggataatcaagccatagctgttaccacaagagtacctaataattatgattataaagttaagaaagagattgtttataaagatgtggtaagattttttgattccttgagagaggtgatggatgatctgggtcatgagctcaaactaaatgaaactataataagtagtaaaatgtttatatatagcaaaaggatatactatgacggaagaatccttcctcaggcattaaaagcattgtctagatgtgttttttggtctgaaacaatcatagatgagacaagatcagcatcctcaaatctggctacatcgtttgcaaaggccattgagaatggctactcacctgtattgggatatgtatgctcaatcttcaaaaatatccaacagttgtatatagcgcttggaatgaatataaacccaactataacccaaaatattaaagatcaatatttcaggaatattcattggatgcaatatgcctccttaatccctgctagtgtcggaggatttaattatatggccatgtcaaggtgttttgtcagaaacattggagatcctacagtcgctgcgttagccgatattaaaagatttataaaagcaaatttgttagatcgaggtgtcctttacagaattatgaatcaagaaccaggcgagtcttcttttttagactgggcctcagatccctattcatgtaacttaccacaatctcaaaatataaccaccatgataaagaatataactgcaagaaatgtactacaggactcaccaaacccattactatctggattatttacaagtacaatgatagaagaggatgaggaattagctgagttcctaatggacaggagaataatcctcccaagagttgcacatgacattttagataattctcttactggaattaggaatgctatagctggtatgttggatacaacaaaatcactaattcgagtagggataagcagaggaggattaacctataacttattaagaaagataagcaactatgatcttgtacaatatgagacacttagtaaaactttaagactaatagtcagtgacaagattaagtatgaagatatgtgctcagtagacctagccatatcattaagacaaaaaatgtggatgcatttatcaggaggaagaatgataaatggacttgaaactccagatcctttagagttactgtctggagtaataataacaggatctgaacattgtaggatatgttattcaactgaaggtgaaagcccatatacatggatgtatttaccaggcaatcttaatataggatcagctgagacaggaatagcatcattaagggtcccttactttggatcagttacagatgagagatctgaagcacaattagggtatatcaaaaatctaagcaaaccagctaaggctgctataagaatagcaatgatatatacttgggcatttgggaatgacgaaatatcttggatggaagcatcacagattgcacaaacacgtgcaaactttacattggatagcttaaagattttgacaccagtgacaacatcaacaaatctatcacacaggttaaaagatactgctactcagatgaaattttctagtacatcacttattagagtaagcaggttcatcacaatatctaatgataatatgtctattaaagaagcaaatgaaactaaagatacaaatcttatttatcaacaggtaatgttaacaggattaagtgtatttgaatatctatttaggttagaggagagtacaggacataaccctatggtcatgcatctacatatagaggatggatgttgtataaaagagagttacaatgatgagcatatcaatccggagtctacattagagttaatcaaataccctgagagtaatgaatttatatatgataaggaccctttaaaggatatagatctatcaaaattaatggttataagagatcattcttatacaattgacatgaattactgggatgacacagatattgtacatgcaatatcaatatgtactgcagttacaatagcagatacaatgtcgcagctagatcgggataatcttaaggagctggttgtgattgcaaatgatgatgatattaacagtctgataactgaatttctgaccctagatatactagtgtttctcaaaacatttggagggttactcgtgaatcaatttgcatataccctttatggattgaaaatagaaggaagggatcccatttgggattatataatgagaacattaaaagacacctcacattcagtacttaaagtattatctaatgcactatctcatccaaaagtgtttaagagattttgggattgtggagttttgaatcctatttatggtcctaatactgctagtcaagatcaagttaagcttgctctctcgatttgcgagtactccttggatctatttatgagagaatggttgaatggagcatcacttgagatctatatctgtgatagtgacatggaaatagcaaatgacagaagacaagcatttctctcaagacatcttgcctttgtgtgttgtttagcagagatagcatcttttggaccaaatttattaaatctaacatatctagagagacttgatgaattaaaacaatacttagatctgaacatcaaagaagatcctactcttaaatatgtgcaagtatcaggactgttaattaaatcattcccctcaactgttacgtatgtaaggaaaactgcgattaagtatctgaggattcgtggtattaatccgcctgaaacgattgaagattgggatcccatagaagatgagaatatcttagacaatattgttaaaactgtaaatgacaattgcagtgataatcaaaagagaaataaaagtagttatttctggggattagctctaaagaattatcaagtcgtgaaaataagatccataacgagtgattctgaagttaatgaagcttcgaatgttactacacatggaatgacacttcctcagggaggaagttatctatcacatcagctgaggttatttggagtaaacagtacaagttgtcttaaagctcttgaattatcacaaatcttaatgagggaagttaaaaaagataaagatagactctttttaggagaaggagcaggagctatgttagcatgttatgatgctacactcggtcctgcaataaattattataattctggtttaaatattacagatgtaattggtcaacgggaattaaaaatcttcccatcagaagtatcattagtaggtaaaaaactaggaaatgtaacacagattcttaatcgggtgagggtgttatttaatgggaatcccaattcaacatggataggaaatatggaatgtgagagtttaatatggagtgaattaaatgataagtcaattggtttagtacattgtgacatggagggagcgataggcaaatcagaagaaactgttctacatgaacattatagtattattaggattacatatttaatcggggatgatgatgttgtcctagtatcaaaaattataccaactattactccgaattggtctaaaatactctatctatacaagttgtattggaaggatgtaagtgtagtgtcccttaaaacatccaatcctgcctcaacagagctttatttaatttcaaaagatgcttactgtactgtaatggaacccagtaatcttgttttatcaaaacttaaaaggatatcatcaatagaagaaaataatctattaaagtggataatcttatcaaaaaggaagaataacgagtggttacagcatgaaatcaaagaaggagaaagggattatgggataatgaggccatatcatacagcactgcaaatttttggattccaaattaacttaaatcacttagctagagaatttttatcaactcctgatttaaccaacattaataatataattcaaagttttacaagaacaattaaagatgttatgttcgaatgggtcaatatcactcatgacaataaaagacataaattaggaggaagatataatctattcccgcttaaaaataaggggaaattaagattattatcacgaagattagtactaagctggatatcattatccttatcaaccagattactgacgggccgttttccagatgaaaaatttgaaaatagggcacagaccggatatgtatcattggctgatattgatttagaatccttaaagttattatcaagaaatattgtcaaaaattacaaagaacacataggattaatatcatactggtttttgaccaaagaggtcaaaatactaatgaagcttataggaggagtcaaactactaggaattcctaaacagtacaaagagttagaggatcgatcatctcagggttatgaatatgataatgaatttgatattgattaatacataaaaacataaaataaaacacctattcctcacccattcacttccaacaaaatgaaaagtaagaaaaacatgtaatatatatataccaaacagagtttttctcttgtttggt

SEQ ID NO: 37 - DNA, 15462 bp, Human parainfluenza virus 3
accaaacaagagaagaaacttgtctgggaatataaatttaactttaaattaacttaggattaaagacattgactagaaggtcaagaaaagggaactctataatttcaaaaatgttgagcctatttgatacatttaatgcacgtaggcaagaaaacataacaaaatcagccggtggagctatcattcctggacagaaaaatactgtctctatattcgcccttggaccgacaataactgatgataatgagaaaatgacattagctcttctatttctatctcattcactagataatgagaaacaacatgcacaaagggcagggttcttggtgtctttattgtcaatggcttatgccaatccagagctctacctaacaacaaatggaagtaatgcagatgtcaagtatgtcatatacatgattgagaaagatctaaaacggcaaaagtatggaggatttgtggttaagacgagagagatgatatatgaaaagacaactgattggatatttggaagtgacctggattatgatcaggaaactatgttgcagaacggcaggaacaattcaacaattgaagaccttgtccacacatttgggtatccatcatgtttaggagctcttataatacagatctggatagttctggtcaaagctatcactagtatctcagggttaagaaaaggctttttcacccgattggaagctttcagacaagatggaacagtgcaggcagggctggtattgagcggtgacacagtggatcagattgggtcaatcatgcggtctcaacagagcttggtaactcttatggttgaaacattaataacaatgaataccagcagaaatgacctcacaaccatagaaaagaatatacaaattgttggcaactacataagagatgcaggtctcgcttcattcttcaatacaatcagatatggaattgagaccagaatggcagctttgactctatccactctcagaccagatatcaatagattaaaagctttgatggaactgtatttatcaaagggaccacgcgctcctttcatctgtatcctcagagatcctatacatggtgagttcgcaccaggcaactatcctgccatatggagctatgcaatgggggtggcagttgtacaaaatagagccatgcaacagtatgtgacgggaagatcatatctagacattgatatgttccagctaggacaagcagtagcacgtgatgccgaagctcaaatgagctcaacactggaagatgaacttggagtgacacacgaatctaaagaaagcttgaagagacatataaggaacataaacagttcagagacatctttccacaaaccgacaggtggatcagccatagagatggcaatagatgaagagccagaacaattcgaacatagagcagatcaagaacaaaatggagaacctcaatcatccataattcaatatgcctgggcagaaggaaatagaagcgatgatcagactgagcaagctacagaatctgacaatatcaagaccgaacaacaaaacatcagagacagactaaacaagagactcaacgacaagaagaaacaaagcagtcaaccacccactaatcccacaaacagaacaaaccaggacgaaatagatgatctgtttaacgcatttggaagcaactaatcgaatcaacattttaatctaaatcaataataaataagaaaaacttaggattaaagaatcctatcataccggaatatagggtggtaaatttagagtctgcttgaaactcaatcaatagagagttgatggaaagcgatgctaaaaactatcaaatcatggattcttgggaagaggaatcaagagataaatcaactaatatctcctcggccctcaacatcattgaattcatactcagcaccgacccccaagaagacttatcggaaaacgacacaatcaacacaagaacccagcaactcagtgccaccatctgtcaaccagaaatcaaaccaacagaaacaagtgagaaagatagtggatcaactgacaaaaatagacagtccgggtcatcacacgaatgtacaacagaagcaaaagatagaaatattgatcaggaaactgtacagagaggacctgggagaagaagcagctcagatagtagagctgagactgtggtctctggaggaatccccagaagcatcacagattctaaaaatggaacccaaaacacggaggatattgatctcaatgaaattagaaagatggataaggactctattgaggggaaaatgcgacaatctgcaaatgttccaagcgagatatcaggaagtgatgacatatttacaacagaacaaagtagaaacagtgatcatggaagaagcctggaatctatcagtacacctgatacaagatcaataagtgttgttactgctgcaacaccagatgatgaagaagaaatactaatgaaaaatagtaggacaaagaaaagttcttcaacacatcaagaagatgacaaaagaattaaaaaagggggaaaagggaaagactggtttaagaaatcaaaagataccgacaaccagataccaacatcagactacagatccacatcaaaagggcagaagaaaatctcaaagacaacaaccaccaacaccgacacaaaggggcaaacagaaatacagacagaatcatcagaaacacaatcctcatcatggaatctcatcatcgacaacaacaccgaccggaacgaacagacaagcacaactcctccaacaacaacttccagatcaacttatacaaaagaatcgatccgaacaaactctgaatccaaacccaagacacaaaagacaaatggaaaggaaaggaaggatacagaagagagcaatcgatttacagagagggcaattactctattgcagaatcttggtgtaattcaatccacatcaaaactagatttatatcaagacaaacgagttgtatgtgtagcaaatgtactaaacaatgtagatactgcatcaaagatagatttcctggcaggattagtcataggggtttcaatggacaacgacacaaaattaacacagatacaaaatgaaatgctaaacctcaaagcagatctaaagaaaatggacgaatcacatagaagattgatagaaaatcaaagagaacaactgtcattgatcacgtcactaatttcaaatctcaaaattatgactgagagaggaggaaagaaagaccaaaatgaatccaatgagagagtatccatgatcaaaacaaaattgaaagaagaaaagatcaagaagaccaggtttgacccacttatggaggcacaaggcattgacaagaatatacccgatctatatcgacatgcaggagatacactagagaacgatgtacaagttaaatcagagatattaagttcatacaatgagtcaaatgcaacaagactaatacccaaaaaagtgagcagtacaatgagatcactagttgcagtcatcaacaacagcaatctctcacaaagcacaaaacaatcatacataaacgaactcaaacgttgcaaaaatgatgaagaagtatctgaattaatggacatgttcaatgaagatgtcaacaattgccaatgatccaacaaagaaacgacaccgaacaaacagacaagaaacaacagtagatcaaaacctgtcaacacacacaaaatcaagcagaatgaaacaacagatatcaatcaatatacaaataagaaaaacttaggattaaagaataaattaatccttgtccaaaatgagtataactaactctgcaatatacacattcccagaatcatcattctctgaaaatggtcatatagaaccattaccactcaaagtcaatgaacagaggaaagcagtaccccacattagagttgccaagatcggaaatccaccaaaacacggatcccggtatttagatgtcttcttactcggcttcttcgagatggaacgaatcaaagacaaatacgggagtgtgaatgatctcgacagtgacccgagttacaaagtttgtggctctggatcattaccaatcggattggctaagtacactgggaatgaccaggaattgttacaagccgcaaccaaactggatatagaagtgagaagaacagtcaaagcgaaagagatggttgtttacacggtacaaaatataaaaccagaactgtacccatggtccaatagactaagaaaaggaatgctgttcgatgccaacaaagttgctcttgctcctcaatgtcttccactagataggagcataaaatttagagtaatcttcgtgaattgtacggcaattggatcaataaccttgttcaaaattcctaagtcaatggcatcactatctctacccaacacaatatcaatcaatctgcaggtacacataaaaacaggggttcagactgattctaaagggatagttcaaattttggatgagaaaggcgaaaaatcactgaatttcatggtccatctcggattgatcaaaagaaaagtaggcagaatgtactctgttgaatactgtaaacagaaaatcgagaaaatgagattgatattttctttaggactagttggaggaatcagtcttcatgtcaatgcaactgggtccatatcaaaaacactagcaagtcagctggtattcaaaagagagatttgttatcctttaatggatctaaatccgcatctcaatctagttatctgggcttcatcagtagagattacaagagtggatgcaattttccaaccttctttacctggcgagttcagatactatcctaatattattgcaaaaggagttgggaaaatcaaacaatggaactagtaatctctattttagtccggacgtatctattaagccgaagcaaataaaggataatcaaaaacttaggacaaaagaggtcaataccaacaactattagcagtcacactcgcaagaataagagagaagggaccaaaaaagtcaaataggagaaatcaaaacaaaaggtacagaacaccagaacaacaaaatcaaaacatccaactcactcaaaacaaaaattccaaaagagaccggcaacacaacaagcactgaacacaatgccaacttcaatactgctaattattacaaccatgatcatggcatctttctgccaaatagatatcacaaaactacagcacgtaggtgtattggtcaacagtcccaaagggatgaagatatcacaaaactttgaaacaagatatctaattttgagcctcataccaaaaatagaagactctaactcttgtggtgaccaacagatcaagcaatacaagaagttattggatagactgatcatccctttatatgatggattaagattacagaaagatgtgatagtaaccaatcaagaatccaatgaaaacactgatcccagaacaaaacgattctttggaggggtaattggaaccattgctctgggagtagcaacctcagcacaaattacagcggcagttgctctggttgaagccaagcaggcaagatcagacatcgaaaaactcaaagaagcaattagggacacaaacaaagcagtgcagtcagttcagagctccataggaaatttaatagtagcaattaaatcagtccaggattatgttaacaaagaaatcgtgccatcgattgcgaggctaggttgtgaagcagcaggacttcaattaggaattgcattaacacagcattactcagaattaacaaacatatttggtgataacataggatcgttacaagaaaaaggaataaaattacaaggtatagcatcattataccgcacaaatatcacagaaatattcacaacatcaacagttgataaatatgatatctatgatctgttatttacagaatcaataaaggtgagagttatagatgttgacttgaatgattactcaatcaccctccaagtcagactccctttattaactaggctgctgaacactcagatctacaaagtagattccatatcatataacatccaaaacagagaatggtatatccctcttcccagccatatcatgacgaaaggggcatttctaggtggagcagacgtcaaagaatgtatagaagcattcagcagctatatatgcccttctgatccaggatttgtattaaaccatgaaatagagagctgcttatcaggaaacatatcccaatgtccaagaacaacggtcacatcagacattgttccaagatatgcatttgtcaatggaggagtggttgcaaactgtataacaaccacctgtacatgcaacggaattggtaatagaatcaatcaaccacctgatcaaggagtaaaaattataacacataaagaatgtagtacaataggtatcaacggaatgctgttcaatacaaataaagaaggaactcttgcattctatacaccaaatgatataacactaaacaattctgttgcacttgatccaattgacatatcaatcgagctcaacaaggccaaatcagatctagaagaatcaaaagaatggataagaaggtcaaatcaaaaactagattctattggaaattggcatcaatctagcactacaatcataattattttgataatgatcattatattgtttataattaatataacgataattacaattgcaattaagtattacagaattcaaaagagaaatcgagtggatcaaaatgacaagccatatgtactaacaaacaaataacatatctacagatcattagatattaaaattataaaaaacttaggagtaaagttacgcaatccaactctactcatataattgaggaaggacccaatagacaaatccaaattcgagatggaatactggaagcataccaatcacggaaaggatgctggtaatgagctggagacgtctatggctactcatggcaacaagctcactaataagataatatacatattatggacaataatcctggtgttattatcaatagtcttcatcatagtgctaattaattccatcaaaagtgaaaaggcccacgaatcattgctgcaagacataaataatgagtttatggaaattacagaaaagatccaaatggcatcggataataccaatgatctaatacagtcaggagtgaatacaaggcttcttacaattcagagtcatgtccagaattacataccaatatcattgacacaacagatgtcagatcttaggaaattcattagtgaaattacaattagaaatgataatcaagaagtgctgccacaaagaataacacatgatgtaggtataaaacctttaaatccagatgatttttggagatgcacgtctggtcttccatctttaatgaaaactccaaaaataaggttaatgccagggccgggattattagctatgccaacgactgttgatggctgtgttagaactccgtctttagttataaatgatctgatttatgcttatacctcaaatctaattactcgaggttgtcaggatataggaaaatcatatcaagtcttacagatagggataataactgtaaactcagacttggtacctgacttaaatcctaggatctctcatacctttaacataaatgacaataggaagtcatgttctctagcactcctaaatacagatgtatatcaactgtgttcaactcccaaagttgatgaaagatcagattatgcatcatcaggcatagaagatattgtacttgatattgtcaattatgatggttcaatctcaacaacaagatttaagaataataacataagctttgatcaaccatatgctgcactatacccatctgttggaccagggatatactacaaaggcaaaataatatttctcgggtatggaggtcttgaacatccaataaatgagaatgtaatctgcaacacaactgggtgccccgggaaaacacagagagactgtaatcaagcgtctcatagtccatggttttcagataggaggatggtcaactccatcattgttgttgacaaaggcttaaactcaattccaaaattgaaagtatggacgatatctatgcgacaaaattactgggggtcagaaggaaggttacttctactaggtaacaagatctatatatatacaagatctacaagttggcatagcaagttacaattaggaataattgatattactgattacagtgatataaggataaaatggacatggcataatgtgctatcaagaccaggaaacaatgaatgtccatggggacattcatgtccagatggatgtataacaggagtatatactgatgcatatccactcaatcccacagggagcattgtgtcatctgtcatattagactcacaaaaatcgagagtgaacccagtcataacttactcaacagcaaccgaaagagtaaacgagctggccatcctaaacagaacactctcagctggatatacaacaacaagctgcattacacactataacaaaggatattgttttcatatagtagaaataaatcataaaagcttaaacacatttcaacccatgttgttcaaaacagagattccaaaaagctgcagttaatcataattaaccataatatgcatcaatctatctataatacaagtatatgataagtaatcagcaatcagacaatagacaaaagggaaatataaaaaacttaggagcaaagcgtgctcgggaaatggacactgaatctaacaatggcactgtatctgacatactctatcctgagtgtcaccttaactctcctatcgttaaaggtaaaatagcacaattacacactattatgagtctacctcagccttatgatatggatgacgactcaatactagttatcactagacagaaaataaaacttaataaattggataaaagacaacgatctattagaagattaaaattaatattaactgaaaaagtgaatgacttaggaaaatacacatttatcagatatccagaaatgtcaaaagaaatgttcaaattatatatacctggtattaacagtaaagtgactgaattattacttaaagcagatagaacatatagtcaaatgactgatggattaagagatctatggattaatgtgctatcaaaattagcctcaaaaaatgatggaagcaattatgatcttaatgaagaaattaataatatatcgaaagttcacacaacctataaatcagataaatggtataatccattcaaaacatggtttactatcaagtatgatatgagaagattacaaaaagctcgaaatgagatcacttttaatgttgggaaggattataacttgttagaagaccagaagaatttcttattgatacatccagaattggttttgatattagataaacaaaactataatggttatctaattactcctgaattagtattgatgtattgtgacgtagtcgaaggccgatggaatataagtgcatgtgctaagttagatccaaaattacaatctatgtatcagaaaggtaataacctgtgggaagtgatagataaattgtttccaattatgggagaaaagacatttgatgtgatatcgttattagaaccacttgcattatccttaattcaaactcatgatcctgttaaacaactaagaggagcttttttaaatcatgtgttatccgagatggaattaatatttgaatctagagaatcgattaaggaatttctgagtgtagattacattgataaaattttagatatatttaataagtctacaatagatgaaatagcagagattttctctttttttagaacatttgggcatcctccattagaagctagtattgcagcagaaaaggttagaaaatatatgtatattggaaaacaattaaaatttgacactattaataaatgtcatgctatcttctgtacaataataattaacggatatagagagaggcatggtggacagtggcctcctgtgacattacctgatcatgcacacgaattcatcataaatgcttacggttcaaactctgcgatatcatatgaaaatgctgttgattattaccagagctttataggaataaaattcaataaattcatagagcctcagttagatgaggatttgacaatttatatgaaagataaagcattatctccaaaaaaatcaaattgggacacagtttatcctgcatctaatttactgtaccgtactaacgcatccaacgaatcacgaagattagttgaagtatttatagcagatagtaaatttgatcctcatcagatattggattatgtagaatctggggactggttagatgatccagaatttaatatttcttatagtcttaaagaaaaagagatcaaacaggaaggtagactctttgcaaaaatgacatacaaaatgagagctacacaagttttatcagagacactacttgcaaataacataggaaaattctttcaagaaaatgggatggtgaagggagagattgaattacttaagagattaacaaccatatcaatatcaggagttccacggtataatgaagtgtacaataattctaaaagccatacagatgaccttaaaacctacaataaaataagtaatcttaatttgtcttctaatcagaaatcaaagaaatttgaattcaagtcaacggatatctacaatgatggatacgagactgtgagctgtttcctaacaacagatctcaaaaaatactgtcttaattggagatatgaatcaacagctctatttggagaaacttgcaaccaaatatttggattaaataaattgtttaattggttacaccctcgtcttgaaggaagtacaatctatgtaggtgatccttactgtcctccatcagataaagaacatatatcattagaggatcaccctgattctggtttttacgttcataacccaagagggggtatagaaggattttgtcaaaaattatggacactcatatctataagtgcaatacatctagcagctgttagaataggcgtgagggtgactgcaatggttcaaggagacaatcaagctatagctgtaaccacaagagtacccaacaattatgactacagagttaagaaggagatagtttataaagatgtagtgagattttttgattcattaagagaagtgatggatgatctaggtcatgaacttaaattaaatgaaacgattataagtagcaagatgttcatatatagcaaaagaatctattatgatgggagaattcttcctcaagctctaaaagcattatctagatgtgtcttctggtcagagacagtaatagacgaaacaagatcagcatcttcaaatttggcaacatcatttgcaaaagcaattgagaatggttattcacctgttctaggatatgcatgctcaatttttaagaacattcaacaactatatattgcccttgggatgaatatcaatccaactataacacagaatatcagagatcagtattttaggaatccaaattggatgcaatatgcctctttaatacctgctagtgttgggggattcaattacatggccatgtcaagatgttttgtaaggaatattggtgatccatcagttgccgcattggctgatattaaaagatttattaaggcgaatctattagaccgaagtgttctttataggattatgaatcaagaaccaggtgagtcatcttttttggactgggcttcagatccatattcatgcaatttaccacaatctcaaaatataaccaccatgataaaaaatataacagcaaggaatgtattacaagattcaccaaatccattattatctggattattcacaaatacaatgatagaagaagatgaagaattagctgagttcctgatggacaggaaggtaattctccctagagttgcacatgatattctagataattctctcacaggaattagaaatgccatagctggaatgttagatacgacaaaatcactaattcgggttggcataaatagaggaggactgacatatagtttgttgaggaaaatcagtaattacgatctagtacaatatgaaacactaagtaggactttgcgactaattgtaagtgataaaatcaagtatgaagatatgtgttcggtagaccttgccatagcattgcgacaaaagatgtggattcatttatcaggaggaaggatgataagtggacttgaaacgcctgacccattagaattactatctggggtagtaataacaggatcagaacattgtaaaatatgttattcttcagatggcacaaacccatatacttggatgtatttacccggtaatatcaaaataggatcagcagaaacaggtatatcgtcattaagagttccttattttggatcagtcactgatgaaagatctgaagcacaattaggatatatcaagaatcttagtaaacctgcaaaagccgcaataagaatagcaatgatatatacatgggcatttggtaatgatgagatatcttggatggaagcctcacagatagcacaaacacgtgcaaattttacactagatagtctcaaaattttaacaccggtagctacatcaacaaatttatcacacagattaaaggatactgcaactcagatgaaattctccagtacatcattgatcagagtcagcagattcataacaatgtccaatgataacatgtctatcaaagaagctaatgaaaccaaagatactaatcttatttatcaacaaataatgttaacaggattaagtgttttcgaatatttatttagattaaaagaaaccacaggacacaaccctatagttatgcatctgcacatagaagatgagtgttgtattaaagaaagttttaatgatgaacatattaatccagagtctacattagaattaattcgatatcctgaaagtaatgaatttatttatgataaagacccactcaaagatgtggacttatcaaaacttatggttattaaagaccattcttacacaattgatatgaattattgggatgatactgacatcatacatgcaatttcaatatgtactgcaattacaatagcagatactatgtcacaattagatcgagataatttaaaagagataatagttattgcaaatgatgatgatattaatagcttaatcactgaatttttgactcttgacatacttgtatttctcaagacatttggtggattattagtaaatcaatttgcatacactctttatagtctaaaaatagaaggtagggatctcatttgggattatataatgagaacactgagagatacttcccattcaatattaaaagtattatctaatgcattatctcatcctaaagtattcaagaggttctgggattgtggagttttaaaccctatttatggtcctaatactgctagtcaagaccagataaaacttgccctatctatatgtgaatattcactagatctatttatgagagaatggttgaatggtgtatcacttgaaatatacatttgtgacagcgatatggaagttgcaaatgataggaaacaagcctttatttctagacacctttcatttgtttgttgtttagcagaaattgcatctttcggacctaacctgttaaacttaacatacttggagagacttgatctattgaaacaatatcttgaattaaatattaaagaagaccctactcttaaatatgtacaaatatctggattattaattaaatcgttcccatcaactgtaacatacgtaagaaagactgcaatcaaatatctaaggattcgcggtattagtccacctgaggtaattgatgattgggatccggtagaagatgaaaatatgctggataacattgtcaaaactataaatgataactgtaataaagataataaagggaataaaattaacaatttctggggactagcacttaagaactatcaagtccttaaaatcagatctataacaagtgattctgatgataatgatagactagatgctaatacaagtggtttgacacttcctcaaggagggaattatctatcgcatcaattgagattattcggaatcaacagcactagttgtctgaaagctcttgagttatcacaaattttaatgaaggaagtcaataaagacaaggacaggctcttcctgggagaaggagcaggagctatgctagcatgttatgatgccacattaggacctgcagttaattattataattcaggtttgaatataacagatgtaattggtcaacgagaattgaaaatatttccttcagaggtatcattagtaggtaaaaaattaggaaatgtgacacagattcttaacagggtaaaagtactgttcaatgggaatcctaattcaacatggataggaaatatggaatgtgagagcttaatatggagtgaattaaatgataagtccattggattagtacattgtgatatggaaggagctatcggtaaatcagaagaaactgttctacatgaacattatagtgttataagaattacatacttgattggggatgatgatgttgttttagtttccaaaattatacctacaatcactccgaattggtctagaatactttatctatataaattatattggaaagatgtaagtataatatcactcaaaacttctaatcctgcatcaacagaattatatctaatttcgaaagatgcatattgtactataatggaacctagtgaaattgttttatcaaaacttaaaagattgtcactcttggaagaaaataatctattaaaatggatcattttatcaaagaagaggaataatgaatggttacatcatgaaatcaaagaaggagaaagagattatggaatcatgagaccatatcatatggcactacaaatctttggatttcaaatcaatttaaatcatctggcgaaagaatttttatcaaccccagatctgactaatatcaacaatataatccaaagttttcagcgaacaataaaggatgttttatttgaatggattaatataactcatgatgataagagacataaattaggcggaagatataacatattcccactgaaaaataagggaaagttaagactgctatcgagaagactagtattaagttggatttcattatcattatcgactcgattacttacaggtcgctttcctgatgaaaaatttgaacatagagcacagactggatatgtatcattagctgatactgatttagaatcattaaagttattgtcgaaaaacatcattaagaattacagagagtgtataggatcaatatcatattggtttctaaccaaagaagttaaaatacttatgaaattgattggtggtgctaaattattaggaattcccagacaatataaagaacccgaagaccagttattagaaaactacaatcaacatgatgaatttgatatcgattaaaacataaatacaatgaagatatatcctaacctttatctttaagcctaggaatagacaaaaagtaagaaaaacatgtaatatatatataccaaacagagttcttctcttgtttggt

SEQ ID NO: 38 - protein, 1271 aa, Artificial Sequence
  Recombinant protein
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 39 - protein, 1270 aa, Artificial Sequence
  Recombinant protein
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 40 - DNA, 3816 bp, Artificial Sequence
  Recombinant nucleic acid
atgttcgtgtttctggtgctgctgcctctggtgagctcccagtgcgtgaacctgaggacaaggacccagctgccccctgcctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagcggttcgacaatccagtgctgccctttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcggcgtgtattcctctgccaacaattgcacatttgagtacgtgtcccagcccttcctgatggacctggagggcaagcagggcaatttcaagaacctgagggagttcgtgtttaagaatatcgatggctacttcaagatctactccaagcacaccccaatcaacctggtgcgcgacctgccacagggcttctctgccctggagccactggtggatctgcccatcggcatcaacatcacccggtttcagacactgctggccctgcacagaagctacctgacaccaggcgacagctcctctggatggaccgcaggagctgccgcctactatgtgggctatctgcagcccaggaccttcctgctgaagtacaacgagaatggcaccatcacagacgccgtggattgcgccctggatcccctgtctgagaccaagtgtacactgaagagctttaccgtggagaagggcatctatcagacaagcaatttcagggtgcagcctaccgagtccatcgtgcgctttcccaatatcacaaacctgtgcccttttggcgaggtgttcaacgcaacccgcttcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcccctggccagacaggcaagatcgccgactacaattataagctgcctgacgatttcaccggctgcgtgatcgcctggaactctaacaatctggatagcaaagtgggcggcaactacaattatcggtaccggctgtttagaaagtctaatctgaagccattcgagagggacatctccacagagatctaccaggccggctctaagccctgcaatggcgtggagggctttaactgttatttccctctgcagagctacggcttccagccaacaaacggcgtgggctatcagccctaccgcgtggtggtgctgtcttttgagctgctgcacgcacctgcaacagtgtgcggaccaaagaagagcaccaatctggtgaagaacaagtgcgtgaacttcaacttcaacggactgaccggcacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcagggcgtgaattgtaccgaggtgcccgtggcaatccacgcagatcagctgacccctacatggcgggtgtactctaccggcagcaacgtgttccagacaagagccggatgcctgatcggagccgagcacgtgaacaatagctatgagtgcgacatccctatcggcgccggcatctgtgcctcctaccagacccagacaaactccagagggtctgcctcctctgtggccagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggctctttttgtacccagctgaatagagccctgacaggcatcgccgtggagcaggacaagaacacacaggaggtgttcgcccaggtgaagcagatctacaagaccccacccatcaaggactttggcggcttcaacttcagccagatcctgcccgatcctagcaagccatccaagcggtctcctatcgaggacctgctgttcaacaaggtgaccctggccgatgccggcttcatcaagcagtatggcgattgcctgggcgacatcgccgccagagacctgatctgtgcccagaagtttaatggcctgaccgtgctgcctccactgctgacagatgagatgatcgcccagtacacatctgccctgctggccggcaccatcacaagcggatggaccttcggcgcaggacccgccctgcagatcccctttcccatgcagatggcctatcggttcaacggcatcggcgtgacccagaatgtgctgtacgagaaccagaagctgatcgccaatcagtttaactccgccatcggcaagatccaggactctctgagctccacacccagcgccctgggcaagctgcagaacgtggtgaatcagaacgcccaggccctgaataccctggtgaagcagctgtctagcaacttcggcgccatctcctctgtgctgaatgatatcctgagcaggctggaccctccagaggcagaggtgcagatcgaccggctgatcacaggcagactgcagtccctgcagacctacgtgacacagcagctgatcagggcagcagagatcagggcctctgccaatctggccgccaccaagatgagcgagtgcgtgctgggccagtccaagagagtggacttttgtggcaagggctatcacctgatgagcttcccacagtccgcccctcacggagtggtgtttctgcacgtgacctacgtgccagcccaggagaagaacttcaccacagcaccagcaatctgccacgatggcaaggcacactttcctagggagggcgtgttcgtgagcaacggcacccactggtttgtgacacagcgcaatttctacgagccacagatcatcaccacagacaatacattcgtgtccggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgacaggctgaacgaggtggccaagaatctgaacgagtccctgatcgatctgcaggagctgggcaagtatgagcagtacatcaagtggccctggtatatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactacacctga

SEQ ID NO: 41 - DNA, 3813 bp, Artificial Sequence
  Recombinant nucleic acid
atgttcgtgtttctggtgctgctgcctctggtgagctcccagtgcgtgaacctgaccacaaggacccagctgccccctgcctataccaattccttcacacggggcgtgtactatcccgacaaggtgtttagatctagcgtgctgcactccacacaggatctgtttctgcctttcttttctaacgtgacctggttccacgtgatcagcggcaccaatggcacaaagcggttcgacaatccagtgctgccctttaacgatggcgtgtacttcgcctccatcgagaagtctaacatcatcagaggctggatctttggcaccacactggacagcaagacacagtccctgctgatcgtgaacaatgccaccaacgtggtcatcaaggtgtgcgagttccagttttgtaatgatccattcctggaccacaagaacaataagtcttggatggagagcgagtttcgcgtgtattcctctgccaacaattgcacatttgagtacgtgtcccagcccttcctgatggacctggagggcaagcagggcaatttcaagaacctgagggagttcgtgtttaagaatatcgatggctacttcaagatctactccaagcacaccccaatcatcgtgcgcgagccagaagacctgccacagggcttctctgccctggagccactggtggatctgcccatcggcatcaacatcacccggtttcagacactgctggccctgcacagaagctacctgacaccaggcgacagctcctctggatggaccgcaggagctgccgcctactatgtgggctatctgcagcccaggaccttcctgctgaagtacaacgagaatggcaccatcacagacgccgtggattgcgccctggatcccctgtctgagaccaagtgtacactgaagagctttaccgtggagaagggcatctatcagacaagcaatttcagggtgcagcctaccgagtccatcgtgcgctttcccaatatcacaaacctgtgcccttttgacgaggtgttcaacgcaacccgcttcgccagcgtgtacgcctggaataggaagcgcatctccaactgcgtggccgactattctgtgctgtacaaccttgctccattcttcacctttaagtgctatggcgtgagccccacaaagctgaatgacctgtgctttaccaacgtgtacgccgattccttcgtgatcaggggcgacgaggtgcgccagatcgcccctggccagacaggcaacatcgccgactacaattataagctgcctgacgatttcaccggctgcgtgatcgcctggaactctaacaagctggatagcaaagtgagcggcaactacaattatctgtaccggctgtttagaaagtctaatctgaagccattcgagagggacatctccacagagatctaccaggccggcaacaagccctgcaatggcgtggccggctttaactgttatttccctctgaggagctacagcttcaggccaacatacggcgtgggacatcagccctaccgcgtggtggtgctgtcttttgagctgctgcacgcacctgcaacagtgtgcggaccaaagaagagcaccaatctggtgaagaacaagtgcgtgaacttcaacttcaacggactgaagggcacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcagggcgtgaattgtaccgaggtgcccgtggcaatccacgcagatcagctgacccctacatggcgggtgtactctaccggcagcaacgtgttccagacaagagccggatgcctgatcggagccgagtacgtgaacaatagctatgagtgcgacatccctatcggcgccggcatctgtgcctcctaccagacccagacaaagtcccacgggtctgcctcctctgtggccagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggctctttttgtacccagctgaagagagccctgacaggcatcgccgtggagcaggacaagaacacacaggaggtgttcgcccaggtgaagcagatctacaagaccccacccatcaagtactttggcggcttcaacttcagccagatcctgcccgatcctagcaagccatccaagcggtctcctatcgaggacctgctgttcaacaaggtgaccctggccgatgccggcttcatcaagcagtatggcgattgcctgggcgacatcgccgccagagacctgatctgtgcccagaagtttaagggcctgaccgtgctgcctccactgctgacagatgagatgatcgcccagtacacatctgccctgctggccggcaccatcacaagcggatggaccttcggcgcaggacccgccctgcagatcccctttcccatgcagatggcctatcggttcaacggcatcggcgtgacccagaatgtgctgtacgagaaccagaagctgatcgccaatcagtttaactccgccatcggcaagatccaggactctctgagctccacacccagcgccctgggcaagctgcaggatgtggtgaatcacaacgcccaggccctgaataccctggtgaagcagctgtctagcaagttcggcgccatctcctctgtgctgaatgatatcttcagcaggctggaccctccagaggcagaggtgcagatcgaccggctgatcacaggcagactgcagtccctgcagacctacgtgacacagcagctgatcagggcagcagagatcagggcctctgccaatctggccgccaccaagatgagcgagtgcgtgctgggccagtccaagagagtggacttttgtggcaagggctatcacctgatgagcttcccacagtccgcccctcacggagtggtgtttctgcacgtgacctacgtgccagcccaggagaagaacttcaccacagcaccagcaatctgccacgatggcaaggcacactttcctagggagggcgtgttcgtgagcaacggcacccactggtttgtgacacagcgcaatttctacgagccacagatcatcaccacagacaatacattcgtgtccggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgacaggctgaacgaggtggccaagaatctgaacgagtccctgatcgatctgcaggagctgggcaagtatgagcagtacatcaagtggccctggtatatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactacacctga

SEQ ID NO: 42 - DNA, 19308 bp, Artificial Sequence
  Recombinant nucleic acid
accaaacaagagaagagactggtttgggaatattaattcaaataaaaattaacttaggattaaagaactttaccgaaaggtaaggggaaagaaatcctaagagcttagccatgttgagtctattcgacacattcagtgcgcgtaggcaggagaacataacgaaatcagctggtggggctgttattcccgggcaaaaaaacactgtgtctatatttgctcttggaccatcaataacagatgacaatgataaaatgacattggctcttctctttttgtctcattctttagacaatgaaaagcagcatgcgcaaagagctggatttttagtttctctgttatcaatggcttatgccaacccagaattatatttaacatcaaatggtagtaatgcagatgttaaatatgttatctacatgatagagaaagacccaggaagacagaaatatggtgggtttgtcgtcaagactagagagatggtttatgaaaagacaactgattggatgttcgggagtgatcttgagtatgatcaagacaatatgttgcaaaatggtagaagcacttctacaatcgaggatcttgttcatacttttggatatccatcgtgtcttggagcccttataatccaagtttggataatacttgttaaggctataaccagtatatcaggattgaggaaaggattctttactcggttagaagcatttcgacaagatggaacagttaaatccagtctagtgttgagcggtgatgcagtagaacaaattggatcaattatgaggtcccaacagagcttggtaacactcatggttgaaacactgataacaatgaacacaggcaggaatgatctgacaacaatagaaaagaatatacagattgtaggaaactacatcagagatgcaggtcttgcttcatttttcaacacaatcagatatggcattgagactagaatggcagctctaactctgtctacccttagaccggatatcaacagactcaaggcactgatcgagttatatctatcaaaggggccacgtgctccttttatatgcattttgagagatcccgtgcatggtgagtttgcaccaggcaactatcctgccctctggagttatgcgatgggtgtagcagttgtacaaaacaaggccatgcaacagtatgtaacaggaaggtcttatctggatattgaaatgttccaacttggtcaagcagtggcacgtgatgccgagtcgcagatgagttcaatattagaggatgaactgggggtcacacaagaagccaagcaaagcttgaagaaacacatgaagaacatcagcagttcagatacaacctttcataagcctacagggggatcagccatagaaatggcgatagatgaagaagcagggcagcctgaatccagaggagatcaggatcaaggagatgagcctcggtcatccatagttccttatgcatgggcagacgaaaccgggaatgacaatcaaactgaatcaactacagaaattgacagcatcaaaactgaacaaagaaacatcagagacaggctgaacaaaagactcaacgagaaaaggaaacagagtgacccgagatcaactgacatcacaaacaacacaaatcaaactgaaatagatgatttgttcagtgcattcggaagcaactagtcacaaagagatgaccaggcgcgccaagtaagaaaaacttaggattaatggacctgcaggatgttcgtgtttctggtgctgctgcctctggtgagctcccagtgcgtgaacctgaggacaaggacccagctgccccctgcctataccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagcggttcgacaatccagtgctgccctttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcggcgtgtattcctctgccaacaattgcacatttgagtacgtgtcccagcccttcctgatggacctggagggcaagcagggcaatttcaagaacctgagggagttcgtgtttaagaatatcgatggctacttcaagatctactccaagcacaccccaatcaacctggtgcgcgacctgccacagggcttctctgccctggagccactggtggatctgcccatcggcatcaacatcacccggtttcagacactgctggccctgcacagaagctacctgacaccaggcgacagctcctctggatggaccgcaggagctgccgcctactatgtgggctatctgcagcccaggaccttcctgctgaagtacaacgagaatggcaccatcacagacgccgtggattgcgccctggatcccctgtctgagaccaagtgtacactgaagagctttaccgtggagaagggcatctatcagacaagcaatttcagggtgcagcctaccgagtccatcgtgcgctttcccaatatcacaaacctgtgcccttttggcgaggtgttcaacgcaacccgcttcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcccctggccagacaggcaagatcgccgactacaattataagctgcctgacgatttcaccggctgcgtgatcgcctggaactctaacaatctggatagcaaagtgggcggcaactacaattatcggtaccggctgtttagaaagtctaatctgaagccattcgagagggacatctccacagagatctaccaggccggctctaagccctgcaatggcgtggagggctttaactgttatttccctctgcagagctacggcttccagccaacaaacggcgtgggctatcagccctaccgcgtggtggtgctgtcttttgagctgctgcacgcacctgcaacagtgtgcggaccaaagaagagcaccaatctggtgaagaacaagtgcgtgaacttcaacttcaacggactgaccggcacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcagggcgtgaattgtaccgaggtgcccgtggcaatccacgcagatcagctgacccctacatggcgggtgtactctaccggcagcaacgtgttccagacaagagccggatgcctgatcggagccgagcacgtgaacaatagctatgagtgcgacatccctatcggcgccggcatctgtgcctcctaccagacccagacaaactccagagggtctgcctcctctgtggccagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggctctttttgtacccagctgaatagagccctgacaggcatcgccgtggagcaggacaagaacacacaggaggtgttcgcccaggtgaagcagatctacaagaccccacccatcaaggactttggcggcttcaacttcagccagatcctgcccgatcctagcaagccatccaagcggtctcctatcgaggacctgctgttcaacaaggtgaccctggccgatgccggcttcatcaagcagtatggcgattgcctgggcgacatcgccgccagagacctgatctgtgcccagaagtttaatggcctgaccgtgctgcctccactgctgacagatgagatgatcgcccagtacacatctgccctgctggccggcaccatcacaagcggatggaccttcggcgcaggacccgccctgcagatcccctttcccatgcagatggcctatcggttcaacggcatcggcgtgacccagaatgtgctgtacgagaaccagaagctgatcgccaatcagtttaactccgccatcggcaagatccaggactctctgagctccacacccagcgccctgggcaagctgcagaacgtggtgaatcagaacgcccaggccctgaataccctggtgaagcagctgtctagcaacttcggcgccatctcctctgtgctgaatgatatcctgagcaggctggaccctccagaggcagaggtgcagatcgaccggctgatcacaggcagactgcagtccctgcagacctacgtgacacagcagctgatcagggcagcagagatcagggcctctgccaatctggccgccaccaagatgagcgagtgcgtgctgggccagtccaagagagtggacttttgtggcaagggctatcacctgatgagcttcccacagtccgcccctcacggagtggtgtttctgcacgtgacctacgtgccagcccaggagaagaacttcaccacagcaccagcaatctgccacgatggcaaggcacactttcctagggagggcgtgttcgtgagcaacggcacccactggtttgtgacacagcgcaatttctacgagccacagatcatcaccacagacaatacattcgtgtccggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgacaggctgaacgaggtggccaagaatctgaacgagtccctgatcgatctgcaggagctgggcaagtatgagcagtacatcaagtggccctggtatatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactacacctgatagtaactagcggcgcgccagcaacaagtaagaaaaacttaggattaatggaaattatccaatccagagacggaaggacaaatccagaatccaaccacaactcaatcaaccaaagattcatggaagacaatgttcaaaacaatcaaatcatggattcttgggaagagggatcaggagataaatcatctgacatctcatcggccctcgacatcattgaattcatactcagcaccgactcccaagagaacacggcagacagcaatgaaatcaacacaggaaccacaagacttagcacgacaatctaccaacctgaatccaaaacaacagaaacaagcaaggaaaatagtggaccagctaacaaaaatcgacagtttggggcatcacacgaacgtgccacagagacaaaagatagaaatgttaatcaggagactgtacagggaggatataggagaggaagcagcccagatagtagaactgagactatggtcactcgaagaatctccagaagcagcccagatcctaacaatggaacccaaatccaggaagatattgattacaatgaagttggagagatggataaggactctactaagagggaaatgcgacaatttaaagatgttccagtcaaggtatcaggaagtgatgccattcctccaacaaaacaagatggagacggtgatgatggaagaggcctggaatctatcagtacatttgattcaggatataccagtatagtgactgccgcaacactagatgacgaagaagaactccttatgaagaacaacaggccaagaaagtatcaatcaacaccccagaacagtgacaagggaattaaaaaaggggttggaaggccaaaagacacagacaaacaatcatcaatattggactacgaactcaacttcaaaggatcgaagaagagccagaaaatcctcaaagccagcacgaatacaggagaaccaacaagaccacagaatggatcccaggggaagagaatcacatcctggaacatcctcaacagcgagagcggcaatcgaacagaatcaacaaaccaaacccatcagacatcaacctcgggacagaaccacacaatgggaccaagcagaacaacctccgaaccaaggatcaagacacaaaagacggatggaaaggaaagagaggacacagaagagagcactcgatttacagaaagggcgattacattattacagaatcttggtgtaatccaatctgcagcaaaattagacctataccaagacaagagagttgtgtgtgtggcgaatgtcctaaacaatgcagatactgcatcaaagatagacttcctagcaggtttgatgataggagtgtcaatggatcatgataccaaattaaatcagattcagaacgagatattaagtttgaaaactgatcttaaaaagatggatgaatcacatagaagactaattgagaatcaaaaagaacaattatcactgatcacatcattaatctcaaatcttaaaattatgacagagagaggagggaagaaggaccaaccagaacctagcgggaggacatccatgatcaagacaaaagcaaaagaagagaaaataaagaaagtcaggtttgaccctcttatggaaacacagggcatcgagaaaaacatccctgacctctatagatcaatagagaaaacaccagaaaacgacacacagatcaaatcagaaataaacagattgaatgatgaatccaatgccactagattagtacctagaagaataagcagtacaatgagatcattaataataatcattaacaacagcaatttatcatcaaaagcaaagcaatcatacatcaacgaactcaagctctgcaagagtgacgaggaagtgtctgagttgatggacatgttcaatgaggatgtcagctcccagtaaaccgccaaccaagggtcaacaccaagaaaaccaatagcacaaaacagccaatcagagaccaccccaatacaccaaaccaatcaacacataacaaagatcgcggccgcatagatgattaagaaaaacttaggatgaaaggactaatcaatcctccgaaacaatgagcatcaccaactccacaatctacacattcccagaatcctctttctccgagaatggcaacatagagccgttaccactcaaggtcaatgaacagagaaaggccatacctcatattagggttgtcaagataggagatccgcccaaacatggatccagatatctggatgtctttttactgggcttctttgagatggaaaggtcaaaagacaggtatgggagcataagtgatctagatgatgatccaagttacaaggtttgtggctctggatcattgccacttgggttggctagatacaccggaaatgatcaggaactcctacaggctgcaaccaagctcgatatagaagtaagaagaactgtaaaggctacggagatgatagtttacactgtacaaaacatcaaacctgaactatatccatggtccagtagattaagaaaagggatgttatttgacgctaataaggttgcacttgctcctcaatgtcttccactagatagagggataaaattcagggtgatatttgtgaactgcacagcaattggatcaataactctattcaaaatccctaagtccatggcattgttatcattgcctaatacaatatcaataaatctacaagtacatatcaaaacaggagttcagacagattccaaaggagtagttcagattctagatgaaaaaggtgaaaaatcactaaatttcatggttcatctcgggttgatcaaaaggaagatgggcagaatgtactcagttgaatattgtaagcagaagatcgagaagatgagattattattctcattgggattagttggagggatcagcttccacgtcaacgcaactggctctatatcaaagacattagcaagtcaattagcattcaaaagagaaatctgctatcccctaatggatctgaatccacacttaaattcagttatatgggcatcatcagttgaaattacaagggtagatgcagttctccagccttcattacctggcgaattcagatactacccaaacatcatagcaaaaggggtcgggaaaatcagacagtaaaatcaacaaccctgatatccaccggtgtattaagccgaagcaaataaaggataatcaaaaacttaggacaaaagaggtcaataccaacaactattagcagtcacactcgcaagaataagagagaagggaccaaaaaagtcaaataggagaaatcaaaacaaaaggtacagaacaccagaacaacaaaatcaaaacatccaactcactcaaaacaaaaattccaaaagagaccggcaacacaacaagcactgaacacaatgccaacttcaatactgctaattattacaaccatgatcatggcatctttctgccaaatagatatcacaaaactacagcacgtaggtgtattggtcaacagtcccaaagggatgaagatatcacaaaactttgaaacaagatatctaattttgagcctcataccaaaaatagaagactctaactcttgtggtgaccaacagatcaagcaatacaagaagttattggatagactgatcatccctttatatgatggattaagattacagaaagatgtgatagtaaccaatcaagaatccaatgaaaacactgatcccagaacaaaacgattctttggaggggtaattggaaccattgctctgggagtagcaacctcagcacaaattacagcggcagttgctctggttgaagccaagcaggcaagatcagacatcgaaaaactcaaagaagcaattagggacacaaacaaagcagtgcagtcagttcagagctccataggaaatttaatagtagcaattaaatcagtccaggattatgttaacaaagaaatcgtgccatcgattgcgaggctaggttgtgaagcagcaggacttcaattaggaattgcattaacacagcattactcagaattaacaaacatatttggtgataacataggatcgttacaagaaaaaggaataaaattacaaggtatagcatcattataccgcacaaatatcacagaaatattcacaacatcaacagttgataaatatgatatctatgatctgttatttacagaatcaataaaggtgagagttatagatgttgacttgaatgattactcaatcaccctccaagtcagactccctttattaactaggctgctgaacactcagatctacaaagtagattccatatcatataacatccaaaacagagaatggtatatccctcttcccagccatatcatgacgaaaggggcatttctaggtggagcagacgtcaaagaatgtatagaagcattcagcagctatatatgcccttctgatccaggatttgtattaaaccatgaaatagagagctgcttatcaggaaacatatcccaatgtccaagaacaacggtcacatcagacattgttccaagatatgcatttgtcaatggaggagtggttgcaaactgtataacaaccacctgtacatgcaacggaattggtaatagaatcaatcaaccacctgatcaaggagtaaaaattataacacataaagaatgtagtacaataggtatcaacggaatgctgttcaatacaaataaagaaggaactcttgcattctatacaccaaatgatataacactaaacaattctgttgcacttgatccaattgacatatcaatcgagctcaacaaggccaaatcagatctagaagaatcaaaagaatggataagaaggtcaaatcaaaaactagattctattggaaattggcatcaatctagcactacaatcataattattttgataatgatcattatattgtttataattaatataacgataattacaattgcaattaagtattacagaattcaaaagagaaatcgagtggatcaaaatgacaagccatatgtactaacaaacaaataacatatctacagatcattagatattaaaattataaaaaacttaggagtaaagttacgcaatccaactctactcatataattgaggaaggacccaatagacaaatccaaattcgagatggaatactggaagcataccaatcacggaaaggatgctggtaatgagctggagacgtctatggctactcatggcaacaagctcactaataagataatatacatattatggacaataatcctggtgttattatcaatagtcttcatcatagtgctaattaattccatcaaaagtgaaaaggcccacgaatcattgctgcaagacataaataatgagtttatggaaattacagaaaagatccaaatggcatcggataataccaatgatctaatacagtcaggagtgaatacaaggcttcttacaattcagagtcatgtccagaattacataccaatatcattgacacaacagatgtcagatcttaggaaattcattagtgaaattacaattagaaatgataatcaagaagtgctgccacaaagaataacacatgatgtaggtataaaacctttaaatccagatgatttttggagatgcacgtctggtcttccatctttaatgaaaactccaaaaataaggttaatgccagggccgggattattagctatgccaacgactgttgatggctgtgttagaactccgtctttagttataaatgatctgatttatgcttatacctcaaatctaattactcgaggttgtcaggatataggaaaatcatatcaagtcttacagatagggataataactgtaaactcagacttggtacctgacttaaatcctaggatctctcatacctttaacataaatgacaataggaagtcatgttctctagcactcctaaatacagatgtatatcaactgtgttcaactcccaaagttgatgaaagatcagattatgcatcatcaggcatagaagatattgtacttgatattgtcaattatgatggttcaatctcaacaacaagatttaagaataataacataagctttgatcaaccatatgctgcactatacccatctgttggaccagggatatactacaaaggcaaaataatatttctcgggtatggaggtcttgaacatccaataaatgagaatgtaatctgcaacacaactgggtgccccgggaaaacacagagagactgtaatcaagcatctcatagtccatggttttcagataggaggatggtcaactccatcattgttgttgacaaaggcttaaactcaattccaaaattgaaagtatggacgatatctatgcgacaaaattactgggggtcagaaggaaggttacttctactaggtaacaagatctatatatatacaagatctacaagttggcatagcaagttacaattaggaataattgatattactgattacagtgatataaggataaaatggacatggcataatgtgctatcaagaccaggaaacaatgaatgtccatggggacattcatgtccagatggatgtataacaggagtatatactgatgcatatccactcaatcccacagggagcattgtgtcatctgtcatattagactcacaaaaatcgagagtgaacccagtcataacttactcaacagcaaccgaaagagtaaacgagctggccatcctaaacagaacactctcagctggatatacaacaacaagctgcattacacactataacaaaggatattgttttcatatagtagaaataaatcataaaagcttaaacacatttcaacccatgttgttcaaaacagagattccaaaaagctgcagttaatcataattaaccataatatgcatcaatctatctataatacaagtatatgataagtaatcagcaatcagacaatagacgtacggaaataataaaaaacttaggagaaaagtgtgcaagaaaaatggacaccgagtcccacagcggcacaacatctgacattctgtaccctgaatgtcacctcaattctcctatagttaaaggaaagatagcacaactgcatacaataatgagtttgcctcagccctacgatatggatgatgattcaatactgattattactagacaaaaaattaaactcaataaattagataaaagacaacggtcaattaggaaattaagatcagtcttaatggaaagagtaagtgatctaggtaaatatacctttatcagatatccagagatgtctagtgaaatgttccaattatgtatacccggaattaataataaaataaatgaattgctaagtaaagcaagtaaaacatataatcaaatgactgatggattaagagatctatgggttactatactatcgaagttagcatcgaaaaatgatggaagtaattatgatatcaatgaagatattagcaatatatcaaatgttcacatgacttatcaatcagacaaatggtataatccattcaagacatggtttactattaagtatgacatgagaagattacaaaaagccaaaaatgagattacattcaataggcataaagattataatctattagaagaccaaaagaatatattgctgatacatccagaactcgtcttaatattagataaacaaaattacaatgggtatataatgactcctgaattggtactaatgtattgtgatgtagttgaagggaggtggaatataagttcatgtgcaaaattggatcctaagttacaatcaatgtattataagggtaacaatttatgggaaataatagatggactattctcgaccttaggagaaagaacatttgacataatatcactattagaaccacttgcattatcgctcattcaaacttatgacccggttaaacagctcaggggggcttttttaaatcacgtgttatcagaaatggaattaatatttgcagctgagtgtacaacagaggaaatacctaatgtggattatatagataaaattttagatgtgttcaaagaatcaacaatagatgaaatagcagaaattttctctttcttccgaacttttggacaccctccattagaggcgagtatagcagcagagaaagttagaaagtatatgtatactgagaaatgcttgaaatttgatactatcaataaatgtcatgctattttttgtacaataattataaatggatatagagaaagacatggtggtcaatggcctccagttacattacctgtccatgcacatgaatttatcataaatgcatacggatcaaattctgccatatcatatgagaatgctgtagattattataagagcttcataggaataaaatttgacaagtttatagagcctcaattggatgaagacttaactatttatatgaaagataaagcattatccccaaagaaatcaaactgggacacagtctatccagcttcaaacctgttataccgcactaatgtgtctcatgattcacgaagattggttgaagtatttatagcagatagtaaatttgatccccaccaagtattagattacgtagaatcaggatattggctggatgatcctgaatttaatatctcatatagtttaaaagagaaagaaataaaacaagaaggtagactttttgcaaaaatgacatacaagatgagggctacacaagtattatcagaaacattattggcgaataatatagggaaattcttccaagagaatgggatggttaaaggagaaattgaattactcaagagactaacaacaatatctatgtctggagttccgcggtataatgaggtatacaataattcaaaaagtcacacagaagaacttcaagcttataatgcaattagcagttccaatttatcttctaatcagaagtcaaagaagtttgaatttaaatctacagatatatacaatgatggatacgaaaccgtaagctgcttcttaacgacagatcttaaaaaatattgtttaaattggaggtatgaatcaacagctttattcggtgatacttgtaatcagatatttgggttaaaggaattatttaattggctgcaccctcgccttgaaaagagtacaatatatgttggagatccttattgcccgccatcagatattgaacatttaccacttgatgaccatcctgattcaggattttatgttcataatcctaaaggaggaatagaagggttttgccaaaagttatggacactcatatctatcagtgcaatacatttagcagctgtcaaaatcggtgtaagagttactgcaatggttcaaggggataatcaagccatagctgttaccacaagagtacctaataattatgattataaagttaagaaagagattgtttataaagatgtggtaagattttttgattccttgagagaggtgatggatgatctgggtcatgagctcaaactaaatgaaactataataagtagtaaaatgtttatatatagcaaaaggatatactatgacggaagaatccttcctcaggcattaaaagcattgtctagatgtgttttttggtctgaaacaatcatagatgagacaagatcagcatcctcaaatctggctacatcgtttgcaaaggccattgagaatggctactcacctgtattgggatatgtatgctcaatcttcaaaaatatccaacagttgtatatagcgcttggaatgaatataaacccaactataacccaaaatattaaagatcaatatttcaggaatattcattggatgcaatatgcctccttaatccctgctagtgtcggaggatttaattatatggccatgtcaaggtgttttgtcagaaacattggagatcctacagtcgctgcgttagccgatattaaaagatttataaaagcaaatttgttagatcgaggtgtcctttacagaattatgaatcaagaaccaggcgagtcttcttttttagactgggcctcagatccctattcatgtaacttaccacaatctcaaaatataaccaccatgataaagaatataactgcaagaaatgtactacaggactcaccaaacccattactatctggattatttacaagtacaatgatagaagaggatgaggaattagctgagttcctaatggacaggagaataatcctcccaagagttgcacatgacattttagataattctcttactggaattaggaatgctatagctggtatgttggatacaacaaaatcactaattcgagtagggataagcagaggaggattaacctataacttattaagaaagataagcaactatgatcttgtacaatatgagacacttagtaaaactttaagactaatagtcagtgacaagattaagtatgaagatatgtgctcagtagacctagccatatcattaagacaaaaaatgtggatgcatttatcaggaggaagaatgataaatggacttgaaactccagatcctttagagttactgtctggagtaataataacaggatctgaacattgtaggatatgttattcaactgaaggtgaaagcccatatacatggatgtatttaccaggcaatcttaatataggatcagctgagacaggaatagcatcattaagggtcccttactttggatcagttacagatgagagatctgaagcacaattagggtatatcaaaaatctaagcaaaccagctaaggctgctataagaatagcaatgatatatacttgggcatttgggaatgacgaaatatcttggatggaagcatcacagattgcacaaacacgtgcaaactttacattggatagcttaaagattttgacaccagtgacaacatcaacaaatctatcacacaggttaaaagatactgctactcagatgaaattttctagtacatcacttattagagtaagcaggttcatcacaatatctaatgataatatgtctattaaagaagcaaatgaaactaaagatacaaatcttatttatcaacaggtaatgttaacaggattaagtgtatttgaatatctatttaggttagaggagagtacaggacataaccctatggtcatgcatctacatatagaggatggatgttgtataaaagagagttacaatgatgagcatatcaatccggagtctacattagagttaatcaaataccctgagagtaatgaatttatatatgataaggaccctttaaaggatatagatctatcaaaattaatggttataagagatcattcttatacaattgacatgaattactgggatgacacagatattgtacatgcaatatcaatatgtactgcagttacaatagcagatacaatgtcgcagctagatcgggataatcttaaggagctggttgtgattgcaaatgatgatgatattaacagtctgataactgaatttctgaccctagatatactagtgtttctcaaaacatttggagggttactcgtgaatcaatttgcatataccctttatggattgaaaatagaaggaagggatcccatttgggattatataatgagaacattaaaagacacctcacattcagtacttaaagtattatctaatgcactatctcatccaaaagtgtttaagagattttgggattgtggagttttgaatcctatttatggtcctaatactgctagtcaagatcaagttaagcttgctctctcgatttgcgagtactccttggatctatttatgagagaatggttgaatggagcatcacttgagatctatatctgtgatagtgacatggaaatagcaaatgacagaagacaagcatttctctcaagacatcttgcctttgtgtgttgtttagcagagatagcatcttttggaccaaatttattaaatctaacatatctagagagacttgatgaattaaaacaatacttagatctgaacatcaaagaagatcctactcttaaatatgtgcaagtatcaggactgttaattaaatcattcccctcaactgttacgtatgtaaggaaaactgcgattaagtatctgaggattcgtggtattaatccgcctgaaacgattgaagattgggatcccatagaagatgagaatatcttagacaatattgttaaaactgtaaatgacaattgcagtgataatcaaaagagaaataaaagtagttatttctggggattagctctaaagaattatcaagtcgtgaaaataagatccataacgagtgattctgaagttaatgaagcttcgaatgttactacacatggaatgacacttcctcagggaggaagttatctatcacatcagctgaggttatttggagtaaacagtacaagttgtcttaaagctcttgaattatcacaaatcttaatgagggaagttaaaaaagataaagatagactctttttaggagaaggagcaggagctatgttagcatgttatgatgctacactcggtcctgcaataaattattataattctggtttaaatattacagatgtaattggtcaacgggaattaaaaatcttcccatcagaagtatcattagtaggtaaaaaactaggaaatgtaacacagattcttaatcgggtgagggtgttatttaatgggaatcccaattcaacatggataggaaatatggaatgtgagagtttaatatggagtgaattaaatgataagtcaattggtttagtacattgtgacatggagggagcgataggcaaatcagaagaaactgttctacatgaacattatagtattattaggattacatatttaatcggggatgatgatgttgtcctagtatcaaaaattataccaactattactccgaattggtctaaaatactctatctatacaagttgtattggaaggatgtaagtgtagtgtcccttaaaacatccaatcctgcctcaacagagctttatttaatttcaaaagatgcttactgtactgtaatggaacccagtaatcttgttttatcaaaacttaaaaggatatcatcaatagaagaaaataatctattaaagtggataatcttatcaaaaaggaagaataacgagtggttacagcatgaaatcaaagaaggagaaagggattatgggataatgaggccatatcatacagcactgcaaatttttggattccaaattaacttaaatcacttagctagagaatttttatcaactcctgatttaaccaacattaataatataattcaaagttttacaagaacaattaaagatgttatgttcgaatgggtcaatatcactcatgacaataaaagacataaattaggaggaagatataatctattcccgcttaaaaataaggggaaattaagattattatcacgaagattagtactaagctggatatcattatccttatcaaccagattactgacgggccgttttccagatgaaaaatttgaaaatagggcacagaccggatatgtatcattggctgatattgatttagaatccttaaagttattatcaagaaatattgtcaaaaattacaaagaacacataggattaatatcatactggtttttgaccaaagaggtcaaaatactaatgaagcttataggaggagtcaaactactaggaattcctaaacagtacaaagagttagaggatcgatcatctcagggttatgaatatgataatgaatttgatattgattaatacataaaaacaaaaaataaaacacctattcctcacccattcacttccaacaaaatgaaaagtaagaaaaacatgtaatatatatataccaaacagagtttttctcttgtttggt

SEQ ID NO: 43 - DNA, 19302 bp, Artificial Sequence
  Recombinant nucleic acid
accaaacaagagaagagactggtttgggaatattaattcaaataaaaattaacttaggattaaagaactttaccgaaaggtaaggggaaagaaatcctaagagcttagccatgttgagtctattcgacacattcagtgcgcgtaggcaggagaacataacgaaatcagctggtggggctgttattcccgggcaaaaaaacactgtgtctatatttgctcttggaccatcaataacagatgacaatgataaaatgacattggctcttctctttttgtctcattctttagacaatgaaaagcagcatgcgcaaagagctggatttttagtttctctgttatcaatggcttatgccaacccagaattatatttaacatcaaatggtagtaatgcagatgttaaatatgttatctacatgatagagaaagacccaggaagacagaaatatggtgggtttgtcgtcaagactagagagatggtttatgaaaagacaactgattggatgttcgggagtgatcttgagtatgatcaagacaatatgttgcaaaatggtagaagcacttctacaatcgaggatcttgttcatacttttggatatccatcgtgtcttggagcccttataatccaagtttggataatacttgttaaggctataaccagtatatcaggattgaggaaaggattctttactcggttagaagcatttcgacaagatggaacagttaaatccagtctagtgttgagcggtgatgcagtagaacaaattggatcaattatgaggtcccaacagagcttggtaacactcatggttgaaacactgataacaatgaacacaggcaggaatgatctgacaacaatagaaaagaatatacagattgtaggaaactacatcagagatgcaggtcttgcttcatttttcaacacaatcagatatggcattgagactagaatggcagctctaactctgtctacccttagaccggatatcaacagactcaaggcactgatcgagttatatctatcaaaggggccacgtgctccttttatatgcattttgagagatcccgtgcatggtgagtttgcaccaggcaactatcctgccctctggagttatgcgatgggtgtagcagttgtacaaaacaaggccatgcaacagtatgtaacaggaaggtcttatctggatattgaaatgttccaacttggtcaagcagtggcacgtgatgccgagtcgcagatgagttcaatattagaggatgaactgggggtcacacaagaagccaagcaaagcttgaagaaacacatgaagaacatcagcagttcagatacaacctttcataagcctacagggggatcagccatagaaatggcgatagatgaagaagcagggcagcctgaatccagaggagatcaggatcaaggagatgagcctcggtcatccatagttccttatgcatgggcagacgaaaccgggaatgacaatcaaactgaatcaactacagaaattgacagcatcaaaactgaacaaagaaacatcagagacaggctgaacaaaagactcaacgagaaaaggaaacagagtgacccgagatcaactgacatcacaaacaacacaaatcaaactgaaatagatgatttgttcagtgcattcggaagcaactagtcacaaagagatgaccaggcgcgccaagtaagaaaaacttaggattaatggacctgcaggatgttcgtgtttctggtgctgctgcctctggtgagctcccagtgcgtgaacctgaccacaaggacccagctgccccctgcctataccaattccttcacacggggcgtgtactatcccgacaaggtgtttagatctagcgtgctgcactccacacaggatctgtttctgcctttcttttctaacgtgacctggttccacgtgatcagcggcaccaatggcacaaagcggttcgacaatccagtgctgccctttaacgatggcgtgtacttcgcctccatcgagaagtctaacatcatcagaggctggatctttggcaccacactggacagcaagacacagtccctgctgatcgtgaacaatgccaccaacgtggtcatcaaggtgtgcgagttccagttttgtaatgatccattcctggaccacaagaacaataagtcttggatggagagcgagtttcgcgtgtattcctctgccaacaattgcacatttgagtacgtgtcccagcccttcctgatggacctggagggcaagcagggcaatttcaagaacctgagggagttcgtgtttaagaatatcgatggctacttcaagatctactccaagcacaccccaatcatcgtgcgcgagccagaagacctgccacagggcttctctgccctggagccactggtggatctgcccatcggcatcaacatcacccggtttcagacactgctggccctgcacagaagctacctgacaccaggcgacagctcctctggatggaccgcaggagctgccgcctactatgtgggctatctgcagcccaggaccttcctgctgaagtacaacgagaatggcaccatcacagacgccgtggattgcgccctggatcccctgtctgagaccaagtgtacactgaagagctttaccgtggagaagggcatctatcagacaagcaatttcagggtgcagcctaccgagtccatcgtgcgctttcccaatatcacaaacctgtgcccttttgacgaggtgttcaacgcaacccgcttcgccagcgtgtacgcctggaataggaagcgcatctccaactgcgtggccgactattctgtgctgtacaaccttgctccattcttcacctttaagtgctatggcgtgagccccacaaagctgaatgacctgtgctttaccaacgtgtacgccgattccttcgtgatcaggggcgacgaggtgcgccagatcgcccctggccagacaggcaacatcgccgactacaattataagctgcctgacgatttcaccggctgcgtgatcgcctggaactctaacaagctggatagcaaagtgagcggcaactacaattatctgtaccggctgtttagaaagtctaatctgaagccattcgagagggacatctccacagagatctaccaggccggcaacaagccctgcaatggcgtggccggctttaactgttatttccctctgaggagctacagcttcaggccaacatacggcgtgggacatcagccctaccgcgtggtggtgctgtcttttgagctgctgcacgcacctgcaacagtgtgcggaccaaagaagagcaccaatctggtgaagaacaagtgcgtgaacttcaacttcaacggactgaagggcacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtatcagggcgtgaattgtaccgaggtgcccgtggcaatccacgcagatcagctgacccctacatggcgggtgtactctaccggcagcaacgtgttccagacaagagccggatgcctgatcggagccgagtacgtgaacaatagctatgagtgcgacatccctatcggcgccggcatctgtgcctcctaccagacccagacaaagtcccacgggtctgcctcctctgtggccagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggctctttttgtacccagctgaagagagccctgacaggcatcgccgtggagcaggacaagaacacacaggaggtgttcgcccaggtgaagcagatctacaagaccccacccatcaagtactttggcggcttcaacttcagccagatcctgcccgatcctagcaagccatccaagcggtctcctatcgaggacctgctgttcaacaaggtgaccctggccgatgccggcttcatcaagcagtatggcgattgcctgggcgacatcgccgccagagacctgatctgtgcccagaagtttaagggcctgaccgtgctgcctccactgctgacagatgagatgatcgcccagtacacatctgccctgctggccggcaccatcacaagcggatggaccttcggcgcaggacccgccctgcagatcccctttcccatgcagatggcctatcggttcaacggcatcggcgtgacccagaatgtgctgtacgagaaccagaagctgatcgccaatcagtttaactccgccatcggcaagatccaggactctctgagctccacacccagcgccctgggcaagctgcaggatgtggtgaatcacaacgcccaggccctgaataccctggtgaagcagctgtctagcaagttcggcgccatctcctctgtgctgaatgatatcttcagcaggctggaccctccagaggcagaggtgcagatcgaccggctgatcacaggcagactgcagtccctgcagacctacgtgacacagcagctgatcagggcagcagagatcagggcctctgccaatctggccgccaccaagatgagcgagtgcgtgctgggccagtccaagagagtggacttttgtggcaagggctatcacctgatgagcttcccacagtccgcccctcacggagtggtgtttctgcacgtgacctacgtgccagcccaggagaagaacttcaccacagcaccagcaatctgccacgatggcaaggcacactttcctagggagggcgtgttcgtgagcaacggcacccactggtttgtgacacagcgcaatttctacgagccacagatcatcaccacagacaatacattcgtgtccggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgacaggctgaacgaggtggccaagaatctgaacgagtccctgatcgatctgcaggagctgggcaagtatgagcagtacatcaagtggccctggtatatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactacacctgataactagcggcgcgccagcaacaagtaagaaaaacttaggattaatggaaattatccaatccagagacggaaggacaaatccagaatccaaccacaactcaatcaaccaaagattcatggaagacaatgttcaaaacaatcaaatcatggattcttgggaagagggatcaggagataaatcatctgacatctcatcggccctcgacatcattgaattcatactcagcaccgactcccaagagaacacggcagacagcaatgaaatcaacacaggaaccacaagacttagcacgacaatctaccaacctgaatccaaaacaacagaaacaagcaaggaaaatagtggaccagctaacaaaaatcgacagtttggggcatcacacgaacgtgccacagagacaaaagatagaaatgttaatcaggagactgtacagggaggatataggagaggaagcagcccagatagtagaactgagactatggtcactcgaagaatctccagaagcagcccagatcctaacaatggaacccaaatccaggaagatattgattacaatgaagttggagagatggataaggactctactaagagggaaatgcgacaatttaaagatgttccagtcaaggtatcaggaagtgatgccattcctccaacaaaacaagatggagacggtgatgatggaagaggcctggaatctatcagtacatttgattcaggatataccagtatagtgactgccgcaacactagatgacgaagaagaactccttatgaagaacaacaggccaagaaagtatcaatcaacaccccagaacagtgacaagggaattaaaaaaggggttggaaggccaaaagacacagacaaacaatcatcaatattggactacgaactcaacttcaaaggatcgaagaagagccagaaaatcctcaaagccagcacgaatacaggagaaccaacaagaccacagaatggatcccaggggaagagaatcacatcctggaacatcctcaacagcgagagcggcaatcgaacagaatcaacaaaccaaacccatcagacatcaacctcgggacagaaccacacaatgggaccaagcagaacaacctccgaaccaaggatcaagacacaaaagacggatggaaaggaaagagaggacacagaagagagcactcgatttacagaaagggcgattacattattacagaatcttggtgtaatccaatctgcagcaaaattagacctataccaagacaagagagttgtgtgtgtggcgaatgtcctaaacaatgcagatactgcatcaaagatagacttcctagcaggtttgatgataggagtgtcaatggatcatgataccaaattaaatcagattcagaacgagatattaagtttgaaaactgatcttaaaaagatggatgaatcacatagaagactaattgagaatcaaaaagaacaattatcactgatcacatcattaatctcaaatcttaaaattatgacagagagaggagggaagaaggaccaaccagaacctagcgggaggacatccatgatcaagacaaaagcaaaagaagagaaaataaagaaagtcaggtttgaccctcttatggaaacacagggcatcgagaaaaacatccctgacctctatagatcaatagagaaaacaccagaaaacgacacacagatcaaatcagaaataaacagattgaatgatgaatccaatgccactagattagtacctagaagaataagcagtacaatgagatcattaataataatcattaacaacagcaatttatcatcaaaagcaaagcaatcatacatcaacgaactcaagctctgcaagagtgacgaggaagtgtctgagttgatggacatgttcaatgaggatgtcagctcccagtaaaccgccaaccaagggtcaacaccaagaaaaccaatagcacaaaacagccaatcagagaccaccccaatacaccaaaccaatcaacacataacaaagatcgcggccgcatagatgattaagaaaaacttaggatgaaaggactaatcaatcctccgaaacaatgagcatcaccaactccacaatctacacattcccagaatcctctttctccgagaatggcaacatagagccgttaccactcaaggtcaatgaacagagaaaggccatacctcatattagggttgtcaagataggagatccgcccaaacatggatccagatatctggatgtctttttactgggcttctttgagatggaaaggtcaaaagacaggtatgggagcataagtgatctagatgatgatccaagttacaaggtttgtggctctggatcattgccacttgggttggctagatacaccggaaatgatcaggaactcctacaggctgcaaccaagctcgatatagaagtaagaagaactgtaaaggctacggagatgatagtttacactgtacaaaacatcaaacctgaactatatccatggtccagtagattaagaaaagggatgttatttgacgctaataaggttgcacttgctcctcaatgtcttccactagatagagggataaaattcagggtgatatttgtgaactgcacagcaattggatcaataactctattcaaaatccctaagtccatggcattgttatcattgcctaatacaatatcaataaatctacaagtacatatcaaaacaggagttcagacagattccaaaggagtagttcagattctagatgaaaaaggtgaaaaatcactaaatttcatggttcatctcgggttgatcaaaaggaagatgggcagaatgtactcagttgaatattgtaagcagaagatcgagaagatgagattattattctcattgggattagttggagggatcagcttccacgtcaacgcaactggctctatatcaaagacattagcaagtcaattagcattcaaaagagaaatctgctatcccctaatggatctgaatccacacttaaattcagttatatgggcatcatcagttgaaattacaagggtagatgcagttctccagccttcattacctggcgaattcagatactacccaaacatcatagcaaaaggggtcgggaaaatcagacagtaaaatcaacaaccctgatatccaccggtgtattaagccgaagcaaataaaggataatcaaaaacttaggacaaaagaggtcaataccaacaactattagcagtcacactcgcaagaataagagagaagggaccaaaaaagtcaaataggagaaatcaaaacaaaaggtacagaacaccagaacaacaaaatcaaaacatccaactcactcaaaacaaaaattccaaaagagaccggcaacacaacaagcactgaacacaatgccaacttcaatactgctaattattacaaccatgatcatggcatctttctgccaaatagatatcacaaaactacagcacgtaggtgtattggtcaacagtcccaaagggatgaagatatcacaaaactttgaaacaagatatctaattttgagcctcataccaaaaatagaagactctaactcttgtggtgaccaacagatcaagcaatacaagaagttattggatagactgatcatccctttatatgatggattaagattacagaaagatgtgatagtaaccaatcaagaatccaatgaaaacactgatcccagaacaaaacgattctttggaggggtaattggaaccattgctctgggagtagcaacctcagcacaaattacagcggcagttgctctggttgaagccaagcaggcaagatcagacatcgaaaaactcaaagaagcaattagggacacaaacaaagcagtgcagtcagttcagagctccataggaaatttaatagtagcaattaaatcagtccaggattatgttaacaaagaaatcgtgccatcgattgcgaggctaggttgtgaagcagcaggacttcaattaggaattgcattaacacagcattactcagaattaacaaacatatttggtgataacataggatcgttacaagaaaaaggaataaaattacaaggtatagcatcattataccgcacaaatatcacagaaatattcacaacatcaacagttgataaatatgatatctatgatctgttatttacagaatcaataaaggtgagagttatagatgttgacttgaatgattactcaatcaccctccaagtcagactccctttattaactaggctgctgaacactcagatctacaaagtagattccatatcatataacatccaaaacagagaatggtatatccctcttcccagccatatcatgacgaaaggggcatttctaggtggagcagacgtcaaagaatgtatagaagcattcagcagctatatatgcccttctgatccaggatttgtattaaaccatgaaatagagagctgcttatcaggaaacatatcccaatgtccaagaacaacggtcacatcagacattgttccaagatatgcatttgtcaatggaggagtggttgcaaactgtataacaaccacctgtacatgcaacggaattggtaatagaatcaatcaaccacctgatcaaggagtaaaaattataacacataaagaatgtagtacaataggtatcaacggaatgctgttcaatacaaataaagaaggaactcttgcattctatacaccaaatgatataacactaaacaattctgttgcacttgatccaattgacatatcaatcgagctcaacaaggccaaatcagatctagaagaatcaaaagaatggataagaaggtcaaatcaaaaactagattctattggaaattggcatcaatctagcactacaatcataattattttgataatgatcattatattgtttataattaatataacgataattacaattgcaattaagtattacagaattcaaaagagaaatcgagtggatcaaaatgacaagccatatgtactaacaaacaaataacatatctacagatcattagatattaaaattataaaaaacttaggagtaaagttacgcaatccaactctactcatataattgaggaaggacccaatagacaaatccaaattcgagatggaatactggaagcataccaatcacggaaaggatgctggtaatgagctggagacgtctatggctactcatggcaacaagctcactaataagataatatacatattatggacaataatcctggtgttattatcaatagtcttcatcatagtgctaattaattccatcaaaagtgaaaaggcccacgaatcattgctgcaagacataaataatgagtttatggaaattacagaaaagatccaaatggcatcggataataccaatgatctaatacagtcaggagtgaatacaaggcttcttacaattcagagtcatgtccagaattacataccaatatcattgacacaacagatgtcagatcttaggaaattcattagtgaaattacaattagaaatgataatcaagaagtgctgccacaaagaataacacatgatgtaggtataaaacctttaaatccagatgatttttggagatgcacgtctggtcttccatctttaatgaaaactccaaaaataaggttaatgccagggccgggattattagctatgccaacgactgttgatggctgtgttagaactccgtctttagttataaatgatctgatttatgcttatacctcaaatctaattactcgaggttgtcaggatataggaaaatcatatcaagtcttacagatagggataataactgtaaactcagacttggtacctgacttaaatcctaggatctctcatacctttaacataaatgacaataggaagtcatgttctctagcactcctaaatacagatgtatatcaactgtgttcaactcccaaagttgatgaaagatcagattatgcatcatcaggcatagaagatattgtacttgatattgtcaattatgatggttcaatctcaacaacaagatttaagaataataacataagctttgatcaaccatatgctgcactatacccatctgttggaccagggatatactacaaaggcaaaataatatttctcgggtatggaggtcttgaacatccaataaatgagaatgtaatctgcaacacaactgggtgccccgggaaaacacagagagactgtaatcaagcatctcatagtccatggttttcagataggaggatggtcaactccatcattgttgttgacaaaggcttaaactcaattccaaaattgaaagtatggacgatatctatgcgacaaaattactgggggtcagaaggaaggttacttctactaggtaacaagatctatatatatacaagatctacaagttggcatagcaagttacaattaggaataattgatattactgattacagtgatataaggataaaatggacatggcataatgtgctatcaagaccaggaaacaatgaatgtccatggggacattcatgtccagatggatgtataacaggagtatatactgatgcatatccactcaatcccacagggagcattgtgtcatctgtcatattagactcacaaaaatcgagagtgaacccagtcataacttactcaacagcaaccgaaagagtaaacgagctggccatcctaaacagaacactctcagctggatatacaacaacaagctgcattacacactataacaaaggatattgttttcatatagtagaaataaatcataaaagcttaaacacatttcaacccatgttgttcaaaacagagattccaaaaagctgcagttaatcataattaaccataatatgcatcaatctatctataatacaagtatatgataagtaatcagcaatcagacaatagacgtacggaaataataaaaaacttaggagaaaagtgtgcaagaaaaatggacaccgagtcccacagcggcacaacatctgacattctgtaccctgaatgtcacctcaattctcctatagttaaaggaaagatagcacaactgcatacaataatgagtttgcctcagccctacgatatggatgatgattcaatactgattattactagacaaaaaattaaactcaataaattagataaaagacaacggtcaattaggaaattaagatcagtcttaatggaaagagtaagtgatctaggtaaatatacctttatcagatatccagagatgtctagtgaaatgttccaattatgtatacccggaattaataataaaataaatgaattgctaagtaaagcaagtaaaacatataatcaaatgactgatggattaagagatctatgggttactatactatcgaagttagcatcgaaaaatgatggaagtaattatgatatcaatgaagatattagcaatatatcaaatgttcacatgacttatcaatcagacaaatggtataatccattcaagacatggtttactattaagtatgacatgagaagattacaaaaagccaaaaatgagattacattcaataggcataaagattataatctattagaagaccaaaagaatatattgctgatacatccagaactcgtcttaatattagataaacaaaattacaatgggtatataatgactcctgaattggtactaatgtattgtgatgtagttgaagggaggtggaatataagttcatgtgcaaaattggatcctaagttacaatcaatgtattataagggtaacaatttatgggaaataatagatggactattctcgaccttaggagaaagaacatttgacataatatcactattagaaccacttgcattatcgctcattcaaacttatgacccggttaaacagctcaggggggcttttttaaatcacgtgttatcagaaatggaattaatatttgcagctgagtgtacaacagaggaaatacctaatgtggattatatagataaaattttagatgtgttcaaagaatcaacaatagatgaaatagcagaaattttctctttcttccgaacttttggacaccctccattagaggcgagtatagcagcagagaaagttagaaagtatatgtatactgagaaatgcttgaaatttgatactatcaataaatgtcatgctattttttgtacaataattataaatggatatagagaaagacatggtggtcaatggcctccagttacattacctgtccatgcacatgaatttatcataaatgcatacggatcaaattctgccatatcatatgagaatgctgtagattattataagagcttcataggaataaaatttgacaagtttatagagcctcaattggatgaagacttaactatttatatgaaagataaagcattatccccaaagaaatcaaactgggacacagtctatccagcttcaaacctgttataccgcactaatgtgtctcatgattcacgaagattggttgaagtatttatagcagatagtaaatttgatccccaccaagtattagattacgtagaatcaggatattggctggatgatcctgaatttaatatctcatatagtttaaaagagaaagaaataaaacaagaaggtagactttttgcaaaaatgacatacaagatgagggctacacaagtattatcagaaacattattggcgaataatatagggaaattcttccaagagaatgggatggttaaaggagaaattgaattactcaagagactaacaacaatatctatgtctggagttccgcggtataatgaggtatacaataattcaaaaagtcacacagaagaacttcaagcttataatgcaattagcagttccaatttatcttctaatcagaagtcaaagaagtttgaatttaaatctacagatatatacaatgatggatacgaaaccgtaagctgcttcttaacgacagatcttaaaaaatattgtttaaattggaggtatgaatcaacagctttattcggtgatacttgtaatcagatatttgggttaaaggaattatttaattggctgcaccctcgccttgaaaagagtacaatatatgttggagatccttattgcccgccatcagatattgaacatttaccacttgatgaccatcctgattcaggattttatgttcataatcctaaaggaggaatagaagggttttgccaaaagttatggacactcatatctatcagtgcaatacatttagcagctgtcaaaatcggtgtaagagttactgcaatggttcaaggggataatcaagccatagctgttaccacaagagtacctaataattatgattataaagttaagaaagagattgtttataaagatgtggtaagattttttgattccttgagagaggtgatggatgatctgggtcatgagctcaaactaaatgaaactataataagtagtaaaatgtttatatatagcaaaaggatatactatgacggaagaatccttcctcaggcattaaaagcattgtctagatgtgttttttggtctgaaacaatcatagatgagacaagatcagcatcctcaaatctggctacatcgtttgcaaaggccattgagaatggctactcacctgtattgggatatgtatgctcaatcttcaaaaatatccaacagttgtatatagcgcttggaatgaatataaacccaactataacccaaaatattaaagatcaatatttcaggaatattcattggatgcaatatgcctccttaatccctgctagtgtcggaggatttaattatatggccatgtcaaggtgttttgtcagaaacattggagatcctacagtcgctgcgttagccgatattaaaagatttataaaagcaaatttgttagatcgaggtgtcctttacagaattatgaatcaagaaccaggcgagtcttcttttttagactgggcctcagatccctattcatgtaacttaccacaatctcaaaatataaccaccatgataaagaatataactgcaagaaatgtactacaggactcaccaaacccattactatctggattatttacaagtacaatgatagaagaggatgaggaattagctgagttcctaatggacaggagaataatcctcccaagagttgcacatgacattttagataattctcttactggaattaggaatgctatagctggtatgttggatacaacaaaatcactaattcgagtagggataagcagaggaggattaacctataacttattaagaaagataagcaactatgatcttgtacaatatgagacacttagtaaaactttaagactaatagtcagtgacaagattaagtatgaagatatgtgctcagtagacctagccatatcattaagacaaaaaatgtggatgcatttatcaggaggaagaatgataaatggacttgaaactccagatcctttagagttactgtctggagtaataataacaggatctgaacattgtaggatatgttattcaactgaaggtgaaagcccatatacatggatgtatttaccaggcaatcttaatataggatcagctgagacaggaatagcatcattaagggtcccttactttggatcagttacagatgagagatctgaagcacaattagggtatatcaaaaatctaagcaaaccagctaaggctgctataagaatagcaatgatatatacttgggcatttgggaatgacgaaatatcttggatggaagcatcacagattgcacaaacacgtgcaaactttacattggatagcttaaagattttgacaccagtgacaacatcaacaaatctatcacacaggttaaaagatactgctactcagatgaaattttctagtacatcacttattagagtaagcaggttcatcacaatatctaatgataatatgtctattaaagaagcaaatgaaactaaagatacaaatcttatttatcaacaggtaatgttaacaggattaagtgtatttgaatatctatttaggttagaggagagtacaggacataaccctatggtcatgcatctacatatagaggatggatgttgtataaaagagagttacaatgatgagcatatcaatccggagtctacattagagttaatcaaataccctgagagtaatgaatttatatatgataaggaccctttaaaggatatagatctatcaaaattaatggttataagagatcattcttatacaattgacatgaattactgggatgacacagatattgtacatgcaatatcaatatgtactgcagttacaatagcagatacaatgtcgcagctagatcgggataatcttaaggagctggttgtgattgcaaatgatgatgatattaacagtctgataactgaatttctgaccctagatatactagtgtttctcaaaacatttggagggttactcgtgaatcaatttgcatataccctttatggattgaaaatagaaggaagggatcccatttgggattatataatgagaacattaaaagacacctcacattcagtacttaaagtattatctaatgcactatctcatccaaaagtgtttaagagattttgggattgtggagttttgaatcctatttatggtcctaatactgctagtcaagatcaagttaagcttgctctctcgatttgcgagtactccttggatctatttatgagagaatggttgaatggagcatcacttgagatctatatctgtgatagtgacatggaaatagcaaatgacagaagacaagcatttctctcaagacatcttgcctttgtgtgttgtttagcagagatagcatcttttggaccaaatttattaaatctaacatatctagagagacttgatgaattaaaacaatacttagatctgaacatcaaagaagatcctactcttaaatatgtgcaagtatcaggactgttaattaaatcattcccctcaactgttacgtatgtaaggaaaactgcgattaagtatctgaggattcgtggtattaatccgcctgaaacgattgaagattgggatcccatagaagatgagaatatcttagacaatattgttaaaactgtaaatgacaattgcagtgataatcaaaagagaaataaaagtagttatttctggggattagctctaaagaattatcaagtcgtgaaaataagatccataacgagtgattctgaagttaatgaagcttcgaatgttactacacatggaatgacacttcctcagggaggaagttatctatcacatcagctgaggttatttggagtaaacagtacaagttgtcttaaagctcttgaattatcacaaatcttaatgagggaagttaaaaaagataaagatagactctttttaggagaaggagcaggagctatgttagcatgttatgatgctacactcggtcctgcaataaattattataattctggtttaaatattacagatgtaattggtcaacgggaattaaaaatcttcccatcagaagtatcattagtaggtaaaaaactaggaaatgtaacacagattcttaatcgggtgagggtgttatttaatgggaatcccaattcaacatggataggaaatatggaatgtgagagtttaatatggagtgaattaaatgataagtcaattggtttagtacattgtgacatggagggagcgataggcaaatcagaagaaactgttctacatgaacattatagtattattaggattacatatttaatcggggatgatgatgttgtcctagtatcaaaaattataccaactattactccgaattggtctaaaatactctatctatacaagttgtattggaaggatgtaagtgtagtgtcccttaaaacatccaatcctgcctcaacagagctttatttaatttcaaaagatgcttactgtactgtaatggaacccagtaatcttgttttatcaaaacttaaaaggatatcatcaatagaagaaaataatctattaaagtggataatcttatcaaaaaggaagaataacgagtggttacagcatgaaatcaaagaaggagaaagggattatgggataatgaggccatatcatacagcactgcaaatttttggattccaaattaacttaaatcacttagctagagaatttttatcaactcctgatttaaccaacattaataatataattcaaagttttacaagaacaattaaagatgttatgttcgaatgggtcaatatcactcatgacaataaaagacataaattaggaggaagatataatctattcccgcttaaaaataaggggaaattaagattattatcacgaagattagtactaagctggatatcattatccttatcaaccagattactgacgggccgttttccagatgaaaaatttgaaaatagggcacagaccggatatgtatcattggctgatattgatttagaatccttaaagttattatcaagaaatattgtcaaaaattacaaagaacacataggattaatatcatactggtttttgaccaaagaggtcaaaatactaatgaagcttataggaggagtcaaactactaggaattcctaaacagtacaaagagttagaggatcgatcatctcagggttatgaatatgataatgaatttgatattgattaatacataaaaacaaaaaataaaacacctattcctcacccattcacttccaacaaaatgaaaagtaagaaaaacatgtaatatatatataccaaacagagtttttctcttgtttggt